(19) United States
(12) Patent Application Publication
(10) Pub. No.: US 2026/0112450 A1
(43) Pub. Date: Apr. 23, 2026

(54) METHODS FOR SELECTION OF CANCER PATIENTS FOR ANTI-ANGIOGENIC AND IMMUNE CHECKPOINT BLOCKAGE THERAPIES AND COMBINATIONS THEREOF

(71) Applicant: Washington University, St. Louis, MO (US)

(72) Inventors: Kyunghee Choi, St. Louis, MO (US); Madhav Subramanian, St. Louis, MO (US); Ashraf Ul Kabir, St. Louis, MO (US)

(21) Appl. No.: 18/998,425

(22) PCT Filed: Jul. 26, 2023
(86) PCT No.: PCT/US2023/028649

§ 371 Date: Jan. 24, 2025

Related U.S. Application Data
(60) Provisional application No. 63/392,297, filed on Jul. 26, 2022.

(51) Int. Cl. G16B 25/10; A61K 45/06; A61P 35/00; C12Q 1/48; C12Q 1/686; C12Q 1/6874; G16B 20/50; G16H 10/40; G16H 10/60; G16H 20/10

(52) U.S. Cl. CPC G16B 25/10; A61K 45/06; A61P 35/00; C12Q 1/48; C12Q 1/686; C12Q 1/6874; C12Y 207/07; G16B 20/50; G16H 10/40; G16H 10/60; G16H 20/10

(57) ABSTRACT

The present disclosure provides methods for assessing responsiveness or non-responsiveness to a therapeutic agent (e.g., ICB therapy and/or anti-angiogenic) in subjects having a cancer or tumor based on angio-immune panel signatures. The methods may further comprise identifying suitable treatment for the subject based on the angio-immune panel.

### CROSS REFERENCE TO RELATED APPLICATIONS

This application claims the benefit of the U.S. provisional application No. 63/392,297 filed Jul. 26, 2022, the disclosure of which is herein incorporated by reference in its entirety.

### FIELD OF THE TECHNOLOGY

The present disclosure relates, in general, methods for predicting outcome of cancer therapeutics and identifying patient populations. More specifically, the present disclosure provides compositions and methods for selecting subjects for anti-angiogenic therapies, immune checkpoint therapies or a combination thereof, thereby improving therapeutic outcomes.

### BACKGROUND

Immune checkpoint blockade (ICB) has revolutionized the treatment of metastatic and solid tumors by providing lasting regression to subsets of patients. However, a sizable majority of patients fail to mount responses to ICB. Accurate prediction of patients to likely respond to ICB would maximize the efficacy of ICB therapy.

Angiogenesis and immune tolerance are both normal physiologic mechanisms that are hijacked by tumors. Angiogenesis involves the formation of new vessels from preexisting ones during development and wound healing. The modulation of angiogenesis is highly regulated by proangiogenic and antiangiogenic factors, a process that becomes disrupted and dysregulated in cancer. Tumor-driven hypoxia increases the expression of proangiogenic factors leading to the formation of new vessels that are vital to the tumor survival and proliferation. The tumor microenvironment (TME) greatly dictates tumor progression and therapy outcome. Angiogenesis can greatly vary between TME. Similarly, T-cell activity can also vary considerably in the TME. Other variations in the tumor microenvironment may include mutations burden in expressed genes.

There is therefore an unmet need in the field to use variations in the TME to develop models that can help predict patient responsiveness to ICB greatly reducing wasted treatment time, morbidity, and financial burden on patients.

### SUMMARY

In an aspect, the current disclosure encompasses a method of predicting responsiveness of a subject to an immune checkpoint blockade (ICB) therapy, comprising: (i) providing expression levels of two or more genes in a biological sample obtained from tumor microenvironment (TME) of the subject, wherein the two or more genes encode effectors that impact angiogenesis or T-cell function or both; (ii) determining an angio-immune score based on the expression levels of the two or more genes in step (i); (iii) comparing the angio-immune score to an angio-immune baseline score for a corresponding tumor microenvironment to determine an angio-immune subtype; and (iv) predicting the subject's responsiveness to an ICB therapy based on the angio-immune subtype.

In an aspect, the current disclosure encompasses a method of treating a cancer in a subject in need thereof, comprising: administering to the subject an ICB therapeutic, wherein the subject has been identified as having an angio-immune subtype corresponding to a low angiogenic signature and a high T cell function signature, and wherein the angio-immune subtype was determined by a method comprising: (i) providing expression levels of two or more genes in a biological sample obtained from tumor microenvironment (TME) of the subject, wherein the two or more genes encode effectors that impact angiogenesis or T-cell function or both; (ii) determining an angio-immune score based on the expression levels of the two or more genes in step (i); and (iii) comparing the angio-immune score to an angio-immune baseline score for a corresponding tumor microenvironment to determine an angio-immune subtype.

In an aspect, the current disclosure encompasses a method of identifying a subject as responsive to ICB therapy, comprising: (i) providing expression levels of two or more genes in a biological sample obtained from the tumor microenvironment (TME) of the subject, wherein the two or more genes encode effectors that impact angiogenesis or T-cell function or both; (ii) determining an angio-immune score based on the expression levels of the two or more genes in step (i); (iii) comparing the angio-immune score to an angio-immune baseline score for a corresponding tumor microenvironment to determine an angio-immune subtype; and (iv) predicting the subject as responsive to an ICB therapy based on the angio-immune subtype.

In an aspect of a disclosed method, the two or more genes may comprise:
(i) genes that impact angiogenesis selected from:
   (a) MYCT1, APOH, APP, CCND2 COL3A1, COL5A2, CXCL6, FGFR1, FSTL1, ITGAV, JAG1, JAG2, KCNJ8, LPL, LRPAP1, LUM, MSX1, NRP1, OLR1, PDGFA, PF4, PGLYRP1, POSTN, PRG2, PTK2, S100A4, SERPINA5, SLCO2A1, SPP1, STC1, THBD, TIMP1, TNFRSF21, VAV2, CAN, VEGFA, VTN, CDH5, ELTD1, CLEC14A, LDB2, ECSCR, RHOJ, VWF, TIE1, KDR, ESAM, CD93, PTPRB, GPR116, SPARCL1, EMCN, ROBO4, ENG, TEK, SIPR1; or
(b) AGGF1, AMOT, ANGPTL3, ANGPTL4, BTG1, CHRNA7, RHOB, RUNX1, SPHK1, TNFSF12; or
(c) APOH, APP, CCND2, COL3A1, COL5A2, CXCL6, FGFR1, FSTL1, ITGAV, JAG1, JAG2, KCNJ8, LPL, LRPAP1, LUM, MSX1, NRP1, OLR1, PDGFA, PF4, PGLYRP1, POSTN, PRG2, PTK2, S100A4, SERPINA5, SLCO2A1, SPP1, STC1, THBD, TIMP1, TNFRSF21, VAV2, CAN, VEGFA, VTN; or
(d) ABL1, ADAMTS9, AGTR1, AKT3, ALOX5, ANXA1, APELA, APLNR, BMPER, CARD10, CEACAM1, CEMIP2, CIB1, CREB3L1, DLL1, DLL4, E2F2, EPN1, EPN2, FBXW7, FGF1, FGF2, FGFBP1, FOXC2, FUT1, GATA2, GHRL, GHSR, GLUL, HDAC5, HDAC7, HDAC9, HMOX1, IL10, IL12A, IL12B, ITGA5, ITGB1BP1, JAK1, JCAD, JMJD8, KDR, KLF2, KLF4, MAP2K5, MAP3K3, MEOX2, MIR1-1, MIR1-2, MIR101-1, MIR101-2, MIR10A, MIR10B, MIR1224, MIR125A, MIR126, MIR132, MIR138-1, MIR138-2, MIR146A, MIR149, MIR150, MIR15A, MIR15B, MIR16-1, MIR16-2, MIR17, MIR188, MIR18A, MIR193A, MIR196A1, MIR196A2, MIR199A1, MIR199A2, MIR199B, MIR19A, MIR19B1, MIR19B2, MIR2000, MIR206, MIR20A. MIR21, MIR22, MIR221, MIR222, MIR2355, MIR23A, MIR24-1, MIR24-2, MIR26A1, MIR26A2, MIR27A, MIR27B, MIR296, MIR29C, MIR30B, MIR30C1, MIR30C2, MIR30E, MIR31, MIR320A, MIR329-1, MIR329-2, MIR342, MIR34A, MIR34B, MIR34C, MIR361, MIR375, MIR377, MIR410, MIR424, MIR483, MIR487B, MIR494, MIR495, MIR497, MIR503, MIR92A1, MIR92A2, MIRLET7B, MIRLET7F1, MIRLET7F2, MMRN2, NGFR, NOTCH1, NR2E1, NRP1, PDCD10, PDPK1, PIK3C2A, PKM, PLK2, PPP1R16B, PTGS2, RHOA, RHOJ, S100A1, SEMA6A, SMAD1, SPRED1, SRPX2, STARD13, SYNJ2BP, TBXA2R, THBS1, TJP1, VEGFA; or
(e) ABL1, AGTR1, AKT3, ANXA1, APELA, APLNR, BMPER, CIB1, DLL1, FGF1, FGF2, FGFBP1, FOXC2, FUT1, GATA2, GHRL, GHSR, HDAC7, HDAC9, HMOX1, IL10, ITGA5, JAK1, JCAD, JMJD8, KDR, KLF4, MAP3K3, MIR1-1, MIR1-2, MIR101-1, MIR101-2, MIR10A, MIR10B, MIR1224, MIR125A, MIR126, MIR132, MIR146A, MIR150, MIR21, MIR23A, MIR27A, MIR27B, MIR296, MIR30B, MIR31, MIR487B, MIR503, MIR92A1, MIR92A2, MIRLET7B, MIRLET7F1, MIRLET7F2, NRP1, PDPK1, PIK3C2A, PKM, PLK2, PPP1R16B, PTGS2, RHOJ, S100A1, SMAD1, SRPX2, TJP1, VEGFA; or
(f) ABL1, AKT3, ANXA1, CIB1, FGF2, FGFBP1, FOXC2, GATA2, HDAC7, HDAC9, HMOX1, JCAD, KDR, MAP3K3, MIR101-1, MIR101-2, MIR10A, MIR10B, MIR126, MIR132, MIR146A, MIR150, MIR23A, MIR27A, MIR27B, MIR296, MIR31, MIR487B, MIRLET7F1, MIRLET7F2, NRP1, PIK3C2A, PLK2, PTGS2, RHOJ, SRPX2, VEGFA; or
(g) ABL1, ADTRP, AKT1, KT3, ANXA1, CARD10, CIB1, CLEC14A, DLL4, EFNB2, EGR3, EPHB4, FBXW7, FGF2, FGFBP1, FOXC2, GATA2, GPLD1, GREM1, HDAC5, HDAC7, HDAC9, HMOX1, ITGB1, ITGB1BP1, JCAD, KDR, KLF4, MAP2K5, MAP3K3, MEOX2, MIA3, MIR101-1, MIR101-2, MIR10A, MIR10B, MIR126, MIR132, MIR146A, MIR149, MIR150, MIR15A, MIR16-1, MIR16-2, MIR188, MIR193A, MIR196A1, MIR196A2, MIR199A1, MIR199A2, MIR199B, MIR19B1, MIR19B2, MIR2000, MIR206, MIR20A, MIR22, MIR221, MIR2355, MIR23A, MIR26A1, MIR26A2, MIR27A, MIR27B, MIR296, MIR29C, MIR31, MIR320A, MIR329-1, MIR329-2, MIR361, MIR410, MIR424, MIR483, MIR487B, MIR494, MIR495, MIR497, MIR503, MIRLET7F1, MIRLET7F2, MMRN2, NOTCH1, NR2E1, NR4A1, NRP1, PDCD10, PIK3C2A, PIK3R3, PLK2, PTGS2, RHOA, RHOJ, ROBO1, SLIT2, SPRED1, SRF, SRPX2, STARD13, TBXA2R, TDGF1, THBS1, VEGFA; or
(h) ACVRL1, AGGF1, AMOT, ANG, ANGPTL3, ANGPTL4, ATP5IF1, BTG1, C1GALT1, CANX, CDH13, CHRNA7, COL4A2, COL4A3, CXCL8, EGF, EMCN, EPGN, ERAP1, FOXO4, HTATIP2, IL17F, IL18, MYH9, NCL, NF1, NOTCH4, NPPB, NPR1, PF4, PLG, PML, PROK2, RHOB, RNH1, ROBO4, RUNX1, SCG2, SERPINF1, SHH, SPHK1, SPINK5, STAB1, TGFB2, THY1, TNFSF12, TNNI3, VEGFA; or
(i) ACTB, ACTG1, ACTN1, ACTN2, ACTN3, ACTN4, AFDN, ARHGAP35, ARHGAP5, BCAR1, CD99, CDC42, CDH5, CLDN1, CLDN10, CLDN11, CLDN14, CLDN15, CLDN16, CLDN17, CLDN18, CLDN19, CLDN2, CLDN20, CLDN22, CLDN23, CLDN3, CLDN4, CLDN5, CLDN6, CLDN7, CLDN8, CLDN9, CTNNA1, CTNNA2, CTNNA3, CTNNB1, CTNND1, CXCL12, CXCR4, CYBA, CYBB, ESAM, EZR, F11R, GNAI1, GNAI2, GNAI3, ICAM1, ITGA4, ITGAL, ITGAM, ITGB1, ITGB2, ITK, JAM2, JAM3, MAPK11, MAPK12, MAPK13, MAPK14, MMP2, MMP9, MSN, MYL10, MYL12A, MYL12B, MYL2, MYL5, MYL7, MYL9, MYLPF, NCF1, NCF2, NCF4, NOX1, NOX3, OCLN, PECAM1, PIK3CA, PIK3CB, PIK3CD, PIK3CG, PIK3R1, PIK3R2, PIK3R3, PIK3R5, PLCG1, PLCG2, PRKCA, PRKCB, PRKCG, PTK2, PTK2B, PTPN11, PXN, RAC1, RAC2, RAP1A, RAP1B, RAPGEF3, RAPGEF4, RASSF5, RHOA, RHOH, ROCK1, ROCK2, SIPA1, THY1, TXK, VASP, VAV1, VAV2, VAV3, VCAM1, VCL; or
(j) ADAM12, ADAP2, CHN1, COL5A3, EGFL6, ZD10, OPX, HSPA6, LCP2, MMP9, MXRA5, PAMR1, PLXDC1, PMAIP1, POSTN, RPS11, STC1, TNFAIP6, TNFRSF21, TWIST1, CAN, WDR77; or
(k) FLT1, FLT3, FLT4, KDR, NRP1, NRP2, PDGFRA; or
(l) ADAM17, AKT3, APLN, CCL2, COL4A3, FGF2, FGFR1, FLT1, GHRL, GHSR, HMGB1, IGF2, ITGA4, MDK, MEF2C, MIR126, MIR129-1, MIR129-2, MIR130A, MIR132, MIR133B, MIR135B, MIR152, MIR15B, MIR193A, MIR20B, MIR21, MIR24-1, MIR24-2, MIR27A, MIR29A, MIR29C, MIR30B, MIR30E, MIR329-1, MIR329-2, MIR34A, MIR424, MIR487B, MIR492, MIR495, MIR499A, MIR503, MIR98, PDPK1, PLCG1, PPARG, SIRT6, SP1, STAT3;
(m) ARNT, CADM4, CNMD, DAB2IP, EMILIN1, EPN2, FGF10, FGF18, FGF9, FZD4, GRB10, HGS, HHEX, HIF1A, IL1B, ITGA5, ITGB3, MIR10A, MIR10B, MIR1224, MIR2000, MIR296, MMRN2, MT3, MYOF, NEDD4, NIBAN2, PDCD6, PRKCB, PRKD2, PTPN1, TMEM204, TNMD, VEGFC, VTN; or
(n) ALOX5, CCL21, CCL25, CCL28, CCR2, CXCL12, ELANE, ETS1, FUT4, FUT7, FUT9, ICAM1, IL6, IRAK1, ITGA4, ITGB2, KLF4, MDK, MIR125A, MIR141, MIR146A, MIR21, MIR221, MIR222, MIR31, MIR92A1, MIR92A2, MIRLET7E, MIRLET7G, NFAT5, PTAFR, RELA, RHOA, SELE, SELP, TNF, TRAF6; or
(o) ALOX5, KLF4, MIR92A1, MIR92A2, TNF; or
(p) ADAMTS12, ALOX12, CXCL10, FGF1, FOXP1, ITGAX, MIR21; or
(q) CXCL13, FGF1, FGF16, FGF18, FGF2, FGF4, FGFR1, HRG, HSPB1, KDR, LGMN, MET, MIR149, MIR16-1, MIR16-2, MIR424, NOTCH1, P2RX4, PRKD1, PRKD2, SEMA5A, SMOC2, THBS1, TMSB4X, VEGFA; or
(r) ADAMTS12, ADAMTS3, ADGRA2, ATP2B4, CADM4, CCBE1, CD63, DAB2IP, DCN, DLL1, HRG, IL12A, IL12B, JCAD, MIR16-1, MIR16-2, MIR199A1, MIR199A2, MIR199B, MIR21, MIR329-1, MIR329-2, MIR342, MIR424, MYO1C, PTP4A3, ROBO1, SEMA6A, SMOC2, SPRY2, TSPAN12, XDH; or
(s) ADAMTS3, CCBE1, JCAD, MIR21, MYO1C, ROBO1, SMOC2;
(t) ARNT, FGF10, FGF18, FGF9, GRB10, HIF1A, IL1B, ITGA5, ITGB3, MIR10A, MIR10B, MIR1224, MIR296, MT3, PRKCB, PRKD2, VTN;
(u) ADORA2B, ARNT, ATF4, BRCA1, C3, C3AR1, C5, C5AR1, CCBE1, CXCL17, CYP1B1, EIF2AK3, FLT4, GATA4, HIF1A, HPSE, IL1A, IL1B, IL6, IL6ST, ISL1, NODAL, NOX1, PTGS2, RORA, SULF1, SULF2, TGFB1; or
(v) ADAM17, AKT3, APLN, FGF2, FGFR1, GHRL, HSR, HMGB1, IGF2, ITGA4, MDK, MIR126, MIR130A, MIR132, MIR135B, MIR21, MIR27A, MIR29A, MIR495, MIR499A, PDPK1, PLCG1, SIRT6, SP1, STAT3; or
(w) ALOX5, CCR2, ELANE, ETS1, FUT4, FUT7, ICAM1, IL6, IRAK1, ITGA4, ITGB2, MDK, MIR21, MIR92A1, MIR92A2, NFAT5, PTAFR, RELA, RHOA, SELE, SELP, TNF, TRAF6; or
(x) ACVRL1, ADAM17, AGGF1, AGTR1, AKT1, AKT3, ANG, APELA, APLN, APLNR, ARG1, ARNT, BMP2, BMP4, BMP6, BMPR2, CAV2, CCL11, CCL24, CCL26, CCR3, CDH13, CXCL12, CYBA, DYSF, ECM1, EGFL7, EGR3, EMC10, F3, GF2, FGFBP1, GFR1, LT4, GATA2, GDF2, GHRL, GHSR, HIF1A, HMGB1, HMGB2, HMOX1, HTR2B, IGF2, IL10, ITGA4, ITGB3, JCAD, JUN, KDR. LRG1. MDK. MIR101-1, MIR101-2, MIR10A, MIR10B, MIR126, MIR130A, MIR132, MIR135B, MIR146A, MIR21, MIR23A, MIR27A, MIR27B, MIR29A, MIR487B, MIR495, MIR499A, MIR503, MIRLET7B, MTOR, MYDGF, NF1, NR4A1, NRARP, NRAS, NRP1, NRP2, PDCD6, PDCL3, PDGFB, PDPK1, PGF, PLCG1, PLXNB3, PPP1R16B, PRKCA, PRKD1, PRKD2, PROX1, RICTOR, RPTOR, SCG2, SEMA5A, SIRT1, SIRT6, SP1, STAT3, STAT5A, TEK, TGFBR1, THBS4, TNFSF12, VASH2, VEGFA, VEGFB, VEGFC, VEGFD, VIP, WNT2, WNT5A, ZNF580; or
(y) AAMP, ABL1, ADAM17, ADGRA2, AGT, AKT1, AKT3, ALOX12, AMOTL1, ANGPT1, ANGPT4, ANXA1, ANXA3, ATOH8, ATP5F1A, ATP5F1B, BCAR1, BCAS3, BMP4, BMPR2, CALR, CCBE1, CD40, CIB1, EDN1, EMC10, ETS1, FGF1, FGF16, FGF18, FGF2, FGFBP1, FGFR1, FLT4, FOXC2, FOXP1, FUT1, GATA2, GATA3, GPI, GPLD1, GRN, HDAC7, HDAC9, HIF1A, HMGB1, HMOX1, HSPB1, ITGB1BP1, ITGB3, JCAD, KDR, LGMN, MAP2K3, MAP3K3, MET, MIR101-1, MIR101-2, MIR10A, MIR10B, MIR126, MIR132, MIR135B, MIR143, MIR146A, MIR150, MIR1908, MIR199A1, MIR199A2, MIR199B, MIR200A, MIR21, MIR210, MIR221, MIR23A, MIR27A, MIR27B, MIR296, MIR29A, MIR30A, MIR31, MIR342, MIR487B, MIR499A, MIR939, MIRLET7F1, MIRLET7F2, NFE2L2, NOS3, NRP1, NRP2, NUS1, P2RX4, PDCD6, PDGFB, PDPK1, PIK3C2A, PLCG1, PLK2, PLPP3, PRKCA, PRKD1, PRKD2, PROX1, PTGS2, PTK2B, RHOB, RHOJ, RIN2, ROCK2, RRAS, SASH1, SCARB1, SEMA5A, SIRT1, SMOC2, SP1, SPARC, SRPX2, STAT5A, TDGF1, TEK, TGFB1, THBS1, TMSB4X, TSTA3, VEGFA, VEGFC, WNT5A, WNT7A, ZC3H12A, ZNF580; or
(z) HSPB1, KDR, PRKD1, PRKD2, VEGFA; or
(aa) FGF16, FGF18, FGF2, FGFR1, HSPB1, KDR, LGMN, MET, P2RX4, PRKD1, PRKD2, SEMA5A, SMOC2, TMSB4X, VEGFA; or
(bb) GAB1, HSPB1, KDR, MYO1C, PRKD1, PRKD2, VEGFA; or
(cc) AGTR1, APELA, APLNR, FGFBP1, GATA2, HMOX1, JCAD, MIR101-1, MIR101-2, MIR10A, MIR10B, MIR126, MIR132, MIR146A, MIR21, MIR23A, MIR27A, MIR27B, MIR487B, MIR503, MIRLET7B, PPP1R16B, VEGFA; or
(dd) ADAM17, AKT1, AKT3, ALOX12, AMOTL1, ANGPT1, ANGPT4, ANXA1, ATP5F1A, ATP5F1B, CD40, CIB1, ETS1, FGF18, FGF2, FGFBP1, FGFR1, FOXC2, GATA2, HDAC7, HDAC9, HIF1A, HMGB1, HMOX1, HSPB1, JCAD, KDR, MAP2K3, MAP3K3, MIR101-1, MIR101-2, MIR10A, MIR10B, MIR126, MIR132, MIR135B, MIR143, MIR146A, MIR150, MIR200A, MIR210, MIR221, MIR23A, MIR27A, MIR27B, MIR296, MIR30A, MIR31, MIR342, MIR487B, MIR499A, MIR939, MIRLET7F1, MIRLET7F2, NFE2L2, NOS3, NRP1, NUS1, P2RX4, PDGFB, PDPK1, PIK3C2A, PLCG1, PLK2, PRKCA, PRKD1, PRKD2, PTGS2, RHOJ, SIRT1, SP1, SRPX2, STAT5A, TGFB1, THBS1, TMSB4X, VEGFA, VEGFC; or
(ee) ADD2, ALOX5, CCL21, CCL25, CCL28, CCR2, CX3CR1, CXCL12, ELANE, ETS1, FUT4, FUT7, FUT9, GCNT1, GOLPH3, ICAM1, IL6, IRAK1, ITGA4, ITGB1, ITGB2, ITGB7, JAM2, KLF4, LEP, MADCAM1, MDK, MIR125A, MIR141, MIR146A, MIR21, MIR221, MIR222, MIR31, MIR92A1, MIR92A2, MIRLET7E, MIRLET7G, NFAT5, PODXL2, PTAFR, RELA, RHOA, ROCK1, SELE, SELL, SELP, SELPLG, SLC39A8, SPN, TNF, TRAF6, VCAM1; or
(ff) ALOX5, KLF4, MIR92A1, MIR92A2, SLC39A8, TNF; or
(gg) AAMP, ABL1, ACVRL1, ADAM17, ADAMTS9, ADGRA2, ADGRB1, ADTRP, AGT, AGTR2, AKT1, AKT3, LOX12, AMOT, AMOTL1, ANGPT1, ANGPT2, ANGPT4, ANXA1, ANXA3, APOA1, APOE, APOH, ATOH8, ATP2B4, ATP5F1A, ATP5F1B, BCAR1, BCAS3, BMP10, BMP4, BMPER, BMPR2, CALR, CARD10, CCBE1, CCN3, CD40, CDH13, CDH5, CEACAM1, CIB1, CLEC14A, CORO1B, CSNK2B, CXCL13, CYP1B1, DAB2IP, DCN, DLL4, DNAJA4, DPP4, EDN1, EFNA1, EFNB2, EGR3, EMC10, EMP2, EPHA2, EPHB4, ETS1, FAP, FBXW7, FGF1, FGF16, FGF18, FGF2, FGF4, FGFBP1, FGFR1, FLT4, FOXC2, FOXP1, FUT1, GADD45A, GATA2, GATA3, GDF2, GIPC1, GLUL, GPI, GPLD1, GPX1, GREM1, GRN, HDAC5, HDAC7, HDAC9, HIF1A, HMGB1, HMOX1, HRG, HSPB1, ID1, ITGB1, ITGB1BP1, ITGB2, ITGB3, JCAD, JUP, KDR, KLF4, KRIT1, LGALS12, LGALS8, LGMN, LOXL2, LPXN, MAP2K3, MAP2K5, MAP3K3, MECP2, MEF2C, MEOX2, MET, MIA3, MIR101-1, MIR101-2, MIR10A, MIR10B, MIR126, MIR129-1, MIR129-2, MIR132, MIR133B, MIR135B, MIR137, MIR143, MIR146A, MIR149, MIR150, MIR152, MIR15A, MIR15B, MIR16-1, MIR16-2, MIR188, MIR1908, MIR193A, MIR196A1, MIR196A2, MIR199A1, MIR199A2, MIR199B, MIR19B1, MIR19B2, MIR200A, MIR200B, MIR2000, MIR204, MIR206, MIR20A, MIR21, MIR210, MIR212, MIR22, MIR221, MIR2355, MIR23A, MIR24-1, MIR24-2, MIR26A1, MIR26A2, MIR27A, MIR27B, MIR296, MIR29A, MIR29C, MIR30A, MIR31, MIR320A, MIR329-1, MIR329-2, MIR342, MIR361, MIR410, MIR424, MIR483, MIR487B, MIR492, MIR494, MIR495, MIR497, MIR499A, MIR503, MIR505, MIR640, MIR92A1, MIR92A2, MIR939, MIRLET7F1, MIRLET7F2, MMRN2, MYH9, NF1, NFE2L2, NOS3, NOTCH1, NR2E1, NR2F2, NR4A1, NRP1, NRP2, NUS1, P2RX4, PAXIP1, PDCD10, PDCD6, PDGFB, PDPK1, PECAM1, PIK3C2A, PIK3CA, PIK3R3, PLCG1, PLEKHG5, PLK2, PLPP3, PLXND1, PPARG, PRCP, PRKCA, PRKD1, PRKD2, PRKX, PROX1, PRSS3, PRSS3P2, PTEN, PTGS2, PTK2, PTK2B, PTN, PTP4A3, PTPRM, PXN, RAB13, RGCC, RHOA, RHOB, RHOJ, RIN2, ROBO1, ROCK2, RRAS, S100A2, S100P, SASH1, SCARB1, SCG2, SEMA4A, SEMA5A, SERPINF1, SH3BP1, SIRT1, SLIT2, SMOC2, SOX18, SP1, SP100, SPARC, SPRED1, SRF, SRPX2, STARD13, STAT5A, STC1, SVBP, SYNJ2BP, TBXA2R, TDGF1, TEK, TGFB1, TGFBR1, THBS1, TMSB4X, TNF, TNFSF12, TSTA3, VASH1, VEGFA, VEGFC, WNT5A, WNT7A, ZC3H12A, ZNF580; or
(hh) CCN3, CORO1B, CXCL13, EGR3, FGF1, FGF16, FGF18, FGF2, FGF4, GFR1, HRG, HSPB1, KDR, LGMN, MET, MIR149, MIR16-1, MIR16-2, MIR424, NOTCH1, NR4A1, NRP1, P2RX4, PLEKHG5, PRKD1, PRKD2, RAB13, SEMA5A, SMOC2, THBS1, TMSB4X, VEGFA; or
(ii) APOLD1, BMPER, CXCL10, FOXP1, HOXA9, MIR92A1, MIR92A2, P2RX4, PRMT5, SMAD4, TCIM, TGFBR1;
(jj) ABL1, ACVRL1, ADAM17, ADTRP, AGTR2, AKT1, AKT3, ALOX12, AMOT, AMOTL1, ANGPT1, ANGPT2, ANGPT4, ANXA1, APOA1, APOE, ATP2B4, ATP5F1A, ATP5F1B, CARD10, CD40, CDH5, CIB1, CLEC14A, CSNK2B, DLL4, EFNA1, EFNB2, EGR3, EMP2, EPHA2, EPHB4, ETS1, FBXW7, FGF18, FGF2, FGFBP1, FGFR1, FOXC2, GADD45A, GATA2, GDF2, GPLD1, GPX1, GREM1, HDAC5, HDAC7, HDAC9, HIF1A, HMGB1, HMOX1, HRG, HSPB1, ID1, ITGB1, ITGB1BP1, JCAD, JUP, KDR, KLF4, MAP2K3, MAP2K5, MAP3K3, MECP2, MEF2C, MEOX2, MIA3, MIR101-1, MIR101-2, MIR10A, MIR10B, MIR126, MIR129-1, MIR129-2, MIR132, MIR133B, MIR135B, MIR137, MIR143, MIR146A, MIR149, MIR150. MIR152, MIR15A, MIR15B, MIR16-1, MIR16-2, MIR188, MIR193A, MIR196A1, MIR196A2, MIR199A1, MIR199A2, MIR199B, MIR19B1, MIR19B2, MIR200A, MIR200B, MIR2000, MIR204, MIR206, MIR20A, MIR210, MIR212, MIR22, MIR221, MIR2355, MIR23A, MIR24-1, MIR24-2, MIR26A1, MIR26A2, MIR27A, MIR27B, MIR296, MIR29C, MIR30A, MIR31, MIR320A, MIR329-1, MIR329-2, MIR342, MIR361, MIR410, MIR424, MIR483, MIR487B, MIR492, MIR494, MIR495, MIR497, MIR499A, MIR503, MIR505, MIR640, MIR92A1, MIR92A2, MIR939, MIRLET7F1, MIRLET7F2, MMRN2, MYH9, NF1, NFE2L2, NOS3, NOTCH1, NR2E1, NR4A1, NRP1, NUS1, P2RX4, PDCD10, PDGFB, PDPK1, PIK3C2A, PIK3R3, PLCG1, PLK2, PPARG, PRCP, PRKCA, PRKD1, PRKD2, PTGS2, PTK2B, RGCC, RHOA, RHOJ, ROBO1, SCARB1, SH3BP1, SIRT1, SLIT2, SOX18, SP1, SPRED1, SRF, SRPX2, STARD13, STAT5A, TBXA2R, TDGF1, TGFB1, THBS1, TMSB4X, TNF, VASH1, VEGFA, VEGFC; or any combination thereof;
   and (ii) genes that impact T-cell function selected from:
(a) ADAM10, ADAM17, CCL21, CCL26, CCL27, CCL3, CCL5, CCR2, CXCL10, CXCL11, CXCL13, CXCL16, CXCR3, DEFA1, DEFA1B, GPR183, OXSR1, PIK3CD, PIK3CG, S100A7, SLC12A2, STK39, TMEM102, TNFSF14, WNK1, WNT5A, XCL1, XCL2; or
(b) AGER, AIRE, ARG1, AZGP1, B2M, BTN3A2, BTN3A3, CCR2, CD1A, CD1B, CD1C, CD1D, CD1E, CD46, CD55, CD70, CD81, CD8A, CEACAM1, CLC, CLEC4G, CTSC, CTSH, CYRIB, DENND1B, DLG1, DUSP22, EMP2, FADD, FBXO38, FCGR2B, FOXP3, FUT7, FZD5, GATA3, GNL1, GZMM, HFE, HLA-A, HLA-B, HLA-E, HLA-F, HLA-G, HLA-H, HMGB1, HPRT1, HSPD1, ICAM1, IFNA2, IFNB1, IL12A, IL12B, IL12RB1, IL18, IL18R1, IL18RAP, IL1B, IL1R1, IL20RB, IL23A, IL23R, IL31RA, IL4, IL6, IL7R, JAG1, KDELR1, LILRB1, MALT1, MAP3K7, MICA, MR1, MYO1G, NECTIN2, NLRP3, P2RX7, PPP3CB, PRF1, PRKCZ, PTPRC, PVR, RAB27A, RFTN1, RIPK3, RSAD2, SASH3, SCART1, SLC11A1, SLC22A13, SMAD7, STARD7, STX7, TBX21, TNFRSF1B, TNFSF4, TRAF2, TRAF6, TREX1, TRPM4, TSTA3, WAS, XCL1, ZBTB1, ZP3; or
(c) FBXO38, HLA-A, HMGB1, HSPD1, MR1, SLC22A13; or
(d) AGER, AZGP1, B2M, CD1A, CD1, CD1C, CD1D, CD1E, CEACAM1, CTSC, CTSH, CYRIB, EMP2, FADD, FCGR2B, GZMM, HLA-A, HLA-B, HLA-E, HLA-F, HLA-G, HLA-H, HPRT1, IL12A, IL12B, IL12RB1, IL23A, IL23R, IL7R, LILRB1, MICA, MR1, NECTIN2, P2RX7, PPP3CB, PRF1, PTPRC, PVR, RAB27A, RIPK3, SLC22A13, STX7, TSTA3, XCL1; or
(e) CCL2, CCR2, CD99, CD99L2, CRK, CRKL, F11R, FADD, ICAM1, IL27RA, ITGAL, RIPK3, XG; or
(f) ANXA1, ATP7A, BATF, BCL3, BCL6, CCL19, CD46, CD80, CD86, CLEC4D, CLEC4E, EOMES, FCER1G, FGL2, FOXP1, FOXP3, GATA3, GPR183, HLX, HMGB1, IFNG, IFNL1, IL12B, IL12RB1, IL18, IL18R1, IL2, IL21, IL23A, IL23R, IL27, IL4, IL4R, IL6, IRF4, JAK3, LEF1, LGALS9, LOXL3, LY9, MALT1, MIR21, MTOR, MYB, NFKBID, NFKBIZ, NLRP3, PCK1, PRKCZ, PTGER4, RARA, RC3H1, RC3H2, RELB, RIPK2, RORA, RORC, SEMA4A, SLAMF6, SMAD7, SOCS5, SPN, STAT3, STAT6, TBX21, TMEM98, TNFSF4, TSC1, ZBTB7B, ZC3H12A, ZFPM1; or
(g) ADA, ADAM8, AKT1, ARG2, BAK1, BAX, BCL10, BCL2L11, BMP4, CCL5, CD27, CD274, CLC, DFFA, DNAJA3, DOCK8, EFNA1, FADD, FAS, FASLG, GIMAP8, GLI3, GPAM, HIF1A, IDO1, IL2RA, IL7R, JAK3, KDELR1, LGALS13, LGALS14, LGALS16, LGALS3, LGALS9, LMBR1L, NFKBID, PDCD1, PIP, PRELID1, PRKCQ, PTCRA, RAG1, RIPK1, RIPK3, SLC46A2, TP53, TSC22D3, WNT5A, ZC3H8; or
(h) HFE, ICAM1, RFTN1, TREX1, WAS; or
(i) ANXA1, APBB1IP, ATP7A, BATF, BCL3, BCL6, CCL19, CD1C, CD46, CD74, CD80, CD81, CD86, CEACAM1, CLEC4D, CLEC4E, EIF2AK4, EOMES, F2RL1, FCER1G, FCGR2B, FGL2, FOXP1, FOXP3, GATA3, GPR183, HAVCR2, HLA-DMB, HLX, HMGB1, ICAM1, IFNA1, IFNA10, IFNA13, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNW1, IL12B, IL12RB1, IL18, IL18R1, IL2, IL21, IL23A, IL23R, IL27, IL4, IL4R, IL6, IRF4, ITGAL, JAK3, LCP1, LEF1, LGALS3, LGALS9, LILRB1, LOXL3, LY9, MALT1, MDK, MIR21, MTOR, MYB, NFKBID, NFKBIZ, NLRP3, PCK1, PRKCZ, PSEN1, PTGER4, RAB27A, RARA, RC3H1, RC3H2, RELB, RIPK2, RORA, RORC, SEMA4A, SLAMF6, SLC11A1, SMAD7, SOCS5, SPN, STAT3, STAT6, TBX21, TMEM98, TNFSF18, TNFSF4, TP53, TSC1, ZBTB7B, ZC3H12A, ZFPM1; or
(j) ADA, BCL10, BTN2A2, CACNA1F, CARD11, CCR7, CD160, CD226, CD300A, CD81, CEACAM1, CYLD, DGKZ, DUSP22, DUSP3, ELF1, EZR, GBP1, KCNN4, LCK, LGALS3, LILRB4, MALT1, NECTIN2, PAWR, PHPT1, PRKD2, PRNP, PTPN2, PTPN22, PTPN6, PTPRJ, PVRIG, RAB29, RC3H1, RELA, RPS3, SH2D1A, SLA2, TESPA1, THY1, TRAT1, UBASH3A; or
(k) ADAM10, ADAM17, ADAM8, AIF1, AIRE, APOD, APP, C10orf99, CCL20, CCL21, CCL27, CCL5, CCR2, CCR6, CD200, CD200R1, CD99L2, CRK, CRKL, CXCL10, CXCL12, CXCL13, DOCK8, ECM1, FADD, IL27RA, ITGA4, LRCH1, OXSR1, PYCARD, RHOA, RIPK3, RIPOR2, S100A7, SELENOK, SPN, STK39, TMEM102, TNFRSF14, TNFSF14, WNK1, WNT5A, XCL1, XCL2; or
(l) ARG1, AZGP1, B2M, CCR2, CD1A, CD1B, CD1C, CD1D, CD1E, CD81, CEACAM1, CLC, CLEC4G, CYRIB, DUSP22, FADD, FBXO38, FCGR2B, FOXP3, FUT7, FZD5, GATA3, HFE, HLA-A, HLA-B, HLA-E, HLA-F, HLA-G, HLA-H, HMGB1, HSPD1, IFNA2, IFNB1, IL12A, IL12B, IL12RB1, IL18, IL18R1, IL1B, IL1R1, IL20RB, IL23A, IL23R, IL6, IL7R, LILRB1, MALT1, MAP3K7, MR1, NECTIN2, NLRP3, P2RX7, PPP3CB, PRKCZ, PTPRC, PVR, RIPK3, RSAD2, SASH3, SLC22A13, SMAD7, STX7, TBX21, TNFRSF1B, TNFSF4, TRAF2, TRAF6, TRPM4, WAS, XCL1, ZBTB1, ZP3;
(m) FBXO38, HMGB1, HSPD1, MR1, SLC22A13; or
(n) AGER, AZGP1, B2M, CD1A, CD1B, CD1C, CD1D, CD1E, CEACAM1, CYRIB, FADD, FCGR2B, HLA-A, HLA-B, HLA-E, HLA-F, HLA-G, HLA-H, IL12A, IL12B, IL12RB1, IL23A, IL23R, IL7R, LILRB1, MR1, NECTIN2, P2RX7, PPP3CB, PTPRC, PVR, RIPK3, SLC22A13, STX7, XCL1; or
(o) CCR2, CD99L2, FADD, IL27RA, RIPK3; or
(p) ABL1, ADA, ADAM8, ANXA1, AP3B1, AP3D1, BAD, BCL6, BMP4, BTN2A2, CAMK4, CARD11, CBFB, CCL19, CCR2, CD2, CD27, CD28, CD46, CD74, CD80, CD83, CD86, CLPTM1, CR1, CRTAM, CTLA4, CYLD, CYP26B1, DROSHA, DTX1, DUSP10, EGR3, ERBB2, FANCA, FANCD2, FGL2, FOXJ1, FOXN1, FOXP3, GATA3, GLI2, GLI3, HLA-DOA, HLA-G, HLX, HMGB1, IFNA2, IFNB1, IFNG, IFNL1, IHH, IL12A, IL12B, IL12RB1, IL15, IL18, IL1A, IL1B, IL1RL2, IL2, IL23A, IL23R, IL27, IL2RA, IL36B, IL4, IL4R, IL7, IL7R, IRF1, IRF4, ITPKB, JAK3, KAT2A, LAG3, LEF1, LGALS9, LILRB2, LILRB4, LOXL3, MALT1, MDK, METTL3, MIR21, MIR30B, MYB, NCKAP1L, NFATC2, NFKBID, NFKBIZ, NKAP, NLRP3, NRARP, PCK1, PIK3R6, PNP, PRDM1, PRDX2, PRELID1, PRKCZ, PTPN2, PTPRC, RAG1, RARA, RASGRP1, RC3H1, RC3H2, RHOA, RHOH, RIPK2, RUNX1, RUNX3, SART1, SASH3, SH3RF1, SHH, SLC46A2, SMAD7, SOCS1, SOCS5, SOD1, SOS1, SOX12, SOX13, SOX4, SPINK5, STAT5B, SYK, TBX21, TCF7, TESPA1, TGFBR2, TMEM131L, TNFRSF18, TNFSF4, TNFSF9, TOX, VNN1, VSIR, XBP1, ZAP70, ZBTB1, ZBTB16, ZBTB7B, ZC3H12A, ZC3H8, ZEB1, ZMIZ1, ZNF683; or
(q) B2M, CCR2, CD81, CLC, FOXP3, FZD5, GATA3, HFE, HLA-A, HLA-F, IFNA2, IFNB1, IL18, IL18R1, IL1B, IL1R1, IL6, MALT1, MAP3K7, NLRP3, PRKCZ, RSAD2, SASH3, SMAD7, TBX21, TNFRSF1B, TNFSF4, TRAF2, TRAF6, TRPM4, XCL1; or
(r) ADAM10, ADAM17, CCL21, CCL27, CCL5, CCR2, CXCL10, CXCL13, OXSR1, S100A7, STK39, TMEM102, TNFSF14, WNK1, WNT5A, XCL1, XCL2; or
(s) CD81, HAVCR2, HLA-DMB, LGALS3, LGALS9, LILRB1; or
(t) ABL1, ADA, ADAM8, ADORA2A, AGER, AIF1, AKT1, ANXA1, AP3B1, AP3D1, ARG1, ARG2, BAD, BCL10, BCL6, BMP4, BTLA, BTN2A2, CAMK4, CARD11, CASP3, CAV1, CBFB, CCDC88B, CCL19, CCL2, CCL21, CCL5, CCR2, CCR7, CD160, CD1D, CD2, CD209, CD24, CD27, CD274, CD276, CD28, CD300A, CD3E, CD4, CD40LG, CD46, CD47, CD5, CD55, CD6, CD70, CD74, CD80, CD81, CD83, CD86, CDC42, CEACAM1, CEBPB, CGAS, CLC, CLEC4G, CLECL1, CLPTM1, CORO1A, CR1, CRTAM, CSK, CTLA4, CTNNB1, CYLD, CYP26B1, CYRIB, DLG1, DLG5, DNAJA3, DOCK8, DPP4, DROSHA, DTX1, DUSP10, DUSP22, DUSP3, EBI3, EFNB1, EFNB2, EFNB3, EGR3, EPO, ERBB2, FADD, FANCA, FANCD2, FCGR2B, FGL1, FGL2, FLOT2, FOXJ1, FOXN1, FOXP3, FYN, GATA3, L12, GLI3, GLMN, GNRH1, GPAM, GPNMB, GRAP2, GRB2, GSN, HAVCR2, HES1, HFE, HHLA2, HLA-A, HLA-DMB, HLA-DOA, HLA-DPA1, HLA-DPB1, HLA-DRB1, HLA-E, HLA-G, HLX, HMGB1, HSPD1, HSPH1, ICOS, ICOSLG, IDO1, IFNA2, IFNB1, IFNG, IFNL1, IGF1, IGF2, IGFBP2, IHH, IL10, IL12A, IL12B, IL12RB1, IL15, IL18, IL1A, IL1B, IL1RL2, IL2, IL20RB, IL21, IL23A, IL23R, IL27, IL27RA, IL2RA, IL36B, IL4, IL4R, IL6, IL6ST, IL7, IL7R, IRF1, IRF4, ITPKB, JAK3, KAT2A, KLRC4-KLRK1, KLRK1, LAG3, LAT, LAX1, LCK, LEF1, LEP, LGALS1, LGALS3, LGALS7B, LGALS9, LGALS9B, LGALS9C, LILRB1, LILRB2, LILRB4, LMO1, LOXL3, LRRC32, LYN, MAD1L1, MALT1, MAP3K8, MAPK8IP1, MARCHF7, MDK, METTL3, MIR181C, MIR21, MIR27A, MIR30B, MYB, NCK1, NCK2, NCKAP1L, NDFIP1, NFATC2, NFKBID, NFKBIZ, NKAP, NLRP3, NOD2, NRARP, PAG1, PAK1, PAK2, PAK3, PAWR, PCK1, PDCD1, PDCD1LG2, PDE5A, PDPK1, PEI1, PIK3CA, PIK3R1, PIK3R6, PLA2G2D, PLA2G2F, PNP, PRDM1, PRDX2, PRELID1, PRKAR1A, PRKCQ, PRKCZ, PRNP, PTPN11, PTPN2, PTPN22, PTPN6, PTPRC, PYCARD, RAC1, RAC2, RAG1, RARA, RASAL3, RASGRP1, RC3H1, RC3H2, RHOA, RHOH, RIPK2, RIPK3, RIPOR2, RPS3, RUNX1, RUNX3, SART1, SASH3, SCGB1A1, SCRIB, SDC4, SELENOK, SFTPD, SH3RF1, SHH, SIRPA, SIRPB1, SIRPG, SIT1, SLC46A2, SLC7A1, SMAD7, SOCS1, SOCS5, SOCS6, SOD1, SOS1, SOX12, SOX13, SOX4, SPINK5, SPN, SPTA1, SRC, STAT5B, SYK, TARM1, TBX21, TCF7, TESPA1, TFRC, TGFBR2, THY1, TIGIT, TMEM131L, TMIGD2, TNFAIP8L2, TNFRSF13C, TNFRSF14, TNFRSF18, TNFRSF1B, TNFRSF21, TNFSF11, TNFSF13B, TNFSF14, TNFSF18, TNFSF4, TNFSF8, TNFSF9, TOX, TRAF6, TREX1, TWSG1, VAV1, VCAM1, VNN1, VSIG4, VSIR, VTCN1, XBP1, XCL1, YES1, ZAP70, ZBTB1, ZBTB16, ZBTB7B, ZC3H12A, ZC3H8, ZEB1, ZMIZ1, ZNF683, ZP3, ZP4; or
(u) EGR3, LEF1, LILRB1, NCKAP1L, NOD2, PTPRC, SOX13, SOX4, STAT5B, SYK, TCF7; or
(v) CBFB, NCKAP1L, RUNX1, RUNX3, SOCS1, ZBTB7B; or
(w) ANXA1, BCL6, CBFB, CCL19, CD80, CD83, CD86, FOXP3, GATA3, HLX, HMGB1, IFNG, IL12B, IL12RB1, IL18, IL2, IL23A, IL23R, IL27, IL4, IL4R, IRF4, JAK3, LGALS9, LOXL3, MALT1, MIR21, MYB, NCKAP1L, NFKBID, NFKBIZ, NLRP3, PRKCZ, RARA, RC3H1, RC3H2, RIPK2, RUNX1, RUNX3, SASH3, SH3RF1, SMAD7, SOCS1, SOCS5, TBX21, TNFSF4, ZBTB7B, ZC3H12A; or
(x) AGER, ANXA1, ARG2, BCL6, CBFB, CCL19, CD160, CD274, CD55, CD80, CD81, CD83, CD86, FOXP3, GATA3, HLX, HMGB1, IFNG, IL12B, IL12RB1, IL18, IL2, IL23A, IL23R, IL27, IL4, IL4R, IRF4, JAK3, LGALS7B, LGALS9, LGALS9B, LGALS9C, LOXL3, MALT1, MIR21, MYB, NCKAP1L, NFKBID, NFKBIZ, NLRP3, PRKCQ, PRKCZ, RARA, RC3H1, RC3H2, RIPK2, RUNX1, RUNX3, SASH3, SH3RF1, SMAD7, SOCS1, SOCS5, TARM1, TBX21, TGFBR2, TNFSF4, TWSG1, VSIR, XCL1, ZBTB7B, ZC3H12A; or
(y) CBFB, CD274, CRTAM, HFE, HLA-A, HLA-E, IRF1, LILRB1, MAPK8IP1, NCKAP1L, PTPN22, RUNX1, RUNX3, SH3RF1, SOCS1, VSIR, XCL1, ZBTB7B; or
(z) ADA, ANXA1, AP3B1, AP3D1, BCL6, CBFB, CCL19, CD80, CD83, CD86, FOXP3, GATA3, GLI3, HLX, HMGB1, IFNG, IHH, IL12B, IL12RB1, IL18, IL2, IL23A, IL23R, IL27, IL4, IL4R, IRF4, ITPKB, JAK3, LGALS9, LOXL3, MALT1, MIR21, MYB, NCKAP1L, NFKBID, NFKBIZ, NKAP, NLRP3, PNP, PRDM1, PRKCZ, RARA, RC3H1, RC3H2, RHOA, RIPK2, RUNX1, RUNX3, SASH3, SH3RF1, SHH, SMAD7, SOCS1, SOCS5, SYK, TBX21, TGFBR2, TNFSF4, ZAP70, ZBTB16, ZBTB7B, ZC3H12A, ZNF683; or
(aa) ADA, ADORA2A, AGER, ANXA1, AP3B1, AP3D1, ARG2, BCL6, CBFB, CCL19, CCR2, CD160, CD274, CD28, CD300A, CD3E, CD55, CD80, CD81, CD83, CD86, CRTAM, EB13, FOXP3, GATA3, GL13, HFE, HLA-A, HLA-E, HLX, HMGB1, HSPH1, IFNG, IHH, IL12A, IL12B, IL12RB1, IL18, IL2, IL23A, IL23R, IL27, IL4, IL4R, IRF1, IRF4, ITPKB, JAK3, LGALS7B, LGALS9, LGALS9B, LGALS9C, LILRB1, LOXL3, MALT1, MAPK81P1, MIR21, MYB, NCKAP1L, NFKBID, NFKBIZ, NKAP, NLRP3, PNP, PRDM1, PRKCQ, PRKCZ, PTPN22, PTPRC, RARA, RASAL3, RC3H1, RC3H2, RHOA, RIPK2, RUNX1, RUNX3, SASH3, SH3RF1, SHH, SMAD7, SOCS1, SOCS5, SYK, TARM1, TBX21, TGFBR2, TNFRSF14, TNFSF4, TWSG1, VSIR, XCL1, ZAP70, ZBTB16, ZBTB7B, ZC3H12A, ZNF683; or
(bb) BATF, BCL11B, BCL2, CD3D, CD3E, CD3G, CD74, CTSL, CYLD, DOCK2, FOXN1, FOXP3, IL12B, IL12RB1, IL23A, IL23R, IL6, IRF4, ITPKB, LOXL3, LY9, MTOR, PTPN2, PTPRC, SHH, SLAMF6, SPN, SRF, STAT3, STAT6, STK11, TBX21, THEMIS, TOX, ZAP70, ZFPM1; or
(cc) CD3E, FOXP3, HLA-G, IDO1, LILRB2, TGFBR2; or
(dd) ADA, CARD11, CCR7, CD226, CD81, CYLD, KCNN4, LCK, NECTIN2, PRKD2, RAB29, RELA, RPS3, TESPA1, TRAT1; or
(ee) AGER, AIF1, ANXA1, CARD11, CCDC88B, CCL19, CCL5, CCR2, CD1D, CD209, CD24, CD274, CD276, CD28, CD3E, CD4, CD40LG, CD46, CD55, CD6, CD70, CD80, CD81, CD86, CLECL1, CORO1A, DNAJA3, EB13, EFNB1, EPO, FADD, FOXP3, GPAM, HAVCR2, HES1, HHLA2, HLA-A, HLA-DMB, HLA-DPA1, HLA-DPB1, HLA-E, HMGB1, ICOSLG, IGF1, IGF2, IGFBP2, IL12A, IL12B, IL12RB1, IL15, IL18, IL1A, IL1B, IL2, IL21, IL23A, IL23R, IL27RA, IL2RA, IL4, IL6, IL6ST, JAK3, LEP, LGALS9, LILRB2, MIR21, MIR30B, NCK1, NCK2, NCKAP1L, PDCD1LG2, PNP, PRKCQ, PTPN22, PTPRC, PYCARD, RASAL3, RIPK2, RPS3, SASH3, SELENOK, SHH, SLC7A1, SPTA1, STAT5B, SYK, TFRC, TGFBR2, TMIGD2, TNFRSF13C, TNFSF13B, TNFSF4, TNFSF9, TRAF6, VCAM1, VTCN1, XCL1, ZAP70, ZP3, ZP4; or
(ff) ADAM10, ADAM17, ADAM8, AIF1, APP, CCL20, CCL21, CCL27, CCL5, CCR2, CD99L2, CXCL10, CXCL12, CXCL13, DOCK8, FADD, ITGA4, OXSR1, PYCARD, RHOA, S100A7, SELENOK, SPN, STK39, TMEM102, TNFRSF14, TNFSF14, WNK1, WNT5A, XCL1, XCL2; or
(gg) AZGP1, B2M, CD1A, CD1B, CD1C, CD1D, CD1E, CD81, CYRIB, FADD, FBXO38, FOXP3, FZD5, GATA3, HLA-A, HLA-B, HLA-E, HLA-F, HLA-G, HLA-H, HSPD1, IL12A, IL12B, IL12RB1, IL18, IL18R1, IL1B, IL1R1, IL23A, IL23R, IL6, MALT1, MAP3K7, MR1, NECTIN2, NLRP3, P2RX7, PRKCZ, PTPRC, PVR, RSAD2, SASH3, SLC22A13, STX7, TNFSF4, TRAF2, TRAF6, XCL1, ZBTB1, ZP3; or
(hh) AZGP1, B2M, CD1A, CD1B, CD1C, CD1D, CD1E, CYRIB, FADD, HLA-A, HLA-B, HLA-E, HLA-F, HLA-G, HLA-H, IL12A, IL12B, IL12RB1, IL23A, IL23R, MR1, NECTIN2, P2RX7, PTPRC, PVR, SLC22A13, STX7, XCL1; or
(ii) B2M, CD81, FZD5, GATA3, HLA-A, IL18, IL18R1, IL1B, IL1R1, IL6, MALT1, MAP3K7, NLRP3, PRKCZ, RSAD2, SASH3, TNFSF4, TRAF2, TRAF6, XCL1; or
(jj) BCL6, BTN2A2, CD46, DUSP10, FOXP3, HLA-G, IFNG, IL2, LGALS9, LILRB2, LILRB4, SOCS1, SOX12, VSIR; or
(kk) FOXP3, IL1A, IL1B, RIPK2, SHH; or
(ll) LEF1, NCKAP1L, PTPRC, SOX13, SOX4, STAT5B, SYK; or
(mm) LEF1, LILRB1, NCKAP1L, NOD2, PTPRC, SOX13, SOX4, STAT5B, SYK; or
(nn) ADA, ANXA1, AP3B1, AP3D1, CBFB, CCL19, CD80, CD83, CD86, FOXP3, GLI3, HLX, IFNG, IHH, IL12B, IL12RB1, IL18, IL23A, IL23R, IL4R, ITPKB, LGALS9, MALT1, MIR21, MYB, NCKAP1L, NFKBID, NFKBIZ, NKAP, NLRP3, PNP, PRKCZ, RARA, RHOA, RIPK2, RUNX1, RUNX3, SASH3, SHH, SOCS1, SOCS5, SYK, TGFBR2, TNFSF4, ZAP70, ZBTB16, ZBTB7B; or
(oo) ADA, ANXA1, AP3B1, AP3D1, CBFB, CCL19, CCR2, CD160, CD28, CD3E, CD55, CD80, CD81, CD83, CD86, EBI3, FOXP3, GLI3, HLA-A, HLA-E, HLX, HSPH1, IFNG, IHH, IL12A, IL12B, IL12RB1, IL18, IL23A, IL23R, IL4R, ITPKB, LGALS9, MALT1, MIR21, MYB, NCKAP1L, NFKBID, NFKBIZ, NKAP, NLRP3, PNP, PRKCQ, PRKCZ, PTPN22, PTPRC, RARA, RASAL3, RHOA, RIPK2, RUNX1, RUNX3, SASH3, SHH, SOCS1, SOCS5, SYK, TGFBR2, TNFSF4, XCL1, ZAP70, ZBTB16, ZBTB7B; or
(pp) AGER, CD24, EPO, FADD, GPAM, HHLA2, HMGB1, ICOSLG, IGF1, IGF2, IGFBP2, IL12B, IL12RB1, IL18, IL2, IL23A, IL23R, IL27RA, IL2RA, MIR21, MIR30B, PYCARD, RPS3, STAT5B, TMIGD2, TNFSF9; or
(qq) ABL1, ADA, ADAM17, ADAM8, ADORA2A, AGER, AIF1, AIRE, AKT1, ANXA1, AP3B1, AP3D1, APBB1IP, ARG1, ARG2, ATG5, ATP7A, AZI2, B2M, BAD, BATF, BAX, BCL10, BCL11B, BCL2, BCL3, BCL6, BMP4, BTLA, BTN2A2, BTN3A1, CAMK4, CARD11, CASP3, CASP8, CAV1, CBFB, CCDC88B, CCL19, CCL2, CCL21, CCL5, CCND3, CCR2, CCR6, CCR7, CCR9, CD151, CD160, CD1C, CD1D, CD2, CD209, CD24, CD27, CD274, CD276, CD28, CD300A, CD3D, CD3E, CD3G, CD4, CD40LG, CD44, CD46, CD47, CD5, CD55, CD6, CD7, CD70, CD74, CD80, CD81, CD83, CD86, CD8A, CD8B, CDC42, CDH26, CDK6, CEACAM1, CEBPB, CGAS, CHD7, CLC, CLEC4A, CLEC4D, CLEC4E, CLEC4G, CLEC7A, CLECL1, CLPTM1, CORO1A, CR1, CRTAM, CSK, CTLA4, CTNNB1, CTPS1, CTSL, CXADR, CYLD, CYP26B1, CYRIB, DDOST, DLG1, DLG5, DLL4, DNAJA3, DOCK2, DOCK8, DPP4, DROSHA, DTX1, DUSP10, DUSP22, DUSP3, EB13, EFNB1, EFNB2, EFNB3, EGR1, EGR3, EIF2AK4, ELF4, EOMES, EPO, ERBB2, F2RL1, FADD, FANCA, FANCD2, FCER1G, FCGR2B, FGL1, FGL2, FKBP1A, FKBP1B, FLOT2, FOXJ1, FOXN1, FOXP1, FOXP3, FUT7, FYN, FZD5, FZD7, FZD8, GATA3, GLI2, GLI3, GLMN, GNRH1, GPAM, GPNMB, GPR18, GPR183, GPR89A, GPR89B, GRAP2, GRB2, GSN, HAVCR2, HES1, HFE, HHLA2, HLA-A, HLA-DMB, HLA-DOA, HLA-DPA1, HLA-DPB1, HLA-DRB1, HLA-E, HLA-G, HLX, HMGB1, HSH2D, HSPD1, HSPH1, ICAM1, ICOS, ICOSLG, IDO1, IFNA1, IFNA10, IFNA13, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNW1, IGF1, IGF2, IGFBP2, IHH, IL10, IL12A, IL12B, IL12RB1, IL15, IL18, IL18R1, IL1A, IL1B, IL1RL2, IL2, IL20RB, 1L21, 1L23A, 1L23R, 1L27, IL27RA, IL2RA, 1L36B, 1L4, IL4R, 1L6, IL6ST, IL7, IL7R, INS, IRF1, IRF4, ITGAL, ITK, ITPKB, JAG2, JAK3, JAML, JMJD6, KAT2A, KDELR1, KIF13B, KIT, KLRC4-KLRK1, KLRK1, LAG3, LAT, LAX1, LCK, LCP1, LEF1, LEP, LEPR, LFNG, LGALS1, LGALS3, LGALS7B, LGALS9, LGALS9B, LGALS9C, LIG4, LILRB1, LILRB2, LILRB4, LMBR1L, LMO1, LOXL3, LRRC32, LY9, LYN, MAD111, MAFB, MALT1, MAP3K8, MAPK8IP1, MARCHF7, MDK, METTL3, MICA, MICB, MIR181C, MIR21, MIR27A, MIR30B, MR1, MSN, MTOR, MYB, MYH9, NCAPH2, NCK1, NCK2, NCKAP1L, NCSTN, NDFIP1, NEDD4, NFATC2, NFKBID, NFKBIZ, NHEJ1, NKAP, NKX2-3, NLRC3, NLRP3, NOD2, NRARP, P2RX7, PAG1, PAK1, PAK2, PAK3, PATZ1, PAWR, PAX1, PCK1, PDCD1, PDCD1LG2, PDE5A, PDPK1, PELI1, PIK3CA, PIK3CD, PIK3CG, PIK3R1, PIK3R6, PKNOX1, PLA2G2D, PLA2G2F, PNP, PPP3CA, PPP3CB, PRDM1, PRDX2, PRELID1, PREX1, PRKAR1A, PRKCQ, PRKCZ, PRKDC, PRNP, PRR7, PSAP, PSEN1, PSMB10, PSMB11, PTGER4, PTPN11, PTPN2, PTPN22, PTPN6, PTPRC, PYCARD, RAB27A, RAB29, RABL3, RAC1, RAC2, RAG1, RAG2, RARA, RASAL3, RASGRP1, RC3H1, RC3H2, RELB, RHOA, RHOH, RIPK2, RIPK3, RIPOR2, RORA, RORC, RPS3, RSAD2, RUNX1, RUNX2, RUNX3, SART1, SASH3, SATB1, SCGB1A1, SCRIB, SDC4, SELENOK, SEMA4A, SFTPD, SH3RF1, SHH, SIRPA, SIRPB1, SIRPG, SIT1, SLA2, SLAMF6, SLC11A1, SLC46A2, SLC7A1, SMAD3, SMAD7, SOCS1, SOCS5, SOCS6, SOD1, SOS1, SOX12, SOX13, SOX4, SP3, SPINK5, SPN, SPTA1, SRC, SRF, STAT3, STAT5B, STAT6, STK11, STOML2, SYK, TARM1, TBX21, TCF7, TCIRG1, TESPA1, TFRC, TGFBR2, THEMIS, THY1, TIGIT, TMEM131L, TMEM98, TMIGD2, TNFAIP8L2, TNFRSF13C, TNFRSF14, TNFRSF18, TNFRSF1B, TNFRSF21, TNFRSF4, TNFSF11, TNFSF13B, TNFSF14, TNFSF18, TNFSF4, TNFSF8, TNFSF9, TOX, TP53, TRAF6, TREML2, TREX1, TSC1, TWSG1, VAV1, VCAM1, VNN1, VSIG4, VSIR, VTCN1, WAS, WNT1, WNT4, XBP1, XCL1, YES1, ZAP70, ZBTB1, ZBTB16, ZBTB7B, ZC3H12A, ZC3H8, ZEB1, ZFP36L1, ZFP36L2, ZFPM1, ZMIZ1, ZNF683, ZP3, ZP4; or
(rr) BMP4, ERBB2, FOXP3, GNRH1, IHH, IL1A, IL1B, RIPK2, SHH, TMEM131L, WNT4; or
(ss) HLA-A, HLA-E, IRF1, MAPK8IP1, PTPN22, SH3RF1, VSIR, XCL1; or
(tt) BCL2, CBFB, EOMES, GPR18, IRF1, NCKAP1L, PAX1, PSMB11, RUNX1, RUNX3, SATB1, SOCS1, TNFSF8, TOX, ZBTB7B; or
(uu) BCL2, CBFB, CD274, CLEC4A, CRTAM, EOMES, GPR18, HFE, HLA-A, HLA-E, IRF1, LILRB1, MAPK8IP1, NCKAP1L, PAX1, PSMB11, PTPN22, RUNX1, RUNX3, SATB1, SH3RF1, SOCS1, TNFSF8, TOX, VSIR, XCL1, ZBTB7B; or
(vv) BATF, BCL2, CTSL, CYLD, FOXP3, IL12B, IL12RB1, IL23A, IL23R, IL6, IRF4, LOXL3, LY9, MTOR, SHH, SLAMF6, SPN, STAT3, STAT6, TBX21, TOX, ZFPM1; or
(ww) FOXP3, FUT7, IFNG, LGALS9, PLA2G2D, TOX; or
(xx) ARG2, CD274, CD55, CD81, LGALS7B, LGALS9, LGALS9B, LGALS9C, TGFBR2, TWSG1, VSIR, XCL1; or
(yy) ARG1, CD55, CD81, DENND1B, GATA3, IFNA2, IFNB1, IL12A, IL12B, IL18, IL18R1, IL18RAP, IL1B, IL1R1, IL31RA, IL4, IL6, NLRP3, PRKCZ, RSAD2, STARD7, TBX21, XCL1; or
(zz) AGER, ANXA1, ARG2, ATP7A, BATF, BCL3, BCL6, CBFB, CCL19, CD160, CD274, CD55, CD80, CD81, CD83, CD86, CDH26, CTSL, FOXP1, FOXP3, FUT7, GATA3, GPR183, HLX, HMGB1, IFNG, IL12A, IL12B, IL12RB1, IL18, IL18R1, IL2, L21, IL23A, IL23R, IL27, IL4, IL4R, IL6, IRF4, JAK3, LEF1, LGALS7B, LGALS9, LGALS9B, LGALS9C, LOXL3, LY9, MALT1, MIR21, MTOR, MYB, NCKAP1L, NFKBID, NFKBIZ, NKX2-3, NLRP3, PAX1, PLA2G2D, PRKCQ, PRKCZ, PTGER4, RARA, RC3H1, RC3H2, RELB, RIPK2, RORA, RORC, RSAD2, RUNX1, RUNX3, SASH3, SATB1, SEMA4A, SH3RF1, SLAMF6, SMAD7, SOCS1, SOCS5, SPN, STAT3, STAT6, STOML2, TARM1, TBX21, TCIRG1, TGFBR2, TMEM98, TNFSF4, TOX, TWSG, VSIR, XCL1, ZBTB7B, ZC3H12A, ZFPM1; or
(aaa) ARG2, CCR2, CD274, CD28, CD3E, CD55, CD81, DOCK2, EBI3, ELF4, HLA-A, HLA-E, IL12B, IL15, IL18, IL23A, IRF1, LGALS7B, LGALS9, LGALS9B, LGALS9C, MAPK8IP1, PTPN22, PTPRC, RASAL3, RIPK2, SH3RF1, SYK, TGFBR2, TNFRSF14, TNFSF4, TWSG1, VSIR, XCL1, ZAP70, ZBTB7B; or
(bbb) ANXA1, ATP7A, BATF, BCL3, BCL6, CCL19, CD80, CD86, EOMES, FOXP1, FOXP3, GATA3, GPR183, HLX, HMGB1, IFNG, IL12B, IL12RB1, IL18, IL18R1, IL2, IL21, IL23A, IL23R, IL27, IL4, IL4R, IL6, IRF4, JAK3, LEF1, LGALS9, LOXL3, LY9, MALT1, MIR21, MTOR, MYB, NFKBID, NFKBIZ, NLRP3, PRKCZ, PTGER4, RARA, RC3H1, RC3H2, RELB, RIPK2, RORA, RORC, SEMA4A, SLAMF6, SMAD7, SOCS5, SPN, STAT3, STAT6, TBX21, TMEM98, TNFSF4, ZBTB7B, ZC3H12A, ZFPM1; or
(ccc) ABL1, AGER, ARG1, BTN2A2, BTN3A1, CASP3, CD24, CD274, CLC, CRTAM, EPO, FADD, FOXP3, FYN, GPAM, HHLA2, HMGB1, ICOSLG, IGF1, IGF2, IGFBP2, IL12B, IL12RB1, IL18, IL2, IL23A, IL23R, IL27RA, IL2RA, LGALS9, LRRC32, MIR181C, MIR21, MIR30B, PDCD1LG2, PRKAR1A, PRNP, PYCARD, RC3H1, RIPK3, RPS3, SATB1, SCRIB, STAT5B, TMIGD2, TNFSF9; or any combination thereof.

In an aspect, the subject is diagnosed with a cancer. In an aspect, the subject has a non-hematological solid tumor. Non-limiting examples of solid cancer are Adrenocortical carcinoma (ACC), Bladder Urothelial Carcinoma (BLCA), Brain Lower Grade Glioma (LGG), Breast invasive carcinoma (BRCA), Colon adenocarcinoma (COAD), Cervical squamous cell carcinoma and endocervical adenocarcinoma (CESC), Cholangiocarcinoma (CHOL), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Uterine Carcinosarcoma (UCS), Uterine Corpus Endometrial Carcinoma (UCEC), and Uveal Melanoma (UVM). In an aspect, the subject is determined to be responsive to ICB when the angio-immune subtype corresponds to a low angiogenic signature and high T cell function signature. In an aspect, the two or more genes comprises genes involved in two different biological pathways, and wherein the two different biological pathways impact angiogenic function and T cell function. In an aspect the two or more genes are all genes provided in (i) and (ii). In an aspect, the biological sample is a cancer or tumor biopsy.

In an aspect, the biological sample is a tumor microenvironment (TME) aspirate. In an aspect of the method disclosed herein, the ICB therapy is a PD-1 inhibitor therapy or a CTLA4 inhibitor therapy. In an aspect, the ICB therapy is a PD-1 inhibitor therapy.

In an aspect, the expression levels of the two or more genes are measured by RNA sequencing, RT-PCR and microarray analysis.

In an aspect of the method disclosed herein, the angio-immune subtype is determined by computational analysis. In an aspect, the angio-immune subtype is determined by baseline angio-immune score calculated by the computational analysis based on the provided expression levels of the group of genes, and wherein deviation of the score from a reference value indicates whether the subject would respond to or not respond to the therapy.

In an aspect of a disclosed method the step (iv), is further based on one or more clinical factors. In an aspect, the one or more clinical factors comprise mutation status of one or more genes, stromal score, fibroblast score, and mutational burden. In an aspect, the one or more genes is selected from CSMD3, IDH1, BRAF, PTEN, ATRX, LRP1B, ZFHX4, USH2A, and FAT4.

In an aspect, the current disclosure also encompasses a method of predicting responsiveness of a subject to an immune checkpoint blockade (ICB) therapy, comprising: (a) obtaining or having obtained the expression and mutational profile of the two or more genes as provided in Table 1; (b) implementing a gene set variation analysis (GSVA) to obtain an enrichment score of the two or more genes; (c) generating a correlation matrix across the enrichment scores; (d) identifying the angio-immune subtype of the subject based on the correlation matrix; and (e) predicting the responsiveness of the subject to an ICB therapy based on the angio-immune subtype, wherein the expression and mutational profile of the two or more genes was obtained by RNA sequencing, RT-PCR, or microarray analysis or a combination thereof of a biological sample from the subject.

In an aspect, the subject is predicted to be responsive to ICB therapy if the subjects angio-immune subtype corresponds to a low angiogenic signature and high T cell function signature. In an aspect, the subject is diagnosed with a cancer. In an aspect, the subject has a non-hematological solid tumor. In an aspect, the cancer is Adrenocortical carcinoma (ACC), Bladder Urothelial Carcinoma (BLCA), Brain Lower Grade Glioma (LGG), Breast invasive carcinoma (BRCA), Colon adenocarcinoma (COAD), Cervical squamous cell carcinoma and endocervical adenocarcinoma (CESC), Cholangiocarcinoma (CHOL), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Uterine Carcinosarcoma (UCS), Uterine Corpus Endometrial Carcinoma (UCEC), and Uveal Melanoma (UVM). In an aspect of a disclosed method, the biological sample is a cancer or tumor biopsy. In an aspect, the biological sample is a tumor microenvironment (TME) aspirate. In an aspect, the ICB therapy is a PD-1 inhibitor therapy or a CTLA4 inhibitor therapy. In an aspect, the ICB therapy is a PD-1 inhibitor therapy. In an aspect, the subject is a mammal. In an aspect, the subject is a human.

### BRIEF DESCRIPTION OF THE FIGURES

The patent or patent application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A shows a schematic depicting generation of enrichment matrices used for angio-immune subtype identification. RNA-sequencing data was used to score the enrichment of 91 gene signatures using GSVA. Results of GSVA are used to identify angio-immune subtypes.

FIG. 1B provides a heatmap depicting Pearson correlation of gene sets across 11,069 TCGA tumor samples. Gene sets are bound by positive correlation and separated by negative correlation.

FIG. 2A provides a heatmap of Pearson Correlation of patients with non-hematological tumor types across 91 gene sets corresponding T-cell and angiogenesis activity. Distance based clustering revealed three distinct clusters across patients. The clusters were labelled as C1 (red outline), C2 (black outline), and C3 (green outline).

FIG. 2B shows bar graphs depicting the average enrichment of angiogenesis signatures in the three angio-immune subtypes. Enrichment of pathways was conducted using gene set variation analysis (GSVA). The enrichment of representative gene sets are plotted. One way ANOVA was used to determine statistical significance.

FIG. 2C shows bar graphs depicting the average enrichment of T-cell signatures in the three angio-immune subtypes. Enrichment of pathways was conducted using gene set variation analysis (GSVA). The enrichment of representative gene sets are plotted. One way ANOVA was used to determine statistical significance.

FIG. 2D shows stacked bar plots depicting relative proportion of each angio-immune subtype in all cancers queried. The proportion of all tumors of a particular cancer type belonging to individual angio-immune subtypes was calculated.

FIG. 2E shows a comparison of membership to previously established molecular subtypes and angio-immune clusters in skin cutaneous melanoma (SKCM).

FIG. 2F shows a comparison of membership to previously established molecular subtypes and angio-immune clusters in ovarian carcinoma (OV).

FIG. 2G shows a comparison of membership to previously established molecular subtypes and angio-immune clusters in adrenocortical carcinoma (ACC).

FIG. 2H shows the overall survival of patients belonging to 3 angio-immune subtypes among skin cutaneous melanoma (SKCM) patients. Survival data was derived from publicly available clinical records of TCGA patients.

FIG. 2I-2J shows the overall survival for tumor types among patients belonging to the three angio-immune clusters. Survival differences observed in bladder adenocarcinoma (BLCA), lung adenocarcinoma (LUAD), head and neck squamous cell carcinoma (HNSC), clear cell renal carcinoma (KIRC), low grade glioma (LGG), pancreatic adenocarcinoma (PAAD), stomach adenocarcinoma (STAD), and renal adenocarcinoma (READ).

FIG. 3A shows a bar plots showing xCell enrichment results for major tumor hematopoietic cells across angio-immune subtypes. The enrichment of classical dendritic cells (cDC), plasmacytoid dendritic cells (pDC), activated dendritic cells (aDC), B cells, plasma cells, CD4+ and CD8+ T cells, Tregs, Th1 cells, Th2 cells, and M1 and M2 macrophages as derived from XCell were compared across angio-immune subtypes. One way ANOVA was used to determine statistical significance.

FIG. 3B shows a heatmap of the z-scored expression co-stimulatory molecules across angio-immune subtypes.

FIG. 3C shows a heatmap of the z-scored expression T cell inhibitory molecules across angio-immune subtypes.

FIG. 3D shows a bar plot showing xCell enrichment results for endothelial cells, fibroblasts, and pericytes across angio-immune subtypes. One way ANOVA was used to determine statistical significance.

FIG. 3E shows a vessel normalization scores across angio-immune subtypes. XCell enrichment for pericytes and endothelial cells was normalized and the ratio of pericytes to endothelial was evaluated and termed the vessel normalization score and is plotted in a bar graph. One way ANOVA was used to determine statistical significance.

FIG. 3F provides violin plots depicting silent and non-silent mutational burden across three angio-immune clusters. Data was obtained from the GDC pan-cancer atlas. One way ANOVA was used to determine statistical significance.

FIG. 3G shows a violin plot of neoantigen counts across three angio-immune clusters. Neoantigen counts were log transformed for visualization purposes. One way ANOVA was used to determine statistical significance.

FIG. 3H shows a violin plot of TCR richness across three angio-immune clusters. TCR richness data was obtained from the GDC pan-cancer atlas. One way ANOVA was used to determine statistical significance.

FIG. 3I shows violin plots showing xCell enrichment results of CD8 T cell, M1 macrophage, classical dendritic cell (cDC), B cells, Th1 cells, and Th2 cells across angio-immune subtypes among skin cutaneous melanoma patients from the TCGA. One way ANOVA was used to determine statistical significance.

FIG. 3J shows violin plots showing xCell enrichment results of CD8 T cell, M1 macrophage, classical dendritic cell (cDC), B cells, Th1 cells, and Th2 cells across angio-immune subtypes among bladder cancer patients from the TCGA. One way ANOVA was used to determine statistical significance.

FIG. 3K shows violin plots showing xCell enrichment results of CD8 T cell, M1 macrophage, classical dendritic cell (cDC), B cells, Th1 cells, and Th2 cells across angio-immune subtypes among stomach adenocarcinoma patients from the TCGA. One way ANOVA was used to determine statistical significance.

FIG. 3L shows violin plots showing xCell enrichment results of CD8 T cell, M1 macrophage, classical dendritic cell (cDC), B cells, Th1 cells, and Th2 cells across anglo-immune subtypes among clear cell renal carcinoma patients from the TCGA. One way ANOVA was used to determine statistical significance.

FIG. 4A shows a heatmap of Pearson Correlation of 145 patients with Melanoma across 91 gene sets corresponding T-cell and angiogenesis activity. Angio-immune subtypes are preserved in the melanoma cohort. Response status is depicted for each patient on top of the heatmap. Green depicts responders to treatment and red depicts non-responders.

FIG. 4B shows a heatmap of Pearson Correlation of 45 patients with Gastric cancer across 91 gene sets corresponding T-cell and angiogenesis activity. Response status is depicted for each patient on the top of the heatmap. Angio-immune subtypes are preserved in the gastric cancer cohort. Response status is depicted for each patient on top of the heatmap. Green depicts responders to treatment and red depicts non-responders.

FIG. 4C shows a heatmap of Pearson Correlation of 348 patients with Bladder cancer across 91 gene sets corresponding T-cell and angiogenesis activity. Angio-immune subtypes are preserved in the gastric cancer cohort.

FIG. 4D shows a heatmap of Pearson Correlation of 51 patients with Melanoma treated with anti-CTLA4 across 91 gene sets corresponding T-cell and angiogenesis activity. Angio-immune subtypes were conserved in this melanoma cohort.

FIG. 4E shows bar graphs depicting the average enrichment of angiogenesis signature and T-cell signature in the three angio-immune subtypes in Melanoma cohort. Enrichment of pathways was conducted using gene set variation analysis (GSVA). The enrichment of representative gene sets are plotted. One way ANOVA was used to determine statistical significance.

FIG. 4F shows bar graphs depicting the average enrichment of angiogenesis signature and T-cell signature in the three angio-immune subtypes in Gastric cancer cohort. Enrichment of pathways was conducted using gene set variation analysis (GSVA). The enrichment of representative gene sets are plotted. One way ANOVA was used to determine statistical significance.

FIG. 4G shows bar graphs depicting the average enrichment of angiogenesis signature and T-cell signature in the three angio-immune subtypes in Bladder cancer cohort. Enrichment of pathways was conducted using gene set variation analysis (GSVA). The enrichment of representative gene sets are plotted. One way ANOVA was used to determine statistical significance.

FIG. 4H shows bar graphs depicting the average enrichment of angiogenesis signature and T-cell signature in the three angio-immune subtypes in the anti-CTLA4 treated Melanoma cohort. One way ANOVA was used to determine statistical significance.

FIG. 4I shows overall survival and progression free survival for patients in different angio-immune clusters upon treatment with anti-PD1 in melanoma patients

FIG. 4J shows response rate for patients in different angio-immune clusters upon treatment with anti-PD1 in gastric cancer patients.

FIG. 4K shows progression free survival for patients in different angio-immune clusters upon treatment with anti-PDL1 in Bladder cancer patients

FIG. 4L shows progression free survival of patients with melanoma treated with anti-PD1 split by high (>50^{th} percentile) and low (<50^{th} percentile) angiogenesis and T cell cytotoxicity.

FIG. 4M shows progression free survival of patients with bladder cancer treated with anti-PDL1 split by high (>50^{th} percentile) and low (<50^{th} percentile) angiogenesis and T cell cytotoxicity.

FIG. 4N shows overall survival for patients in different angio-immune clusters upon treatment with anti-CTLA4 in melanoma patients (*=p<0.05, **=p<0.01, ***=p<0.001, ****=p<0.0001)

FIG. 5A shows a heatmap of Pearson Correlation of 726 patients with Renal Cell Carcinoma across 91 gene sets corresponding T-cell and angiogenesis activity. Angio-immune subtypes are preserved in the renal cancer cohort.

FIG. 5B shows heatmap of Pearson Correlation of 311 patients with Renal Cell Carcinoma across 91 gene sets corresponding T-cell and angiogenesis activity. Angio-immune subtypes were conserved in this renal cancer cohort.

FIG. 5C shows bar graphs depicting the average enrichment of angiogenesis signatures and T-cell signatures in the three angio-immune subtypes in Renal Cell Carcinoma cohort. Enrichment of pathways was conducted using gene set variation analysis (GSVA). The enrichment of representative gene sets are plotted. One way ANOVA was used to determine statistical significance.

FIG. 5D shows bar graphs depicting the average enrichment of angiogenesis signatures and T-cell signatures in the three angio-immune subtypes in the Braun Renal Cell Carcinoma cohort. One way ANOVA was used to determine statistical significance.

FIG. 5E shows progression free survival of patients treated with Sunitinib vs the combination of Axitinib+Avelumab.

FIG. 5F shows progression free survival of patients treated with Sunitinib belonging to different angio-immune clusters.

FIG. 5G shows progression free survival of patients treated with combination of Axitinib+Avelumab belonging to different angio-immune clusters.

FIG. 5H shows progression free survival of patients belonging in C1 treated with Sunitinib vs the combination of Axitinib+Avelumab.

FIG. 5I shows progression free survival of patients belonging in C2 treated with Sunitinib vs the combination of Axitinib+Avelumab.

FIG. 5J shows progression free survival of patients belonging in C3 treated with Sunitinib vs the combination of Axitinib+Avelumab.

FIG. 5K shows overall survival of patients treated with everolimus vs nivolumab.

FIG. 5L shows overall survival of patients treated with everolimus belonging to different angio-immune clusters.

FIG. 5M shows overall survival of patients treated with nivolumab belonging to different angio-immune clusters.

FIG. 5N shows overall survival of patients belonging in C1 treated with everolimus vs nivolumab.

FIG. 5O shows overall survival of patients belonging in C2 treated with everolimus vs nivolumab.

FIG. 5P shows overall survival of patients belonging in C3 treated with everolimus vs nivolumab.

FIG. 6A shows overall survival (OS) tracked for anti-PD1 treated melanoma, anti-PDL1 treated bladder cancer, and anti-PD1 treated renal cancer (Braun) and progression-free survival (PFS) tracked for anti-PDL1 treated renal cancer (Javelin) based on CXCL9 expression. Median expression was used to separate “High” and “Low” expressers.

FIG. 6B shows overall survival (OS) tracked for anti-PD1 treated melanoma, anti-PDL1 treated bladder cancer, and anti-PD1 treated renal cancer (Braun) and progression-free survival (PFS) tracked for anti-PDL1 treated renal cancer (Javelin) based on IFNG signature enrichment. Median enrichment was used to separate “High” and “Low” expressers.

FIG. 6C shows overall survival (OS) tracked for anti-PD1 treated melanoma, anti-PDL1 treated bladder cancer, and anti-PD1 treated renal cancer (Braun) and progression-free survival (PFS) tracked for anti-PDL1 treated renal cancer (Javelin) based on IMPRES scores. Median scores were used to separate “High” and “Low” scores.

FIG. 6D shows overall survival (OS) tracked for anti-PD1 treated melanoma, anti-PDL1 treated bladder cancer, and anti-PD1 treated renal cancer (Braun) and progression-free survival (PFS) tracked for anti-PDL1 treated renal cancer (Javelin) based on PD-L1 expression. Median expression was used to separate “High” and “Low” expressers.

FIG. 6E shows overall survival (OS) tracked for anti-PD1 treated melanoma, anti-PDL1 treated bladder cancer, and anti-PD1 treated renal cancer (Braun) and progression-free survival (PFS) tracked for anti-PDL1 treated renal cancer (Javelin) based on MHCI expression. Median expression was used to separate “High” and “Low” expressers.

FIG. 6F shows overall survival (OS) tracked for anti-PD1 treated melanoma, anti-PDL1 treated bladder cancer, and anti-PD1 treated renal cancer (Braun) and progression-free survival (PFS) tracked for anti-PDL1 treated renal cancer (Javelin) based on MHCII expression. Median expression was used to separate “High” and “Low” expressers.

FIG. 6G shows overall survival of anti-PDL1 treated bladder cancer patients separated by tumor mutational burden status. Survival was plotted for patients across the cohort and belonging to the three angio-immune clusters separated by high (>50th percentile) and low (<50th percentile) tumor mutational burden.

FIG. 6H shows progression-free survival of anti-PDL1 treated renal cancer patients separated by tumor mutational burden status. Survival was plotted for patients across the cohort and patients belonging to the three angio-immune clusters separated by high (>50th percentile) and low (<50th percentile) tumor mutational burden.

### DETAILED DESCRIPTION

The present disclosure is based, at least in part, on the discovery that baseline angiogenic state is a determinant of cytotoxic T-cell function and infiltration in the tumor microenvironment (TME). Indeed, the present disclosure provides a transcriptional profile analysis pipeline to stratify patients based on baseline angiogenic and immune activity using endothelial cell and T-cell functional gene sets. This highly interpretable tool provides insight into the intimate interaction between angiogenic and immune processes and enables the identification of pan-cancer molecular subtypes of tumor TME. Importantly, pan-cancer angio-immune TME subtypes are prognostic of response and survival of patients treated with ICB. Retrospective analysis of pre-treatment datasets revealed that patients showing low angiogenic TME significantly benefit from FDA-approved first-line ICB strategies. The present disclosure provides a reformed understanding of how angiogenesis and T-cell immunity are linked across tumor types, laying the foundation for more efficient treatment decision-making approaches.

Aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which aspects of the invention pertain. Many methods and materials similar, modified, or equivalent to those described herein can be used in the practice of the aspects of the present invention without undue experimentation, the preferred materials and methods are described herein. In describing and claiming the aspects of the present invention, the following terminology will be used in accordance with the definitions set out below.

The term “a” or “an” entity refers to one or more of that entity; for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone).

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

### I. Predicting Therapeutic Responsiveness/Non-responsiveness to Immune Checkpoint Blockade (ICB)

One aspect of the present disclosure relates to systems and methods for predicting whether a subject (e.g., a patient) will respond positively to an immune checkpoint blockade therapy (e.g., a responder) or the subject will not respond positively to an immune checkpoint blockade therapy (e.g., a non-responder) based on patient-specific information such as a patient's expression data (e.g., expression levels and/or expression level differences). In some aspects, the current disclosure encompasses assessing responsiveness or non-responsiveness of a subject with a cancer or tumor to a therapeutic agent (e.g., immune checkpoint blockade and/or anti-angiogenesis) based on an angio-immune subtype of the tumor microenvironment (TME) as disclosed herein. As used herein, assessing “responsiveness” or “non-responsiveness” to a therapeutic agent refers to the determination of the likelihood of a subject for responding or not responding to the therapeutic agent.

Thus, in one aspect, the current disclosure encompasses a method of predicting responsiveness of a subject to an immune checkpoint blockade (ICB) therapy, comprising: (i) providing expression levels of two or more genes in a biological sample obtained from tumor microenvironment (TME) of the subject, wherein the two or more genes encode effectors that impact angiogenesis or T-cell function or both; (ii) determining an angio-immune score based on the expression levels of the two or more genes in step (i); (iii) comparing the angio-immune score to an angio-immune baseline score for a corresponding tumor microenvironment to determine an angio-immune subtype; and (iv) predicting the subject's responsiveness to an ICB therapy based on the angio-immune subtype. In some aspects, the current disclosure also encompasses a method of identifying patients who would be responsive to ICB therapy using a method comprising steps (i)-(iv). In some aspects, the current disclosure also encompasses method of predicting responsiveness of a subject to an immune checkpoint blockade (ICB) therapy, comprising: (i) obtaining or having obtained a sample from the subject; (ii) determining the expression and mutational profile of the two or more genes as provided in Table 1; (iii) implementing a gene set variation analysis (GSVA) to obtain an enrichment score of the two or more genes; (iv) generating a correlation matrix across the enrichment scores; (v) identifying the angio-immune subtype of the subject based on the correlation matrix.

### (A) Angio-Immune Profile

An angio-immune profile refers to a characteristic expression profile of a single or a group of genes that is indicative of an altered or unaltered biological process, medical condition, or a patient's responsiveness/non-responsiveness to a specific therapy. In some aspects, the gene or set of genes relate to angiogenic or T cell function or both in the tumor microenvironment (TME). As used herein the term “tumor microenvironment” refers to the ecosystem that surrounds a tumor inside the body. It includes immune cells, the extracellular matrix, blood vessels and other cells, like fibroblasts. A tumor and its microenvironment constantly interact and influence each other, either positively or negatively. Gene expression profile in the tumor microenvironment can vary both spatially and temporally and based on tumor type.

The angio-immune profile disclosed herein may encompass characteristic expression profiles of two or more genes listed in Table 1 below, which are identified as differentially expressed in tumor microenvironment of various cancers.

TABLE 1
List of gene sets corresponding to endothelial cells and T-cell activity

| | |
|---|---|
| Angiogenesis Signature | MYCT1, APOH, APP, CCND2 COL3A1. COL5A2, CXCL6, FGFR1. |
| | FSTL1, ITGAV, JAG1, JAG2, KCNJ8, LPL, LRPAP1, LUM, MSX1, |
| | NRP1, OLR1, PDGFA, PF4, PGLYRP1, POSTN, PRG2, PTK2, |
| | S100A4, SERPINA5, SLCO2A1, SPP1, STC1, THBD, TIMP1, |
| | TNFRSF21, VAV2, CAN, VEGFA, VTN, CDH5, ELTD1, CLEC14A, |
| | LDB2, ECSCR, RHOJ, VWF, TIE1, KDR, ESAM, CD93, PTPRB, |
| | GPR116, SPARCL1, EMCN, ROBO4, ENG, TEK, SIPR1 |
| Positive_Regulation_of_Angiogenesis | AGGF1, AMOT, ANGPTL3, ANGPTL4, BTG1, CHRNA7, RHOB, |
| | RUNX1, SPHK1, TNFSF12 |
| Hallmark_Angiogenesis | APOH, APP, CCND2, COL3A1, COL5A2, CXCL6, FGFR1, FSTL1, |
| | ITGAV, JAG1, JAG2, KCNJ8, LPL, LRPAP1, LUM, MSX1, NRP1, |
| | OLR1, PDGFA, PF4, PGLYRP1, POSTN, PRG2, PTK2, S100A4, |
| | SERPINA5, SLCO2A1, SPP1, STC1, THBD, TIMP1, TNFRSF21, |
| | VAV2, CAN, VEGFA, VTN |
| Go_Regulation_Of_Sprouting_Angiogenesis | ABL1, ADAMTS9, AGTR1, AKT3, ALOX5, ANXA1, APELA, APLNR, |
| | BMPER, CARD10, CEACAM1, CEMIP2, CIB1, CREB3L1, DLL1, DLL4, |
| | E2F2, EPN1, EPN2, FBXW7, FGF1, FGF2, FGFBP1, FOXC2, FUT1, |
| | GATA2, GHRL, GHSR, GLUL, HDAC5, HDAC7, HDAC9, HMOX1, |
| | IL10, IL12A, IL12B, ITGA5, ITGB1BP1, JAK1, JCAD, JMJD8, KDR, |
| | KLF2, KLF4, MAP2K5, MAP3K3, MEOX2, MIR1-1, MIR1-2, MIR101-1, |
| | MIR101-2, MIR10A, MIR10B, MIR1224, MIR125A, MIR126, MIR132, |
| | MIR138-1, MIR138-2, MIR146A, MIR149, MIR150, MIR15A, MIR15B, |
| | MIR16-1, MIR16-2, MIR17, MIR188, MIR18A, MIR193A, MIR196A1, |
| | MIR196A2, MIR199A1, MIR199A2, MIR199B, MIR19A, MIR19B1, |
| | MIR19B2, MIR200C, MIR206, MIR20A. MIR21, MIR22, MIR221, |
| | MIR222, MIR2355, MIR23A, MIR24-1, MIR24-2, MIR26A1, MIR26A2, |
| | MIR27A, MIR27B, MIR296, MIR29C, MIR30B, MIR30C1, MIR30C2, |
| | MIR30E, MIR31, MIR320A, MIR329-1, MIR329-2, MIR342, MIR34A, |
| | MIR34B, MIR34C, MIR361, MIR375, MIR377, MIR410, MIR424, |
| | MIR483, MIR487B, MIR494, MIR495, MIR497, MIR503, MIR92A1, |
| | MIR92A2, MIRLET7B, MIRLET7F1, MIRLET7F2, MMRN2, NGFR, |
| | NOTCH1, NR2E1, NRP1, PDCD10, PDPK1, PIK3C2A, PKM, PLK2, |
| | PPP1R16B, PTGS2, RHOA, RHOJ, S100A1, SEMA6A, SMAD1, |
| | SPRED1, SRPX2, STARD13, SYNJ2BP, TBXA2R, THBS1, TJP1, |
| | VEGFA |
| Go_Positive_Regulation_Of_{—} | ABL1, AGTR1, AKT3, ANXA1, APELA, APLNR, BMPER, CIB1, DLL1, |
| Sprouting_Angiogenesis | FGF1, FGF2, FGFBP1, FOXC2, FUT1, GATA2, GHRL, GHSR, |
| | HDAC7, HDAC9, HMOX1, IL10, ITGA5, JAK1, JCAD, JMJD8, KDR, |
| | KLF4, MAP3K3, MIR1-1, MIR1-2, MIR101-1, MIR101-2, MIR10A, |
| | MIR10B, MIR1224, MIR125A, MIR126, MIR132, MIR146A, MIR150, |
| | MIR21, MIR23A, MIR27A, MIR27B, MIR296, MIR30B, MIR31, |
| | MIR487B, MIR503, MIR92A1, MIR92A2, MIRLET7B, MIRLET7F1, |
| | MIRLET7F2, NRP1, PDPK1, PIK3C2A, PKM, PLK2, PPP1R16B, |
| | PTGS2, RHOJ, S100A1, SMAD1, SRPX2, TJP1, VEGFA |
| Go_Positive_Regulation_Of_{—} | ABL1, AKT3, ANXA1, CIB1, FGF2, FGFBP1, FOXC2, GATA2, HDAC7, |
| Cell_Migration_Involved_{—} | HDAC9, HMOX1, JCAD, KDR, MAP3K3, MIR101-1, MIR101-2, |
| In_Sprouting_Angiogenesis | MIR10A, MIR10B, MIR126, MIR132, MIR146A, MIR150, MIR23A, |
| | MIR27A, MIR27B, MIR296, MIR31, MIR487B, MIRLET7F1, |
| | MIRLET7F2, NRP1, PIK3C2A, PLK2, PTGS2, RHOJ, SRPX2, VEGFA |
| Go_Cell_Migration_Involved_In_{—} | ABL1, ADTRP, AKT1, KT3, ANXA1, CARD10, CIB1, CLEC14A, DLL4, |
| Sprouting_Angiogenesis | EFNB2, EGR3, EPHB4, FBXW7, FGF2, FGFBP1, FOXC2, GATA2, |
| | GPLD1, GREM1, HDAC5, HDAC7, HDAC9, HMOX1, ITGB1, |
| | ITGB1BP1, JCAD, KDR, KLF4, MAP2K5, MAP3K3, MEOX2, MIA3, |
| | MIR101-1, MIR101-2, MIR10A, MIR10B, MIR126, MIR132, MIR146A, |
| | MIR149, MIR150, MIR15A, MIR16-1, MIR16-2, MIR188, MIR193A, |
| | MIR196A1, MIR196A2, MIR199A1, MIR199A2, MIR199B, MIR19B1, |
| | MIR19B2, MIR200C, MIR206, MIR20A, MIR22, MIR221, MIR2355, |
| | MIR23A, MIR26A1, MIR26A2, MIR27A, MIR27B, MIR296, MIR29C, |
| | MIR31, MIR320A, MIR329-1, MIR329-2, MIR361, MIR410, MIR424, |
| | MIR483, MIR487B, MIR494, MIR495, MIR497, MIR503, MIRLET7F1, |
| | MIRLET7F2, MMRN2, NOTCH1, NR2E1, NR4A1, NRP1, PDCD10, |
| | PIK3C2A, PIK3R3, PLK2, PTGS2, RHOA, RHOJ, ROBO1, SLIT2, |
| | SPRED1, SRF, SRPX2, STARD13, TBXA2R, TDGF1, THBS1, VEGFA |
| Angiogenesis | ACVRL1, AGGF1, AMOT, ANG, ANGPTL3, ANGPTL4, ATP5IF1, |
| | BTG1, C1GALT1, CANX, CDH13, CHRNA7, COL4A2, COL4A3, |
| | CXCL8, EGF, EMCN, EPGN, ERAP1, FOXO4, HTATIP2, IL17F, IL18, |
| | MYH9, NCL, NF1, NOTCH4, NPPB, NPR1, PF4, PLG, PML, PROK2, |
| | RHOB, RNH1, ROBO4, RUNX1, SCG2, SERPINF1, SHH, SPHK1, |
| | SPINK5, STAB1, TGFB2, THY1, TNFSF12, TNNI3, VEGFA |
| Kegg_Leukocyte_Transendothelial_{—} | ACTB, ACTG1, ACTN1, ACTN2, ACTN3, ACTN4, AFDN, ARHGAP35, |
| Migration | ARHGAP5, BCAR1, CD99, CDC42, CDH5, CLDN1, CLDN10, |
| | CLDN11, CLDN14, CLDN15, CLDN16, CLDN17, CLDN18, CLDN19, |
| | CLDN2, CLDN20, CLDN22, CLDN23, CLDN3, CLDN4, CLDN5, |
| | CLDN6, CLDN7, CLDN8, CLDN9, CTNNA1, CTNNA2, CTNNA3, |
| | CTNNB1, CTNND1, CXCL12, CXCR4, CYBA, CYBB, ESAM, EZR, |
| | F11R, GNAI1, GNAI2, GNAI3, ICAM1, ITGA4, ITGAL, ITGAM, ITGB1, |
| | ITGB2, ITK, JAM2, JAM3, MAPK11, MAPK12, MAPK13, MAPK14, |
| | MMP2, MMP9, MSN, MYL10, MYL12A, MYL12B, MYL2, MYL5, MYL7, |
| | MYL9, MYLPF, NCF1, NCF2, NCF4, NOX1, NOX3, OCLN, PECAM1, |
| | PIK3CA, PIK3CB, PIK3CD, PIK3CG, PIK3R1, PIK3R2, PIK3R3, |
| | PIK3R5, PLCG1, PLCG2, PRKCA, PRKCB, PRKCG, PTK2, PTK2B, |
| | PTPN11, PXN, RAC1, RAC2, RAP1A, RAP1B, RAPGEF3, RAPGEF4, |
| | RASSF5, RHOA, RHOH, ROCK1, ROCK2, SIPA1, THY1, TXK, VASP, |
| | VAV1, VAV2, VAV3, VCAM1, VCL |
| Lu_Tumor_Endothelial_Markers_Up | ADAM12, ADAP2, CHN1, COL5A3, EGFL6, ZD10, OPX, HSPA6, |
| | LCP2, MMP9, MXRA5, PAMR1, PLXDC1, PMAIP1, POSTN, RPS11, |
| | STC1, TNFAIP6, TNFRSF21, TWIST1, CAN, WDR77 |
| Go_Vascular_Endothelial_Growth_Factor_{—} | FLT1, FLT3, FLT4, KDR, NRP1, NRP2, PDGFRA |
| Activated_Receptor_Activity | |
| Go_Vascular_Endothelial_{—} | ADAM17, AKT3, APLN, CCL2, COL4A3, FGF2, FGFR1, FLT1, GHRL, |
| Cell_Proliferation | GHSR, HMGB1, IGF2, ITGA4, MDK, MEF2C, MIR126, MIR129-1, |
| | MIR129-2, MIR130A, MIR132, MIR133B, MIR135B, MIR152, MIR15B, |
| | MIR193A, MIR20B, MIR21, MIR24-1, MIR24-2, MIR27A, MIR29A, |
| | MIR29C, MIR30B, MIR30E, MIR329-1, MIR329-2, MIR34A, MIR424, |
| | MIR487B, MIR492, MIR495, MIR499A, MIR503, MIR98, PDPK1, |
| | PLCG1, PPARG, SIRT6, SP1, STAT3 |
| Go_Regulation_Of_Vascular_{—} | ARNT, CADM4, CNMD, DAB2IP, EMILIN1, EPN2, FGF10, FGF18, |
| Endothelial_Growth_Factor_{—} | FGF9, FZD4, GRB10, HGS, HHEX, HIF1A, IL1B, ITGA5, ITGB3, |
| Receptor_Signaling_Pathway | MIR10A, MIR10B, MIR1224, MIR200C, MIR296, MMRN2, MT3, |
| | MYOF, NEDD4, NIBAN2, PDCD6, PRKCB, PRKD2, PTPN1, |
| | TMEM204, TNMD, VEGFC, VTN |
| Go_Regulation_Of_Leukocyte_{—} | ALOX5, CCL21, CCL25, CCL28, CCR2, CXCL12, ELANE, ETS1, |
| Adhesion_To_Vascular_{—} | FUT4, FUT7, FUT9, ICAM1, IL6, IRAK1, ITGA4, ITGB2, KLF4, MDK, |
| Endothelial_Cell | MIR125A, MIR141, MIR146A, MIR21, MIR221, MIR222, MIR31, |
| | MIR92A1, MIR92A2, MIRLET7E, MIRLET7G, NFAT5, PTAFR, RELA, |
| | RHOA, SELE, SELP, TNF, TRAF6 |
| Go_Regulation_Of_Leukocyte_{—} | ALOX5, KLF4, MIR92A1, MIR92A2, TNF |
| Adhesion_To_Arterial_{—} | |
| Endothelial_Cell | |
| Go_Regulation_Of_Endothelial_{—} | ADAMTS12, ALOX12, CXCL10, FGF1, FOXP1, ITGAX, MIR21 |
| Tube_Morphogenesis | |
| Go_Regulation_Of_Endothelial_{—} | CXCL13, FGF1, FGF16, FGF18, FGF2, FGF4, FGFR1, HRG, HSPB1, |
| Cell_Chemotaxis | KDR, LGMN, MET, MIR149, MIR16-1, MIR16-2, MIR424, NOTCH1, |
| | P2RX4, PRKD1, PRKD2, SEMA5A, SMOC2, THBS1, TMSB4X, |
| | VEGFA |
| Go_Regulation_Of_Cellular_{—} | ADAMTS12, ADAMTS3, ADGRA2, ATP2B4, CADM4, CCBE1, CD63, |
| Response_To_Vascular_{—} | DAB2IP, DCN, DLL1, HRG, IL12A, IL12B, JCAD, MIR16-1, MIR16-2, |
| Endothelial_Growth_Factor_{—} | MIR199A1, MIR199A2, MIR199B, MIR21, MIR329-1, MIR329-2, |
| Stimulus | MIR342, MIR424, MYO1C, PTP4A3, ROBO1, SEMA6A, SMOC2, |
| | SPRY2, TSPAN12, XDH |
| Go_Positive_Regulation_Of_{—} | ADAMTS3, CCBE1, JCAD, MIR21, MYO1C, ROBO1, SMOC2 |
| Vascular_Endothelial_Growth_{—} | |
| Factor_Signaling_Pathway | |
| Go_Positive_Regulation_Of_{—} | ARNT, FGF10, FGF18, FGF9, GRB10, HIF1A, IL1B, ITGA5, ITGB3, |
| Vascular_Endothelial_Growth_{—} | MIR10A, MIR10B, MIR1224, MIR296, MT3, PRKCB, PRKD2, VTN |
| Factor_Receptor_Signaling_Pathway | |
| Go_Positive_Regulation_Of_{—} | ADORA2B, ARNT, ATF4, BRCA1, C3, C3AR1, C5, C5AR1, CCBE1, |
| Vascular_Endothelial_Growth_{—} | CXCL17, CYP1B1, EIF2AK3, FLT4, GATA4, HIF1A, HPSE, IL1A, IL1B, |
| Factor_Production | IL6, IL6ST, ISL1, NODAL, NOX1, PTGS2, RORA, SULF1, SULF2, |
| | TGFB1 |
| Go_Positive_Regulation_Of_{—} | ADAM17, AKT3, APLN, FGF2, FGFR1, GHRL, HSR, HMGB1, IGF2, |
| Vascular_Endothelial_Cell_{—} | ITGA4, MDK, MIR126, MIR130A, MIR132, MIR135B, MIR21, MIR27A, |
| Proliferation | MIR29A, MIR495, MIR499A, PDPK1, PLCG1, SIRT6, SP1, STAT3 |
| Go_Positive_Regulation_Of_{—} | ALOX5, CCR2, ELANE, ETS1, FUT4, FUT7, ICAM1, IL6, IRAK1, |
| Leukocyte_Adhesion_To_{—} | ITGA4, ITGB2, MDK, MIR21, MIR92A1, MIR92A2, NFAT5, PTAFR, |
| Vascular_Endothelial_Cell | RELA, RHOA, SELE, SELP, TNF, TRAF6 |
| Go_Positive_Regulation_Of_{—} | ACVRL1, ADAM17, AGGF1, AGTR1, AKT1, AKT3, ANG, APELA, |
| Endothelial_Cell_Proliferation | APLN, APLNR, ARG1, ARNT, BMP2, BMP4, BMP6, BMPR2, CAV2, |
| | CCL11, CCL24, CCL26, CCR3, CDH13, CXCL12, CYBA, DYSF, |
| | ECM1, EGFL7, EGR3, EMC10, F3, GF2, FGFBP1, GFR1, LT4, |
| | GATA2, GDF2, GHRL, GHSR, HIF1A, HMGB1, HMGB2, HMOX1, |
| | HTR2B, IGF2, IL10, ITGA4, ITGB3, JCAD, JUN, KDR. LRG1. MDK. |
| | MIR101-1, MIR101-2, MIR10A, MIR10B, MIR126, MIR130A, MIR132, |
| | MIR135B, MIR146A, MIR21, MIR23A, MIR27A, MIR27B, MIR29A, |
| | MIR487B, MIR495, MIR499A, MIR503, MIRLET7B, MTOR, MYDGF, |
| | NF1, NR4A1, NRARP, NRAS, NRP1, NRP2, PDCD6, PDCL3, PDGFB, |
| | PDPK1, PGF, PLCG1, PLXNB3, PPP1R16B, PRKCA, PRKD1, |
| | PRKD2, PROX1, RICTOR, RPTOR, SCG2, SEMA5A, SIRT1, SIRT6, |
| | SP1, STAT3, STAT5A, TEK, TGFBR1, THBS4, TNFSF12, VASH2, |
| | VEGFA, VEGFB, VEGFC, VEGFD, VIP, WNT2, WNT5A, ZNF580 |
| Go_Positive_Regulation_Of_{—} | AAMP, ABL1, ADAM17, ADGRA2, AGT, AKT1, AKT3, ALOX12, |
| Endothelial_Cell_Migration | AMOTL1, ANGPT1, ANGPT4, ANXA1, ANXA3, ATOH8, ATP5F1A, |
| | ATP5F1B, BCAR1, BCAS3, BMP4, BMPR2, CALR, CCBE1, CD40, |
| | CIB1, EDN1, EMC10, ETS1, FGF1, FGF16, FGF18, FGF2, FGFBP1, |
| | FGFR1, FLT4, FOXC2, FOXP1, FUT1, GATA2, GATA3, GPI, GPLD1, |
| | GRN, HDAC7, HDAC9, HIF1A, HMGB1, HMOX1, HSPB1, ITGB1BP1, |
| | ITGB3, JCAD, KDR, LGMN, MAP2K3, MAP3K3, MET, MIR101-1, |
| | MIR101-2, MIR10A, MIR10B, MIR126, MIR132, MIR135B, MIR143, |
| | MIR146A, MIR150, MIR1908, MIR199A1, MIR199A2, MIR199B, |
| | MIR200A, MIR21, MIR210, MIR221, MIR23A, MIR27A, MIR27B, |
| | MIR296, MIR29A, MIR30A, MIR31, MIR342, MIR487B, MIR499A, |
| | MIR939, MIRLET7F1, MIRLET7F2, NFE2L2, NOS3, NRP1, NRP2, |
| | NUS1, P2RX4, PDCD6, PDGFB, PDPK1, PIK3C2A, PLCG1, PLK2, |
| | PLPP3, PRKCA, PRKD1, PRKD2, PROX1, PTGS2, PTK2B, RHOB, |
| | RHOJ, RIN2, ROCK2, RRAS, SASH1, SCARB1, SEMA5A, SIRT1, |
| | SMOC2, SP1, SPARC, SRPX2, STAT5A, TDGF1, TEK, TGFB1, |
| | THBS1, TMSB4X, TSTA3, VEGFA, VEGFC, WNT5A, WNT7A, |
| | ZC3H12A, ZNF580 |
| Go_Positive_Regulation_Of_{—} | HSPB1, KDR, PRKD1, PRKD2, VEGFA |
| Endothelial_Cell_Chemotaxis_{—} | |
| By_Vegf_Activated_Vascular_{—} | |
| Endothelial_Growth_Factor_{—} | |
| Receptor_Signaling_Pathway | |
| Go_Positive_Regulation_Of_{—} | FGF16, FGF18, FGF2, FGFR1, HSPB1, KDR, LGMN, MET, P2RX4, |
| Endothelial_Cell_Chemotaxis | PRKD1, PRKD2, SEMA5A, SMOC2, TMSB4X, VEGFA |
| Go_Positive_Regulation_Of_{—} | GAB1, HSPB1, KDR, MYO1C, PRKD1, PRKD2, VEGFA |
| Cell_Migration_By_Vascular_{—} | |
| Endothelial_Growth_Factor_{—} | |
| Signaling_Pathway | |
| Go_Positive_Regulation_Of_{—} | AGTR1, APELA, APLNR, FGFBP1, GATA2, HMOX1, JCAD, MIR101- |
| Blood_Vessel_Endothelial_Cell_{—} | 1, MIR101-2, MIR10A, MIR10B, MIR126, MIR132, MIR146A, MIR21, |
| Proliferation_Involved_In_{—} | MIR23A, MIR27A, MIR27B, MIR487B, MIR503, MIRLET7B, |
| Sprouting_Angiogenesis | PPP1R16B, VEGFA |
| Go_Positive_Regulation_Of_{—} | ADAM17, AKT1, AKT3, ALOX12, AMOTL1, ANGPT1, ANGPT4, |
| Blood_Vessel_Endothelial_{—} | ANXA1, ATP5F1A, ATP5F1B, CD40, CIB1, ETS1, FGF18, FGF2, |
| Cell_Migration | FGFBP1, FGFR1, FOXC2, GATA2, HDAC7, HDAC9, HIF1A, HMGB1, |
| | HMOX1, HSPB1, JCAD, KDR, MAP2K3, MAP3K3, MIR101-1, MIR101- |
| | 2, MIR10A, MIR10B, MIR126, MIR132, MIR135B, MIR143, MIR146A, |
| | MIR150, MIR200A, MIR210, MIR221, MIR23A, MIR27A, MIR27B, |
| | MIR296, MIR30A, MIR31, MIR342, MIR487B, MIR499A, MIR939, |
| | MIRLET7F1, MIRLET7F2, NFE2L2, NOS3, NRP1, NUS1, P2RX4, |
| | PDGFB, PDPK1, PIK3C2A, PLCG1, PLK2, PRKCA, PRKD1, PRKD2, |
| | PTGS2, RHOJ, SIRT1, SP1, SRPX2, STAT5A, TGFB1, THBS1, |
| | TMSB4X, VEGFA, VEGFC |
| Go_Leukocyte_Adhesion_To_{—} | ADD2, ALOX5, CCL21, CCL25, CCL28, CCR2, CX3CR1, CXCL12, |
| Vascular_Endothelial_Cell | ELANE, ETS1, FUT4, FUT7, FUT9, GCNT1, GOLPH3, ICAM1, IL6, |
| | IRAK1, ITGA4, ITGB1, ITGB2, ITGB7, JAM2, KLF4, LEP, MADCAM1, |
| | MDK, MIR125A, MIR141, MIR146A, MIR21, MIR221, MIR222, MIR31, |
| | MIR92A1, MIR92A2, MIRLET7E, MIRLET7G, NFAT5, PODXL2, |
| | PTAFR, RELA, RHOA, ROCK1, SELE, SELL, SELP, SELPLG, |
| | SLC39A8, SPN, TNF, TRAF6, VCAM1 |
| Go_Leukocyte_Adhesion_To_{—} | ALOX5, KLF4, MIR92A1, MIR92A2, SLC39A8, TNF |
| Arterial_Endothelial_Cell | |
| Go_Endothelial_Cell_Migration | AAMP, ABL1, ACVRL1, ADAM17, ADAMTS9, ADGRA2, ADGRB1, |
| | ADTRP, AGT, AGTR2, AKT1, AKT3, LOX12, AMOT, AMOTL1, |
| | ANGPT1, ANGPT2, ANGPT4, ANXA1, ANXA3, APOA1, APOE, APOH, |
| | ATOH8, ATP2B4, ATP5F1A, ATP5F1B, BCAR1, BCAS3, BMP10, |
| | BMP4, BMPER, BMPR2, CALR, CARD10, CCBE1, CCN3, CD40, |
| | CDH13, CDH5, CEACAM1, CIB1, CLEC14A, CORO1B, CSNK2B, |
| | CXCL13, CYP1B1, DAB2IP, DCN, DLL4, DNAJA4, DPP4, EDN1 |
| | EFNA1, EFNB2, EGR3, EMC10, EMP2, EPHA2, EPHB4, ETS1, FAP, |
| | FBXW7, FGF1, FGF16, FGF18, FGF2, FGF4, FGFBP1, FGFR1, FLT4, |
| | FOXC2, FOXP1, FUT1, GADD45A, GATA2, GATA3, GDF2, GIPC1, |
| | GLUL, GPI, GPLD1, GPX1, GREM1, GRN, HDAC5, HDAC7, HDAC9, |
| | HIF1A, HMGB1, HMOX1, HRG, HSPB1, ID1, ITGB1, ITGB1BP1, |
| | ITGB2, ITGB3, JCAD, JUP, KDR, KLF4, KRIT1, LGALS12, LGALS8, |
| | LGMN, LOXL2, LPXN, MAP2K3, MAP2K5, MAP3K3, MECP2, MEF2C, |
| | MEOX2, MET, MIA3, MIR101-1, MIR101-2, MIR10A, MIR10B, MIR126, |
| | MIR129-1, MIR129-2, MIR132, MIR133B, MIR135B, MIR137, MIR143, |
| | MIR146A, MIR149, MIR150, MIR152, MIR15A, MIR15B, MIR16-1, |
| | MIR16-2, MIR188, MIR1908, MIR193A, MIR196A1, MIR196A2, |
| | MIR199A1, MIR199A2, MIR199B, MIR19B1, MIR19B2, MIR200A, |
| | MIR200B, MIR200C, MIR204, MIR206, MIR20A, MIR21, MIR210, |
| | MIR212, MIR22, MIR221, MIR2355, MIR23A, MIR24-1, MIR24-2, |
| | MIR26A1, MIR26A2, MIR27A, MIR27B, MIR296, MIR29A, MIR29C, |
| | MIR30A, MIR31, MIR320A, MIR329-1, MIR329-2, MIR342, MIR361, |
| | MIR410, MIR424, MIR483, MIR487B, MIR492, MIR494, MIR495, |
| | MIR497, MIR499A, MIR503, MIR505, MIR640, MIR92A1, MIR92A2, |
| | MIR939, MIRLET7F1, MIRLET7F2, MMRN2, MYH9, NF1, NFE2L2, |
| | NOS3, NOTCH1, NR2E1, NR2F2, NR4A1, NRP1, NRP2, NUS1, |
| | P2RX4, PAXIP1, PDCD10, PDCD6, PDGFB, PDPK1, PECAM1, |
| | PIK3C2A, PIK3CA, PIK3R3, PLCG1, PLEKHG5, PLK2, PLPP3, |
| | PLXND1, PPARG, PRCP, PRKCA, PRKD1, PRKD2, PRKX, PROX1, |
| | PRSS3, PRSS3P2, PTEN, PTGS2, PTK2, PTK2B, PTN, PTP4A3, |
| | PTPRM, PXN, RAB13, RGCC, RHOA, RHOB, RHOJ, RIN2, ROBO1, |
| | ROCK2, RRAS, S100A2, S100P, SASH1, SCARB1, SCG2, SEMA4A, |
| | SEMA5A, SERPINF1, SH3BP1, SIRT1, SLIT2, SMOC2, SOX18, SP1, |
| | SP100, SPARC, SPRED1, SRF, SRPX2, STARD13, STAT5A, STC1, |
| | SVBP, SYNJ2BP, TBXA2R, TDGF1, TEK, TGFB1, TGFBR1, THBS1, |
| | TMSB4X, TNF, TNFSF12, TSTA3, VASH1, VEGFA, VEGFC, WNT5A, |
| | WNT7A, ZC3H12A, ZNF580 |
| Go_Endothelial_Cell_Chemotaxis | CCN3, CORO1B, CXCL13, EGR3, FGF1, FGF16, FGF18, FGF2, |
| | FGF4, GFR1, HRG, HSPB1, KDR, LGMN, MET, MIR149, MIR16-1, |
| | MIR16-2, MIR424, NOTCH1, NR4A1, NRP1, P2RX4, PLEKHG5, |
| | PRKD1, PRKD2, RAB13, SEMA5A, SMOC2, THBS1, TMSB4X, |
| | VEGFA |
| Go_Endothelial_Cell_Activation | APOLD1, BMPER, CXCL10, FOXP1, HOXA9, MIR92A1, MIR92A2, |
| | P2RX4, PRMT5, SMAD4, TCIM, TGFBR1 |
| Go_Blood_Vessel_Endothelial_Cell_Migration | ABL1, ACVRL1, ADAM17, ADTRP, AGTR2, AKT1, AKT3, ALOX12, |
| | AMOT, AMOTL1, ANGPT1, ANGPT2, ANGPT4, ANXA1, APOA1, |
| | APOE, ATP2B4, ATP5F1A, ATP5F1B, CARD10, CD40, CDH5, CIB1, |
| | CLEC14A, CSNK2B, DLL4, EFNA1, EFNB2, EGR3, EMP2, EPHA2, |
| | EPHB4, ETS1, FBXW7, FGF18, FGF2, FGFBP1, FGFR1, FOXC2, |
| | GADD45A, GATA2, GDF2, GPLD1, GPX1, GREM1, HDAC5, HDAC7, |
| | HDAC9, HIF1A, HMGB1, HMOX1, HRG, HSPB1, ID1, ITGB1, |
| | ITGB1BP1, JCAD, JUP, KDR, KLF4, MAP2K3, MAP2K5, MAP3K3, |
| | MECP2, MEF2C, MEOX2, MIA3, MIR101-1, MIR101-2, MIR10A, |
| | MIR10B, MIR126, MIR129-1, MIR129-2, MIR132, MIR133B, MIR135B, |
| | MIR137, MIR143, MIR146A, MIR149, MIR150. MIR152, MIR15A, |
| | MIR15B, MIR16-1, MIR16-2, MIR188, MIR193A, MIR196A1, |
| | MIR196A2, MIR199A1, MIR199A2, MIR199B, MIR19B1, MIR19B2, |
| | MIR200A, MIR200B, MIR200C, MIR204, MIR206, MIR20A, MIR210, |
| | MIR212, MIR22, MIR221, MIR2355, MIR23A, MIR24-1, MIR24-2, |
| | MIR26A1, MIR26A2, MIR27A, MIR27B, MIR296, MIR29C, MIR30A, |
| | MIR31, MIR320A, MIR329-1, MIR329-2, MIR342, MIR361, MIR410, |
| | MIR424, MIR483, MIR487B, MIR492, MIR494, MIR495, MIR497, |
| | MIR499A, MIR503, MIR505, MIR640, MIR92A1, MIR92A2, MIR939, |
| | MIRLET7F1, MIRLET7F2, MMRN2, MYH9, NF1, NFE2L2, NOS3, |
| | NOTCH1, NR2E1, NR4A1, NRP1, NUS1, P2RX4, PDCD10, PDGFB, |
| | PDPK1, PIK3C2A, PIK3R3, PLCG1, PLK2, PPARG, PRCP, PRKCA, |
| | PRKD1, PRKD2, PTGS2, PTK2B, RGCC, RHOA, RHOJ, ROBO1, |
| | SCARB1, SH3BP1, SIRT1, SLIT2, SOX18, SP1, SPRED1, SRF, |
| | SRPX2, STARD13, STAT5A, TBXA2R, TDGF1, TGFB1, THBS1, |
| | TMSB4X, TNF, VASH1, VEGFA, VEGFC |
| Go_T_Cell_Chemotaxis | ADAM10, ADAM17, CCL21, CCL26, CCL27, CCL3, CCL5, CCR2, |
| | CXCL10, CXCL11, CXCL13, CXCL16, CXCR3, DEFA1, DEFA1B, |
| | GPR183, OXSR1, PIK3CD, PIK3CG, S100A7, SLC12A2, STK39, |
| | TMEM102, TNFSF14, WNK1, WNT5A, XCL1, XCL2 |
| Go_T_Cell_Mediated_Immunity | AGER, AIRE, ARG1, AZGP1, B2M, BTN3A2, BTN3A3, CCR2, CD1A, |
| | CD1B, CD1C, CD1D, CD1E, CD46, CD55, CD70, CD81, CD8A, |
| | CEACAM1, CLC, CLEC4G, CTSC, CTSH, CYRIB, DENND1B, DLG1, |
| | DUSP22, EMP2, FADD, FBXO38, FCGR2B, FOXP3, FUT7, FZD5, |
| | GATA3, GNL1, GZMM, HFE, HLA-A, HLA-B, HLA-E, HLA-F, HLA-G, |
| | HLA-H, HMGB1, HPRT1, HSPD1, ICAM1, IFNA2, IFNB1, IL12A, |
| | IL12B, IL12RB1, IL18, IL18R1, IL18RAP, IL1B, IL1R1, IL20RB, IL23A, |
| | IL23R, IL31RA, IL4, IL6, IL7R, JAG1, KDELR1, LILRB1, MALT1, |
| | MAP3K7, MICA, MR1, MYO1G, NECTIN2, NLRP3, P2RX7, PPP3CB, |
| | PRF1, PRKCZ, PTPRC, PVR, RAB27A, RFTN1, RIPK3, RSAD2, |
| | SASH3, SCART1, SLC11A1, SLC22A13, SMAD7, STARD7, STX7, |
| | TBX21, TNFRSF1B, TNFSF4, TRAF2, TRAF6, TREX1, TRPM4, |
| | TSTA3, WAS, XCL1, ZBTB1, ZP3 |
| Go_T_Cell_Mediated_Immune_Response_To_Tumor_Cell | FBXO38, HLA-A, HMGB1, HSPD1, MR1, SLC22A13 |
| Go_T_Cell_Mediated_Cytotoxicity | AGER, AZGP1, B2M, CD1A, CD1B, CD1C, CD1D, CD1E, CEACAM1, |
| | CTSC, CTSH, CYRIB, EMP2, FADD, FCGR2B, GZMM, HLA-A, HLA-B, |
| | HLA-E, HLA-F, HLA-G, HLA-H, HPRT1, IL12A, IL12B, IL12RB1, IL23A, |
| | IL23R, IL7R, LILRB1, MICA, MR1, NECTIN2, P2RX7, PPP3CB, PRF1, |
| | PTPRC, PVR, RAB27A, RIPK3, SLC22A13, STX7, TSTA3, XCL1 |
| Go_T_Cell_Extravasation | CCL2, CCR2, CD99, CD99L2, CRK, CRKL, F11R, FADD, ICAM1, |
| | IL27RA, ITGAL, RIPK3, XG |
| Go_T_Cell_Differentiation_{—} | ANXA1, ATP7A, BATF, BCL3, BCL6, CCL19, CD46, CD80, CD86, |
| Involved_In_Immune_Response | CLEC4D, CLEC4E, EOMES, FCER1G, FGL2, FOXP1, FOXP3, |
| | GATA3, GPR183, HLX, HMGB1, IFNG, IFNL1, IL12B, IL12RB1, IL18, |
| | IL18R1, IL2, IL21, IL23A, IL23R, IL27, IL4, IL4R, IL6, IRF4, JAK3, |
| | LEF1, LGALS9, LOXL3, LY9, MALT1, MIR21, MTOR, MYB, NFKBID, |
| | NFKBIZ, NLRP3, PCK1, PRKCZ, PTGER4, RARA, RC3H1, RC3H2, |
| | RELB, RIPK2, RORA, RORC, SEMA4A, SLAMF6, SMAD7, SOCS5, |
| | SPN, STAT3, STAT6, TBX21, TMEM98, TNFSF4, TSC1, ZBTB7B, |
| | ZC3H12A, ZFPM1 |
| Go_T_Cell_Apoptotic_Process | ADA, ADAM8, AKT1, ARG2, BAK1, BAX, BCL10, BCL2L11, BMP4, |
| | CCL5, CD27, CD274, CLC, DFFA, DNAJA3, DOCK8, EFNA1, FADD, |
| | FAS, FASLG, GIMAP8, GLI3, GPAM, HIF1A, IDO1, IL2RA, IL7R, |
| | JAK3, KDELR1, LGALS13, LGALS14, LGALS16, LGALS3, LGALS9, |
| | LMBR1L, NFKBID, PDCD1, PIP, PRELID1, PRKCQ, PTCRA, RAG1, |
| | RIPK1, RIPK3, SLC46A2, TP53, TSC22D3, WNT5A, ZC3H8 |
| Go_T_Cell_Antigen_Processing_And_Presentation | HFE, ICAM1, RFTN1, TREX1, WAS |
| Go_T_Cell_Activation_Involved_In_Immune_Response | ANXA1, APBB1IP, ATP7A, BATF, BCL3, BCL6, CCL19, CD1C, CD46, |
| | CD74, CD80, CD81, CD86, CEACAM1, CLEC4D, CLEC4E, EIF2AK4, |
| | EOMES, F2RL1, FCER1G, FCGR2B, FGL2, FOXP1, FOXP3, GATA3, |
| | GPR183, HAVCR2, HLA-DMB, HLX, HMGB1, ICAM1, IFNA1, IFNA10, |
| | IFNA13, IFNA14, IFNA16, IFNA17, IFNA2, IFNA21, IFNA4, IFNA5, |
| | IFNA6, IFNA7, IFNA8, IFNB1, IFNE, IFNG, IFNK, IFNL1, IFNW1, |
| | IL12B, IL12RB1, IL18, IL18R1, IL2, IL21, IL23A, IL23R, IL27, IL4, IL4R, |
| | IL6, IRF4, ITGAL, JAK3, LCP1, LEF1, LGALS3, LGALS9, LILRB1, |
| | LOXL3, LY9, MALT1, MDK, MIR21, MTOR, MYB, NFKBID, NFKBIZ, |
| | NLRP3, PCK1, PRKCZ, PSEN1, PTGER4, RAB27A, RARA, RC3H1, |
| | RC3H2, RELB, RIPK2, RORA, RORC, SEMA4A, SLAMF6, SLC11A1, |
| | SMAD7, SOCS5, SPN, STAT3, STAT6, TBX21, TMEM98, TNFSF18, |
| | TNFSF4, TP53, TSC1, ZBTB7B, ZC3H12A, ZFPM1 |
| Go_Regulation_Of_T_Cell_Receptor_{—} | ADA, BCL10, BTN2A2, CACNA1F, CARD11, CCR7, CD160, CD226, |
| Signaling_Pathway | CD300A, CD81, CEACAM1, CYLD, DGKZ, DUSP22, DUSP3, ELF1, |
| | EZR, GBP1, KCNN4, LCK, LGALS3, LILRB4, MALT1, NECTIN2, |
| | PAWR, PHPT1, PRKD2, PRNP, PTPN2, PTPN22, PTPN6, PTPRJ, |
| | PVRIG, RAB29, RC3H1, RELA, RPS3, SH2D1A, SLA2, TESPA1, |
| | THY1, TRAT1, UBASH3A |
| Go_Regulation_Of_T_Cell_Migration | ADAM10, ADAM17, ADAM8, AIF1, AIRE, APOD, APP, C10orf99, |
| | CCL20, CCL21, CCL27, CCL5, CCR2, CCR6, CD200, CD200R1, |
| | CD99L2, CRK, CRKL, CXCL10, CXCL12, CXCL13, DOCK8, ECM1, |
| | FADD, IL27RA, ITGA4, LRCH1, OXSR1, PYCARD, RHOA, RIPK3, |
| | RIPOR2, S100A7, SELENOK, SPN, STK39, TMEM102, TNFRSF14, |
| | TNFSF14, WNK1, WNT5A, XCL1, XCL2 |
| Go_Regulation_Of_T_Cell_Mediated_{—} | ARG1, AZGP1, B2M, CCR2, CD1A, CD1B, CD1C, CD1D, CD1E, |
| Immunityager | CD81, CEACAM1, CLC, CLEC4G, CYRIB, DUSP22, FADD, FBXO38, |
| | FCGR2B, FOXP3, FUT7, FZD5, GATA3, HFE, HLA-A, HLA-B, HLA-E, |
| | HLA-F, HLA-G, HLA-H, HMGB1, HSPD1, IFNA2, IFNB1, IL12A, IL12B, |
| | IL12RB1, IL18, IL18R1, IL1B, IL1R1, IL20RB, IL23A, IL23R, IL6, IL7R, |
| | LILRB1, MALT1, MAP3K7, MR1, NECTIN2, NLRP3, P2RX7, PPP3CB, |
| | PRKCZ, PTPRC, PVR, RIPK3, RSAD2, SASH3, SLC22A13, SMAD7, |
| | STX7, TBX21, TNFRSF1B, TNFSF4, TRAF2, TRAF6, TRPM4, WAS, |
| | XCL1, ZBTB1, ZP3 |
| Go_Regulation_Of_T_Cell_Mediated_{—} | FBXO38, HMGB1, HSPD1, MR1, SLC22A13 |
| Immune_Response_To_Tumor_Cell | |
| Go_Regulation_Of_T_Cell_Mediated_{—} | AGER, AZGP1, B2M, CD1A, CD1B, CD1C, CD1D, CD1E, CEACAM1, |
| Cytotoxicity | CYRIB, FADD, FCGR2B, HLA-A, HLA-B, HLA-E, HLA-F, HLA-G, HLA- |
| | H, IL12A, IL12B, IL12RB1, IL23A, IL23R, IL7R, LILRB1, MR1, |
| | NECTIN2, P2RX7, PPP3CB, PTPRC, PVR, RIPK3, SLC22A13, STX7, |
| | XCL1 |
| Go_Regulation_Of_T_Cell_Extravasation | CCR2, CD99L2, FADD, IL27RA, RIPK3 |
| Go_Regulation_Of_T_Cell_Differentiation | ABL1, ADA, ADAM8, ANXA1, AP3B1, AP3D1, BAD, BCL6, BMP4, |
| | BTN2A2, CAMK4, CARD11, CBFB, CCL19, CCR2, CD2, CD27, CD28, |
| | CD46, CD74, CD80, CD83, CD86, CLPTM1, CR1, CRTAM, CTLA4, |
| | CYLD, CYP26B1, DROSHA, DTX1, DUSP10, EGR3, ERBB2, FANCA, |
| | FANCD2, FGL2, FOXJ1, FOXN1, FOXP3, GATA3, GLI2, GLI3, HLA- |
| | DOA, HLA-G, HLX, HMGB1, IFNA2, IFNB1, IFNG, IFNL1, IHH, IL12A, |
| | IL12B, IL12RB1, IL15, IL18, IL1A, IL1B, IL1RL2, IL2, IL23A, IL23R, |
| | IL27, IL2RA, IL36B, IL4, IL4R, IL7, IL7R, IRF1, IRF4, ITPKB, JAK3, |
| | KAT2A, LAG3, LEF1, LGALS9, LILRB2, LILRB4, LOXL3, MALT1, |
| | MDK, METTL3, MIR21, MIR30B, MYB, NCKAP1L, NFATC2, NFKBID, |
| | NFKBIZ, NKAP, NLRP3, NRARP, PCK1, PIK3R6, PNP, PRDM1, |
| | PRDX2, PRELID1, PRKCZ, PTPN2, PTPRC, RAG1, RARA, |
| | RASGRP1, RC3H1, RC3H2, RHOA, RHOH, RIPK2, RUNX1, RUNX3, |
| | SART1, SASH3, SH3RF1, SHH, SLC46A2, SMAD7, SOCS1, SOCS5, |
| | SOD1, SOS1, SOX12, SOX13, SOX4, SPINK5, STAT5B, SYK, TBX21, |
| | TCF7, TESPA1, TGFBR2, TMEM131L, TNFRSF18, TNFSF4, TNFSF9, |
| | TOX, VNN1, VSIR, XBP1, ZAP70, ZBTB1, ZBTB16, ZBTB7B, |
| | ZC3H12A, ZC3H8, ZEB1, ZMIZ1, ZNF683 |
| Go_Regulation_Of_T_Cell_Cytokine_{—} | B2M, CCR2, CD81, CLC, FOXP3, FZD5, GATA3, HFE, HLA-A, HLA-F, |
| Productionarg1 | IFNA2, IFNB1, IL18, IL18R1, IL1B, IL1R1, IL6, MALT1, MAP3K7, |
| | NLRP3, PRKCZ, RSAD2, SASH3, SMAD7, TBX21, TNFRSF1B, |
| | TNFSF4, TRAF2, TRAF6, TRPM4, XCL1 |
| Go_Regulation_Of_T_Cell_Chemotaxis | ADAM10, ADAM17, CCL21, CCL27, CCL5, CCR2, CXCL10, CXCL13, |
| | OXSR1, S100A7, STK39, TMEM102, TNFSF14, WNK1, WNT5A, |
| | XCL1, XCL2 |
| Go_Regulation_Of_T_Cell_Activation_{—} | CD81, HAVCR2, HLA-DMB, LGALS3, LGALS9, LILRB1 |
| Via_T_Cell_Receptor_Contact_With_{—} | |
| Antigen_Bound_To_Mhc_Molecule_On_{—} | |
| Antigen_Presenting_Cell | |
| Go_Regulation_Of_T_Cell_Activation | ABL1, ADA, ADAM8, ADORA2A, AGER, AIF1, AKT1, ANXA1, AP3B1, |
| | AP3D1, ARG1, ARG2, BAD, BCL10, BCL6, BMP4, BTLA, BTN2A2, |
| | CAMK4, CARD11, CASP3, CAV1, CBFB, CCDC88B, CCL19, CCL2, |
| | CCL21, CCL5, CCR2, CCR7, CD160, CD1D, CD2, CD209, CD24, |
| | CD27, CD274, CD276, CD28, CD300A, CD3E, CD4, CD40LG, CD46, |
| | CD47, CD5, CD55, CD6, CD70, CD74, CD80, CD81, CD83, CD86, |
| | CDC42, CEACAM1, CEBPB, CGAS, CLC, CLEC4G, CLECL1, |
| | CLPTM1, CORO1A, CR1, CRTAM, CSK, CTLA4, CTNNB1, CYLD, |
| | CYP26B1, CYRIB, DLG1, DLG5, DNAJA3, DOCK8, DPP4, DROSHA, |
| | DTX1, DUSP10, DUSP22, DUSP3, EBI3, EFNB1, EFNB2, EFNB3, |
| | EGR3, EPO, ERBB2, FADD, FANCA, FANCD2, FCGR2B, FGL1, |
| | FGL2, FLOT2, FOXJ1, FOXN1, FOXP3, FYN, GATA3, LI2, GLI3, |
| | GLMN, GNRH1, GPAM, GPNMB, GRAP2, GRB2, GSN, HAVCR2, |
| | HES1, HFE, HHLA2, HLA-A, HLA-DMB, HLA-DOA, HLA-DPA1, HLA- |
| | DPB1, HLA-DRB1, HLA-E, HLA-G, HLX, HMGB1, HSPD1, HSPH1, |
| | ICOS, ICOSLG, IDO1, IFNA2, IFNB1, IFNG, IFNL1, IGF1, IGF2, |
| | IGFBP2, IHH, IL10, IL12A, IL12B, IL12RB1, IL15, IL18, IL1A, IL1B, |
| | IL1RL2, IL2, IL20RB, IL21, IL23A, IL23R, IL27, IL27RA, IL2RA, IL36B, |
| | IL4, IL4R, IL6, IL6ST, IL7, IL7R, IRF1, IRF4, ITPKB, JAK3, KAT2A, |
| | KLRC4-KLRK1, KLRK1, LAG3, LAT, LAX1, LCK, LEF1, LEP, LGALS1, |
| | LGALS3, LGALS7B, LGALS9, LGALS9B, LGALS9C, LILRB1, LILRB2, |
| | LILRB4, LMO1, LOXL3, LRRC32, LYN, MAD1L1, MALT1, MAP3K8, |
| | MAPK8IP1, MARCHF7, MDK, METTL3, MIR181C, MIR21, MIR27A, |
| | MIR30B, MYB, NCK1, NCK2, NCKAP1L, NDFIP1, NFATC2, NFKBID, |
| | NFKBIZ, NKAP, NLRP3, NOD2, NRARP, PAG1, PAK1, PAK2, PAK3, |
| | PAWR, PCK1, PDCD1, PDCD1LG2, PDE5A, PDPK1, PELI1, PIK3CA, |
| | PIK3R1, PIK3R6, PLA2G2D, PLA2G2F, PNP, PRDM1, PRDX2, |
| | PRELID1, PRKAR1A, PRKCQ, PRKCZ, PRNP, PTPN11, PTPN2, |
| | PTPN22, PTPN6, PTPRC, PYCARD, RAC1, RAC2, RAG1, RARA, |
| | RASAL3, RASGRP1, RC3H1, RC3H2, RHOA, RHOH, RIPK2, RIPK3, |
| | RIPOR2, RPS3, RUNX1, RUNX3, SART1, SASH3, SCGB1A1, SCRIB, |
| | SDC4, SELENOK, SFTPD, SH3RF1, SHH, SIRPA, SIRPB1, SIRPG, |
| | SIT1, SLC46A2, SLC7A1, SMAD7, SOCS1, SOCS5, SOCS6, SOD1, |
| | SOS1, SOX12, SOX13, SOX4, SPINK5, SPN, SPTA1, SRC, STAT5B, |
| | SYK, TARM1, TBX21, TCF7, TESPA1, TFRC, TGFBR2, THY1, TIGIT, |
| | TMEM131L, TMIGD2, TNFAIP8L2, TNFRSF13C, TNFRSF14, |
| | TNFRSF18, TNFRSF1B, TNFRSF21, TNFSF11, TNFSF13B, |
| | TNFSF14, TNFSF18, TNFSF4, TNFSF8, TNFSF9, TOX, TRAF6, |
| | TREX1, TWSG1, VAV1, VCAM1, VNN1, VSIG4, VSIR, VTCN1, XBP1, |
| | XCL1, YES1, ZAP70, ZBTB1, ZBTB16, ZBTB7B, ZC3H12A, ZC3H8, |
| | ZEB1, ZMIZ1, ZNF683, ZP3, ZP4 |
| Go_Regulation_Of_Gamma_Delta_T_{—} | EGR3, LEF1, LILRB1, NCKAP1L, NOD2, PTPRC, SOX13, SOX4, |
| Cell_Activation | STAT5B, SYK, TCF7 |
| Go_Regulation_Of_Cd8_Positive_{—} | CBFB, NCKAP1L, RUNX1, RUNX3, SOCS1, ZBTB7B |
| Alpha_Beta_T_Cell_Differentiation | |
| Go_Regulation_Of_Cd4_Positive_{—} | ANXA1, BCL6, CBFB, CCL19, CD80, CD83, CD86, FOXP3, GATA3, |
| Alpha_Beta_T_Cell_Differentiation | HLX, HMGB1, IFNG, IL12B, IL12RB1, IL18, IL2, IL23A, IL23R, IL27, |
| | IL4, IL4R, IRF4, JAK3, LGALS9, LOXL3, MALT1, MIR21, MYB, |
| | NCKAP1L, NFKBID, NFKBIZ, NLRP3, PRKCZ, RARA, RC3H1, |
| | RC3H2, RIPK2, RUNX1, RUNX3, SASH3, SH3RF1, SMAD7, SOCS1, |
| | SOCS5, TBX21, TNFSF4, ZBTB7B, ZC3H12A |
| Go_Regulation_Of_Cd4_Positive_{—} | AGER, ANXA1, ARG2, BCL6, CBFB, CCL19, CD160, CD274, CD55, |
| Alpha_Beta_T_Cell_Activation | CD80, CD81, CD83, CD86, FOXP3, GATA3, HLX, HMGB1, IFNG, |
| | IL12B, IL12RB1, IL18, IL2, IL23A, IL23R, IL27, IL4, IL4R, IRF4, JAK3, |
| | LGALS7B, LGALS9, LGALS9B, LGALS9C, LOXL3, MALT1, MIR21, |
| | MYB, NCKAP1L, NFKBID, NFKBIZ, NLRP3, PRKCQ, PRKCZ, RARA, |
| | RC3H1, RC3H2, RIPK2, RUNX1, RUNX3, SASH3, SH3RF1, SMAD7, |
| | SOCS1, SOCS5, TARM1, TBX21, TGFBR2, TNFSF4, TWSG1, VSIR, |
| | XCL1, ZBTB7B, ZC3H12A |
| Go_Regulation_Of_Cd8_Positive_{—} | CBFB, CD274, CRTAM, HFE, HLA-A, HLA-E, IRF1, LILRB1, |
| Alpha_Beta_T_Cell_Activation | MAPK8IP1, NCKAP1L, PTPN22, RUNX1, RUNX3, SH3RF1, SOCS1, |
| | VSIR, XCL1, ZBTB7B |
| Go_Regulation_Of_Alpha_Beta_T_{—} | ADA, ANXA1, AP3B1, AP3D1, BCL6, CBFB, CCL19, CD80, CD83, |
| Cell_Differentiation | CD86, FOXP3, GATA3, GLI3, HLX, HMGB1, IFNG, IHH, IL12B, |
| | IL12RB1, IL18, IL2, IL23A, IL23R, IL27, IL4, IL4R, IRF4, ITPKB, JAK3, |
| | LGALS9, LOXL3, MALT1, MIR21, MYB, NCKAP1L, NFKBID, NFKBIZ, |
| | NKAP, NLRP3, PNP, PRDM1, PRKCZ, RARA, RC3H1, RC3H2, |
| | RHOA, RIPK2, RUNX1, RUNX3, SASH3, SH3RF1, SHH, SMAD7, |
| | SOCS1, SOCS5, SYK, TBX21, TGFBR2, TNFSF4, ZAP70, ZBTB16, |
| | ZBTB7B, ZC3H12A, ZNF683 |
| Go_Regulation_Of_Alpha_Beta_T_{—} | ADA, ADORA2A, AGER, ANXA1, AP3B1, AP3D1, ARG2, BCL6, |
| Cell_Activation | CBFB, CCL19, CCR2, CD160, CD274, CD28, CD300A, CD3E, CD55, |
| | CD80, CD81, CD83, CD86, CRTAM, EBI3, FOXP3, GATA3, GLI3, |
| | HFE, HLA-A, HLA-E, HLX, HMGB1, HSPH1, IFNG, IHH, IL12A, IL12B, |
| | IL12RB1, IL18, IL2, IL23A, IL23R, IL27, IL4, IL4R, IRF1, IRF4, ITPKB, |
| | JAK3, LGALS7B, LGALS9, LGALS9B, LGALS9C, LILRB1, LOXL3, |
| | MALT1, MAPK8IP1, MIR21, MYB, NCKAP1L, NFKBID, NFKBIZ, |
| | NKAP, NLRP3, PNP, PRDM1, PRKCQ, PRKCZ, PTPN22, PTPRC, |
| | RARA, RASAL3, RC3H1, RC3H2, RHOA, RIPK2, RUNX1, RUNX3, |
| | SASH3, SH3RF1, SHH, SMAD7, SOCS1, SOCS5, SYK, TARM1, |
| | TBX21, TGFBR2, TNFRSF14, TNFSF4, TWSG1, VSIR, XCL1, ZAP70, |
| | ZBTB16, ZBTB7B, ZC3H12A, ZNF683 |
| Go_Positive_T_Cell_Selection | BATF, BCL11B, BCL2, CD3D, CD3E, CD3G, CD74, CTSL, CYLD, |
| | DOCK2, FOXN1, FOXP3, IL12B, IL12RB1, IL23A, IL23R, IL6, IRF4, |
| | ITPKB, LOXL3, LY9, MTOR, PTPN2, PTPRC, SHH, SLAMF6, SPN, |
| | SRF, STAT3, STAT6, STK11, TBX21, THEMIS, TOX, ZAP70, ZFPM1 |
| Go_Positive_Regulation_Of_T_{—} | CD3E, FOXP3, HLA-G, IDO1, LILRB2, TGFBR2 |
| Cell_Tolerance_Induction | |
| Go_Positive_Regulation_Of_T_{—} | ADA, CARD11, CCR7, CD226, CD81, CYLD, KCNN4, LCK, NECTIN2, |
| Cell_Receptor_Signaling_Pathway | PRKD2, RAB29, RELA, RPS3, TESPA1, TRAT1 |
| Go_Positive_Regulation_Of_T_{—} | AGER, AIF1, ANXA1, CARD11, CCDC88B, CCL19, CCL5, CCR2, |
| Cell_Proliferation | CD1D, CD209, CD24, CD274, CD276, CD28, CD3E, CD4, CD40LG, |
| | CD46, CD55, CD6, CD70, CD80, CD81, CD86, CLECL1, CORO1A, |
| | DNAJA3, EBI3, EFNB1, EPO, FADD, FOXP3, GPAM, HAVCR2, HES1, |
| | HHLA2, HLA-A, HLA-DMB, HLA-DPA1, HLA-DPB1, HLA-E, HMGB1, |
| | ICOSLG, IGF1, IGF2, IGFBP2, IL12A, IL12B, IL12RB1, IL15, IL18, |
| | IL1A, IL1B, IL2, IL21, IL23A, IL23R, IL27RA, IL2RA, IL4, IL6, IL6ST, |
| | JAK3, LEP, LGALS9, LILRB2, MIR21, MIR30B, NCK1, NCK2, |
| | NCKAP1L, PDCD1LG2, PNP, PRKCQ, PTPN22, PTPRC, PYCARD, |
| | RASAL3, RIPK2, RPS3, SASH3, SELENOK, SHH, SLC7A1, SPTA1, |
| | STAT5B, SYK, TFRC, TGFBR2, TMIGD2, TNFRSF13C, TNFSF13B, |
| | TNFSF4, TNFSF9, TRAF6, VCAM1, VTCN1, XCL1, ZAP70, ZP3, ZP4 |
| Go_Positive_Regulation_Of_T_{—} | ADAM10, ADAM17, ADAM8, AIF1, APP, CCL20, CCL21, CCL27, |
| Cell_Migration | CCL5, CCR2, CD99L2, CXCL10, CXCL12, CXCL13, DOCK8, FADD, |
| | ITGA4, OXSR1, PYCARD, RHOA, S100A7, SELENOK, SPN, STK39, |
| | TMEM102, TNFRSF14, TNFSF14, WNK1, WNT5A, XCL1, XCL2 |
| Go_Positive_Regulation_Of_T_{—} | AZGP1, B2M, CD1A, CD1B, CD1C, CD1D, CD1E, CD81, CYRIB, |
| Cell_Mediated_Immunity | FADD, FBXO38, FOXP3, FZD5, GATA3, HLA-A, HLA-B, HLA-E, HLA- |
| | F, HLA-G, HLA-H, HSPD1, IL12A, IL12B, IL12RB1, IL18, IL18R1, IL1B, |
| | IL1R1, IL23A, IL23R, IL6, MALT1, MAP3K7, MR1, NECTIN2, NLRP3, |
| | P2RX7, PRKCZ, PTPRC, PVR, RSAD2, SASH3, SLC22A13, STX7, |
| | TNFSF4, TRAF2, TRAF6, XCL1, ZBTB1, ZP3 |
| Go_Positive_Regulation_Of_T_{—} | AZGP1, B2M, CD1A, CD1B, CD1C, CD1D, CD1E, CYRIB, FADD, |
| Cell_Mediated_Cytotoxicity | HLA-A, HLA-B, HLA-E, HLA-F, HLA-G, HLA-H, IL12A, IL12B, IL12RB1, |
| | IL23A, IL23R, MR1, NECTIN2, P2RX7, PTPRC, PVR, SLC22A13, |
| | STX7, XCL1 |
| Go_Positive_Regulation_Of_T_{—} | B2M, CD81, FZD5, GATA3, HLA-A, IL18, IL18R1, IL1B, IL1R1, IL6, |
| Cell_Cytokine_Production | MALT1, MAP3K7, NLRP3, PRKCZ, RSAD2, SASH3, TNFSF4, TRAF2, |
| | TRAF6, XCL1 |
| Go_Positive_Regulation_Of_{—} | BCL6, BTN2A2, CD46, DUSP10, FOXP3, HLA-G, IFNG, IL2, LGALS9, |
| Regulatory_T_Cell_Differentiation | LILRB2, LILRB4, SOCS1, SOX12, VSIR |
| Go_Positive_Regulation_Of_{—} | FOXP3, IL1A, IL1B, RIPK2, SHH |
| Immature_T_Cell_Proliferation | |
| Go_Positive_Regulation_Of_{—} | LEF1, NCKAP1L, PTPRC, SOX13, SOX4, STAT5B, SYK |
| Gamma_Delta_T_Cell_Differentiation | |
| Go_Positive_Regulation_Of_{—} | LEF1, LILRB1, NCKAP1L, NOD2, PTPRC, SOX13, SOX4, STAT5B, |
| Gamma_Delta_T_Cell_Activation | SYK |
| Go_Positive_Regulation_Of_{—} | ADA, ANXA1, AP3B1, AP3D1, CBFB, CCL19, CD80, CD83, CD86, |
| Alpha_Beta_T_Cell_Differentiation | FOXP3, GLI3, HLX, IFNG, IHH, IL12B, IL12RB1, IL18, IL23A, IL23R, |
| | IL4R, ITPKB, LGALS9, MALT1, MIR21, MYB, NCKAP1L, NFKBID, |
| | NFKBIZ, NKAP, NLRP3, PNP, PRKCZ, RARA, RHOA, RIPK2, RUNX1, |
| | RUNX3, SASH3, SHH, SOCS1, SOCS5, SYK, TGFBR2, TNFSF4, |
| | ZAP70, ZBTB16, ZBTB7B |
| Go_Positive_Regulation_Of_{—} | ADA, ANXA1, AP3B1, AP3D1, CBFB, CCL19, CCR2, CD160, CD28, |
| Alpha_Beta_T_Cell_Activation | CD3E, CD55, CD80, CD81, CD83, CD86, EBI3, FOXP3, GLI3, HLA-A, |
| | HLA-E, HLX, HSPH1, IFNG, IHH, IL12A, IL12B, IL12RB1, IL18, IL23A, |
| | IL23R, IL4R, ITPKB, LGALS9, MALT1, MIR21, MYB, NCKAP1L, |
| | NFKBID, NFKBIZ, NKAP, NLRP3, PNP, PRKCQ, PRKCZ, PTPN22, |
| | PTPRC, RARA, RASAL3, RHOA, RIPK2, RUNX1, RUNX3, SASH3, |
| | SHH, SOCS1, SOCS5, SYK, TGFBR2, TNFSF4, XCL1, ZAP70, |
| | ZBTB16, ZBTB7B |
| Go_Positive_Regulation_Of_{—} | AGER, CD24, EPO, FADD, GPAM, HHLA2, HMGB1, ICOSLG, IGF1, |
| Activated_T_Cell_Proliferation | IGF2, IGFBP2, IL12B, IL12RB1, IL18, IL2, IL23A, IL23R, IL27RA, |
| | IL2RA, MIR21, MIR30B, PYCARD, RPS3, STAT5B, TMIGD2, TNFSF9 |
| Go_T_Cell_Activation | ABL1, ADA, ADAM17, ADAM8, ADORA2A, AGER, AIF1, AIRE, AKT1, |
| | ANXA1, AP3B1, AP3D1, APBB1IP, ARG1, ARG2, ATG5, ATP7A, |
| | AZI2, B2M, BAD, BATF, BAX, BCL10, BCL11B, BCL2, BCL3, BCL6, |
| | BMP4, BTLA, BTN2A2, BTN3A1, CAMK4, CARD11, CASP3, CASP8, |
| | CAV1, CBFB, CCDC88B, CCL19, CCL2, CCL21, CCL5, CCND3, |
| | CCR2, CCR6, CCR7, CCR9, CD151, CD160, CD1C, CD1D, CD2, |
| | CD209, CD24, CD27, CD274, CD276, CD28, CD300A, CD3D, CD3E, |
| | CD3G, CD4, CD40LG, CD44, CD46, CD47, CD5, CD55, CD6, CD7, |
| | CD70, CD74, CD80, CD81, CD83, CD86, CD8A, CD8B, CDC42, |
| | CDH26, CDK6, CEACAM1, CEBPB, CGAS, CHD7, CLC, CLEC4A, |
| | CLEC4D, CLEC4E, CLEC4G, CLEC7A, CLECL1, CLPTM1, CORO1A, |
| | CR1, CRTAM, CSK, CTLA4, CTNNB1, CTPS1, CTSL, CXADR, CYLD, |
| | CYP26B1, CYRIB, DDOST, DLG1, DLG5, DLL4, DNAJA3, DOCK2, |
| | DOCK8, DPP4, DROSHA, DTX1, DUSP10, DUSP22, DUSP3, EBI3, |
| | EFNB1, EFNB2, EFNB3, EGR1, EGR3, EIF2AK4, ELF4, EOMES, |
| | EPO, ERBB2, F2RL1, FADD, FANCA, FANCD2, FCER1G, FCGR2B, |
| | FGL1, FGL2, FKBP1A, FKBP1B, FLOT2, FOXJ1, FOXN1, FOXP1, |
| | FOXP3, FUT7, FYN, FZD5, FZD7, FZD8, GATA3, GLI2, GLI3, GLMN, |
| | GNRH1, GPAM, GPNMB, GPR18, GPR183, GPR89A, GPR89B, |
| | GRAP2, GRB2, GSN, HAVCR2, HES1, HFE, HHLA2, HLA-A, HLA- |
| | DMB, HLA-DOA, HLA-DPA1, HLA-DPB1, HLA-DRB1, HLA-E, HLA-G, |
| | HLX, HMGB1, HSH2D, HSPD1, HSPH1, ICAM1, ICOS, ICOSLG, |
| | IDO1, IFNA1, IFNA10, IFNA13, IFNA14, IFNA16, IFNA17, IFNA2, |
| | IFNA21, IFNA4, IFNA5, IFNA6, IFNA7, IFNA8, IFNB1, IFNE, IFNG, |
| | IFNK, IFNL1, IFNW1, IGF1, IGF2, IGFBP2, IHH, IL10, IL12A, IL12B, |
| | IL12RB1, IL15, IL18, IL18R1, IL1A, IL1B, IL1RL2, IL2, IL20RB, IL21, |
| | IL23A, IL23R, IL27, IL27RA, IL2RA, IL36B, IL4, IL4R, IL6, IL6ST, IL7, |
| | IL7R, INS, IRF1, IRF4, ITGAL, ITK, ITPKB, JAG2, JAK3, JAML, |
| | JMJD6, KAT2A, KDELR1, KIF13B, KIT, KLRC4-KLRK1, KLRK1, LAG3, |
| | LAT, LAX1, LCK, LCP1, LEF1, LEP, LEPR, LFNG, LGALS1, LGALS3, |
| | LGALS7B, LGALS9, LGALS9B, LGALS9C, LIG4, LILRB1, LILRB2, |
| | LILRB4, LMBR1L, LMO1, LOXL3, LRRC32, LY9, LYN, MAD1L1, |
| | MAFB, MALT1, MAP3K8, MAPK8IP1, MARCHF7, MDK, METTL3, |
| | MICA, MICB, MIR181C, MIR21, MIR27A, MIR30B, MR1, MSN, MTOR, |
| | MYB, MYH9, NCAPH2, NCK1, NCK2, NCKAP1L, NCSTN, NDFIP1, |
| | NEDD4, NFATC2, NFKBID, NFKBIZ, NHEJ1, NKAP, NKX2-3, NLRC3, |
| | NLRP3, NOD2, NRARP, P2RX7, PAG1, PAK1, PAK2, PAK3, PATZ1, |
| | PAWR, PAX1, PCK1, PDCD1, PDCD1LG2, PDE5A, PDPK1, PELI1, |
| | PIK3CA, PIK3CD, PIK3CG, PIK3R1, PIK3R6, PKNOX1, PLA2G2D, |
| | PLA2G2F, PNP, PPP3CA, PPP3CB, PRDM1, PRDX2, PRELID1, |
| | PREX1, PRKAR1A, PRKCQ, PRKCZ, PRKDC, PRNP, PRR7, PSAP, |
| | PSEN1, PSMB10, PSMB11, PTGER4, PTPN11, PTPN2, PTPN22, |
| | PTPN6, PTPRC, PYCARD, RAB27A, RAB29, RABL3, RAC1, RAC2, |
| | RAG1, RAG2, RARA, RASAL3, RASGRP1, RC3H1, RC3H2, RELB, |
| | RHOA, RHOH, RIPK2, RIPK3, RIPOR2, RORA, RORC, RPS3, |
| | RSAD2, RUNX1, RUNX2, RUNX3, SART1, SASH3, SATB1, |
| | SCGB1A1, SCRIB, SDC4, SELENOK, SEMA4A, SFTPD, SH3RF1, |
| | SHH, SIRPA, SIRPB1, SIRPG, SIT1, SLA2, SLAMF6, SLC11A1, |
| | SLC46A2, SLC7A1, SMAD3, SMAD7, SOCS1, SOCS5, SOCS6, |
| | SOD1, SOS1, SOX12, SOX13, SOX4, SP3, SPINK5, SPN, SPTA1, |
| | SRC, SRF, STAT3, STAT5B, STAT6, STK11, STOML2, SYK, TARM1, |
| | TBX21, TCF7, TCIRG1, TESPA1, TFRC, TGFBR2, THEMIS, THY1, |
| | TIGIT, TMEM131L, TMEM98, TMIGD2, TNFAIP8L2, TNFRSF13C, |
| | TNFRSF14, TNFRSF18, TNFRSF1B, TNFRSF21, TNFRSF4, |
| | TNFSF11, TNFSF13B, TNFSF14, TNFSF18, TNFSF4, TNFSF8, |
| | TNFSF9, TOX, TP53, TRAF6, TREML2, TREX1, TSC1, TWSG1, |
| | VAV1, VCAM1, VNN1, VSIG4, VSIR, VTCN1, WAS, WNT1, WNT4, |
| | XBP1, XCL1, YES1, ZAP70, ZBTB1, ZBTB16, ZBTB7B, ZC3H12A, |
| | ZC3H8, ZEB1, ZFP36L1, ZFP36L2, ZFPM1, ZMIZ1, ZNF683, ZP3, |
| | ZP4 |
| Go_Immature_T_Cell_Proliferation | BMP4, ERBB2, FOXP3, GNRH1, IHH, IL1A, IL1B, RIPK2, SHH, |
| | TMEM131L, WNT4 |
| Go_Cd8_Positive_Alpha_Beta_T_Cell_{—} | HLA-A, HLA-E, IRF1, MAPK8IP1, PTPN22, SH3RF1, VSIR, XCL1 |
| Proliferation | |
| Go_Cd8_Positive_Alpha_Beta_T_Cell_{—} | BCL2, CBFB, EOMES, GPR18, IRF1, NCKAP1L, PAX1, PSMB11, |
| Differentiation | RUNX1, RUNX3, SATB1, SOCS1, TNFSF8, TOX, ZBTB7B |
| Go_Cd8_Positive_Alpha_Beta_T_Cell_{—} | BCL2, CBFB, CD274, CLEC4A, CRTAM, EOMES, GPR18, HFE, HLA- |
| Activation | A, HLA-E, IRF1, LILRB1, MAPK8IP1, NCKAP1L, PAX1, PSMB11, |
| | PTPN22, RUNX1, RUNX3, SATB1, SH3RF1, SOCS1, TNFSF8, TOX, |
| | VSIR, XCL1, ZBTB7B |
| Go_Cd4_Positive_Or_Cd8_Positive_Alpha_{—} | BATF, BCL2, CTSL, CYLD, FOXP3, IL12B, IL12RB1, IL23A, IL23R, |
| Beta_T_Cell_Lineage_Commitment | IL6, IRF4, LOXL3, LY9, MTOR, SHH, SLAMF6, SPN, STAT3, STAT6, |
| | TBX21, TOX, ZFPM1 |
| Go_Cd4_Positive_Cd25_Positive_Alpha_{—} | FOXP3, FUT7, IFNG, LGALS9, PLA2G2D, TOX |
| Beta_Regulatory_T_Cell_Differentiation | |
| Go_Cd4_Positive_Alpha_Beta_T_{—} | ARG2, CD274, CD55, CD81, LGALS7B, LGALS9, LGALS9B, |
| Cell_Proliferation | LGALS9C, TGFBR2, TWSG1, VSIR, XCL1 |
| Go_Cd4_Positive_Alpha_Beta_T_{—} | ARG1, CD55, CD81, DENND1B, GATA3, IFNA2, IFNB1, IL12A, IL12B, |
| Cell_Cytokine_Production | IL18, IL18R1, IL18RAP, IL1B, IL1R1, IL31RA, IL4, IL6, NLRP3, |
| | PRKCZ, RSAD2, STARD7, TBX21, XCL1 |
| Go_Cd4_Positive_Alpha_Beta_T_{—} | AGER, ANXA1, ARG2, ATP7A, BATF, BCL3, BCL6, CBFB, CCL19, |
| Cell_Activation | CD160, CD274, CD55, CD80, CD81, CD83, CD86, CDH26, CTSL, |
| | FOXP1, FOXP3, FUT7, GATA3, GPR183, HLX, HMGB1, IFNG, IL12A, |
| | IL12B, IL12RB1, IL18, IL18R1, IL2, L21, IL23A, IL23R, IL27, IL4, IL4R, |
| | IL6, IRF4, JAK3, LEF1, LGALS7B, LGALS9, LGALS9B, LGALS9C, |
| | LOXL3, LY9, MALT1, MIR21, MTOR, MYB, NCKAP1L, NFKBID, |
| | NFKBIZ, NKX2-3, NLRP3, PAX1, PLA2G2D, PRKCQ, PRKCZ, |
| | PTGER4, RARA, RC3H1, RC3H2, RELB, RIPK2, RORA, RORC, |
| | RSAD2, RUNX1, RUNX3, SASH3, SATB1, SEMA4A, SH3RF1, |
| | SLAMF6, SMAD7, SOCS1, SOCS5, SPN, STAT3, STAT6, STOML2, |
| | TARM1, TBX21, TCIRG1, TGFBR2, TMEM98, TNFSF4, TOX, TWSG, |
| | VSIR, XCL1, ZBTB7B, ZC3H12A, ZFPM1 |
| Go_Alpha_Beta_T_Cell_Proliferation | ARG2, CCR2, CD274, CD28, CD3E, CD55, CD81, DOCK2, EBI3, |
| | ELF4, HLA-A, HLA-E, IL12B, IL15, IL18, IL23A, IRF1, LGALS7B, |
| | LGALS9, LGALS9B, LGALS9C, MAPK8IP1, PTPN22, PTPRC, |
| | RASAL3, RIPK2, SH3RF1, SYK, TGFBR2, TNFRSF14, TNFSF4, |
| | TWSG1, VSIR, XCL1, ZAP70, ZBTB7B |
| Go_Alpha_Beta_T_Cell_Activation_Involved_{—} | ANXA1, ATP7A, BATF, BCL3, BCL6, CCL19, CD80, CD86, EOMES, |
| In_Immune_Response | FOXP1, FOXP3, GATA3, GPR183, HLX, HMGB1, IFNG, IL12B, |
| | IL12RB1, IL18, IL18R1, IL2, IL21, IL23A, IL23R, IL27, IL4, IL4R, IL6, |
| | IRF4, JAK3, LEF1, LGALS9, LOXL3, LY9, MALT1, MIR21, MTOR, |
| | MYB, NFKBID, NFKBIZ, NLRP3, PRKCZ, PTGER4, RARA, RC3H1, |
| | RC3H2, RELB, RIPK2, RORA, RORC, SEMA4A, SLAMF6, SMAD7, |
| | SOCS5, SPN, STAT3, STAT6, TBX21, TMEM98, TNFSF4, ZBTB7B, |
| | ZC3H12A, ZFPM1 |
| Go_Activated_T_Cell_Proliferation | ABL1, AGER, ARG1, BTN2A2, BTN3A1, CASP3, CD24, CD274, CLC, |
| | CRTAM, EPO, FADD, FOXP3, FYN, GPAM, HHLA2, HMGB1, |
| | ICOSLG, IGF1, IGF2, IGFBP2, IL12B, IL12RB1, IL18, IL2, IL23A, |
| | IL23R, IL27RA, IL2RA, LGALS9, LRRC32, MIR181C, MIR21, MIR30B, |
| | PDCD1LG2, PRKAR1A, PRNP, PYCARD, RC3H1, RIPK3, RPS3, |
| | SATB1, SCRIB, STAT5B, TMIGD2, TNFSF9 |

Table 1 lists genes that are differentially expressed (up or down as indicated) in the tumor microenvironment of responders versus non-responders, as well as the biological pathways those genes involve, including angiogenic signatures and T cell functions. For example, the angiogenic signatures can include, but are not limited to, endothelial cell (EC) migration, EC proliferation and/or EC sprouting. In non-limiting examples, T cell function genes include those involved in T cell chemotaxis, T cell cytotoxicity, T cell extravasation, and/or antigen presentation.

The angio-immune panel may represent the expression profile of at least two genes selected from Table 1, for example, at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 30 genes, at least 40 genes, at least 50 genes, at least 60 genes, at least 70 genes, at least 80 genes, at least 90 genes, at least 100 genes or more. In some examples, the angio-immune panel may comprise multiple up-regulated genes as indicated in Table 1. In other examples, the angio-immune panel may comprise multiple down-regulated genes as indicated in Table 1. In yet other examples, the angio-immune panel may comprise both up-regulated and down-regulated genes as indicated in Table 1. In specific examples, the angio-immune panel comprises all genes listed in Table 1.

In some aspects, the angio-immune panel may comprise multiple genes involved in multiple biological pathways, for example, 2 biological pathways, 3 biological pathways, 4 biological pathways, 5 biological pathways, 6 biological pathways, 7 biological pathways, 8 biological pathways, 9 biological pathways, 10 biological pathways, 11 biological pathways, 12 biological pathways, 13 biological pathways, 14 biological pathways, or 15 biological pathways.

In some examples, the angio-immune panel comprises at least one gene that is involved in endothelial cell (EC) migration. Non-limiting examples of genes involved in endothelial cell (EC) migration useful according to the methods described herein include those described in Table 1.

In some examples, the angio-immune panel comprises at least one gene involved in EC proliferation. Examples of EC proliferation genes useful in the methods disclosed herein include those described in Table 1.

In some examples, the angio-immune panel comprises at least one gene involved EC sprouting. Non-limiting examples of genes involved in EC sprouting to be used as biomarkers in the methods described herein include those described in Table 1.

In some examples, the angio-immune panel comprises at least one gene involved in T cell chemotaxis. Examples include but are not limited to those described in Table 1.

In some examples, the angio-immune panel comprises at least one gene involved T cell cytotoxicity. Non-limiting examples of genes involved in T cell cytotoxicity to be used as biomarkers in the methods described herein include those described in Table 1.

In some examples, the angio-immune panel comprises at least one gene involved in T cell extravasation. Examples of T cell extravasation genes useful in the methods disclosed herein include, but are not limited to those described in Table 1.

In some examples, the angio-immune panel comprises at least one gene involved in antigen presentation. Non-limiting examples of genes involved in antigen presentation to be used as biomarkers in the methods described herein include those described in Table 1.

### (B) Determination of Angio-Immune Subtype

In some aspects of the method disclosed herein, the expression of two or more genes or Angio-Immune panel as described in A can be used to determine the Angio-Immune subtype of a subject.

For determining the angio-immune subtype as disclosed herein, the expression levels of the genes involved in the angio-immune panel in a biological sample (e.g. a sample comprising the tumor microenvironment) of a candidate subject can be measured by routine practice or obtained from entities equipped to determine gene expression levels from biological samples. In some examples, the gene expression levels can be mRNA levels of the target genes. Alternatively, the gene expression levels can be represented by the levels of the gene products (encoded proteins). Assays for measuring levels of mRNA or proteins are known in the art and described herein. See, e.g., Molecular Cloning: A Laboratory Manual, J. Sambrook, et al., eds., Third Edition, Cold Spring Harbor Laboratory Press, Cold Spring Harbor, New York, 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York.

In some aspects, the expression level(s) of the genes involved in any of the angio-immune panel as disclosed herein may be represented by the level of the mRNAs. Methods for detecting and/or assessing a level of nucleic acid expression in a sample are well known in the art, and all suitable methods for detecting and/or assessing an amount of nucleic acid expression known to one of skill in the art are contemplated within the scope of the invention. Non-limiting examples of suitable methods to assess an amount of nucleic acid expression may include arrays, such as microarrays, PCR, such as RT-PCR (including quantitative RT-PCR), nuclease protection assays and Northern blot analyses.

The level of expression of the target genes may be normalized to the level of a control nucleic acid. This allows comparisons between assays that are performed on different occasions. For example, the raw data of gene expression levels can be normalized against the expression level of an internal control RNA (e.g., a ribosomal RNA or U6 RNA). The normalized expression level(s) of the genes can then be compared to the expression level(s) of the same genes of a control tissue sample, which can be normalized against the same internal control RNA, to determine whether the subject is likely to be responsive to a therapeutic treatment or non-responsive to a therapeutic treatment.

In another aspect, the levels of the gene expression can be determined by measuring the gene products at the protein level in a biological sample. In an exemplary aspect, protein expression may be measured using an ELISA to determine the expression level of the genes involved in the corticosteroid responsiveness gene signature as disclosed herein in a biological sample as also disclosed herein. Methods for detecting and/or assessing an amount of protein expression are well known in the art, and all suitable methods for detecting and/or assessing an amount of protein expression known to one of skill in the art are contemplated within the scope of the invention. Non-limiting examples of suitable methods to detect and/or assess an amount of protein expression may include epitope binding agent-based methods and mass spectrometry-based methods.

Based on the expression levels of the involved genes as disclosed herein, an angio-immune subtype can be obtained via, e.g., a computational program. Various computational programs can be applied in the methods of this disclosure to aid in analysis of the expression data for producing the gene signature. Examples include, but are not limited to, Prediction Analysis of Microarray (PAM; see Tibshirani et al., PNAS 99(10):6567-6572, 2002); Plausible Neural Network (PNN; see, e.g., U.S. Pat. No. 7,287,014), PNNSolution software and others provided by PNN Technologies Inc., Woodbridge, VA, USA, and Significance Analysis of Microarray (SAM). In some examples, a gene expression profile may be represented by a score that characterizes the expression pattern of the genes involved in the gene signature. For example, a baseline angio-immune score. See also the Examples below.

The terms “subject” or “patient” may be used interchangeably and refer to a subject who needs the analysis as described herein. In some aspects, the subject is a human or a non-human mammal (e.g., a non-human primate, rodent, ovine, bovine, feline, equine, canine). In some aspects, the subject is suspected to have cancer or is at risk for cancer. In some aspects, the subject has (e.g., is known to have) cancer. In some aspects, the cancer is a non-hematological cancer. In some aspects, the cancer is a metastatic cancer. Examples of cancer include, without limitation, Adrenocortical carcinoma (ACC), Bladder Urothelial Carcinoma (BLCA), Brain Lower Grade Glioma (LGG), Breast invasive carcinoma (BRCA), Colon adenocarcinoma (COAD), Cervical squamous cell carcinoma and endocervical adenocarcinoma (CESC), Cholangiocarcinoma (CHOL), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Uterine Carcinosarcoma (UCS), Uterine Corpus Endometrial Carcinoma (UCEC), and Uveal Melanoma (UVM). A subject having a cancer or tumor may be diagnosed based on clinically available tests and/or an assessment of the pattern of symptoms in a subject and response to therapy. Such a subject may exhibit one or more symptoms associated with a cancer. Alternatively, or in addition, such a subject may have one or more risk factors for cancer, for example, an environmental factor associated with cancer (e.g., geographic location or exposure to a mutagen), a family history of cancer, and/or a genetic predisposition to developing cancer.

Alternatively, the subject who needs the analysis described herein may be a patient having cancer or suspected of having cancer. Such a subject may currently be having a relapse or may have suffered from the disease in the past (e.g., may be currently relapse-free), or may have cancer. In some examples, the subject is a human patient who may be on a treatment (i.e., the subject may be receiving treatment) for the disease including, for example, a treatment involving chemotherapy or radiation therapy. In other instances, such a human patient may be free of such a treatment.

As used herein, the term “biological sample” refers to a sample obtained from a subject. Any biological sample from a subject (i.e., a patient or individual) may be analyzed as described herein to obtain expression data. In some aspects, the biological sample may be any sample from a subject known or suspected of having cancerous cells or pre-cancerous cells. In some aspects, the biological sample is obtained from the tumor microenvironment.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine, hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

The biological sample may be any type of sample including, for example, a sample of a bodily fluid, one or more cells, a piece of tissue, or some or all of an organ. In certain aspects, one sample will be taken from a subject for analysis. In some aspects, more than one (e.g., 2, 3, 4, 5, 6, 7, 8, 9, 10, 11, 12, 13, 14, 15, 16, 17, 18, 19, 20, or more) sample may be taken from a subject for analysis. In some aspects, one sample from a subject will be analyzed. In certain aspects, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments and may be taken from the same region or a different region. As an example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor or different tumors prior to and subsequent to a treatment).

Any of the biological samples described herein may be obtained from the subject using any known technique. In some aspects, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy). In some aspects, each of the at least one biological samples is a bodily fluid sample, a cell sample, or a tissue biopsy.

In some aspects, one or more than one cell (i.e., a cell sample) may be obtained from a subject using a scrape or brush method. The cell sample may be obtained from any area in or from the body of a subject including, for example, from one or more of the following areas: the cervix, esophagus, stomach, bronchus, or oral cavity. In some aspects, one or more than one piece of tissue (e.g., a tissue biopsy) from a subject may be used. In certain aspects, the tissue biopsy may comprise one or more than one (e.g., 2, 3, 4, 5, 6, 7, 8, 9, 10, or more than 10) samples from one or more tumors or tissues known or suspected of having cancerous cells.

In some aspects, once a sample is obtained, analysis of the sample can be done to obtain the angio-immune subtype of the cancer.

### (C) Predicting Responsiveness Based on the Angio-Immune Subtypes and Optionally Other Factors

Any of the angio-immune panel signatures or subtype classification as exemplified in the Examples of a candidate subject can be used for assessing whether the subject's responsiveness or non-responsiveness to an immune checkpoint blockade, for example, an anti-PD1 and/or anti-CTLA4 therapeutic. For example, the angio-immune panel of a candidate subject can be compared with a reference value.

A reference value may represent the same angio-immune panel signature of a control subject or represent the same angio-immune panel signature of a control population. In some examples, the same angio-immune panel signature of a control subject or a control population may be determined by the same method as used for determining the angio-immune panel signature of the candidate subject. In some instances, the control subject or control population may refer to a healthy subject or healthy subject population of the same species (e.g., a human subject or human subject population having no tumor or cancer). Alternatively, the control subject or control population may be a tumor or cancer patient or tumor or cancer patient population who are responsive to any of the therapeutic agents disclosed herein. In other instances, the control subject or control population may be a cancer or tumor patient or cancer or tumor patient population who is non-responsive to the therapeutic agent.

It is to be understood that the methods provided herein do not require that a reference value be measured every time a candidate subject is tested. Rather, in some aspects, it is contemplated that the reference value can be obtained and recorded and that any test level can be compared to such a reference level. The reference level may be a single-cutoff value or a range of values.

In an exemplary aspects, the angio-immune subtype of a patient can be determined by (i) obtaining the expression and mutational profile of the two or more genes as provided in Table 1 from, for example a clinical lab, a diagnostic lab, a academic laboratory or any entity equipped to provide such data; (ii) implementing a gene set variation analysis (GSVA) to obtain an enrichment score of the two or more genes by comparing it to a suitable reference as provided herein; (iii) generating a correlation matrix across the enrichment scores; (iv) identifying the angio-immune subtype of the subject based on the correlation matrix. By comparing the correlation matrix of a candidate subject as disclosed herein and a reference value as also described herein, the subject can be classified into an angio-immune subtype and can be identified as responsive or likely to be responsive or as not responsive or not likely to be responsive to treatment.

One or more steps of the determination as provided herein can use a computer implemented method. An exemplary method of calculating the angio-immune subtype is provided in the examples, though one or more steps of the method can be replaced by equivalent computational methods known in the art. Thus, aspects of the technology described herein provide computer implemented methods for using expression data for a subject, gene expression level differences indicative of a patient's response or lack thereof, to an immune checkpoint blockade therapy.

In some aspects, a software program may provide a user with a visual representation presenting information related to a patient's expression data (e.g., expression levels and/or expression level differences), and predicted efficacy or determined efficacy of one or more checkpoint blockade therapies using a graphical user interface (GUI). Such a software program may execute in any suitable computing environment including, but not limited to, a cloud-computing environment, a device co-located with a user (e.g., the user's laptop, desktop, smartphone, etc.), one or more devices remote from the user (e.g., one or more servers), etc. In some particular aspects, the computer implemented methods may include for example, implementing a gene set variation analysis (GSVA) to obtain an enrichment score of the two or more genes by comparing it to a suitable reference as provided herein; generating a correlation matrix across the enrichment scores; identifying the angio-immune subtype of the subject based on the correlation matrix. In some aspects, the angio-immune subtypes may fall into 3 broad categories. For example, a subtype C1 may contain strong positive enrichment for angiogenic gene sets, including endothelial cell (EC) migration, EC proliferation, EC sprouting but marked downregulation of T-cell functional signatures. In contrast, a subtype C2 may have no significant enrichment for functional angiogenesis signatures and no significant enrichment for T-cell-related gene sets. Another subtype C3, may be characterized by the downregulation of functional angiogenesis signatures with strong positive enrichment for T-cell functions. In some aspects, the proportion of the microenvironment subtypes may vary across tumor types. In some aspects, various software and algorithms may be integrated into the computational methods provided herein. Non-limiting examples are provided in Table 2.

In addition to the angio-immune subtype, a subject's responsive or non-responsiveness to the treatment disclosed herein may further take into consideration one or more clinical factors. Exemplary clinical factors include, but are not limited to, mutational status of one or more genes, stromal score, fibroblast score and/or mutational burden. In some examples, mutation status of IDH1, BRAF, PTEN, ATRX, CSMD3, LRP1B, ZFHX4, USH2A, and/or FAT4 are additional clinical factors used to determine a subject's responsiveness.

### II. Therapeutic Application of Angio-Immune Panel Signatures

In an aspect, the current disclosure encompasses a method of treating a cancer in a subject in need thereof, comprising: administering to the subject an ICB therapeutic, wherein the subject has been identified as having an angio-immune subtype corresponding to a low angiogenic signature and a high T cell function signature, and wherein the angio-immune subtype was determined by a method provided herein. In an aspect, the method may comprise: (i) providing expression levels of two or more genes in a biological sample obtained from tumor microenvironment (TME) of the subject, wherein the two or more genes encode effectors that impact angiogenesis or T-cell function or both; (ii) determining an angio-immune score based on the expression levels of the two or more genes in step (i); and (iii) comparing the angio-immune score to an angio-immune baseline score for a corresponding tumor microenvironment to determine an angio-immune subtype.

As used herein, the term “immune checkpoint blockade therapeutic” or “immune checkpoint inhibitor” includes inhibitors and therapeutics that work by blocking immune checkpoint proteins from binding with their partner proteins thus preventing the immune system to switch off prior to the clearance of the cancer. Presence of the inhibitors this allows T-cells to kill cancer cells.

When a subject is determined to be responsive or non-responsive based on any of the angio-immune subtypes disclosed herein, this subject could be subjected to a suitable treatment for a cancer or tumor including any of the immune check blockade and/or anti-angiogenesis treatments known in the art and disclosed herein. Alternatively, when a subject is determined as having or at risk for cancer or a tumor or having active disease, such a subject may be given a suitable therapy, for example, those described herein.

In some aspects, a subject is determined to be responsive or likely responsive to an immune check blockade therapy, using any of the methods described herein, the subject may then be administered an effective amount of an immune checkpoint blockade, for treating a cancer or tumor. In some examples, such a subject may be given an anti-CTLA-4, PD-1 inhibitor, and/or PD-L1 inhibitor compounds. Non limiting examples of immune checkpoint compounds include monoclonal antibodies that target either PD-1 or PD-L1 can block this binding and boost the immune response against cancer cells include Pembrolizumab, Nivolumab, Cemiplimab, Atezolizumab, Avelumab, Durvalumab, and/or Ipilimumab.

In some aspects, the immune checkpoint blockade therapy targets cytotoxic T lymphocyte antigen 4 (CTLA4) or a ligand of CTLA4 such as CD80 and/or CD86. In some aspects, the immune checkpoint blockade therapy is a molecule that inhibits CTLA4. In some aspects, the immune checkpoint blockade therapy is a molecule that inhibits CD80. In some aspects, the immune checkpoint blockade therapy is a molecule that inhibits CD86.

A molecule that inhibits CTLA4, CD80 and/or CD86, in some aspects, is an antibody or antigen binding fragment thereof. Examples of a molecule that inhibits CTLA4, CD80 and/or CD86 include, but are not limited to, ipilimumab or tremelimumab.

An immune checkpoint blockade therapy as described herein may have targets other than PD1 and/or CTLA4 and their ligands. In some aspects, the immune checkpoint blockade therapy targets lymphocyte activating gene 3 (LAG-3, CD223) or a ligand thereof. In some aspects, the immune checkpoint blockade therapy targets killer inhibitory receptors (e.g., KIR2DL-1, KIR2DL-2, and KIR2DL-3) or a ligand thereof. In some aspects, the immune checkpoint blockade therapy targets B7-H3 (CD276) or a ligand thereof. In some aspects, the immune checkpoint blockade therapy targets T cell immunoglobulin and mucin3 (TIM-3) or a ligand thereof. In some aspects, the immune checkpoint blockade therapy targets V-domain Ig-containing suppressor of T cell activation (VISTA) or a ligand thereof. In some aspects, the immune checkpoint blockade therapy targets T cell ITIM Domain (TIGIT) or a ligand thereof. In some aspects, the immune checkpoint blockade therapy targets immune inhibitory enzyme (IDO) or a ligand thereof.

In some aspects, a subject is determined to be unresponsive or unlikely to be responsive to an immune check blockade therapy, using any of the methods described herein, the subject may then be administered an effective amount of an alternative therapeutic agent for treating a cancer or tumor, for example, a chemotherapeutic agent, radiation therapy, and/or anti-angiogenesis therapy.

In some aspects, a subject is determined to have a tumor or cancer and can be treated by a suitable cancer therapy, such as those described herein. Alternatively, a subject is determined to be responsive to therapy and can be treated by a suitable anti-ICB therapy or subject to adjustment of current therapy (e.g., switch to a different therapeutic agent or adjust treatment conditions such as doses or dosing schedules of the current therapeutic agent).

Non-limiting examples of anti-angiogenesis agents include drugs which block vascular endothelial growth factor (VEGF) from attaching to the receptors on the cells that line the blood vessels, drugs stop the VEGF receptors from sending growth signals, thalidomide, lenalidomide, and anti-MYCT1 therapies. In non-limiting examples, the method may comprise administering one or more of axitinib, bevacizumab, cabozantinib, lapatinib, Lenvatinib, pazopanib, ponatinib, ramucirumab, ranibizumab, regorafenib, sorafenib, sunitinib and/or vandetanib.

In some aspects, when the classification reveals a population of highly angiogenic patients who do not mount responses to ICB treatment, the present disclosure provides providing to the subject a vessel normalizing agent (e.g. anti-VEGF and/or anti-MYCT1) to prime subject for subsequent for ICB therapy.

The term “treating” as used herein refers to the application or administration of a composition including one or more active agents to a subject, who has a cancer or tumor, a symptom of a cancer or tumor, or a predisposition towards cancer or tumors, with the purpose to cure, heal, alleviate, relieve, alter, remedy, ameliorate, improve, or affect the disease, the symptoms of the disease, or the predisposition toward the disease. An “effective amount” is that amount of an cancer agent that alone, or together with further doses, produces the desired response, e.g. eliminate or alleviate symptoms, prevent or reduce the risk of recurrence (maintain long-term remission), and/or restore quality of life. The desired response is to inhibit the progression of the disease. This may involve only slowing the progression of the disease temporarily, although more preferably, it involves halting the progression of the disease permanently. This can be monitored by routine methods or can be monitored according to diagnostic and prognostic methods discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Any of the methods described herein can further comprise adjusting the cancer treatment performed to the subject based on the results obtained from the methods disclosed herein (e.g., based on the signatures disclosed herein). Adjusting treatment includes, but are not limited to, changing the dose and/or administration of the cancer agent used in the current treatment, switching the current medication to a different cancer agent, or applying a new cancer therapy to the subject, which can be either in combination with the current therapy or replacing the current therapy.

A composition of the disclosure may optionally comprise one or more additional drug or therapeutically active agent in addition to a compound that modulates MYCT1. For example, a composition of the disclosure may optionally comprise one or more immune checkpoint blockade compounds. Specifically, a composition of the invention may optionally comprise one or more Still further, a composition of the disclosure may optionally comprise one or more anti-VEGF therapies. In non-limiting examples, a composition of the invention may optionally comprise one or more of axitinib, bevacizumab, cabozantinib, lapatinib, Lenvatinib, pazopanib, ponatinib, ramucirumab, ranibizumab, regorafenib, sorafenib, sunitinib and/or vandetanib.

A composition of the invention may further comprise a pharmaceutically acceptable excipient, carrier or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants.

As noted above, the agents or compositions described herein can also be used in combination with other therapeutic agents, as described further below. Thus, in addition to the therapies described herein, one may also provide to the subject other therapies known to be efficacious for treatment of the disease, disorder, or condition. In various examples, a method further comprises administering to the patient an additional cancer treatment. In some examples, the additional cancer treatment is chosen from the group comprising surgery, radiotherapy, chemotherapy, toxin therapy, immunotherapy, cryotherapy, gene therapy, and combinations thereof. Examples of anti-angiogenic therapeutic targets useful in accordance with the disclosure include EGF, VEGF, FGF, and matrix remodeling proteins. In various examples, a chemotherapy agent is a drug or drug formulation. Non-limiting examples of drug formulations include polymeric micelle formulations, liposomal formulations, dendrimer formulations, polymer-based nanoparticle formulations, silica-based nanoparticle formulations, nanoscale coordination polymer formulations, nanoscale metal-organic framework formulations, inorganic nanoparticle formulations, and the like.

Various chemotherapy agents (e.g., chemotherapy drugs) can be used. Any FDA approved chemotherapy agent (e.g., chemotherapy drugs) can be used. Combinations of chemotherapy agents may be used.

In some aspects, the additional drug or therapeutically active agent may be a genotoxic agent (e.g., a DNA-damaging agent or drug). As used herein “genotoxic therapy” refers to a treat of a tumor or cancer which utilizes the destructive properties of the treatment to induce DNA damage into tumor or cancer cells. The treatment is traditionally part of standardized regime. Any damage done to a tumor cancer is passed on to descendent cancer cells as proliferation continues. If this damage is severe enough, it will induce cells to undergo apoptosis. In non-limiting examples, a genotoxic therapy may include γ-irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, mitoxantrone, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine), and estramustine.

Treatment in accord with the methods described herein can be performed prior to, concurrent with, or after conventional treatment modalities for a cancer or tumor.

Dosages of an additional drug or therapeutically active agent can vary between wide limits, depending upon the disease or disorder to be treated, the age and condition of the subject to be treated. In an aspect where the composition further comprising at least one additional drug or therapeutically active agent is contacted with a sample, the concentration of the at least one additional drug or therapeutically active agent may be from about 0.01 μM to about 10 μM. Alternatively, the concentration of the at least one additional drug or therapeutically active agent may be from about 0.01 μM to about 5 μM. For example, the concentration of the at least one additional drug or therapeutically active agent may be about 0.01, about 0.05, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, or about 10 μM. Addition-ally, the concentration of the at least one additional drug or therapeutically active agent be greater than 10 μM. For example, the concentration of the at least one additional agent may be about 10, about 15, about 20, about 25, about 30, about 35, about 40, about 45, about 50, about 55, about 60, about 65, about 70, about 75, about 80, about 85, about 90, about 95, or about 100 μM.

In an aspect where the composition further comprising at least one additional drug or therapeutically active agent administered to a subject, the dose of the additional drug or therapeutically active agent may be from about 0.1 mg/kg to about 500 mg/kg. For example, the dose of the least one additional drug or therapeutically active agent may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of the least one additional drug or therapeutically active agent may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of the least one additional drug or therapeutically active agent may be about 300 mg/kg, about 325 mg/kg, about 350 mg/kg, about 375 mg/kg, about 400 mg/kg, about 425 mg/kg, about 450 mg/kg, about 475 mg/kg, or about 500 mg/kg.

Generally, a safe and effective amount of a composition is administered, for example, that amount that would cause the desired therapeutic effect in a subject while minimizing undesired side effects. In various aspects, an effective amount of a composition described herein can substantially reduce the growth or spread of cancer in a subject. In some aspects, an effective amount is an amount capable of treating a cancer or tumor. In some aspects, an effective amount is an amount capable of treating one or more symptoms associated with a cancer or tumor.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of a composition as disclosed herein can occur as a single event or over a time course of treatment. For example, a composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The disclosure is not limited to dosing intervals that are spaced equally in time, but encompass doses at non-equal intervals, such as a priming schedule consisting of administration at 1 day, 4 days, 7 days, and 25 days, just to provide a non-limiting example.

A dosing schedule of, for example, once/week, twice/week, three times/week, four times/week, five times/week, six times/week, seven times/week, once every two weeks, once every three weeks, once every four weeks, once every five weeks, and the like, is available for the disclosure. The dosing schedules encompass dosing for a total period of time, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The present disclosure provides methods of administrating pharmaceutical compositions, for example an ICB therapeutic agent as disclosed above, so as to facilitate administration and promote stability of the active agent. For example, a compound may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and aspects of the invention, the subject may be a human or any other animal.

Therefore, in one aspect, the methods may encompass administration of a therapeutic to an individual to treat a tumor or cancer. In another aspect, the methods may encompass administration to an individual to prevent or reduce vascular network formation in a target tissue. The tissue can be undesirable tissue that has arisen due to transformation, such as a tumor, cancer. As used herein, the term “cancer” includes a wide variety of malignant neoplasms. These can be caused by viral infection, naturally occurring transformation, or exposure to environmental agents.

In some examples, the methods can be useful for treating a cancer or tumor in a subject. The term “treating a cancer or tumor” includes, but is not limited to, preventing or reducing the development of a cancer or tumor, reducing the symptoms of cancer or tumor, suppressing or inhibiting the growth of an established cancer or tumor, preventing metastasis and/or invasion of an existing cancer or tumor, promoting or inducing regression of the cancer or tumor, inhibiting or suppressing the proliferation of cancerous or tumor cells, reducing angiogenesis or increasing the amount of apoptotic cancer or tumor cells, thereby treating cancer or a tumor.

Non-limiting examples of cancers or tumors that may be treated with a method of the disclosure may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (child-hood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, or Wilms tumor (childhood).

In some aspects, the cancer may be a non-hematological cancer selected from Adrenocortical carcinoma (ACC), Bladder Urothelial Carcinoma (BLCA), Brain Lower Grade Glioma (LGG), Breast invasive carcinoma (BRCA), Colon adenocarcinoma (COAD), Cervical squamous cell carcinoma and endocervical adenocarcinoma (CESC), Cholangiocarcinoma (CHOL), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Uterine Carcinosarcoma (UCS), Uterine Corpus Endometrial Carcinoma (UCEC), and Uveal Melanoma (UVM).

A subject may be a rodent, a human, a livestock animal, a companion animal, or a zoological animal. In one aspect, the subject may be a rodent, e.g. a mouse, a rat, a guinea pig, etc. In another aspect, the subject may be a livestock animal. Non-limiting examples of suitable livestock animals may include pigs, cows, horses, goats, sheep, llamas and alpacas. In still another aspect, the subject may be a companion animal. Non-limiting examples of companion animals may include pets such as dogs, cats, rabbits, and birds. In yet another aspect, the subject may be a zoological animal. As used herein, a “zoological animal” refers to an animal that may be found in a zoo. Such animals may include non-human primates, large cats, wolves, and bears. In a preferred aspect, the subject is a human.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various aspects of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

Immune checkpoint blockade (ICB) therapy has revolutionized cancer treatment. However, only a fraction of the patients responds to ICB therapy. Accurate prediction of patients to likely respond to ICB would maximize the efficacy of ICB therapy. The tumor microenvironment (TME) dictates tumor progression and therapy outcome. The current disclosure is a result of extensive efforts to link the TME with ICB therapy outcomes. The TME was classified by analyzing the transcriptome from 11,069 cancer patients based on angiogenesis and T-cell activity. Three distinct angio-immune TME subtypes conserved across 30 non-hematological cancers were found. A clear inverse relationship between angiogenesis and anti-tumor immunity in TME was identified. Experimental evidence showed surprising results in that patients displaying TME with low angiogenesis with strong anti-tumor immunity show the most significant responses to ICB therapy in four cancers. Re-evaluation of the renal cell carcinoma clinical trials provided compelling evidence that the baseline angio-immune state is robustly predictive of ICB responses. This study offers a method of incorporating baseline angio-immune scores for future ICB treatment strategies, thereby improving patient outcomes.

### Example 1: Material and Methods

Table 2 provides a list of reagents and resources used in the current disclosure.

TABLE 2
Key Resource Table

| | | |
|---|---|---|
| REAGENT or | | |
| RESOURCE | SOURCE | IDENTIFIER |

Deposited Data

| | | |
|---|---|---|
| Javelin Renal 101: | Motzer et | 10.1038/s41591-020-1044-8 |
| Pretreatment RNA-Seq | al, 2020 | |
| Hugo: Melanoma | Hugo et | GSE78220 |
| Pretreatment RNA Seq | al., 2016 | |
| Gide: | Gide et | ERP105482 |
| Melanoma Pretreatment | al., 2019 | |
| RNA Seq | | |
| Liu: Melanoma | Liu et al., | phs000452 |
| Pretreatment RNA Seq | 2019 | |
| Mariathasan: Bladder | Mariathas | EGAS00001002556 |
| Cancer Pretreatment | an et al., | |
| RNA Seq | 2018 | |
| Kim: Gastric cancer | Kim et al., | ERP107734 |
| Pretreatment RNA Seq | 2018 | |
| TCGA Pancancer RNA | Grossman | https://gdc.cancer.gov/about-data/publications/pancanatlas |
| Sequencing, Mutations, | et al., | |
| and clinical Data | 2016 | |
| Braun: Renal Cancer | Braun et | 10.1038/s41591-020-0839-y |
| Pretreatment RNA | al., 2020 | |
| sequencing | | |
| Van Allen: Anti-CTLA4 | Van Allen | phs000452.v2.p1 |
| Melanoma pretreatment | et al., | |
| RNA sequencing | 2015 | |
| Nathonson: Anti-CTLA4 | Nathonson | 10.1158/2326-6066.CIR-16-0019 |
| Melanoma pretreatment | et al., | |
| RNA sequencing | 2017 | |

Software and Algorithms

| | | |
|---|---|---|
| GSVA | Hanzelmann | https://www.bioconductor.org/packages/release/bioc/html/GSVA.html |
| | et al., | |
| | 2013 | |
| pheatmap | Kolde, | https://cran.r-project.org/web/packages/pheatmap/index.html |
| | 2012 | |
| msigdbr | NA | https://cran.r-project.org/web/packages/msigdbr/ |
| maftools | Mayakonda | https://bioconductor.org/packages/release/bioc/html/maftools.html |
| | et al., | |
| | 2018 | |
| xCell | Aran et | https://github.com/dviraran/xCell |
| | al., 2017 | |
| CIBERSORT | Chen et | https://cibersort. stanford.edu |
| | al., 2018 | |

### Resource Availability

All datasets used can be accessed using the references listed. All analysis data, gene sets, and code has been deposited in Dryad (https://doi.org/10.5061/dryad.v41ns1s11).

### Method Details

### I. TCGA Data:

RNA, mutations, and clinical profiles for thirty non-hematological TCGA tumor types were used. Cancer types profiled include: Adrenocortical carcinoma (ACC), Bladder Urothelial Carcinoma (BLCA), Brain Lower Grade Glioma (LGG), Breast invasive carcinoma (BRCA), Colon adenocarcinoma (COAD), Cervical squamous cell carcinoma and endocervical adenocarcinoma (CESC), Cholangiocarcinoma (CHOL), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Uterine Carcinosarcoma (UCS), Uterine Corpus Endometrial Carcinoma (UCEC), and Uveal Melanoma (UVM).

RNA sequencing data: RNA sequencing data for 11,069 patients was downloaded from the GDC pan cancer portal (https://gdc.cancer.gov/about-data/publications/pancanatlas). Data was processed using the Firehose pipeline with upper quantile normalization. For patients with more than one RNA-seq sample, primary tumor sample was favored. RNA sequencing samples from patients with DLBC and LAML were excluded.

Mutations: Version 2.8 of the mutations annotation file (MAF) generated by the MC3 group was downloaded from the GDC pan cancer portal (https://gdc.cancer.gov/about-data/publications/pancanatlas). Samples from patients with DLBC and LAML were excluded. Cluster annotations were added to the MAF files. The maftools R package was used for visualization purposes.

Clinical data: Survival information was derived from TCGA-Clinical Data Resource (CDR) Outcome file provided in the GDC pan cancer portal (https://gdc.cancer.gov/about-data/publications/pancanatlas).

### II. Patient Stratification

Building of Angio-immune score matrix: An unbiased selection of all gene sets relating to endothelial cell activity and T-cell activity from the molecular signatures database was conducted. A total of 91 gene signatures were identified and compiled to curate the angio-immune gene set collection. Gene set variation analysis (GSVA) was implemented to score the enrichment of 91 gene sets among patients of 30 TCGA cohorts to generate matrix of enrichment scores.

Clustering gene sets: Correlation matrix using Pearson coefficients were generated across enrichment scores for individual gene sets. Pheatmap package in R was used to generate heatmap visualization. Two modules of gene sets were identified and characterized based on the gene set membership.

Clustering patients: Correlation matrix using Pearson coefficients were generated across enrichment scores for patients. Pheatmap package in R was used to generate heatmap visualization. Three angio-immune subsets were identified and characterized based on distribution of enrichment of different gene sets.

### III. Immune Characteristics of Tumors

Immune and Stromal Cell Enrichment: xCell, a gene signatures-based enrichment approach, was used to delineate enrichment of 64 immune and stromal cell types as previously described. Briefly, the xCell R package was used to generate raw enrichment scores, transform into linear scale, and apply a spillover compensation to derive corrected enrichment scores. Distribution of enrichment scores for patients belonging to different angio-immune clusters were compared.

Immune Cell Deconvolution: CIBERSORT, which uses a nu-support vector regression algorithm to estimate cell fractions in inputted mixture files, was used to depict immune cell fractions from bulk RNA-Seq data as previously described. Briefly, the leukocyte signature matrix, LM22, containing the expression of 547 genes across 22 immune cell subtypes, was used to deconvolute immune cell fractions for each patient in all cohorts. CIBERSORT was run on the R environment (version 4.04) using 100 permutations to generate leukocyte fractions for each patient. An alpha value of 0.05 was used to filter nonsignificant deconvolution results. Patients were grouped by angio-immune cluster and distribution of cell type fraction was compared. Neoantigen load and TCR richness were downloaded from the GDC pan cancer portal (https://gdc.cancer.gov/about-data/publications/pancanatlas).

Immunotherapy datasets: Pre-treatment TPM normalized RNA sequencing data from anti-PD1/L1 and anti-CTLA4 treated cohorts were downloaded for the studies as described in Table 1. Studies were selected based on the following criteria: >20 samples for enrichment calculation, RNA sequencing of pre-treatment biopsies, and only anti-PD1/PDL1 treated patients. Gene signature enrichment and molecular subtypes were derived as described elsewhere in the manuscript. Survival and response rates to treatment when available were compared among angio-immune clusters.

Quantification and Statistical Analysis: Unless indicated elsewhere, all visualizations were completed in GraphPad Prism 9. GraphPad Prism 9 was used for all statistical analysis. Data is presented as mean±standard error of mean. One way ANOVA was used for comparison across more than two groups. Non-parametric tests were used when data was not normally distributed. Log rank tests were used for all survival analysis. Alpha value of 0.05 was used throughout the study.

### Example 2: Angiogenesis and T-Cell Mediated Immunity are Inversely Correlated Across Cancer Types

To characterize the interplay of angiogenesis and T-cell mediated immunity in tumors, ninety-one functional gene sets corresponding to endothelial cell and T-cell activity were compiled from the Molecular Signatures Database (MSigDB, TABLE 1).

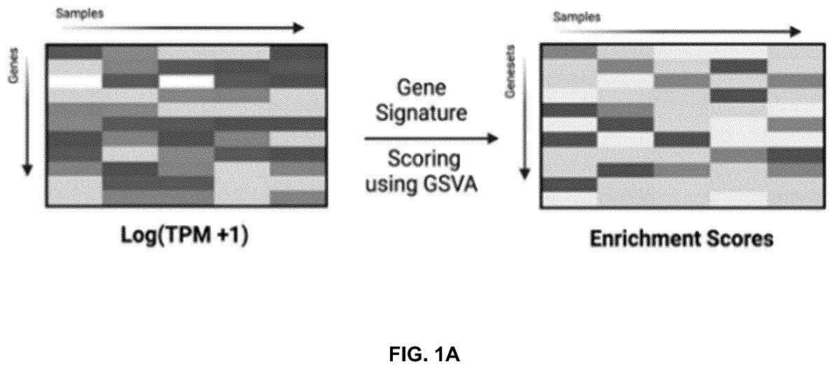

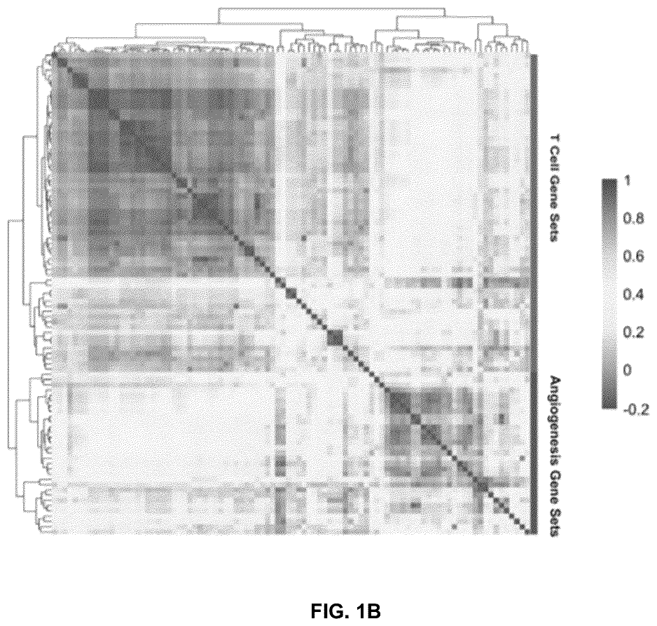

Transcriptomic data from all solid tumor samples from The Cancer Genome Atlas (TCGA) representing 30 non-hematological tumor types were scored for the 91 functional gene sets using Gene Set Variation Analysis (GSVA, FIG. 1A) to deconvolute the TME's angiogenic and T-cell functional landscape. The distributions of gene sets across patients harboring non-hematological tumor types was first investigated. A correlation matrix generated using Pearson Correlation Coefficients (PCC) was built and revealed two functional gene set modules bound by positive correlation (FIG. 1B). We found that T cell gene set scores inversely correlated with angiogenic gene set scores (FIG. 1B). Gene sets corresponding to angiogenic function including, endothelial cell migration, proliferation, sprouting, and angiogenesis, were characteristic of one module. The second module was characterized by gene sets corresponding to T-cell effector function, antigen processing and presentation, and trans-endothelial migration of leukocytes.

### Example 3: Identification of Angio-Immune Subtypes

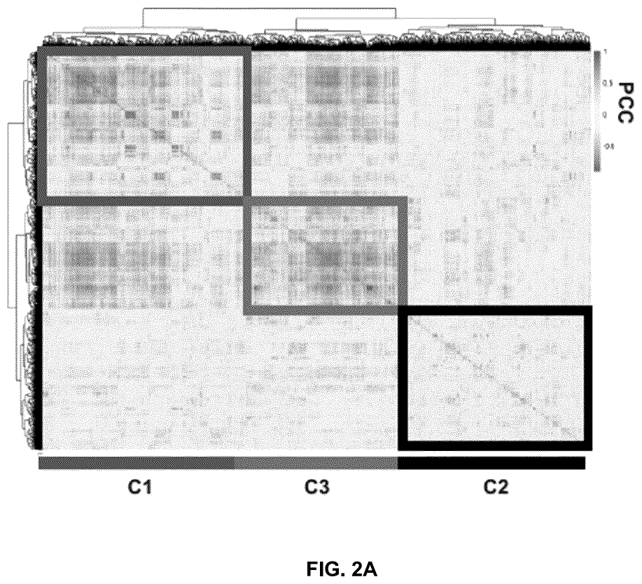

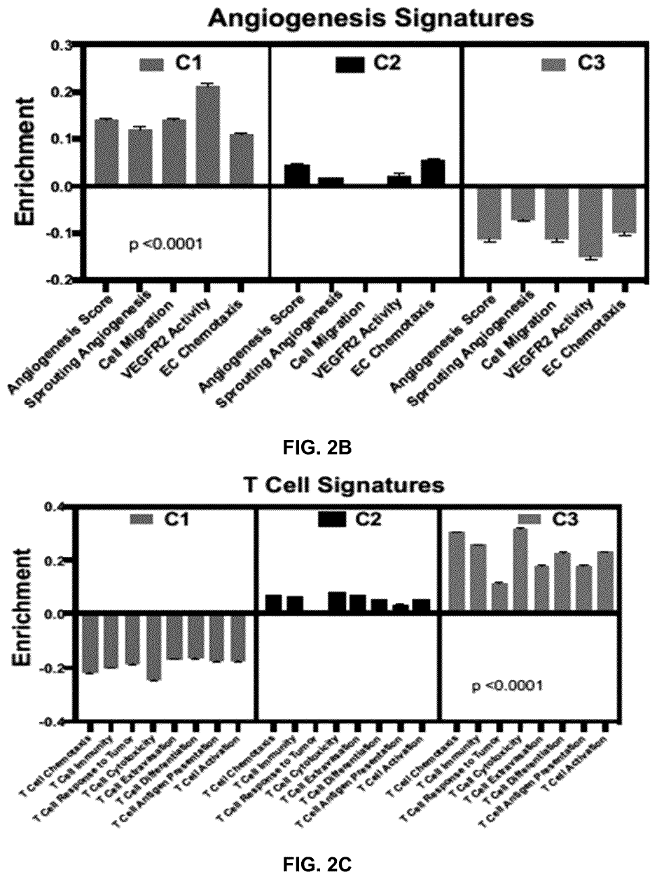

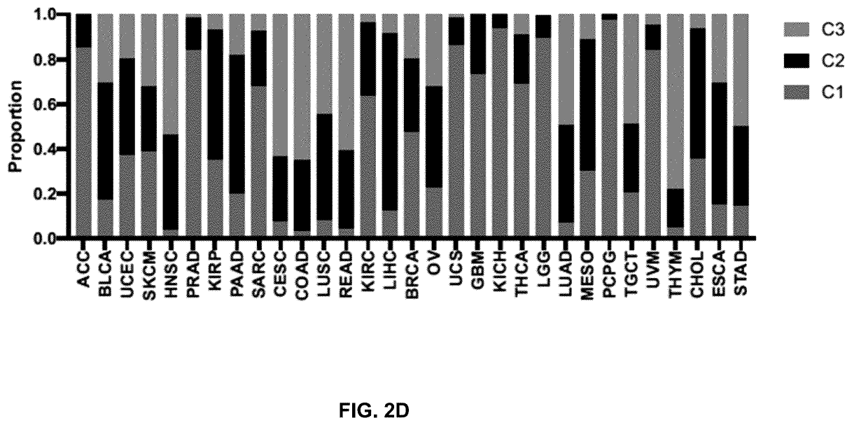

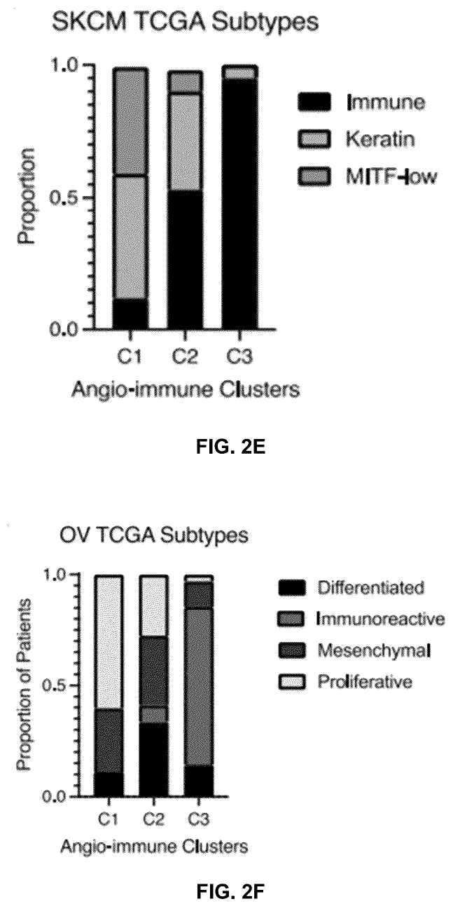

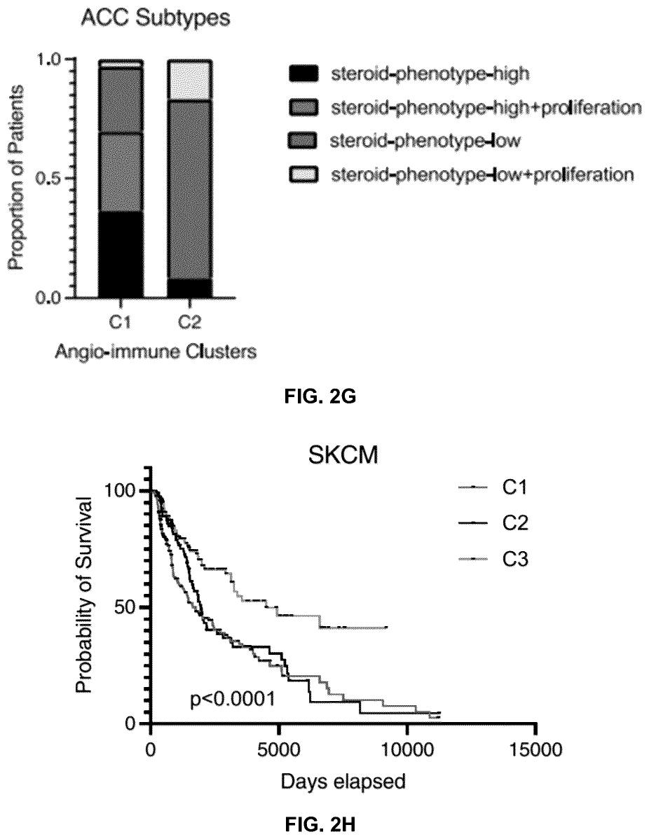

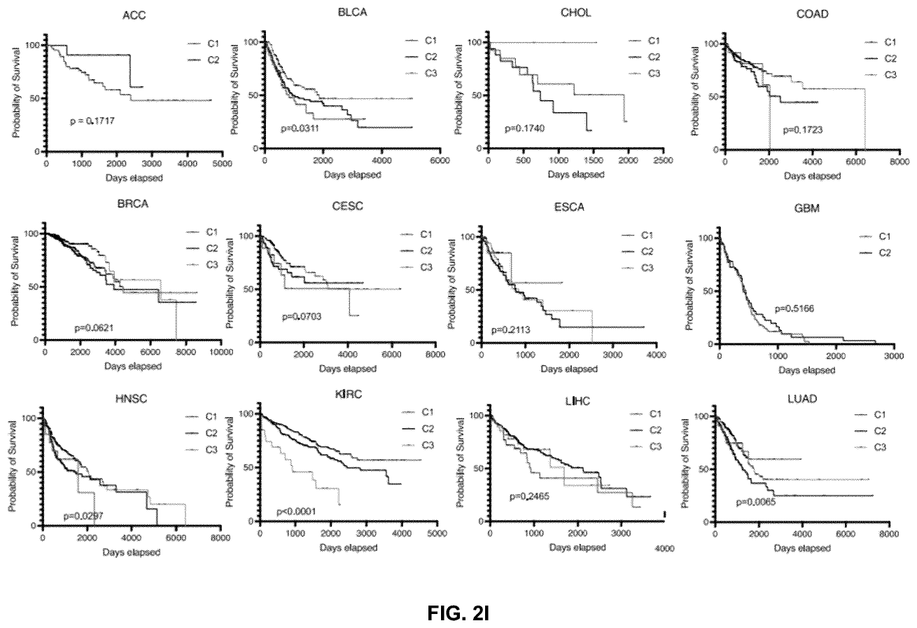

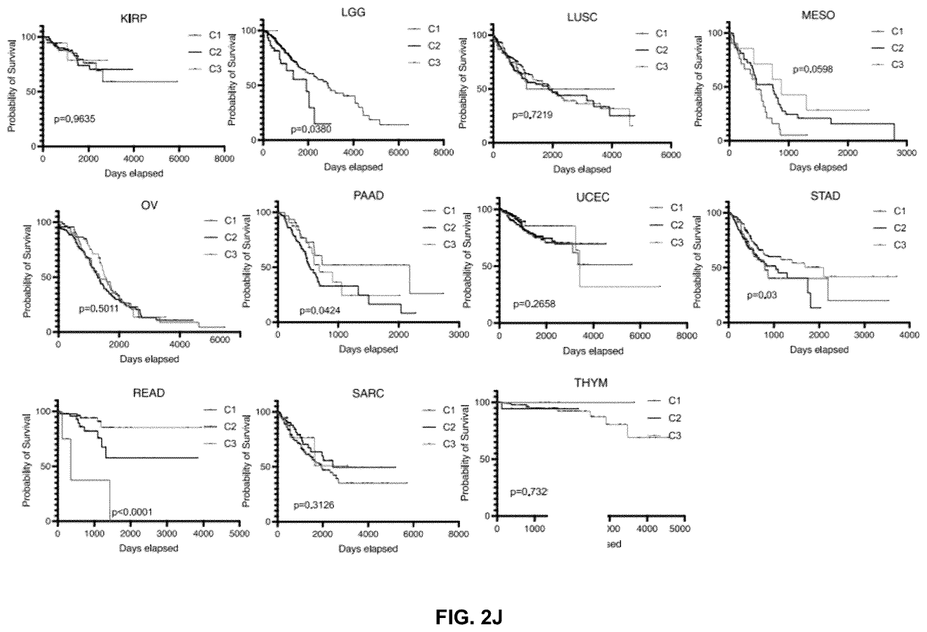

A distinct inverse relationship between angiogenic and immune gene sets suggests that it may be possible to stratify patients based on baseline angiogenic and T-cell activity. To identify angio-immune subtypes of TME based on the distinct distribution of signatures associated with angiogenic and T-cell function, a correlation matrix of patients using the distinct distribution of gene signature enrichment across samples was generated. As a result, distinct subtypes of the TME based on distinct distribution of gene sets characterized the angio-immune scores were uncovered and termed C1, C2, and C3 (FIG. 2A). On one hand, C1 contained strong positive enrichment for angiogenic gene sets, including endothelial cell (EC) migration, EC proliferation, EC sprouting. In contrast, C3 was characterized by the downregulation of functional angiogenesis signatures. C2 had no significant enrichment for functional angiogenesis signatures (FIG. 2B). On the other hand, C1 had marked downregulation of T-cell functional signatures, including T-cell chemotaxis, T-cell mediated cytotoxicity, T-cell extravasation, and antigen presentation to T-cells. Conversely, C3 presented with strong positive enrichment for T-cell functions. Again, C2 had no significant enrichment for T-cell-related gene sets (FIG. 2C). These TME subtypes were conserved across several tumor types. However, the proportion of the microenvironment subtypes varied across tumor types (FIG. 2D).

To ensure pan-cancer clustering is fair to individual tumor types, membership in angio-immune clusters was compared with established RNA-based subtypes of different cancer types, with focus on cancers that contained established RNA based subtypes that directly relate to immune function. mRNA based characterization of skin cutaneous melanoma (SKCM) previously yielded three subtypes: MITF-low, immune, and keratin high. It was noticed in the current study that C3 SKCM tumors were enriched in the immune subtype. Conversely C1 SKCM tumors had the smallest proportion of the immune subtype and was enriched in the keratin high and MITF-low subtypes (FIG. 2E). Similarly, mRNA based characterization of ovarian cancer (OV) previously yielded four subtypes: differentiated, immunoreactive, mesenchymal, and proliferative. Tumors classified as immunoreactive were abundant among C3 OV tumors. C1 OV tumors were enriched for mesenchymal and proliferative tumor subtypes (FIG. 2F). Moreover, previous mRNA based characterization of adrenocortical carcinoma (ACC) previously yielded four subtypes: steroid-phenotype-high, steroid-phenotype-high+proliferation, steroid-phenotype-low, and steroid-phenotype-low+proliferation. Interestingly, C1 ACC tumors contained an abundance of the steroid-high tumors that contain dampened immune response (FIG. 2G). The observed agreement between previously identified molecular clusters in individual cancer types and the angio-immune clusters suggest that the pan-cancer clustering is fair to individual tumor types. Analysis of survival revealed that C3 conferred a marked survival benefit among skin cutaneous melanoma (SKCM) patients who routinely receive immune checkpoint blockade (FIG. 2H). Under challenge with standard of care therapies, survival differences between clusters were observed in bladder adenocarcinoma (BLCA), lung adenocarcinoma (LUAD), head and neck squamous cell carcinoma (HNSC), clear cell renal carcinoma (KIRC), low grade glioma (LGG), pancreatic adenocarcinoma (PAAD), stomach adenocarcinoma (STAD), and renal adenocarcinoma (READ) (FIG. 2I-2J). Together, this analysis provides an easily interpretable framework to classify TMEs independent of the anatomical location of the tumor that may confer survival difference when challenged with treatments like immune checkpoint blockade.

### Example 4: Immune and Somatic Mutation Characteristics of Angio-Immune Subtypes

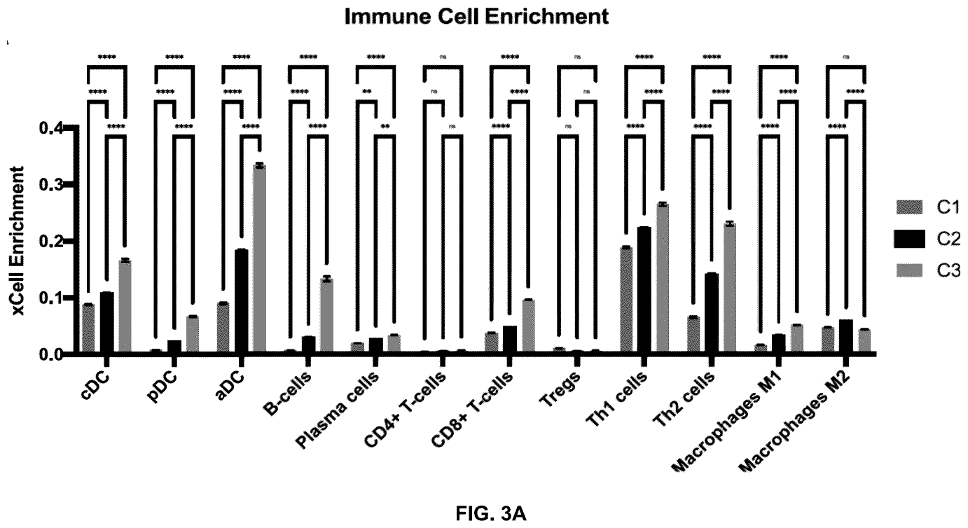

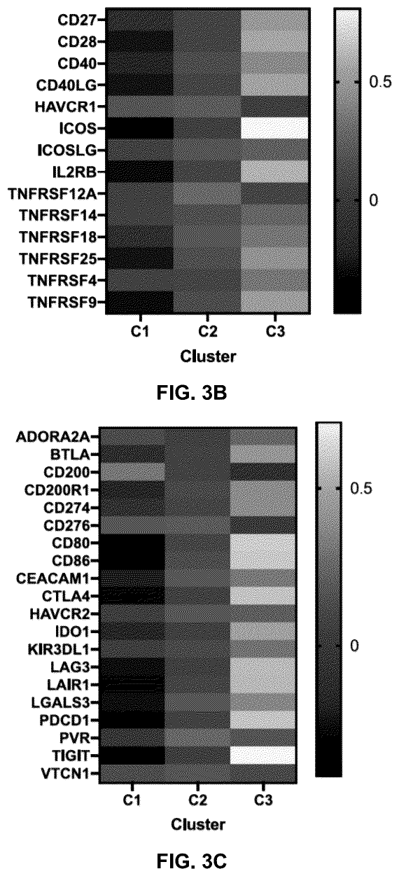

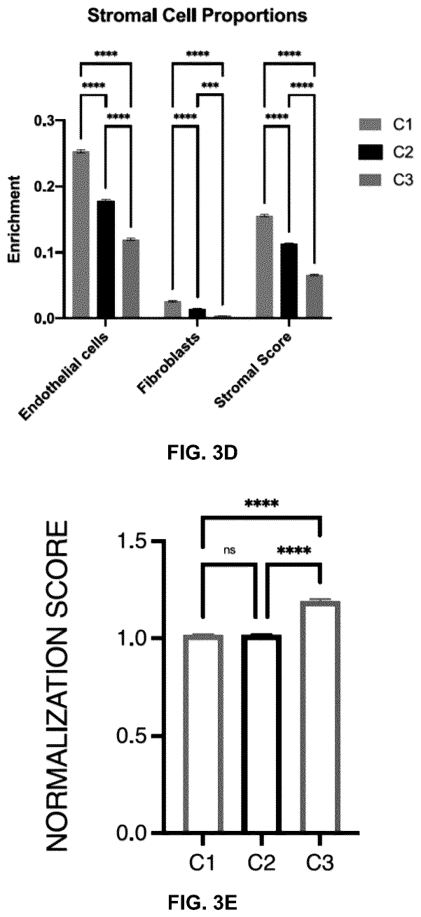

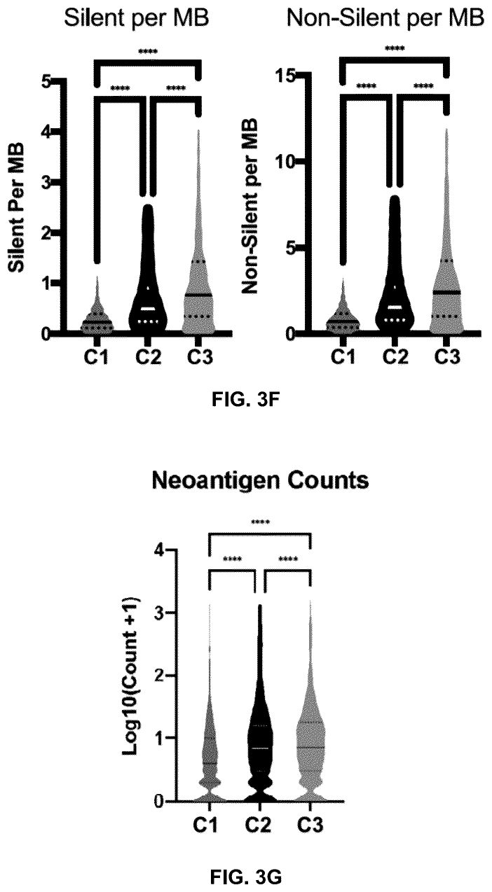

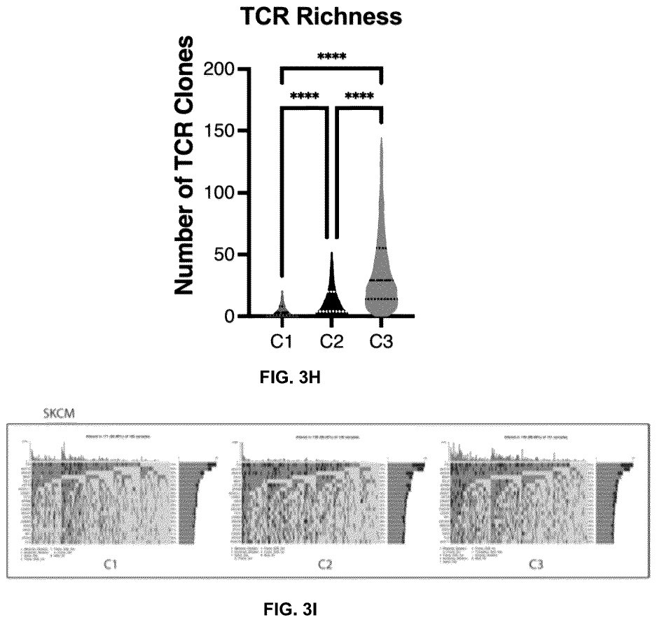

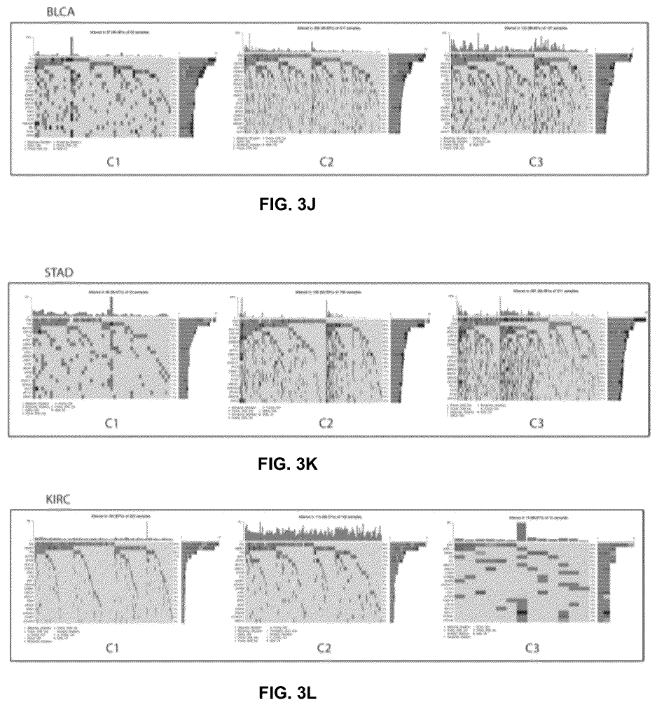

A signature-dependent method to estimate enrichment of cell types in the TME, xCell, was first used to identify the enrichment of 30 different immune and stromal cell types across TME subtypes. Notably, there was a more significant enrichment of dendritic cells, CD8+ T-cells, B cells, Th1 and Th2 cells, and M1 macrophages in C3 (FIG. 3A). Again, to ensure a pan-cancer analysis is fair to individual tumor types, pan-cancer trends in immune cell infiltration were found to be consistent in representative tumors like skin cutaneous melanoma, bladder cancer, gastric cancer, and renal cancer. Infiltrating CD8+ T-cells in C3 have a more significant effector function characteristics as evidenced by increased expression of co-stimulatory molecules in C3 compared to C1 and C2 (FIG. 3B). Infiltrating CD8+ T-cells also display activation as evidenced by the upregulation of exhaustion markers (FIG. 3C). Conversely, C1 was characterized by a distinct increase in enrichment of endothelial cells (FIG. 3D). Fibroblast score was also higher in C1 compared to C2 and C3. C1 may contain a denser stroma, evidenced by a higher stromal score (FIG. 3D). To identify if C3 tumors contained more normalized vasculature, endothelial and pericyte enrichment from XCell were normalized and transformed and the ratio of pericytes to endothelial cells were calculated. Since, normalized vasculature contain a greater coverage of pericytes, the ratio was termed as “vessel normalization score” and it was assessed across angio-immune clusters. Interestingly, C3 tumors displayed a higher ratio of pericyte to endothelial cells suggesting a more normalized vasculature phenotype (FIG. 3E).

Somatic variation in tumor cells can dictate the robustness of immune responses in the TME. A high tumor mutational burden in tumors is suspected of promoting the formation of antigenic peptides that can trigger immune responses. A publicly available MC3 repository for somatic variants derived from whole-exome sequencing data to characterize somatic variations of tumors was analyzed. The silent and non-silent mutational burden was higher in C3 than in C2 and C1 (FIG. 3F). In line with this finding, the mean neoantigen load was greater in C3 than C2 and C1 (FIG. 3G). Infiltrating T-cells also contained greater clonality in TCR in C3 when compared to C2 and C1 (FIG. 3H). Distinct actionable mutations also characterized different angio-immune subtypes in specific tumor types where ICB is considered for first line treatment. Among patients with skin cutaneous melanoma, MGAM and CSMD2 mutations were enriched among C2 and C3 patients (FIG. 3I). Among bladder adenocarcinoma patients, MUC16 and RB1 mutations were enriched among C3 patients, whereas PCLO mutations were enriched among C1 patients (FIG. 3J). In patients with stomach adenocarcinoma, FAT3 and FAT4 mutations were enriched in C2 and C3 patients. C3 patients also had higher rates of TTN mutations (FIG. 3K). Lastly, among clear cell renal carcinoma patients, VHL and BAP1 mutations were enriched in C1 and C3 patients respectively (FIG. 3L). The distinct immune environment of C3 showing higher CD8 T-cell infiltration, increased expression of exhaustion markers, and increased mutational burden suggests that this group of patients may benefit from ICB.

### Example 5: Response and Survival Post ICB

To evaluate if the angio-immune molecular subtypes are prognostic of survival and response upon treatment with ICB, RNA sequencing data from pre-treatment biopsies were queried. Four cohorts of patients were assembled consisting of three different tumor types challenged with ICB: anti-PD1 treated metastatic Melanoma, anti-CTLA4 treated metastatic Melanoma, metastatic gastric cancer, and metastatic bladder cancer.

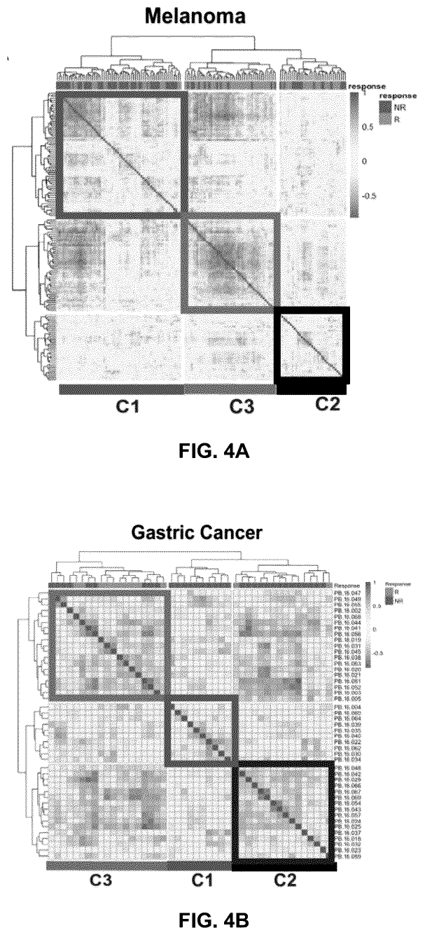

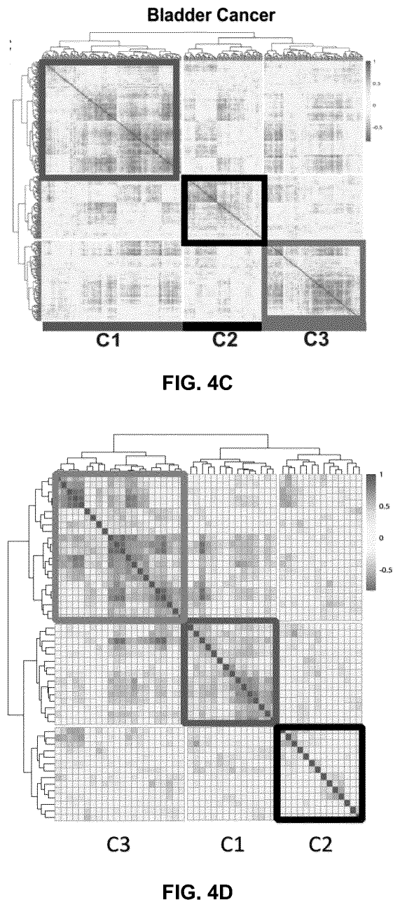

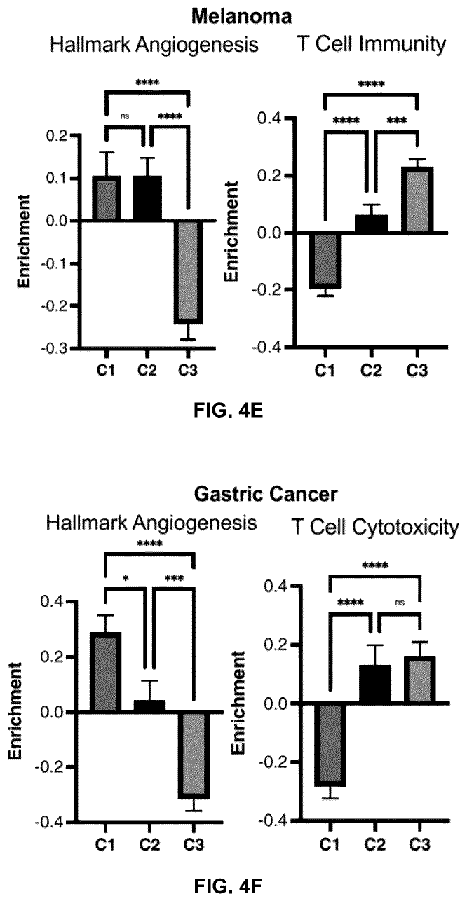

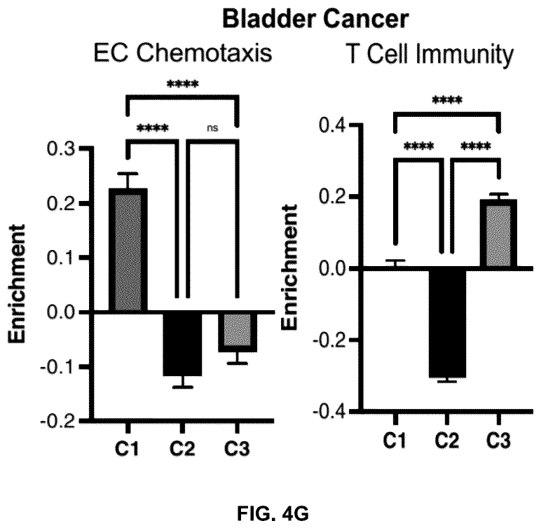

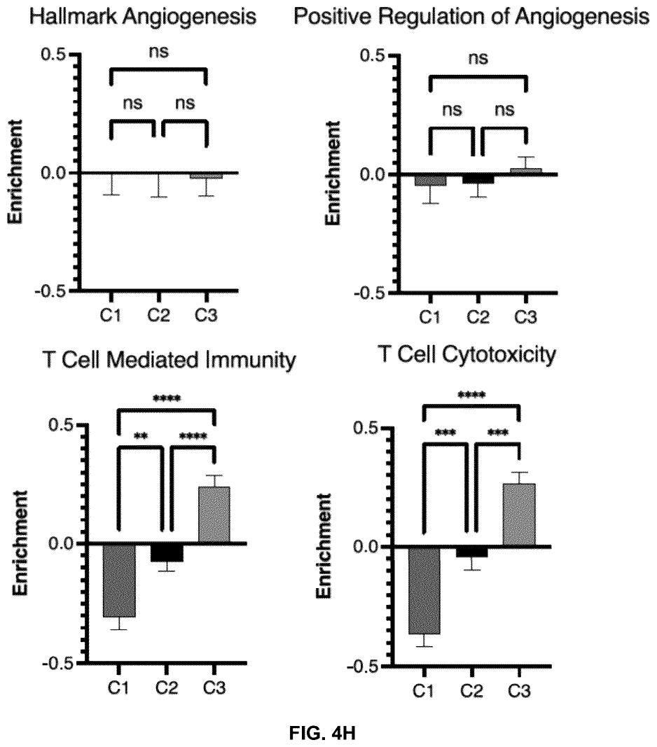

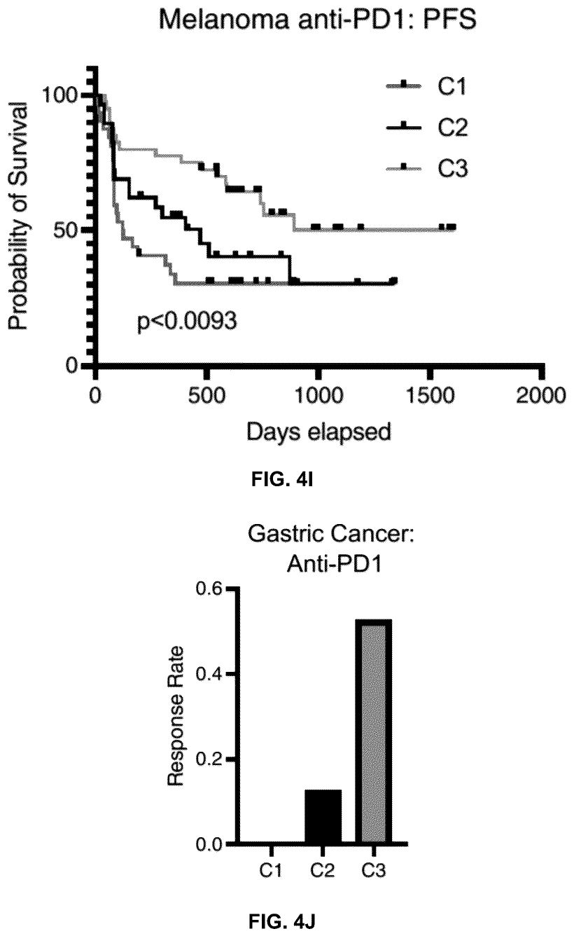

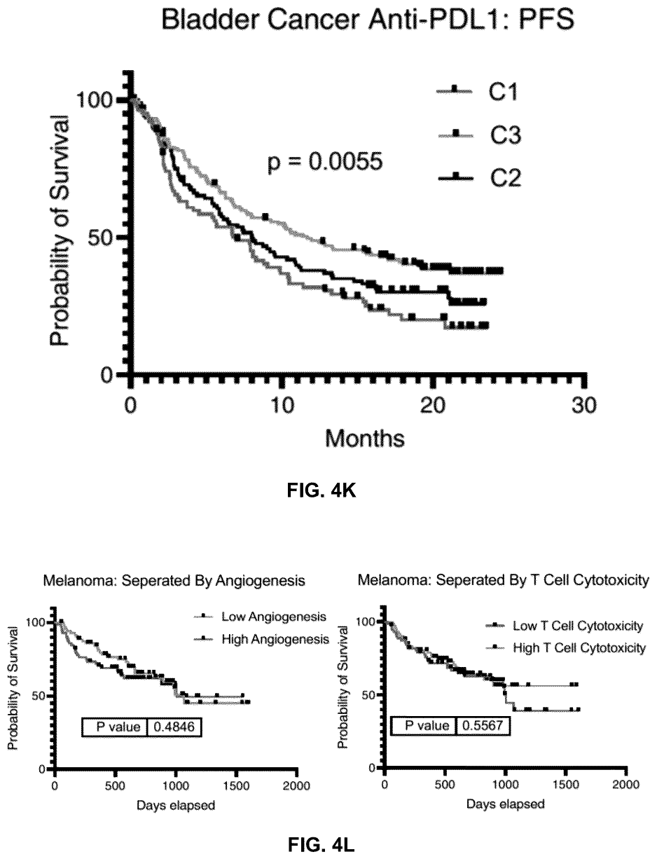

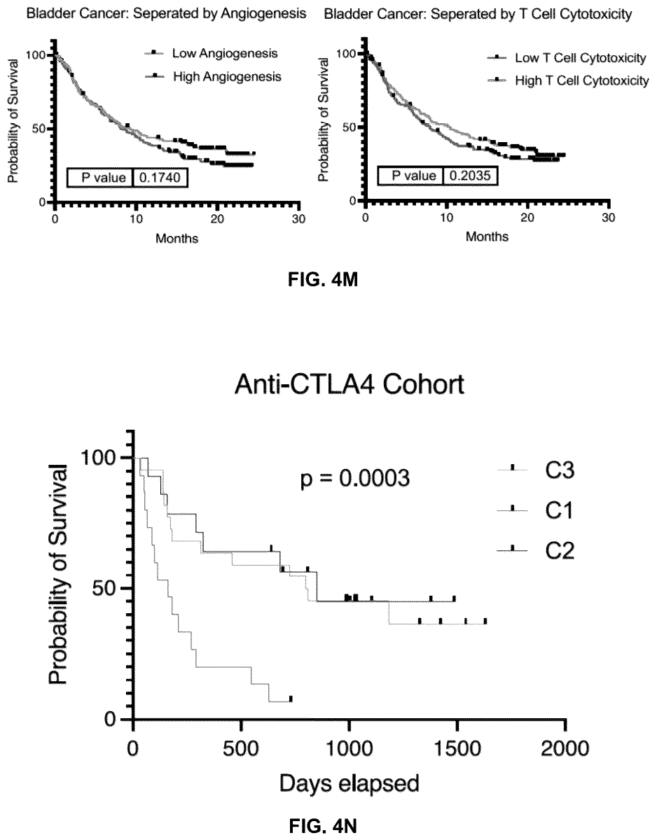

Angio-immune molecular TME subtypes were largely conserved in the anti-PD1 treated metastatic melanoma (FIG. 4A), metastatic gastric cancer (FIG. 4B), metastatic bladder cancer (FIG. 4C) and the anti-CTLA-4 treated metastatic melanoma cohort (FIG. 4D). The distribution of enrichment scores was largely consistent with pan-cancer analysis for anti-PD1 treated metastatic melanoma (FIG. 4E), metastatic gastric cancer (FIG. 4F), metastatic bladder cancer (FIG. 4G), and anti-CTLA-4 treated metastatic melanoma (FIG. 4H). C3 displayed a marked increase in response rate to anti-PD1 therapy among metastatic melanoma tumors. Improved response rate translated to improved progression-free survival (PFS; p<0.0093) and overall survival (OS; p<0.0027) for patients in C3 (FIG. 4I). Melanoma patients in C3 had not reached median PFS. In comparison, the median PFS for C1 was 4.1 months, and the median PFS for C2 was 15.7 months. Similarly, metastatic gastric cancer patients treated with anti-PD1 belonging to C3 displayed a drastic improvement in response rate in comparison to C1 and C2 (FIG. 4J). Likewise, bladder cancer patients belonging to C3 displayed improved PFS than C2 and C1 (p=0.0055; FIG. 4K). Next, to evaluate the relative contribution of each process to the ability of the angio-immune clusters to predict survival of patients treated with ICB, patients were split based on enrichment of an angiogenic signature (Hallmark angiogenesis) and T cell cytoxicity signature. Amongst patients with melanoma and bladder cancer, the two signatures individually failed to be prognostic of survival upon treatment with anti-PD1/PDL1 (FIG. 4I vs FIG. 4L; FIG. 4K vs. FIG. 4M). This suggests that robust T cell activity or poor angiogenic activity alone does not predict response to anti-PD1/PDL1 efficiently, and the synergistic impact of the two is required for a robust prediction. Intriguingly, T cell scores, rather than angiogenic scores, appear to better predict anti-CTLA response. As such, melanoma patients receiving anti-CTLA4 therapy displayed improved survival in C2 and C3 in comparison to C1 (FIG. 4N). The results above demonstrate that angio-immune molecular subtypes can be prognostic of response and survival for patients treated with ICB.

### Example 6: Re-evaluation of Javelin Renal 101, Checkmate 010, and Checkmate 025 Clinical Trials

The approval of the combination of avelumab, an anti-PDL1, and axitinib, a small molecule tyrosine kinase inhibitor, for the first-line treatment of metastatic renal cancer was provided in 2020. The approval was granted on the back of median PFS improvement from 8.4 months with the previous standard of care, sunitinib, a tyrosine kinase inhibitor, to 13.8 months for the combination. Checkmate 010, phase II study, and Checkmate 025, a randomized phase Ill study, demonstrated the benefit of nivolumab, an anti-PD1, over everolimus, a mTOR inhibitor, among clear cell renal carcinoma patients previously treated with anti-angiogenic agents. Next, it was sought to ascertain if stratification by angio-immune subtypes can confer more significant differences in responses and survival.

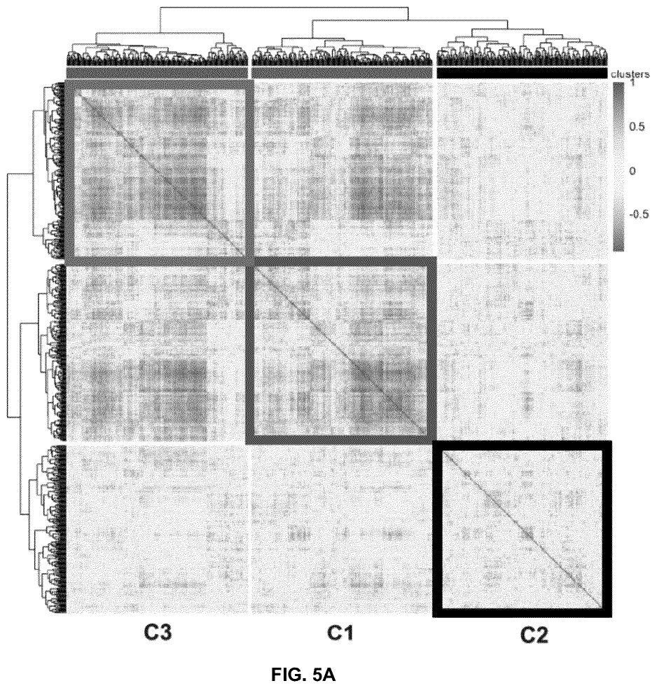

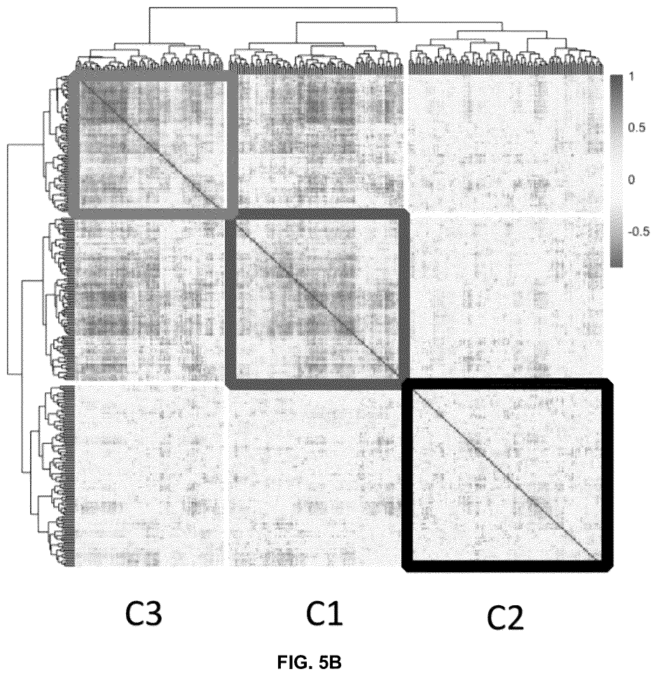

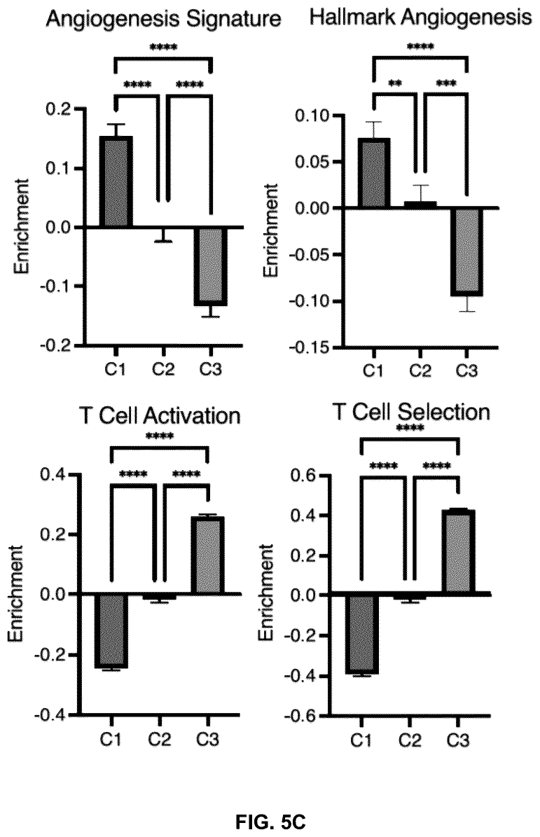

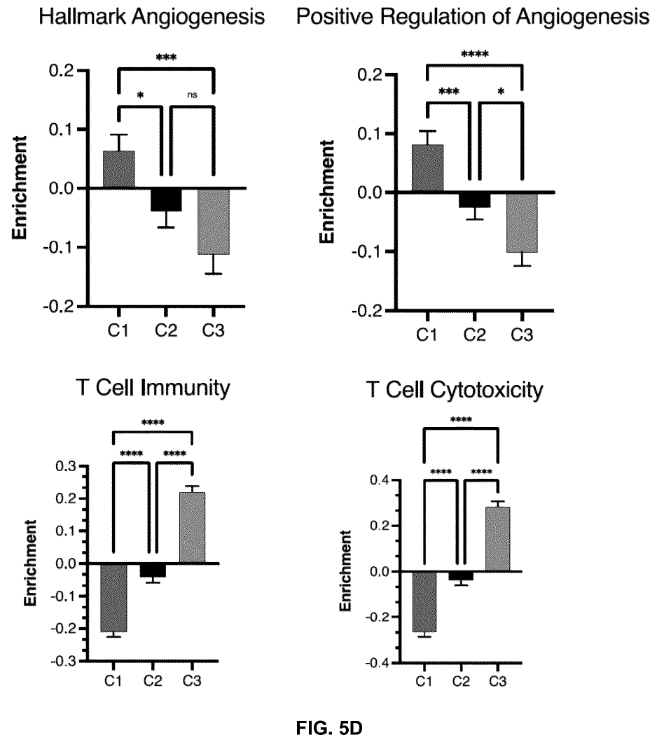

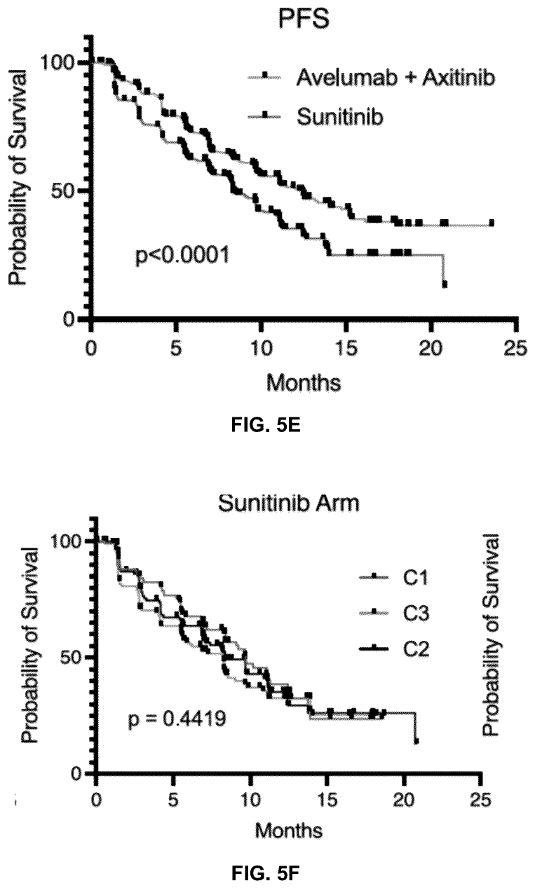

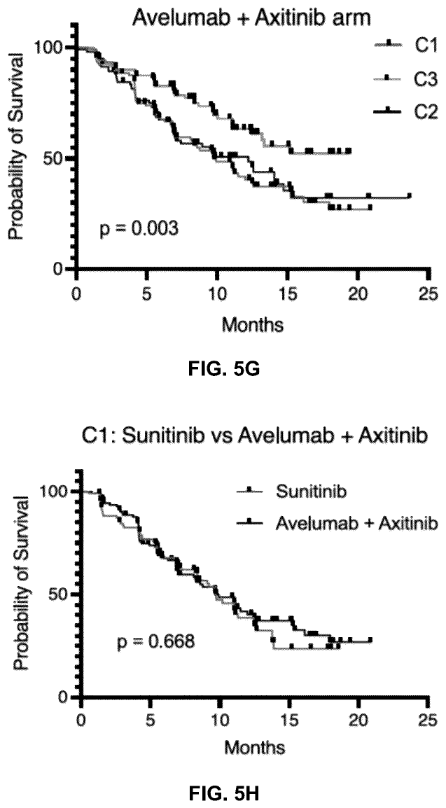

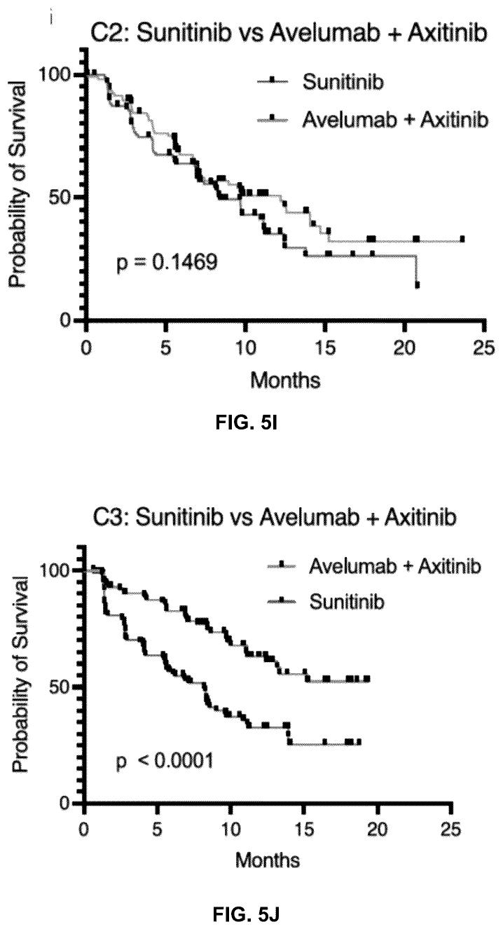

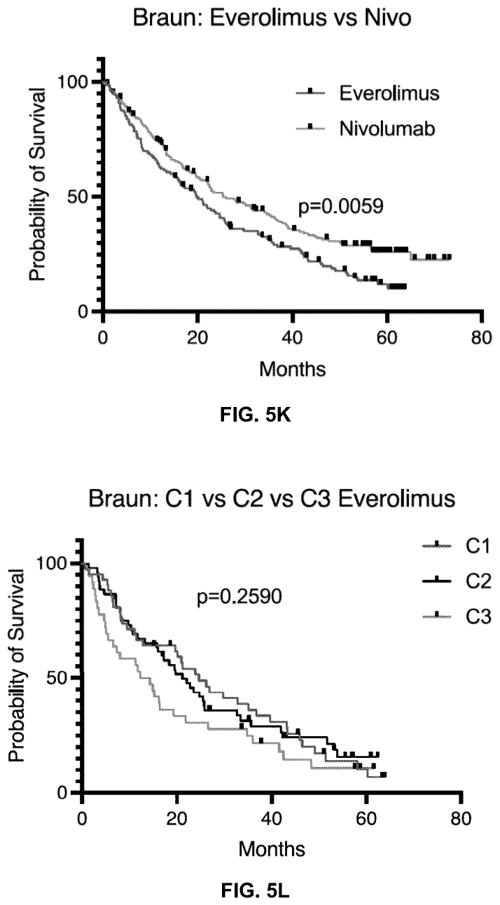

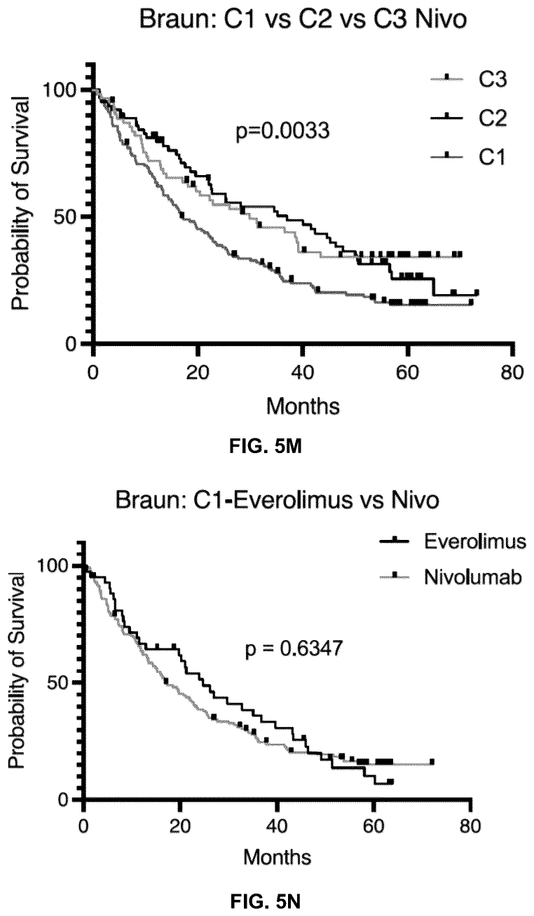

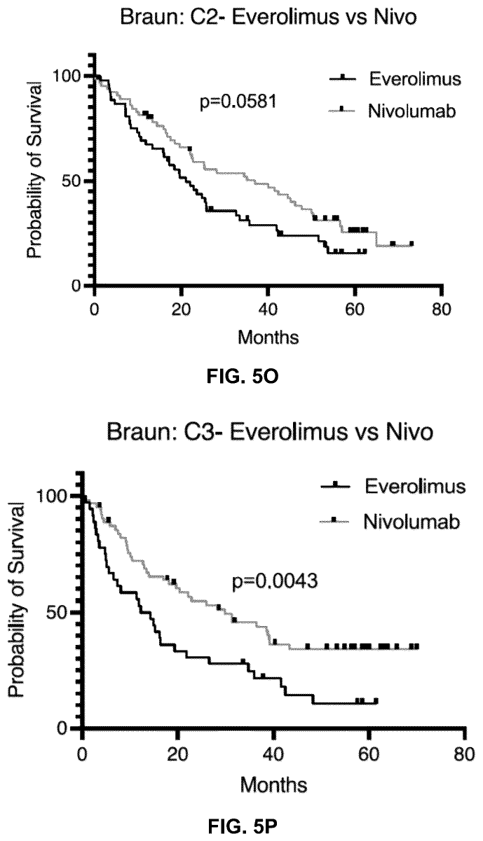

The enrichment of the 91 angio-immune signatures was scored using RNA sequencing data from pre-treatment biopsies. Three angio-immune molecular subtypes were conserved in the Javelin Renal 101 dataset (FIG. 5A) and the Braun dataset (Checkmate 025 and Checkmate 010) (FIG. 5B). Angiogenic and immune gene sets' enrichment distribution was consistent with TCGA data in the Javelin Renal 101 dataset (FIG. 5C) and the Braun dataset (FIG. 5D). As such, C1 presented with an upregulation of angiogenesis functions and a downregulation of T-cell functions. C3 presented with an upregulation of T-cell functions and a downregulation of angiogenesis functions. C2 displayed no significant enrichment in either functional module.

Median PFS improvement for patients with pre-treatment RNA sequencing data in the Javelin Renal 101 dataset was from 8.4 months in the sunitinib arm to 12.5 months in the combination arm (FIG. 5E). Survival was tracked across clusters to identify if angio-immune molecular subtypes can inform treatment choice in this setting. When treated with sunitinib, a tyrosine kinase inhibitor, angio-immune molecular subtypes presented no significant differences in median PFS (FIG. 5F). However, when treated with the combination of avelumab, an anti-PDL1 therapy, and axitinib, a tyrosine kinase inhibitor, significant improvement in survival was observed: the median PFS of C3 had not matured, C2 had a median PFS of 12.2 months, and C1 had a median PFS of 9.7 months (FIG. 5G). To determine if patients belonging to C1, C2, and C3 derive clinical benefit from the combination of avelumab and axitinib, PFS was tracked among angio-immune molecular subtypes across treatment arms. Remarkably, patients belonging to C1 derived no clinical benefit from the combination of avelumab and axitinib compared to sunitinib (Median PFS=9.7 vs. 9.7; p=0.668; FIG. 5H). Patients belonging in C2 also derived no clinical benefit from the combination (Median PFS=12.2 vs 8.3 months; p=0.1469; FIG. 5I). However, among C3, patients treated with avelumab and axitinib displayed significantly improved median PFS than the sunitinib arm (not matured vs. 8.2 months; p<0.001; FIG. 5J).

Median OS improvement for patients with pre-treatment RNA sequencing data in the Braun dataset was from 19.7454 months in the everolimus, a mTOR inhibitor, arm to 25.9877 months in the Nivolumab, an anti-PD1 therapy (FIG. 5K). Survival was tracked across clusters to identify if angio-immune molecular subtypes can inform treatment choice in this setting. When treated with everolimus, angio-immune molecular subtypes presented no significant differences in median OS (FIG. 5L). However, when treated with the Nivolumab, significant improvement in median OS was observed (p=0.0033): the median OS of C3 was 29.8645 months, C2 had a median OS of 36.961 months, and C1 had a median OS of 16.9528 months (FIG. 5M). C3 displayed a better survival than C2 in the long run (FIG. 5M). To determine if patients belonging to C1, C2, and C3 derive clinical benefit from Nivolumab over everolimus, OS was tracked among angio-immune molecular subtypes across treatment arms. Remarkably, patients belonging to C1 derived no clinical benefit from Nivolumab compared to everolimus (Median OS=24.6735 months in the everolimus arm vs 16.9528 months in the Nivolumab arm; p=0.6347;

FIG. 5N). Patients belonging in C2 also derived no statistically significant clinical benefit from the combination (Median OS=36.96 months in the Nivolumab arm vs 21.13 months in the everolimus arm; p=0.0581; FIG. 5O). However, among C3, patients treated with Nivolumab displayed significantly improved median OS than the everolimus arm (Median OS=29.86 months in the Nivolumab arm vs 13.27 months in the everolimus arm; p<0.0043; FIG. 5P). Collectively, this analysis suggests that only patients with low angiogenic status and high T cell activity (cluster C3) mount responses to immune checkpoint blockade. As such, angio-immune molecular subtypes can be used to exclude patients from treatment that would not derive clinical benefit but are faced with adverse toxicities from therapy.

### Example 7: Angio-Immune Clusters Better Predict ICB Survival in Comparison to Previous Methods

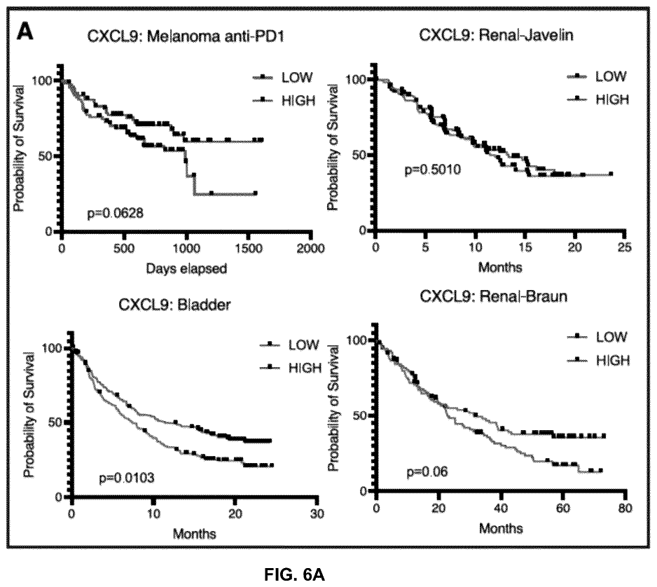

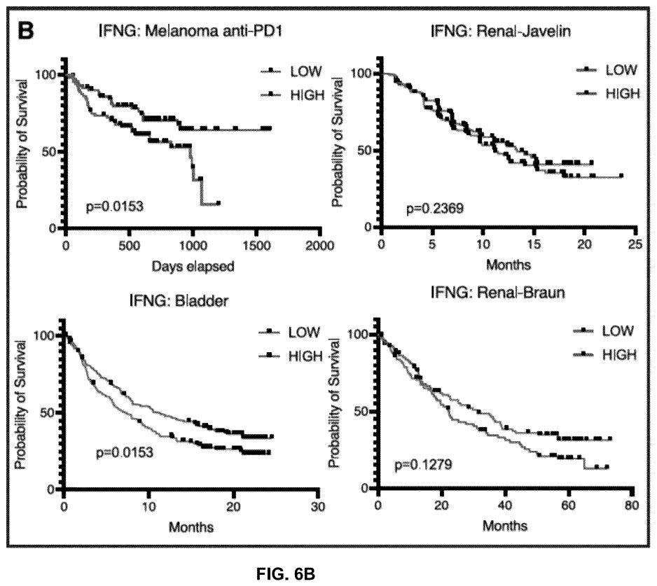

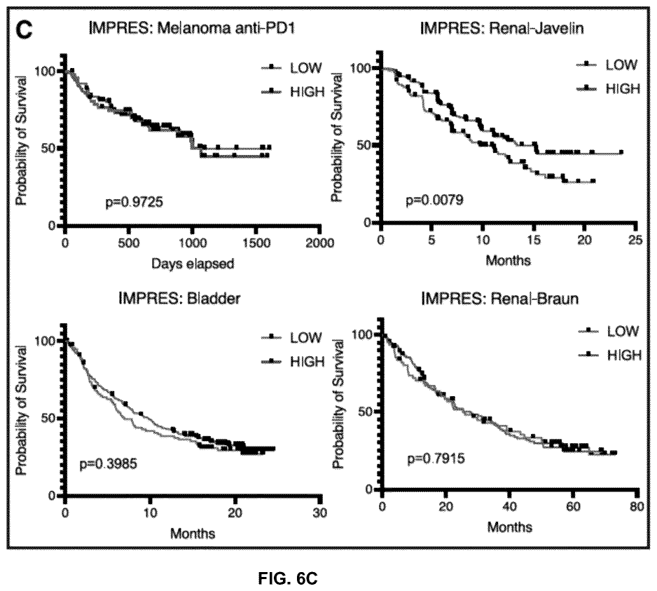

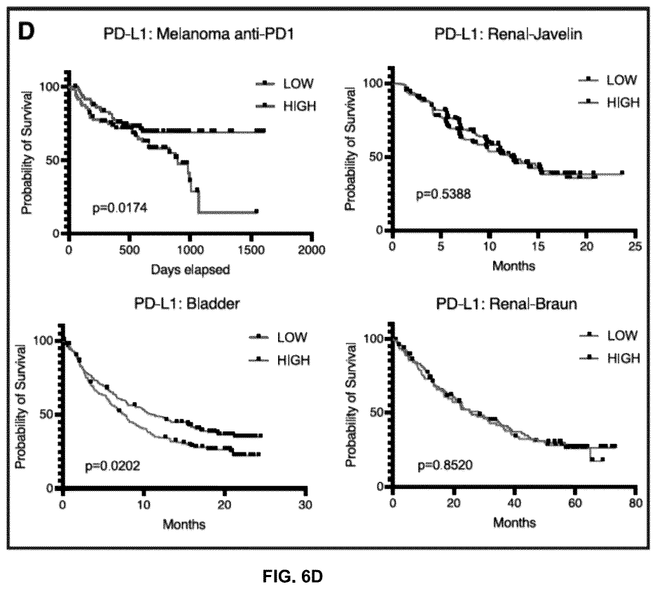

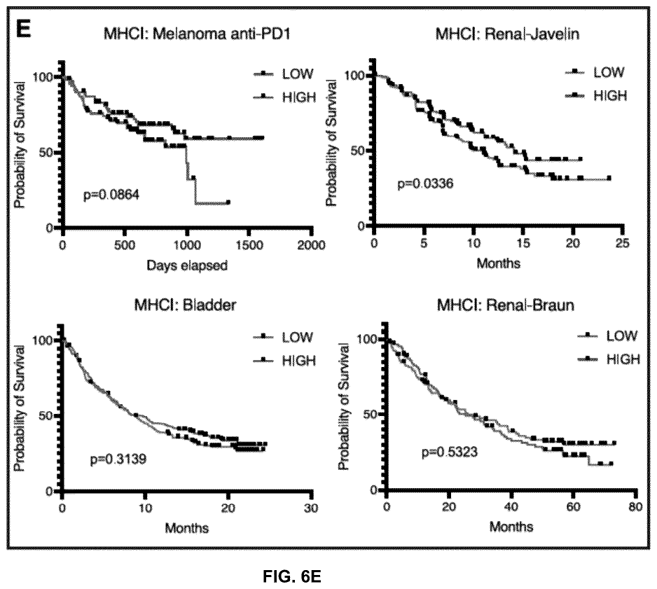

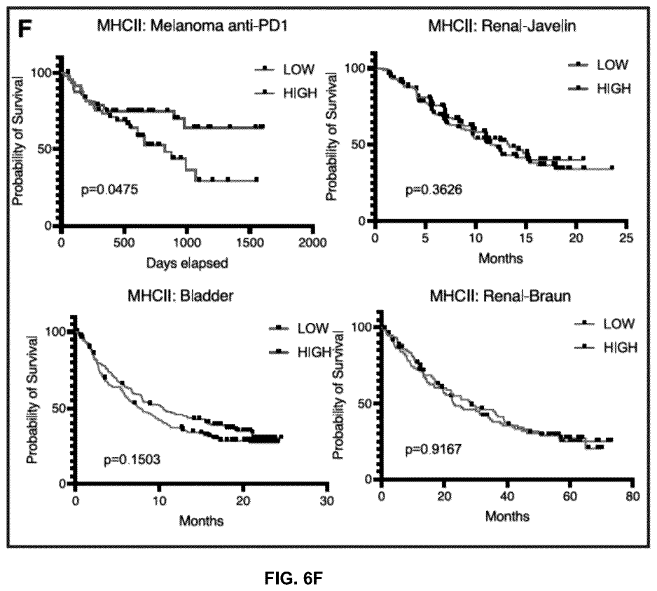

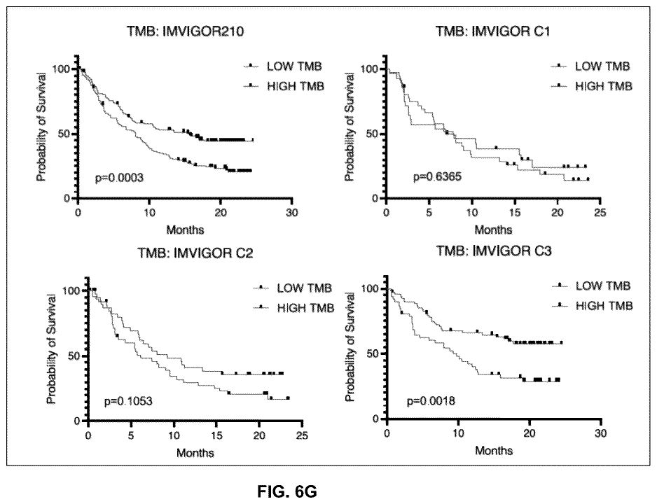

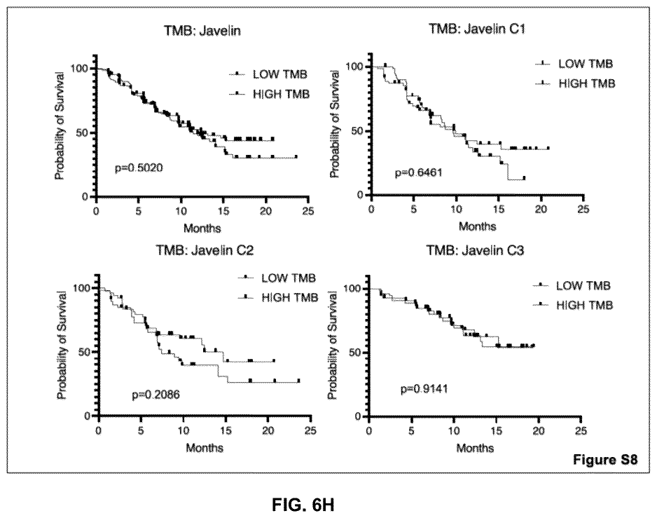

Poor response rate upon immune checkpoint blockade treatment has sparked a range of studies seeking to identify predictors of response among treated patients. The angio-immune subtypes are based on the interaction of the vascular and immune biological systems that is independent of tumor type. As such, to display the universality of the predictive power of the angio-immune subtypes, the predictive power of CXCL9 expression, IFNg signature, IMPRES (an immune-predictive score), PD-L1 expression, MHCI expression, and MHCII expression across anti-PD1 treated melanoma patients, anti-PD1/PD-L1 treated renal cancer patients, and anti-PD-L1 treated bladder cancer patients were comparatively evaluated. Remarkably, none of these tools displayed a universal ability to predict survival upon ICB treatment (FIG. 6A-6H). High CXCL9 expression was predictive of improved survival in melanoma and bladder cancer patients (IMVIGOR210), but failed to predict improved survival in renal cancer (FIG. 6A). High IFNg signature enrichment was predictive of improved survival in melanoma and bladder cancer patients but failed to predict improved survival in renal cancer (FIG. 6B). Intriguingly, high IMPRES scores were predictive of survival only in one cohort of renal cancer patients and failed to predict responses in the melanoma and bladder cancer patient cohort (FIG. 6C). High PD-L1 expression predicts response in anti-PD1 treated melanoma and anti-PD-L1 treated bladder cancer patients but fails to do so among renal cancer patients (FIG. 6D). Similarly, baseline MHC I expression was predictive of survival only in one cohort of renal cancer patients and melanoma but failed to predict responses in the bladder cancer cohort (FIG. 6E). Baseline MHC II expression was predictive of improved survival only in the melanoma cohort (FIG. 6F).

The IMVIGOR210 bladder cancer clinical trial reported the tumor mutational burden (TMB) was predictive of improved survival in bladder cancer patients treated with anti-PDL1. Indeed, patients with high TMB displayed drastically improved survival in comparison with patients with low TMB (FIG. 6G). Intriguingly, the survival benefit conferred by high TMB dissipates in patients belonging to clusters C1 and C2 (FIG. 6G). Only C3 patients with low angiogenic activity and high T cell activity derive clinical benefit when stratified by TMB. This dependence is absent in tumors where TMB does not predict response like renal cell carcinoma (FIG. 6H). This observation suggests that the biology underpinning angio-immune clusters is indispensable for the use of TMB as a predictive biomarker for ICB response. Together, it is shown herein that angio-immune subtypes display a remarkable universality and can effectively predict responses to ICB across different tumor types superior to other prognostic features.

### Example 8: Discussion

ICB has revolutionized cancer treatment outcomes for a subset of patients. However, a large subset of patients fail to show responses. It is imperative to 1) identify strategies to improve response rates for patients upon ICB treatment and 2) identify pre-treatment characteristics of patients that can provide an a priori prediction to patients' response to treatment. Such efforts can minimize toxicity profiles faced by patients while maximizing clinical benefits derived from ICB.

Herein, it was examined whether baseline angiogenic state and corresponding T-cell immune activity can provide tools to better inform treatment decision-making processes and delineate resistance mechanisms to ICB. It was demonstrated that angiogenic activity and T-cell mediated immunity are inversely correlated across patients with 30 non-hematological solid tumor types. Distinct distribution of angiogenic and T-cell activity enrichment across tumor types enabled the stratification of patients into three conserved tumor angio-immune subtypes, high angiogenesis, and low anti-tumor immunity (C1), low angiogenesis and high anti-tumor immunity (C3), and the one in between (C2). The vasculature in C3 tumors are more normalized as evidenced by the higher pericyte to endothelial cell ratio. While tumor heterogeneity plagues efforts to develop overarching rules to define tumors independent of tissue of origin, the remarkable conservation of the distinct relationship between angiogenesis and T-cell mediated immunity across solid tumors allowed to develop highly interpretable rules to predict ICB response across all tumor types.

The importance of baseline angiogenic state provides a strong foundation for a temporal treatment strategy characterized by vascular normalization followed by ICB. As such, patients with low angiogenesis levels and corresponding high T-cell activity belonging to C3 consistently respond better to local acting ICB in melanoma, gastric cancer, bladder cancer, and renal cell cancer. Notably, the immune features alone of the C3 fail to effectively predict responses in ICB patients with Renal, Gastric, and Bladder cancer35, suggesting the essential role angiogenesis may play in dictating the kind of immune response required to confer responses.

Re-evaluation of the Javelin 101, Checkmate 025, and Checkmate 010 renal cancer clinical trials provide therapeutic relevance of the identified TME subtypes. Patients categorized in the C3 angio-immune subtype demonstrated remarkable responses to the combination of axitinib, a small molecule tyrosine kinase inhibitor, and avelumab, an anti-PDL1, and received clinically significant improvements in survival compared to the previous standard of care sunitinib, a tyrosine kinase inhibitor, among the patients in the Javelin Renal 101 clinical trial. Conversely, patients in C2 and C1 did not benefit from axitinib combined with avelumab compared to the previous standard of care sunitinib. Patients harboring C3 angio-immune subtype were the only population to display the benefit of nivolumab, an anti-PD1 therapy, over the previous standard of care everolimus, a mTOR inhibitor, in the Checkmate 025 and Checkmate 010 clinical trials. This analysis suggests that the angio-immune subtypes can reliably identify a subset of patients that would derive clinical benefit from the ICB treatment in renal cell carcinoma.

ICB treatment allows the silencing of inhibitory signals on T-cells to enable reactivity against tumor cells. The current clinical landscape of treatment with ICB targets two key checkpoint molecules: CTLA4 and PD1. CTLA4 is an inhibitory receptor that competes with co-stimulatory receptor CD28 for B7 (CD80/CD86) binding. B7 expression is primarily restricted to antigen-presenting cells in the tumor-draining lymph nodes. As such, the anti-CTLA4 is thought to affect the priming phase of CD8+ T cell activation primarily acting in lymph nodes. The data that anti-CTLA4 response was better predicted predominantly by the immune scores alone probably reflects the mode of anti-CTLA4 action. In comparison, anti-PD1/PDL1 treatments affect during the effector phase in the tumor microenvironment. Tumor or tumor associated macrophage upregulation of PD-L1 engages with inhibitory PD1 receptors on T-cells. TME characteristics may have a more direct impact on anti-PD1/PDL1 efficacy. Immune features in C3 microenvironment include improved infiltration of anti-tumor immune cells, higher cytotoxicity activity of CD8+ T-cells, higher expression of co-stimulatory molecules, and higher expression of markers of T-cell exhaustion. The highly inflamed profile of C3 is partly due to the high mutational burden and neoantigen load in this category of patients. Importantly, distinct differences were identified in mutational profiles of patients belonging to different angio-immune subtypes. The distinct mutations are actionable and may present distinct treatment modalities for patients harboring differing TME subtypes. Local immune characteristics of patients belonging to C3 provide a compelling rationale for exploring treatment with locally acting anti-PD1/PDL1 ICB.

In summary, leveraging the biological relationship between angiogenesis and T cell immunity in the tumor microenvironment allowed the development of a framework with universal predictive power for identifying patients that will derive benefit from ICB treatment. Importantly, angio-immune subtypes outperform previous predictive methods and are remarkably conserved across tumor types.

### EQUIVALENTS

While several inventive aspects have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive aspects described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive aspects described herein. It is, therefore, to be understood that the foregoing aspects are presented by way of example only and that, within the scope of the appended claims and equivalents thereto, inventive aspects may be practiced otherwise than as specifically described and claimed. Inventive aspects of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one aspect, to A only (optionally including elements other than B); in another aspect, to B only (optionally including elements other than A); in yet another aspect, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

1. (canceled)

2. A method of treating a cancer in a subject in need thereof, comprising:
(i) providing expression levels of two or more genes in a biological sample obtained from tumor microenvironment (TME) of the subject, wherein the two or more genes encode effectors that impact angiogenesis or T-cell function or both;
(ii) determining an angio-immune score based on the expression levels of the two or more genes in step (i), wherein a score below an angio-immune baseline score is indicative of the subject as having an angio-immune subtype corresponding to a low angiogenic signature and a high T cell function signature; and
(iii) administering to the subject identified as having the angio-immune subtype corresponding to a low angiogenic signature and a high T cell function signature a treatment comprising an immune checkpoint blockade (ICB) therapeutic.

3. A method of identifying a subject as responsive to ICB therapy, comprising:
(i) providing expression levels of two or more genes in a biological sample obtained from the tumor microenvironment (TME) of the subject, wherein the two or more genes encode effectors that impact angiogenesis or T-cell function or both;
(ii) determining an angio-immune score based on the expression levels of the two or more genes in step (i);
(iii) comparing the angio-immune score to an angio-immune baseline score for a corresponding tumor microenvironment to determine an angio-immune subtype; and
(iv) identifying the subject as responsive to an ICB therapy based on the angio-immune subtype.

4. The method of claim 2, wherein the two or more genes are selected from one or more of:
(i) genes that impact angiogenesis selected from:
(a) MYCT1, APOH, APP, CCND2 COL3A1, COL5A2, CXCL6, FGFR1, FSTL1, ITGAV, JAG1, JAG2, KCNJ8, LPL, LRPAP1, LUM, MSX1, NRP1, OLR1, PDGFA, PF4, PGLYRP1, POSTN, PRG2, PTK2, S100A4, SERPINA5, SLCO2A1, SPP1, STC1, THBD, TIMP1, TNFRSF21, VAV2, CAN, VEGFA, VTN, CDH5, ELTD1, CLEC14A, LDB2, ECSCR, RHOJ, VWF, TIE1, KDR, ESAM, CD93, PTPRB, GPR116, SPARCL1, EMCN, ROBO4, ENG, TEK, SIPR1; or
(b) AGGF1, AMOT, ANGPTL3, ANGPTL4, BTG1, CHRNA7, RHOB, RUNX1, SPHK1, TNFSF12; or
(c) APOH, APP, CCND2, COL3A1, COL5A2, CXCL6, FGFR1, FSTL1, ITGAV, JAG1, JAG2, KCNJ8, LPL, LRPAP1, LUM, MSX1, NRP1, OLR1, PDGFA, PF4, PGLYRP1, POSTN, PRG2, PTK2, S100A4, SERPINA5, SLCO2A1, SPP1, STC1, THBD, TIMP1, TNFRSF21, VAV2, CAN, VEGFA, VTN; or
(d) ABL1, ADAMTS9, AGTR1, AKT3, ALOX5, ANXA1, APELA, APLNR, BMPER, CARD10, CEACAM1, CEMIP2, CIB1, CREB3L1, DLL1, DLL4, E2F2, EPN1, EPN2, FBXW7, FGF1, FGF2, FGFBP1, FOXC2, FUT1, GATA2, GHRL, GHSR, GLUL, HDAC5, HDAC7, HDAC9, HMOX1, IL10, IL12A, IL12B, ITGA5, ITGB1BP1, JAK1, JCAD, JMJD8, KDR, KLF2, KLF4, MAP2K5, MAP3K3, MEOX2, MIR1-1, MIR1-2, MIR101-1, MIR101-2, MIR10A, MIR10B, MIR1224, MIR125A, MIR126, MIR132, MIR138-1, MIR138-2, MIR146A, MIR149, MIR150, MIR15A, MIR15B, MIR16-1, MIR16-2, MIR17, MIR188, MIR18A, MIR193A, MIR196A1, MIR196A2, MIR199A1, MIR199A2, MIR199B, MIR19A, MIR19B1, MIR19B2, MIR2000, MIR206, MIR20A, MIR21, MIR22, MIR221, MIR222, MIR2355, MIR23A, MIR24-1, MIR24-2, MIR26A1, MIR26A2, MIR27A, MIR27B, MIR296, MIR29C, MIR30B, MIR30C1, MIR30C2, MIR30E, MIR31, MIR320A, MIR329-1, MIR329-2, MIR342, MIR34A, MIR34B, MIR34C, MIR361, MIR375, MIR377, MIR410, MIR424, MIR483, MIR487B, MIR494, MIR495, MIR497, MIR503, MIR92A1, MIR92A2, MIRLET7B, MIRLET7F1, MIRLET7F2, MMRN2, NGFR, NOTCH1, NR2E1, NRP1, PDCD10, PDPK1, PIK3C2A, PKM, PLK2, PPP1R16B, PTGS2, RHOA, RHOJ, S100A1, SEMA6A, SMAD1, SPRED1, SRPX2, STARD13, SYNJ2BP, TBXA2R, THBS1, TJP1, VEGFA; or
(e) ABL1, AGTR1, AKT3, ANXA1, APELA, APLNR, BMPER, CIB1, DLL1, FGF1, FGF2, FGFBP1, FOXC2, FUT1, GATA2, GHRL, GHSR, HDAC7, HDAC9, HMOX1, IL10, ITGA5, JAK1, JCAD, JMJD8, KDR, KLF4, MAP3K3, MIR1-1, MIR1-2, MIR101-1, MIR101-2, MIR10A, MIR10B, MIR1224, MIR125A, MIR126, MIR132, MIR146A, MIR150, MIR21, MIR23A, MIR27A, MIR27B, MIR296, MIR30B, MIR31, MIR487B, MIR503, MIR92A1, MIR92A2, MIRLET7B, MIRLET7F1, MIRLET7F2, NRP1, PDPK1, PIK3C2A, PKM, PLK2, PPP1R16B, PTGS2, RHOJ, S100A1, SMAD1, SRPX2, TJP1, VEGFA; or
(f) ABL1, AKT3, ANXA1, CIB1, FGF2, FGFBP1, FOXC2, GATA2, HDAC7, HDAC9, HMOX1, JCAD, KDR, MAP3K3, MIR101-1, MIR101-2, MIR10A, MIR10B, MIR126, MIR132, MIR146A, MIR150, MIR23A, MIR27A, MIR27B, MIR296, MIR31, MIR487B, MIRLET7F1, MIRLET7F2, NRP1, PIK3C2A, PLK2, PTGS2, RHOJ, SRPX2, VEGFA; or
(g) ABL1, ADTRP, AKT1, KT3, ANXA1, CARD10, CIB1, CLEC14A, DLL4, EFNB2, EGR3, EPHB4, FBXW7, FGF2, FGFBP1, FOXC2, GATA2, GPLD1, GREM1, HDAC5, HDAC7, HDAC9, HMOX1, ITGB1, ITGB1BP1, JCAD, KDR, KLF4, MAP2K5, MAP3K3, MEOX2, MIA3, MIR101-1, MIR101-2, MIR10A, MIR10B, MIR126, MIR132, MIR146A, MIR149, MIR150, MIR15A, MIR16-1, MIR16-2, MIR188, MIR193A, MIR196A1, MIR196A2, MIR199A1, MIR199A2, MIR199B, MIR19B1, MIR19B2, MIR2000, MIR206, MIR20A, MIR22, MIR221, MIR2355, MIR23A, MIR26A1, MIR26A2, MIR27A, MIR27B, MIR296, MIR29C, MIR31, MIR320A, MIR329-1, MIR329-2, MIR361, MIR410, MIR424, MIR483, MIR487B, MIR494, MIR495, MIR497, MIR503, MIRLET7F1, MIRLET7F2, MMRN2, NOTCH1, NR2E1, NR4A1, NRP1, PDCD10, PIK3C2A, PIK3R3, PLK2, PTGS2, RHOA, RHOJ, ROBO1, SLIT2, SPRED1, SRF, SRPX2, STARD13, TBXA2R, TDGF1, THBS1, VEGFA; or
(h) ACVRL1, AGGF1, AMOT, ANG, ANGPTL3, ANGPTL4, ATP5IF1, BTG1, C1GALT1, CANX, CDH13, CHRNA7, COL4A2, COL4A3, CXCL8, EGF, EMCN, EPGN, ERAP1, FOXO4, HTATIP2, IL17F, IL18, MYH9, NCL, NF1, NOTCH4, NPPB, NPR1, PF4, PLG, PML, PROK2, RHOB, RNH1, ROBO4, RUNX1, SCG2, SERPINF1, SHH, SPHK1, SPINK5, STAB1, TGFB2, THY1, TNFSF12, TNNI3, VEGFA; or
(i) ACTB, ACTG1, ACTN1, ACTN2, ACTN3, ACTN4, AFDN, ARHGAP35, ARHGAP5, BCAR1, CD99, CDC42, CDH5, CLDN1, CLDN10, CLDN11, CLDN14, CLDN15, CLDN16, CLDN17, CLDN18, CLDN19, CLDN2, CLDN20, CLDN22, CLDN23, CLDN3, CLDN4, CLDN5, CLDN6, CLDN7, CLDN8, CLDN9, CTNNA1, CTNNA2, CTNNA3, CTNNB1, CTNND1, CXCL12, CXCR4, CYBA, CYBB, ESAM, EZR, F11R, GNAI1, GNAI2, GNAI3, ICAM1, ITGA4, ITGAL, ITGAM, ITGB1, ITGB2, ITK, JAM2, JAM3, MAPK11, MAPK12, MAPK13, MAPK14, MMP2, MMP9, MSN, MYL10, MYL12A, MYL12B, MYL2, MYL5, MYL7, MYL9, MYLPF, NCF1, NCF2, NCF4, NOX1, NOX3, OCLN, PECAM1, PIK3CA, PIK3CB, PIK3CD, PIK3CG, PIK3R1, PIK3R2, PIK3R3, PIK3R5, PLCG1, PLCG2, PRKCA, PRKCB, PRKCG, PTK2, PTK2B, PTPN11, PXN, RAC1, RAC2, RAP1A, RAP1B, RAPGEF3, RAPGEF4, RASSF5, RHOA, RHOH, ROCK1, ROCK2, SIPA1, THY1, TXK, VASP, VAV1, VAV2, VAV3, VCAM1, VCL; or
(j) ADAM12, ADAP2, CHN1, COL5A3, EGFL6, ZD10, OPX, HSPA6, LCP2, MMP9, MXRA5, PAMR1, PLXDC1, PMAIP1, POSTN, RPS11, STC1, TNFAIP6, TNFRSF21, TWIST1, CAN, WDR77; or
(k) FLT1, FLT3, FLT4, KDR, NRP1, NRP2, PDGFRA; or
(l) ADAM17, AKT3, APLN, CCL2, COL4A3, FGF2, FGFR1, FLT1, GHRL, GHSR, HMGB1, IGF2, ITGA4, MDK, MEF2C, MIR126, MIR129-1, MIR129-2, MIR130A, MIR132, MIR133B, MIR135B, MIR152, MIR15B, MIR193A, MIR20B, MIR21, MIR24-1, MIR24-2, MIR27A, MIR29A, MIR29C, MIR30B, MIR30E, MIR329-1, MIR329-2, MIR34A, MIR424, MIR487B, MIR492, MIR495, MIR499A, MIR503, MIR98, PDPK1, PLCG1, PPARG, SIRT6, SP1, STAT3;
(m) ARNT, CADM4, CNMD, DAB2IP, EMILIN1, EPN2, FGF10, FGF18, FGF9, FZD4, GRB10, HGS, HHEX, HIF1A, IL1B, ITGA5, ITGB3, MIR10A, MIR10B, MIR1224, MIR2000, MIR296, MMRN2, MT3, MYOF, NEDD4, NIBAN2, PDCD6, PRKCB, PRKD2, PTPN1, TMEM204, TNMD, VEGFC, VTN; or
(n) ALOX5, CCL21, CCL25, CCL28, CCR2, CXCL12, ELANE, ETS1, FUT4, FUT7, FUT9, ICAM1, IL6, IRAK1, ITGA4, ITGB2, KLF4, MDK, MIR125A, MIR141, MIR146A, MIR21, MIR221, MIR222, MIR31, MIR92A1, MIR92A2, MIRLET7E, MIRLET7G, NFAT5, PTAFR, RELA, RHOA, SELE, SELP, TNF, TRAF6; or
(o) ALOX5, KLF4, MIR92A1, MIR92A2, TNF; or
(p) ADAMTS12, ALOX12, CXCL10, FGF1, FOXP1, ITGAX, MIR21; or
(q) CXCL13, FGF1, FGF16, FGF18, FGF2, FGF4, FGFR1, HRG, HSPB1, KDR, LGMN, MET, MIR149, MIR16-1, MIR16-2, MIR424, NOTCH1, P2RX4, PRKD1, PRKD2, SEMA5A, SMOC2, THBS1, TMSB4X, VEGFA; or
(r) ADAMTS12, ADAMTS3, ADGRA2, ATP2B4, CADM4, CCBE1, CD63, DAB2IP, DCN, DLL1, HRG, IL12A, IL12B, JCAD, MIR16-1, MIR16-2, MIR199A1, MIR199A2, MIR199B, MIR21, MIR329-1, MIR329-2, MIR342, MIR424, MYO1C, PTP4A3, ROBO1, SEMA6A, SMOC2, SPRY2, TSPAN12, XDH; or
(s) ADAMTS3, CCBE1, JCAD, MIR21, MYO1C, ROBO1, SMOC2;
(t) ARNT, FGF10, FGF18, FGF9, GRB10, HIF1A, IL1B, ITGA5, ITGB3, MIR10A, MIR10B, MIR1224, MIR296, MT3, PRKCB, PRKD2, VTN;
(u) ADORA2B, ARNT, ATF4, BRCA1, C3, C3AR1, C5, C5AR1, CCBE1, CXCL17, CYP1B1, EIF2AK3, FLT4, GATA4, HIF1A, HPSE, IL1A, IL1B, IL6, IL6ST, ISL1, NODAL, NOX1, PTGS2, RORA, SULF1, SULF2, TGFB1; or
(v) ADAM17, AKT3, APLN, FGF2, FGFR1, GHRL, HSR, HMGB1, IGF2, ITGA4, MDK, MIR126, MIR130A, MIR132, MIR135B, MIR21, MIR27A, MIR29A, MIR495, MIR499A, PDPK1, PLCG1, SIRT6, SP1, STAT3; or
(w) ALOX5, CCR2, ELANE, ETS1, FUT4, FUT7, ICAM1, IL6, IRAK1, ITGA4, ITGB2, MDK, MIR21, MIR92A1, MIR92A2, NFAT5, PTAFR, RELA, RHOA, SELE, SELP, TNF, TRAF6; or
(x) ACVRL1, ADAM17, AGGF1, AGTR1, AKT1, AKT3, ANG, APELA, APLN, APLNR, ARG1, ARNT, BMP2, BMP4, BMP6, BMPR2, CAV2, CCL11, CCL24, CCL26, CCR3, CDH13, CXCL12, CYBA, DYSF, ECM1, EGFL7, EGR3, EMC10, F3, GF2, FGFBP1, GFR1, LT4, GATA2, GDF2, GHRL, GHSR, HIF1A, HMGB1, HMGB2, HMOX1, HTR2B, IGF2, IL10, ITGA4, ITGB3, JCAD, JUN, KDR, LRG1, MDK, MIR101-1, MIR101-2, MIR10A, MIR10B, MIR126, MIR130A, MIR132, MIR135B, MIR146A, MIR21, MIR23A, MIR27A, MIR27B, MIR29A, MIR487B, MIR495, MIR499A, MIR503, MIRLET7B, MTOR, MYDGF, NF1, NR4A1, NRARP, NRAS, NRP1, NRP2, PDCD6, PDCL3, PDGFB, PDPK1, PGF, PLCG1, PLXNB3, PPP1R16B, PRKCA, PRKD1, PRKD2, PROX1, RICTOR, RPTOR, SCG2, SEMA5A, SIRT1, SIRT6, SP1, STAT3, STAT5A, TEK, TGFBR1, THBS4, TNFSF12, VASH2, VEGFA, VEGFB, VEGFC, VEGFD, VIP, WNT2, WNT5A, ZNF580; or
(y) AAMP, ABL1, ADAM17, ADGRA2, AGT, AKT1, AKT3, ALOX12, AMOTL1, ANGPT1, ANGPT4, ANXA1, ANXA3, ATOH8, ATP5F1A, ATP5F1B, BCAR1, BCAS3, BMP4, BMPR2, CALR, CCBE1, CD40, CIB1, EDN1, EMC10, ETS1, FGF1, FGF16, FGF18, FGF2, FGFBP1, FGFR1, FLT4, FOXC2, FOXP1, FUT1, GATA2, GATA3, GPI, GPLD1, GRN, HDAC7, HDAC9, HIF1A, HMGB1, HMOX1, HSPB1, ITGB1BP1, ITGB3, JCAD, KDR, LGMN, MAP2K3, MAP3K3, MET, MIR101-1, MIR101-2, MIR10A, MIR10B, MIR126, MIR132, MIR135B, MIR143, MIR146A, MIR150, MIR1908, MIR199A1, MIR199A2, MIR199B, MIR200A, MIR21, MIR210, MIR221, MIR23A, MIR27A, MIR27B, MIR296, MIR29A, MIR30A, MIR31, MIR342, MIR487B, MIR499A, MIR939, MIRLET7F1, MIRLET7F2, NFE2L2, NOS3, NRP1, NRP2, NUS1, P2RX4, PDCD6, PDGFB, PDPK1, PIK3C2A, PLCG1, PLK2, PLPP3, PRKCA, PRKD1, PRKD2, PROX1, PTGS2, PTK2B, RHOB, RHOJ, RIN2, ROCK2, RRAS, SASH1, SCARB1, SEMA5A, SIRT1, SMOC2, SP1, SPARC, SRPX2, STAT5A, TDGF1, TEK, TGFB1, THBS1, TMSB4X, TSTA3, VEGFA, VEGFC, WNT5A, WNT7A, ZC3H12A, ZNF580; or
(z) HSPB1, KDR, PRKD1, PRKD2, VEGFA; or
(aa) FGF16, FGF18, FGF2, FGFR1, HSPB1, KDR, LGMN, MET, P2RX4, PRKD1, PRKD2, SEMA5A, SMOC2, TMSB4X, VEGFA; or
(bb) GAB1, HSPB1, KDR, MYO1C, PRKD1, PRKD2, VEGFA; or
(cc) AGTR1, APELA, APLNR, FGFBP1, GATA2, HMOX1, JCAD, MIR101-1, MIR101-2, MIR10A, MIR10B, MIR126, MIR132, MIR146A, MIR21, MIR23A, MIR27A, MIR27B, MIR487B, MIR503, MIRLET7B, PPP1R16B, VEGFA; or
(dd) ADAM17, AKT1, AKT3, ALOX12, AMOTL1, ANGPT1, ANGPT4, ANXA1, ATP5F1A, ATP5F1B, CD40, CIB1, ETS1, FGF18, FGF2, FGFBP1, FGFR1, FOXC2, GATA2, HDAC7, HDAC9, HIF1A, HMGB1, HMOX1, HSPB1, JCAD, KDR, MAP2K3, MAP3K3, MIR101-1, MIR101-2, MIR10A, MIR10B, MIR126, MIR132, MIR135B, MIR143, MIR146A, MIR150, MIR200A, MIR210, MIR221, MIR23A, MIR27A, MIR27B, MIR296, MIR30A, MIR31, MIR342, MIR487B, MIR499A, MIR939, MIRLET7F1, MIRLET7F2, NFE2L2, NOS3, NRP1, NUS1, P2RX4, PDGFB, PDPK1, PIK3C2A, PLCG1, PLK2, PRKCA, PRKD1, PRKD2, PTGS2, RHOJ, SIRT1, SP1, SRPX2, STAT5A, TGFB1, THBS1, TMSB4X, VEGFA, VEGFC; or
(ee) ADD2, ALOX5, CCL21, CCL25, CCL28, CCR2, CX3CR1, CXCL12, ELANE, ETS1, FUT4, FUT7, FUT9, GCNT1, GOLPH3, ICAM1, IL6, IRAK1, ITGA4, ITGB1, ITGB2, ITGB7, JAM2, KLF4, LEP, MADCAM1, MDK, MIR125A, MIR141, MIR146A, MIR21, MIR221, MIR222, MIR31, MIR92A1, MIR92A2, MIRLET7E, MIRLET7G, NFAT5, PODXL2, PTAFR, RELA, RHOA, ROCK1, SELE, SELL, SELP, SELPLG, SLC39A8, SPN, TNF, TRAF6, VCAM1; or
(ff) ALOX5, KLF4, MIR92A1, MIR92A2, SLC39A8, TNF; or
(gg) AAMP, ABL1, ACVRL1, ADAM17, ADAMTS9, ADGRA2, ADGRB1, ADTRP, AGT, AGTR2, AKT1, AKT3, LOX12, AMOT, AMOTL1, ANGPT1, ANGPT2, ANGPT4, ANXA1, ANXA3, APOA1, APOE, APOH, ATOH8, ATP2B4, ATP5F1A, ATP5F1B, BCAR1, BCAS3, BMP10, BMP4, BMPER, BMPR2, CALR, CARD10, CCBE1, CCN3, CD40, CDH13, CDH5, CEACAM1, CIB1, CLEC14A, CORO1B, CSNK2B, CXCL13, CYP1B1, DAB2IP, DCN, DLL4, DNAJA4, DPP4, EDN1, EFNA1, EFNB2, EGR3, EMC10, EMP2, EPHA2, EPHB4, ETS1, FAP, FBXW7, FGF1, FGF16, FGF18, FGF2, FGF4, FGFBP1, FGFR1, FLT4, FOXC2, FOXP1, FUT1, GADD45A, GATA2, GATA3, GDF2, GIPC1, GLUL, GPI, GPLD1, GPX1, GREM1, GRN, HDAC5, HDAC7, HDAC9, HIF1A, HMGB1, HMOX1, HRG, HSPB1, ID1, ITGB1, ITGB1BP1, ITGB2, ITGB3, JCAD, JUP, KDR, KLF4, KRIT1, LGALS12, LGALS8, LGMN, LOXL2, LPXN, MAP2K3, MAP2K5, MAP3K3, MECP2, MEF2C, MEOX2, MET, MIA3, MIR101-1, MIR101-2, MIR10A, MIR10B, MIR126, MIR129-1, MIR129-2, MIR132, MIR133B, MIR135B, MIR137, MIR143, MIR146A, MIR149, MIR150, MIR152, MIR15A, MIR15B, MIR16-1, MIR16-2, MIR188, MIR1908, MIR193A, MIR196A1, MIR196A2, MIR199A1, MIR199A2, MIR199B, MIR19B1, MIR19B2, MIR200A, MIR200B, MIR2000, MIR204, MIR206, MIR20A, MIR21, MIR210, MIR212, MIR22, MIR221, MIR2355, MIR23A, MIR24-1, MIR24-2, MIR26A1, MIR26A2, MIR27A, MIR27B, MIR296, MIR29A, MIR29C, MIR30A, MIR31, MIR320A, MIR329-1, MIR329-2, MIR342, MIR361, MIR410, MIR424, MIR483, MIR487B, MIR492, MIR494, MIR495, MIR497, MIR499A, MIR503, MIR505, MIR640, MIR92A1, MIR92A2, MIR939, MIRLET7F1, MIRLET7F2, MMRN2, MYH9, NF1, NFE2L2, NOS3, NOTCH1, NR2E1, NR2F2, NR4A1, NRP1, NRP2, NUS1, P2RX4, PAXIP1, PDCD10, PDCD6, PDGFB, PDPK1, PECAM1, PIK3C2A, PIK3CA, PIK3R3, PLCG1, PLEKHG5, PLK2, PLPP3, PLXND1, PPARG, PRCP, PRKCA, PRKD1, PRKD2, PRKX, PROX1, PRSS3, PRSS3P2, PTEN, PTGS2, PTK2, PTK2B, PTN, PTP4A3, PTPRM, PXN, RAB13, RGCC, RHOA, RHOB, RHOJ, RIN2, ROBO1, ROCK2, RRAS, S100A2, S100P, SASH1, SCARB1, SCG2, SEMA4A, SEMA5A, SERPINF1, SH3BP1, SIRT1, SLIT2, SMOC2, SOX18, SP1, SP100, SPARC, SPRED1, SRF, SRPX2, STARD13, STAT5A, STC1, SVBP, SYNJ2BP, TBXA2R, TDGF1, TEK, TGFB1, TGFBR1, THBS1, TMSB4X, TNF, TNFSF12, TSTA3, VASH1, VEGFA, VEGFC, WNT5A, WNT7A, ZC3H12A, ZNF580; or
(hh) CCN3, CORO1B, CXCL13, EGR3, FGF1, FGF16, FGF18, FGF2, FGF4, GFR1, HRG, HSPB1, KDR, LGMN, MET, MIR149, MIR16-1, MIR16-2, MIR424, NOTCH1, NR4A1, NRP1, P2RX4, PLEKHG5, PRKD1, PRKD2, RAB13, SEMA5A, SMOC2, THBS1, TMSB4X, VEGFA; or
(ii) APOLD1, BMPER, CXCL10, FOXP1, HOXA9, MIR92A1, MIR92A2, P2RX4, PRMT5, SMAD4, TCIM, TGFBR1;
(jj) ABL1, ACVRL1, ADAM17, ADTRP, AGTR2, AKT1, AKT3, ALOX12, AMOT, AMOTL1, ANGPT1, ANGPT2, ANGPT4, ANXA1, APOA1, APOE, ATP2B4, ATP5F1A, ATP5F1B, CARD10, CD40, CDH5, CIB1, CLEC14A, CSNK2B, DLL4, EFNA1, EFNB2, EGR3, EMP2, EPHA2, EPHB4, ETS1, FBXW7, FGF18, FGF2, FGFBP1, FGFR1, FOXC2, GADD45A, GATA2, GDF2, GPLD1, GPX1, GREM1, HDAC5, HDAC7, HDAC9, HIF1A, HMGB1, HMOX1, HRG, HSPB1, ID1, ITGB1, ITGB1BP1, JCAD, JUP, KDR, KLF4, MAP2K3, MAP2K5, MAP3K3, MECP2, MEF2C, MEOX2, MIA3, MIR101-1, MIR101-2, MIR10A, MIR10B, MIR126, MIR129-1, MIR129-2, MIR132, MIR133B, MIR135B, MIR137, MIR143, MIR146A, MIR149, MIR150, MIR152, MIR15A, MIR15B, MIR16-1, MIR16-2, MIR188, MIR193A, MIR196A1, MIR196A2, MIR199A1, MIR199A2, MIR199B, MIR19B1, MIR19B2, MIR200A, MIR200B, MIR2000, MIR204, MIR206, MIR20A, MIR210, MIR212, MIR22, MIR221, MIR2355, MIR23A, MIR24-1, MIR24-2, MIR26A1, MIR26A2, MIR27A, MIR27B, MIR296, MIR29C, MIR30A, MIR31, MIR320A, MIR329-1, MIR329-2, MIR342, MIR361, MIR410, MIR424, MIR483, MIR487B, MIR492, MIR494, MIR495, MIR497, MIR499A, MIR503, MIR505, MIR640, MIR92A1, MIR92A2, MIR939, MIRLET7F1, MIRLET7F2, MMRN2, MYH9, NF1, NFE2L2, NOS3, NOTCH1, NR2E1, NR4A1, NRP1, NUS1, P2RX4, PDCD10, PDGFB, PDPK1, PIK3C2A, PIK3R3, PLCG1, PLK2, PPARG, PRCP, PRKCA, PRKD1, PRKD2, PTGS2, PTK2B, RGCC, RHOA, RHOJ, ROBO1, SCARB1, SH3BP1, SIRT1, SLIT2, SOX18, SP1, SPRED1, SRF, SRPX2, STARD13, STAT5A, TBXA2R, TDGF1, TGFB1, THBS1, TMSB4X, TNF, VASH1, VEGFA, VEGFC;
or any combination thereof; and
(ii) genes that impact T-cell function selected from:
(a) ADAM10, ADAM17, CCL21, CCL26, CCL27, CCL3, CCL5, CCR2, CXCL10, CXCL11, CXCL13, CXCL16, CXCR3, DEFA1, DEFA1B, GPR183, OXSR1, PIK3CD, PIK3CG, S100A7, SLC12A2, STK39, TMEM102, TNFSF14, WNK1, WNT5A, XCL1, XCL2; or
(b) AGER, AIRE, ARG1, AZGP1, B2M, BTN3A2, BTN3A3, CCR2, CD1A, CD1B, CD1C, CD1D, CD1E, CD46, CD55, CD70, CD81, CD8A, CEACAM1, CLC, CLEC4G, CTSC, CTSH, CYRIB, DENND1B, DLG1, DUSP22, EMP2, FADD, FBXO38, FCGR2B, FOXP3, FUT7, FZD5, GATA3, GNL1, GZMM, HFE, HLA-A, HLA-B, HLA-E, HLA-F, HLA-G, HLA-H, HMGB1, HPRT1, HSPD1, ICAM1, IFNA2, IFNB1, IL12A, IL12B, IL12RB1, IL18, IL18R1, IL18RAP, IL1B, IL1R1, IL20RB, IL23A, IL23R, IL31RA, IL4, IL6, IL7R, JAG1, KDELR1, LILRB1, MALT1, MAP3K7, MICA, MR1, MYO1G, NECTIN2, NLRP3, P2RX7, PPP3CB, PRF1, PRKCZ, PTPRC, PVR, RAB27A, RFTN1, RIPK3, RSAD2, SASH3, SCART1, SLC11A1, SLC22A13, SMAD7, STARD7, STX7, TBX21, TNFRSF1B, TNFSF4, TRAF2, TRAF6, TREX1, TRPM4, TSTA3, WAS, XCL1, ZBTB1, ZP3; or
(c) FBXO38, HLA-A, HMGB1, HSPD1, MR1, SLC22A13; or
(d) AGER, AZGP1, B2M, CD1A, CD1B, CD1C, CD1D, CD1E, CEACAM1, CTSC, CTSH, CYRIB, EMP2, FADD, FCGR2B, GZMM, HLA-A, HLA-B, HLA-E, HLA-F, HLA-G, HLA-H, HPRT1, IL12A, IL12B, IL12RB1, IL23A, IL23R, IL7R, LILRB1, MICA, MR1, NECTIN2, P2RX7, PPP3CB, PRF1, PTPRC, PVR, RAB27A, RIPK3, SLC22A13, STX7, TSTA3, XCL1; or
(e) CCL2, CCR2, CD99, CD99L2, CRK, CRKL, F11R, FADD, ICAM1, IL27RA, ITGAL, RIPK3, XG; or
(f) ANXA1, ATP7A, BATF, BCL3, BCL6, CCL19, CD46, CD80, CD86, CLEC4D, CLEC4E, EOMES, FCER1G, FGL2, FOXP1, FOXP3, GATA3, GPR183, HLX, HMGB1, IFNG, IFNL1, IL12B, IL12RB1, IL18, IL18R1, IL2, IL21, IL23A, IL23R, IL27, IL4, IL4R, IL6, IRF4, JAK3, LEF1, LGALS9, LOXL3, LY9, MALT1, MIR21, MTOR, MYB, NFKBID, NFKBIZ, NLRP3, PCK1, PRKCZ, PTGER4, RARA, RC3H1, RC3H2, RELB, RIPK2, RORA, RORC, SEMA4A, SLAMF6, SMAD7, SOCS5, SPN, STAT3, STAT6, TBX21, TMEM98, TNFSF4, TSC1, ZBTB7B, ZC3H12A, ZFPM1; or
(g) ADA, ADAM8, AKT1, ARG2, BAK1, BAX, BCL10, BCL2L11, BMP4, CCL5, CD27, CD274, CLC, DFFA, DNAJA3, DOCK8, EFNA1, FADD, FAS, FASLG, GIMAP8, GLI3, GPAM, HIF1A, IDO1, IL2RA, IL7R, JAK3, KDELR1, LGALS13, LGALS14, LGALS16, LGALS3, LGALS9, LMBR1L, NFKBID, PDCD1, PIP, PRELID1, PRKCQ, PTCRA, RAG1, RIPK1, RIPK3, SLC46A2, TP53, TSC22D3, WNT5A, ZC3H8; or
(h) HFE, ICAM1, RFTN1, TREX1, WAS; or
(i) ANXA1, APBB1IP, ATP7A, BATF, BCL3, BCL6, CCL19, CD1C, CD46, CD74, CD80, CD81, CD86, CEACAM1, CLEC4D, CLEC4E, EIF2AK4, EOMES, F2RL1, FCER1G, FCGR2B, FGL2, FOXP1, FOXP3, GATA3, GPR183, HAVCR2, HLA-DMB, HLX, HMGB1, ICAM1, IFNA1, IFNA10, IFNA13, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNW1, IL12B, IL12RB1, IL18, IL18R1, IL2, IL21, IL23A, IL23R, IL27, IL4, IL4R, IL6, IRF4, ITGAL, JAK3, LCP1, LEF1, LGALS3, LGALS9, LILRB1, LOXL3, LY9, MALT1, MDK, MIR21, MTOR, MYB, NFKBID, NFKBIZ, NLRP3, PCK1, PRKCZ, PSEN1, PTGER4, RAB27A, RARA, RC3H1, RC3H2, RELB, RIPK2, RORA, RORC, SEMA4A, SLAMF6, SLC11A1, SMAD7, SOCS5, SPN, STAT3, STAT6, TBX21, TMEM98, TNFSF18, TNFSF4, TP53, TSC1, ZBTB7B, ZC3H12A, ZFPM1; or
(j) ADA, BCL10, BTN2A2, CACNA1F, CARD11, CCR7, CD160, CD226, CD300A, CD81, CEACAM1, CYLD, DGKZ, DUSP22, DUSP3, ELF1, EZR, GBP1, KCNN4, LCK, LGALS3, LILRB4, MALT1, NECTIN2, PAWR, PHPT1, PRKD2, PRNP, PTPN2, PTPN22, PTPN6, PTPRJ, PVRIG, RAB29, RC3H1, RELA, RPS3, SH2D1A, SLA2, TESPA1, THY1, TRAT1, UBASH3A; or
(k) ADAM10, ADAM17, ADAM8, AIF1, AIRE, APOD, APP, C10orf99, CCL20, CCL21, CCL27, CCL5, CCR2, CCR6, CD200, CD200R1, CD99L2, CRK, CRKL, CXCL10, CXCL12, CXCL13, DOCK8, ECM1, FADD, IL27RA, ITGA4, LRCH1, OXSR1, PYCARD, RHOA, RIPK3, RIPOR2, S100A7, SELENOK, SPN, STK39, TMEM102, TNFRSF14, TNFSF14, WNK1, WNT5A, XCL1, XCL2; or
(l) ARG1, AZGP1, B2M, CCR2, CD1A, CD1B, CD1C, CD1D, CD1E, CD81, CEACAM1, CLC, CLEC4G, CYRIB, DUSP22, FADD, FBXO38, FCGR2B, FOXP3, FUT7, FZD5, GATA3, HFE, HLA-A, HLA-B, HLA-E, HLA-F, HLA-G, HLA-H, HMGB1, HSPD1, IFNA2, IFNB1, IL12A, IL12B, IL12RB1, IL18, IL18R1, IL1B, IL1R1, IL20RB, IL23A, IL23R, IL6, IL7R, LILRB1, MALT1, MAP3K7, MR1, NECTIN2, NLRP3, P2RX7, PPP3CB, PRKCZ, PTPRC, PVR, RIPK3, RSAD2, SASH3, SLC22A13, SMAD7, STX7, TBX21, TNFRSF1B, TNFSF4, TRAF2, TRAF6, TRPM4, WAS, XCL1, ZBTB1, ZP3;
(m) FBXO38, HMGB1, HSPD1, MR1, SLC22A13; or
(n) AGER, AZGP1, B2M, CD1A, CD1B, CD1C, CD1D, CD1E, CEACAM1, CYRIB, FADD, FCGR2B, HLA-A, HLA-B, HLA-E, HLA-F, HLA-G, HLA-H, IL12A, IL12B, IL12RB1, IL23A, IL23R, IL7R, LILRB1, MR1, NECTIN2, P2RX7, PPP3CB, PTPRC, PVR, RIPK3, SLC22A13, STX7, XCL1; or
(o) CCR2, CD99L2, FADD, IL27RA, RIPK3; or
(p) ABL1, ADA, ADAM8, ANXA1, AP3B1, AP3D1, BAD, BCL6, BMP4, BTN2A2, CAMK4, CARD11, CBFB, CCL19, CCR2, CD2, CD27, CD28, CD46, CD74, CD80, CD83, CD86, CLPTM1, CR1, CRTAM, CTLA4, CYLD, CYP26B1, DROSHA, DTX1, DUSP10, EGR3, ERBB2, FANCA, FANCD2, FGL2, FOXJ1, FOXN1, FOXP3, GATA3, GLI2, GLI3, HLA-DOA, HLA-G, HLX, HMGB1, IFNA2, IFNB1, IFNG, IFNL1, IHH, IL12A, IL12B, IL12RB1, IL15, IL18, IL1A, IL1, IL1RL2, IL2, IL23A, IL23R, IL27, IL2RA, IL36B, IL4, IL4R, IL7, IL7R, IRF1, IRF4, ITPKB, JAK3, KAT2A, LAG3, LEF1, LGALS9, LILRB2, LILRB4, LOXL3, MALT1, MDK, METTL3, MIR21, MIR30B, MYB, NCKAP1L, NFATC2, NFKBID, NFKBIZ, NKAP, NLRP3, NRARP, PCK1, PIK3R6, PNP, PRDM1, PRDX2, PRELID1, PRKCZ, PTPN2, PTPRC, RAG1, RARA, RASGRP1, RC3H1, RC3H2, RHOA, RHOH, RIPK2, RUNX1, RUNX3, SART1, SASH3, SH3RF1, SHH, SLC46A2, SMAD7, SOCS1, SOCS5, SOD1, SOS1, SOX12, SOX13, SOX4, SPINK5, STAT5B, SYK, TBX21, TCF7, TESPA1, TGFBR2, TMEM131L, TNFRSF18, TNFSF4, TNFSF9, TOX, VNN1, VSIR, XBP1, ZAP70, ZBTB1, ZBTB16, ZBTB7B, ZC3H12A, ZC3H8, ZEB1, ZMIZ1, ZNF683; or
(q) B2M, CCR2, CD81, CLC, FOXP3, FZD5, GATA3, HFE, HLA-A, HLA-F, IFNA2, IFNB1, IL18, IL18R1, IL1B, IL1R1, IL6, MALT1, MAP3K7, NLRP3, PRKCZ, RSAD2, SASH3, SMAD7, TBX21, TNFRSF1B, TNFSF4, TRAF2, TRAF6, TRPM4, XCL1; or
(r) ADAM10, ADAM17, CCL21, CCL27, CCL5, CCR2, CXCL10, CXCL13, OXSR1, S100A7, STK39, TMEM102, TNFSF14, WNK1, WNT5A, XCL1, XCL2; or
(s) CD81, HAVCR2, HLA-DMB, LGALS3, LGALS9, LILRB1; or
(t) ABL1, ADA, ADAM8, ADORA2A, AGER, AIF1, AKT1, ANXA1, AP3B1, AP3D1, ARG1, ARG2, BAD, BCL10, BCL6, BMP4, BTLA, BTN2A2, CAMK4, CARD11, CASP3, CAV1, CBFB, CCDC88B, CCL19, CCL2, CCL21, CCL5, CCR2, CCR7, CD160, CD1D, CD2, CD209, CD24, CD27, CD274, CD276, CD28, CD300A, CD3E, CD4, CD40LG, CD46, CD47, CD5, CD55, CD6, CD70, CD74, CD80, CD81, CD83, CD86, CDC42, CEACAM1, CEBPB, CGAS, CLC, CLEC4G, CLECL1, CLPTM1, CORO1A, CR1, CRTAM, CSK, CTLA4, CTNNB1, CYLD, CYP26B1, CYRIB, DLG1, DLG5, DNAJA3, DOCK8, DPP4, DROSHA, DTX1, DUSP10, DUSP22, DUSP3, EBI3, EFNB1, EFNB2, EFNB3, EGR3, EPO, ERBB2, FADD, FANCA, FANCD2, FCGR2B, FGL1, FGL2, FLOT2, FOXJ1, FOXN1, FOXP3, FYN, GATA3, L12, GLI3, GLMN, GNRH1, GPAM, GPNMB, GRAP2, GRB2, GSN, HAVCR2, HES1, HFE, HHLA2, HLA-A, HLA-DMB, HLA-DOA, HLA-DPA1, HLA-DPB1, HLA-DRB1, HLA-E, HLA-G, HLX, HMGB1, HSPD1, HSPH1, ICOS, ICOSLG, IDO1, IFNA2, IFNB1, IFNG, IFNL1, IGF1, IGF2, IGFBP2, IHH, IL10, IL12A, IL12B, IL12RB1, IL15, IL18, IL1A, IL1B, IL1RL2, IL2, IL20RB, IL21, IL23A, IL23R, IL27, IL27RA, IL2RA, IL36B, IL4, IL4R, IL6, IL6ST, IL7, IL7R, IRF1, IRF4, ITPKB, JAK3, KAT2A, KLRC4-KLRK1, KLRK1, LAG3, LAT, LAX1, LCK, LEF1, LEP, LGALS1, LGALS3, LGALS7B, LGALS9, LGALS9B, LGALS9C, LILRB1, LILRB2, LILRB4, LMO1, LOXL3, LRRC32, LYN, MAD1L1, MALT1, MAP3K8, MAPK8IP1, MARCHF7, MDK, METTL3, MIR181C, MIR21, MIR27A, MIR30B, MYB, NCK1, NCK2, NCKAP1L, NDFIP1, NFATC2, NFKBID, NFKBIZ, NKAP, NLRP3, NOD2, NRARP, PAG1, PAK1, PAK2, PAK3, PAWR, PCK1, PDCD1, PDCD1LG2, PDE5A, PDPK1, PELI1, PIK3CA, PIK3R1, PIK3R6, PLA2G2D, PLA2G2F, PNP, PRDM1, PRDX2, PRELID1, PRKAR1A, PRKCQ, PRKCZ, PRNP, PTPN11, PTPN2, PTPN22, PTPN6, PTPRC, PYCARD, RAC1, RAC2, RAG1, RARA, RASAL3, RASGRP1, RC3H1, RC3H2, RHOA, RHOH, RIPK2, RIPK3, RIPOR2, RPS3, RUNX1, RUNX3, SART1, SASH3, SCGB1A1, SCRIB, SDC4, SELENOK, SFTPD, SH3RF1, SHH, SIRPA, SIRPB1, SIRPG, SIT1, SLC46A2, SLC7A1, SMAD7, SOCS1, SOCS5, SOCS6, SOD1, SOS1, SOX12, SOX13, SOX4, SPINK5, SPN, SPTA1, SRC, STAT5B, SYK, TARM1, TBX21, TCF7, TESPA1, TFRC, TGFBR2, THY1, TIGIT, TMEM131L, TMIGD2, TNFAIP8L2, TNFRSF13C, TNFRSF14, TNFRSF18, TNFRSF1B, TNFRSF21, TNFSF11, TNFSF13B, TNFSF14, TNFSF18, TNFSF4, TNFSF8, TNFSF9, TOX, TRAF6, TREX1, TWSG1, VAV1, VCAM1, VNN1, VSIG4, VSIR, VTCN1, XBP1, XCL1, YES1, ZAP70, ZBTB1, ZBTB16, ZBTB7B, ZC3H12A, ZC3H8, ZEB1, ZMIZ1, ZNF683, ZP3, ZP4; or
(u) EGR3, LEF1, LILRB1, NCKAP1L, NOD2, PTPRC, SOX13, SOX4, STAT5B, SYK, TCF7; or
(v) CBFB, NCKAP1L, RUNX1, RUNX3, SOCS1, ZBTB7B; or
(w) ANXA1, BCL6, CBFB, CCL19, CD80, CD83, CD86, FOXP3, GATA3, HLX, HMGB1, IFNG, IL12B, IL12RB1, IL18, IL2, IL23A, IL23R, IL27, IL4, IL4R, IRF4, JAK3, LGALS9, LOXL3, MALT1, MIR21, MYB, NCKAP1L, NFKBID, NFKBIZ, NLRP3, PRKCZ, RARA, RC3H1, RC3H2, RIPK2, RUNX1, RUNX3, SASH3, SH3RF1, SMAD7, SOCS1, SOCS5, TBX21, TNFSF4, ZBTB7B, ZC3H12A; or
(x) AGER, ANXA1, ARG2, BCL6, CBFB, CCL19, CD160, CD274, CD55, CD80, CD81, CD83, CD86, FOXP3, GATA3, HLX, HMGB1, IFNG, IL12B, IL12RB1, IL18, IL2, IL23A, IL23R, IL27, IL4, IL4R, IRF4, JAK3, LGALS7B, LGALS9, LGALS9B, LGALS9C, LOXL3, MALT1, MIR21, MYB, NCKAP1L, NFKBID, NFKBIZ, NLRP3, PRKCQ, PRKCZ, RARA, RC3H1, RC3H2, RIPK2, RUNX1, RUNX3, SASH3, SH3RF1, SMAD7, SOCS1, SOCS5, TARM1, TBX21, TGFBR2, TNFSF4, TWSG1, VSIR, XCL1, ZBTB7B, ZC3H12A; or
(y) CBFB, CD274, CRTAM, HFE, HLA-A, HLA-E, IRF1, LILRB1, MAPK8IP1, NCKAP1L, PTPN22, RUNX1, RUNX3, SH3RF1, SOCS1, VSIR, XCL1, ZBTB7B; or
(z) ADA, ANXA1, AP3B1, AP3D1, BCL6, CBFB, CCL19, CD80, CD83, CD86, FOXP3, GATA3, GLI3, HLX, HMGB1, IFNG, IHH, IL12B, IL12RB1, IL18, IL2, IL23A, IL23R, IL27, IL4, IL4R, IRF4, ITPKB, JAK3, LGALS9, LOXL3, MALT1, MIR21, MYB, NCKAP1L, NFKBID, NFKBIZ, NKAP, NLRP3, PNP, PRDM1, PRKCZ, RARA, RC3H1, RC3H2, RHOA, RIPK2, RUNX1, RUNX3, SASH3, SH3RF1, SHH, SMAD7, SOCS1, SOCS5, SYK, TBX21, TGFBR2, TNFSF4, ZAP70, ZBTB16, ZBTB7B, ZC3H12A, ZNF683; or
(aa) ADA, ADORA2A, AGER, ANXA1, AP3B1, AP3D1, ARG2, BCL6, CBFB, CCL19, CCR2, CD160, CD274, CD28, CD300A, CD3E, CD55, CD80, CD81, CD83, CD86, CRTAM, EBI3, FOXP3, GATA3, GLI3, HFE, HLA-A, HLA-E, HLX, HMGB1, HSPH1, IFNG, IHH, IL12A, IL12B, IL12RB1, IL18, IL2, IL23A, IL23R, IL27, IL4, IL4R, IRF1, IRF4, ITPKB, JAK3, LGALS7B, LGALS9, LGALS9B, LGALS9C, LILRB1, LOXL3, MALT1, MAPK8IP1, MIR21, MYB, NCKAP1L, NFKBID, NFKBIZ, NKAP, NLRP3, PNP, PRDM1, PRKCQ, PRKCZ, PTPN22, PTPRC, RARA, RASAL3, RC3H1, RC3H2, RHOA, RIPK2, RUNX1, RUNX3, SASH3, SH3RF1, SHH, SMAD7, SOCS1, SOCS5, SYK, TARM1, TBX21, TGFBR2, TNFRSF14, TNFSF4, TWSG1, VSIR, XCL1, ZAP70, ZBTB16, ZBTB7B, ZC3H12A, ZNF683; or
(bb) BATF, BCL11B, BCL2, CD3D, CD3E, CD3G, CD74, CTSL, CYLD, DOCK2, FOXN1, FOXP3, IL12B, IL12RB1, IL23A, IL23R, IL6, IRF4, ITPKB, LOXL3, LY9, MTOR, PTPN2, PTPRC, SHH, SLAMF6, SPN, SRF, STAT3, STAT6, STK11, TBX21, THEMIS, TOX, ZAP70, ZFPM1; or
(cc) CD3E, FOXP3, HLA-G, IDO1, LILRB2, TGFBR2; or
(dd) ADA, CARD11, CCR7, CD226, CD81, CYLD, KCNN4, LCK, NECTIN2, PRKD2, RAB29, RELA, RPS3, TESPA1, TRAT1; or
(ee) AGER, AIF1, ANXA1, CARD11, CCDC88B, CCL19, CCL5, CCR2, CD1D, CD209, CD24, CD274, CD276, CD28, CD3E, CD4, CD40LG, CD46, CD55, CD6, CD70, CD80, CD81, CD86, CLECL1, CORO1A, DNAJA3, EBI3, EFNB1, EPO, FADD, FOXP3, GPAM, HAVCR2, HES1, HHLA2, HLA-A, HLA-DMB, HLA-DPA1, HLA-DPB1, HLA-E, HMGB1, ICOSLG, IGF1, IGF2, IGFBP2, IL12A, IL12B, IL12RB1, IL15, IL18, IL1A, IL1B, IL2, IL21, IL23A, IL23R, IL27RA, IL2RA, IL4, IL6, IL6ST, JAK3, LEP, LGALS9, LILRB2, MIR21, MIR30B, NCK1, NCK2, NCKAP1L, PDCD1LG2, PNP, PRKCQ, PTPN22, PTPRC, PYCARD, RASAL3, RIPK2, RPS3, SASH3, SELENOK, SHH, SLC7A1, SPTA1, STAT5B, SYK, TFRC, TGFBR2, TMIGD2, TNFRSF13C, TNFSF13B, TNFSF4, TNFSF9, TRAF6, VCAM1, VTCN1, XCL1, ZAP70, ZP3, ZP4; or
(ff) ADAM10, ADAM17, ADAM8, AIF1, APP, CCL20, CCL21, CCL27, CCL5, CCR2, CD99L2, CXCL10, CXCL12, CXCL13, DOCK8, FADD, ITGA4, OXSR1, PYCARD, RHOA, S100A7, SELENOK, SPN, STK39, TMEM102, TNFRSF14, TNFSF14, WNK1, WNT5A, XCL1, XCL2; or
(gg) AZGP1, B2M, CD1A, CD1B, CD1C, CD1D, COlE, CD81, CYRIB, FADD, FBXO38, FOXP3, FZD5, GATA3, HLA-A, HLA-B, HLA-E, HLA-F, HLA-G, HLA-H, HSPD1, IL12A, IL12B, IL12RB1, IL18, IL18R1, IL1B, IL1R1, IL23A, IL23R, IL6, MALT1, MAP3K7, MR1, NECTIN2, NLRP3, P2RX7, PRKCZ, PTPRC, PVR, RSAD2, SASH3, SLC22A13, STX7, TNFSF4, TRAF2, TRAF6, XCL1, ZBTB1, ZP3; or
(hh) AZGP1, B2M, CD1A, CD1B, CD1C, CD1D, COlE, CYRIB, FADD, HLA-A, HLA-B, HLA-E, HLA-F, HLA-G, HLA-H, IL12A, IL12B, IL12RB1, IL23A, IL23R, MR1, NECTIN2, P2RX7, PTPRC, PVR, SLC22A13, STX7, XCL1; or
(ii) B2M, CD81, FZD5, GATA3, HLA-A, IL18, IL18R1, IL1B, IL1R1, IL6, MALT1, MAP3K7, NLRP3, PRKCZ, RSAD2, SASH3, TNFSF4, TRAF2, TRAF6, XCL1; or
(jj) BCL6, BTN2A2, CD46, DUSP10, FOXP3, HLA-G, IFNG, IL2, LGALS9, LILRB2, LILRB4, SOCS1, SOX12, VSIR; or
(kk) FOXP3, IL1A, IL1B, RIPK2, SHH; or
(ll) LEF1, NCKAP1L, PTPRC, SOX13, SOX4, STAT5B, SYK; or
(mm) LEF1, LILRB1, NCKAP1L, NOD2, PTPRC, SOX13, SOX4, STAT5B, SYK; or
(nn) ADA, ANXA1, AP3B1, AP3D1, CBFB, CCL19, CD80, CD83, CD86, FOXP3, GLI3, HLX, IFNG, IHH, IL12B, IL12RB1, IL18, IL23A, IL23R, IL4R, ITPKB, LGALS9, MALT1, MIR21, MYB, NCKAP1L, NFKBID, NFKBIZ, NKAP, NLRP3, PNP, PRKCZ, RARA, RHOA, RIPK2, RUNX1, RUNX3, SASH3, SHH, SOCS1, SOCS5, SYK, TGFBR2, TNFSF4, ZAP70, ZBTB16, ZBTB7B; or
(oo) ADA, ANXA1, AP3B1, AP3D1, CBFB, CCL19, CCR2, CD160, CD28, CD3E, CD55, CD80, CD81, CD83, CD86, EBI3, FOXP3, GLI3, HLA-A, HLA-E, HLX, HSPH1, IFNG, IHH, IL12A, IL12B, IL12RB1, IL18, IL23A, IL23R, IL4R, ITPKB, LGALS9, MALT1, MIR21, MYB, NCKAP1L, NFKBID, NFKBIZ, NKAP, NLRP3, PNP, PRKCQ, PRKCZ, PTPN22, PTPRC, RARA, RASAL3, RHOA, RIPK2, RUNX1, RUNX3, SASH3, SHH, SOCS1, SOCS5, SYK, TGFBR2, TNFSF4, XCL1, ZAP70, ZBTB16, ZBTB7B; or
(pp) AGER, CD24, EPO, FADD, GPAM, HHLA2, HMGB1, ICOSLG, IGF1, IGF2, IGFBP2, IL12B, IL12RB1, IL18, IL2, IL23A, IL23R, IL27RA, IL2RA, MIR21, MIR30B, PYCARD, RPS3, STAT5B, TMIGD2, TNFSF9; or
(qq) ABL1, ADA, ADAM17, ADAM8, ADORA2A, AGER, AIF1, AIRE, AKT1, ANXA1, AP3B1, AP3D1, APBB1IP, ARG1, ARG2, ATG5, ATP7A, AZI2, B2M, BAD, BATF, BAX, BCL10, BCL11B, BCL2, BCL3, BCL6, BMP4, BTLA, BTN2A2, BTN3A1, CAMK4, CARD11, CASP3, CASP8, CAV1, CBFB, CCDC88B, CCL19, CCL2, CCL21, CCL5, CCND3, CCR2, CCR6, CCR7, CCR9, CD151, CD160, CD1C, CD1D, CD2, CD209, CD24, CD27, CD274, CD276, CD28, CD300A, CD3D, CD3E, CD3G, CD4, CD40LG, CD44, CD46, CD47, CD5, CD55, CD6, CD7, CD70, CD74, CD80, CD81, CD83, CD86, CD8A, CD8B, CDC42, CDH26, CDK6, CEACAM1, CEBPB, CGAS, CHD7, CLC, CLEC4A, CLEC4D, CLEC4E, CLEC4G, CLEC7A, CLECL1, CLPTM1, CORO1A, CR1, CRTAM, CSK, CTLA4, CTNNB1, CTPS1, CTSL, CXADR, CYLD, CYP26B1, CYRIB, DDOST, DLG1, DLG5, DLL4, DNAJA3, DOCK2, DOCK8, DPP4, DROSHA, DTX1, DUSP10, DUSP22, DUSP3, EBI3, EFNB1, EFNB2, EFNB3, EGR1, EGR3, EIF2AK4, ELF4, EOMES, EPO, ERBB2, F2RL1, FADD, FANCA, FANCD2, FCER1G, FCGR2B, FGL1, FGL2, FKBP1A, FKBP1B, FLOT2, FOXJ1, FOXN1, FOXP1, FOXP3, FUT7, FYN, FZD5, FZD7, FZD8, GATA3, GLI2, GLI3, GLMN, GNRH1, GPAM, GPNMB, GPR18, GPR183, GPR89A, GPR89B, GRAP2, GRB2, GSN, HAVCR2, HES1, HFE, HHLA2, HLA-A, HLA-DMB, HLA-DOA, HLA-DPA1, HLA-DPB1, HLA-DRB1, HLA-E, HLA-G, HLX, HMGB1, HSH2D, HSPD1, HSPH1, ICAM1, ICOS, ICOSLG, ID01, IFNA1, IFNA10, IFNA13, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNW1, IGF1, IGF2, IGFBP2, IHH, IL10, IL12A, IL12B, IL12RB1, IL15, IL18, IL18R1, IL1A, IL1, IL1RL2, IL2, IL20RB, IL21, IL23A, 1L23R, 1L27, IL27RA, IL2RA, 1L36B, 1L4, IL4R, 1L6, IL6ST, 1L7, IL7R, INS, IRF1, IRF4, ITGAL, ITK, ITPKB, JAG2, JAK3, JAML, JMJD6, KAT2A, KDELR1, KIF13B, KIT, KLRC4-KLRK1, KLRK1, LAG3, LAT, LAX1, LCK, LCP1, LEF1, LEP, LEPR, LFNG, LGALS1, LGALS3, LGALS7B, LGALS9, LGALS9B, LGALS9C, LIG4, LILRB1, LILRB2, LILRB4, LMBR1L, LMO1, LOXL3, LRRC32, LY9, LYN, MAD11, MAFB, MALT1, MAP3K8, MAPK8IP1, MARCHF7, MDK, METTL3, MICA, MICB, MIR181C, MIR21, MIR27A, MIR30B, MR1, MSN, MTOR, MYB, MYH9, NCAPH2, NCK1, NCK2, NCKAP1L, NCSTN, NDFIP1, NEDD4, NFATC2, NFKBID, NFKBIZ, NHEJ1, NKAP, NKX2-3, NLRC3, NLRP3, NOD2, NRARP, P2RX7, PAG1, PAK1, PAK2, PAK3, PATZ1, PAWR, PAX1, PCK1, PDCD1, PDCD1LG2, PDESA, PDPK1, PELI1, PIK3CA, PIK3CD, PIK3CG, PIK3R1, PIK3R6, PKNOX1, PLA2G2D, PLA2G2F, PNP, PPP3CA, PPP3CB, PRDM1, PRDX2, PRELID1, PREX1, PRKAR1A, PRKCQ, PRKCZ, PRKDC, PRNP, PRR7, PSAP, PSEN1, PSMB10, PSMB11, PTGER4, PTPN11, PTPN2, PTPN22, PTPN6, PTPRC, PYCARD, RAB27A, RAB29, RABL3, RAC1, RAC2, RAG1, RAG2, RARA, RASAL3, RASGRP1, RC3H1, RC3H2, RELB, RHOA, RHOH, RIPK2, RIPK3, RIPOR2, RORA, RORC, RPS3, RSAD2, RUNX1, RUNX2, RUNX3, SART1, SASH3, SATB1, SCGB1A1, SCRIB, SDC4, SELENOK, SEMA4A, SFTPD, SH3RF1, SHH, SIRPA, SIRPB1, SIRPG, SIT1, SLA2, SLAMF6, SLC11A1, SLC46A2, SLC7A1, SMAD3, SMAD7, SOCS1, SOCS5, SOCS6, SOD1, SOS1, SOX12, SOX13, SOX4, SP3, SPINK5, SPN, SPTA1, SRC, SRF, STAT3, STAT5B, STAT6, STK11, STOML2, SYK, TARM1, TBX21, TCF7, TCIRG1, TESPA1, TFRC, TGFBR2, THEMIS, THY1, TIGIT, TMEM131L, TMEM98, TMIGD2, TNFAIP8L2, TNFRSF13C, TNFRSF14, TNFRSF18, TNFRSF1B, TNFRSF21, TNFRSF4, TNFSF11, TNFSF13B, TNFSF14, TNFSF18, TNFSF4, TNFSF8, TNFSF9, TOX, TP53, TRAF6, TREML2, TREX1, TSC1, TWSG1, VAV1, VCAM1, VNN1, VSIG4, VSIR, VTCN1, WAS, WNT1, WNT4, XBP1, XCL1, YES1, ZAP70, ZBTB1, ZBTB16, ZBTB7B, ZC3H12A, ZC3H8, ZEB1, ZFP36L1, ZFP36L2, ZFPM1, ZMIZ1, ZNF683, ZP3, ZP4; or
(rr) BMP4, ERBB2, FOXP3, GNRH1, IHH, IL1A, IL1B, RIPK2, SHH, TMEM131L, WNT4; or
(ss) HLA-A, HLA-E, IRF1, MAPK8IP1, PTPN22, SH3RF1, VSIR, XCL1; or
(tt) BCL2, CBFB, EOMES, GPR18, IRF1, NCKAP1L, PAX1, PSMB11, RUNX1, RUNX3, SATB1, SOCS1, TNFSF8, TOX, ZBTB7B; or
(uu) BCL2, CBFB, CD274, CLEC4A, CRTAM, EOMES, GPR18, HFE, HLA-A, HLA-E, IRF1, LILRB1, MAPK8IP1, NCKAP1L, PAX1, PSMB11, PTPN22, RUNX1, RUNX3, SATB1, SH3RF1, SOCS1, TNFSF8, TOX, VSIR, XCL1, ZBTB7B; or
(vv) BATF, BCL2, CTSL, CYLD, FOXP3, IL12B, IL12RB1, IL23A, IL23R, IL6, IRF4, LOXL3, LY9, MTOR, SHH, SLAMF6, SPN, STAT3, STAT6, TBX21, TOX, ZFPM1; or
(ww) FOXP3, FUT7, IFNG, LGALS9, PLA2G2D, TOX; or
(xx) ARG2, CD274, CD55, CD81, LGALS7B, LGALS9, LGALS9B, LGALS9C, TGFBR2, TWSG1, VSIR, XCL1; or
(yy) ARG1, CD55, CD81, DENND1B, GATA3, IFNA2, IFNB1, IL12A, IL12B, IL18, IL18R1, IL18RAP, IL1B, IL1R1, IL31RA, IL4, IL6, NLRP3, PRKCZ, RSAD2, STARD7, TBX21, XCL1; or
(zz) AGER, ANXA1, ARG2, ATP7A, BATF, BCL3, BCL6, CBFB, CCL19, CD160, CD274, CD55, CD80, CD81, CD83, CD86, CDH26, CTSL, FOXP1, FOXP3, FUT7, GATA3, GPR183, HLX, HMGB1, IFNG, IL12A, IL12B, IL12RB1, IL18, IL18R1, IL2, L21, IL23A, IL23R, IL27, IL4, IL4R, IL6, IRF4, JAK3, LEF1, LGALS7B, LGALS9, LGALS9B, LGALS9C, LOXL3, LY9, MALT1, MIR21, MTOR, MYB, NCKAP1L, NFKBID, NFKBIZ, NKX2-3, NLRP3, PAX1, PLA2G2D, PRKCQ, PRKCZ, PTGER4, RARA, RC3H1, RC3H2, RELB, RIPK2, RORA, RORC, RSAD2, RUNX1, RUNX3, SASH3, SATB1, SEMA4A, SH3RF1, SLAMF6, SMAD7, SOCS1, SOCS5, SPN, STAT3, STAT6, STOML2, TARM1, TBX21, TCIRG1, TGFBR2, TMEM98, TNFSF4, TOX, TWSG, VSIR, XCL1, ZBTB7B, ZC3H12A, ZFPM1; or
(aaa) ARG2, CCR2, CD274, CD28, CD3E, CD55, CD81, DOCK2, EBI3, ELF4, HLA-A, HLA-E, IL12B, IL15, IL18, IL23A, IRF1, LGALS7B, LGALS9, LGALS9B, LGALS9C, MAPK8IP1, PTPN22, PTPRC, RASAL3, RIPK2, SH3RF1, SYK, TGFBR2, TNFRSF14, TNFSF4, TWSG1, VSIR, XCL1, ZAP70, ZBTB7B; or
(bbb) ANXA1, ATP7A, BATF, BCL3, BCL6, CCL19, CD80, CD86, EOMES, FOXP1, FOXP3, GATA3, GPR183, HLX, HMGB1, IFNG, IL12B, IL12RB1, IL18, IL18R1, IL2, IL21, IL23A, IL23R, IL27, IL4, IL4R, IL6, IRF4, JAK3, LEF1, LGALS9, LOXL3, LY9, MALT1, MIR21, MTOR, MYB, NFKBID, NFKBIZ, NLRP3, PRKCZ, PTGER4, RARA, RC3H1, RC3H2, RELB, RIPK2, RORA, RORC, SEMA4A, SLAMF6, SMAD7, SOCS5, SPN, STAT3, STAT6, TBX21, TMEM98, TNFSF4, ZBTB7B, ZC3H12A, ZFPM1; or
(ccc) ABL1, AGER, ARG1, BTN2A2, BTN3A1, CASP3, CD24, CD274, CLC, CRTAM, EPO, FADD, FOXP3, FYN, GPAM, HHLA2, HMGB1, ICOSLG, IGF1, IGF2, IGFBP2, IL12B, IL12RB1, IL18, IL2, IL23A, IL23R, IL27RA, IL2RA, LGALS9, LRRC32, MIR181C, MIR21, MIR30B, PDCD1LG2, PRKAR1A, PRNP, PYCARD, RC3H1, RIPK3, RPS3, SATB1, SCRIB, STAT5B, TMIGD2, TNFSF9;
or any combination thereof.

5. (canceled)

6. (canceled)

7. The method of claim 1, wherein the cancer is a non-hematological solid tumor selected from Adrenocortical carcinoma (ACC), Bladder Urothelial Carcinoma (BLCA), Brain Lower Grade Glioma (LGG), Breast invasive carcinoma (BRCA), Colon adenocarcinoma (COAD), Cervical squamous cell carcinoma and endocervical adenocarcinoma (CESC), Cholangiocarcinoma (CHOL), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Uterine Carcinosarcoma (UCS), Uterine Corpus Endometrial Carcinoma (UCEC), and Uveal Melanoma (UVM).

8. (canceled)

9. (canceled)

10. The method of claim 4, wherein the two or more genes comprise the genes provided in (i) and (ii).

11. The method of claim 2, wherein the biological sample is: (a) a cancer or tumor biopsy; or (b) a tumor microenvironment (TME) aspirate.

12. (canceled)

13. The method of claim 2, wherein the ICB therapy is a PD-1 inhibitor therapy or a CTLA4 inhibitor therapy.

14. The method of claim 13, wherein the ICB therapy is a PD-1 inhibitor therapy.

15. The method of claim 2, wherein the expression levels of the two or more genes are measured by RNA sequencing, RT-PCR, or microarray analysis or a combination thereof.

16. The method of claim 2, wherein the angio-immune subtype is determined by computational analysis.

17. The method of claim 16, wherein the angio-immune subtype is determined by baseline angio-immune score calculated by the computational analysis based on the expression levels of the group of genes, and wherein deviation of the score from a reference value indicates whether the subject would respond to or not respond to the therapy.

18. The method of claim 3, wherein the identification or prediction of the subject as responsive to ICB therapy is further determined based on one or more clinical factors selected from mutation status of one or more genes, stromal score, fibroblast score, and mutational burden.

19. (canceled)

20. The method of claim 2, wherein the two or more genes are selected from CSMD3, IDH1, BRAF, PTEN, ATRX, LRP1B, ZFHX4, USH2A and FAT4.

21. A method of predicting responsiveness of a subject diagnosed with cancer to an immune checkpoint blockade (ICB) therapy, comprising:
(i) obtaining or having obtained the expression and mutational profile of two or more genes from a biological sample obtained from the subject;
(ii) implementing a gene set variation analysis (GSVA) to obtain an enrichment score of the two or more genes;
(iii) generating a correlation matrix across the enrichment scores;
(iv) identifying the angio-immune subtype of the subject based on the correlation matrix; and
(v) predicting the responsiveness of the subject to an ICB therapy based on the angio-immune subtype, wherein the subject is predicted to be responsive to ICB therapy when the angio-immune subtype corresponds to a low angiogenic signature and high T cell function,
wherein the expression and mutational profile of the two or more genes is obtained by RNA sequencing, RT-PCR, or microarray analysis or a combination thereof.

22. The method of claim 21, wherein wherein the two or more genes are selected from one or more of:
(i) genes that impact angiogenesis selected from:
(a) MYCT1, APOH, APP, CCND2 COL3A1, COL5A2, CXCL6, FGFR1, FSTL1, ITGAV, JAG1, JAG2, KCNJ8, LPL, LRPAP1, LUM, MSX1, NRP1, OLR1, PDGFA, PF4, PGLYRP1, POSTN, PRG2, PTK2, S100A4, SERPINA5, SLCO2A1, SPP1, STC1, THBD, TIMP1, TNFRSF21, VAV2, CAN, VEGFA, VTN, CDH5, ELTD1, CLEC14A, LDB2, ECSCR, RHOJ, VWF, TIE1, KDR, ESAM, CD93, PTPRB, GPR116, SPARCL1, EMCN, ROBO4, ENG, TEK, SIPR1; or
(b) AGGF1, AMOT, ANGPTL3, ANGPTL4, BTG1, CHRNA7, RHOB, RUNX1, SPHK1, TNFSF12; or
(c) APOH, APP, CCND2, COL3A1, COL5A2, CXCL6, FGFR1, FSTL1, ITGAV, JAG1, JAG2, KCNJ8, LPL, LRPAP1, LUM, MSX1, NRP1, OLR1, PDGFA, PF4, PGLYRP1, POSTN, PRG2, PTK2, S100A4, SERPINA5, SLCO2A1, SPP1, STC1, THBD, TIMP1, TNFRSF21, VAV2, CAN, VEGFA, VTN; or
(d) ABL1, ADAMTS9, AGTR1, AKT3, ALOX5, ANXA1, APELA, APLNR, BMPER, CARD10, CEACAM1, CEMIP2, CIB1, CREB3L1, DLL1, DLL4, E2F2, EPN1, EPN2, FBXW7, FGF1, FGF2, FGFBP1, FOXC2, FUT1, GATA2, GHRL, GHSR, GLUL, HDAC5, HDAC7, HDAC9, HMOX1, IL10, IL12A, IL12B, ITGA5, ITGB1BP1, JAK1, JCAD, JMJD8, KDR, KLF2, KLF4, MAP2K5, MAP3K3, MEOX2, MIR1-1, MIR1-2, MIR101-1, MIR101-2, MIR10A, MIR10B, MIR1224, MIR125A, MIR126, MIR132, MIR138-1, MIR138-2, MIR146A, MIR149, MIR150, MIR15A, MIR15B, MIR16-1, MIR16-2, MIR17, MIR188, MIR18A, MIR193A, MIR196A1, MIR196A2, MIR199A1, MIR199A2, MIR199B, MIR19A, MIR19B1, MIR19B2, MIR2000, MIR206, MIR20A, MIR21, MIR22, MIR221, MIR222, MIR2355, MIR23A, MIR24-1, MIR24-2, MIR26A1, MIR26A2, MIR27A, MIR27B, MIR296, MIR29C, MIR30B, MIR30C1, MIR30C2, MIR30E, MIR31, MIR320A, MIR329-1, MIR329-2, MIR342, MIR34A, MIR34B, MIR34C, MIR361, MIR375, MIR377, MIR410, MIR424, MIR483, MIR487B, MIR494, MIR495, MIR497, MIR503, MIR92A1, MIR92A2, MIRLET7B, MIRLET7F1, MIRLET7F2, MMRN2, NGFR, NOTCH1, NR2E1, NRP1, PDCD10, PDPK1, PIK3C2A, PKM, PLK2, PPP1R16B, PTGS2, RHOA, RHOJ, S100A1, SEMA6A, SMAD1, SPRED1, SRPX2, STARD13, SYNJ2BP, TBXA2R, THBS1, TJP1, VEGFA; or
(e) ABL1, AGTR1, AKT3, ANXA1, APELA, APLNR, BMPER, CIB1, DLL1, FGF1, FGF2, FGFBP1, FOXC2, FUT1, GATA2, GHRL, GHSR, HDAC7, HDAC9, HMOX1, IL10, ITGA5, JAK1, JCAD, JMJD8, KDR, KLF4, MAP3K3, MIR1-1, MIR1-2, MIR101-1, MIR101-2, MIR10A, MIR10B, MIR1224, MIR125A, MIR126, MIR132, MIR146A, MIR150, MIR21, MIR23A, MIR27A, MIR27B, MIR296, MIR30B, MIR31, MIR487B, MIR503, MIR92A1, MIR92A2, MIRLET7B, MIRLET7F1, MIRLET7F2, NRP1, PDPK1, PIK3C2A, PKM, PLK2, PPP1R16B, PTGS2, RHOJ, S100A1, SMAD1, SRPX2, TJP1, VEGFA; or
(f) ABL1, AKT3, ANXA1, CIB1, FGF2, FGFBP1, FOXC2, GATA2, HDAC7, HDAC9, HMOX1, JCAD, KDR, MAP3K3, MIR101-1, MIR101-2, MIR10A, MIR10B, MIR126, MIR132, MIR146A, MIR150, MIR23A, MIR27A, MIR27B, MIR296, MIR31, MIR487B, MIRLET7F1, MIRLET7F2, NRP1, PIK3C2A, PLK2, PTGS2, RHOJ, SRPX2, VEGFA; or
(g) ABL1, ADTRP, AKT1, KT3, ANXA1, CARD10, CIB1, CLEC14A, DLL4, EFNB2, EGR3, EPHB4, FBXW7, FGF2, FGFBP1, FOXC2, GATA2, GPLD1, GREM1, HDAC5, HDAC7, HDAC9, HMOX1, ITGB1, ITGB1BP1, JCAD, KDR, KLF4, MAP2K5, MAP3K3, MEOX2, MIA3, MIR101-1, MIR101-2, MIR10A, MIR10B, MIR126, MIR132, MIR146A, MIR149, MIR150, MIR15A, MIR16-1, MIR16-2, MIR188, MIR193A, MIR196A1, MIR196A2, MIR199A1, MIR199A2, MIR199B, MIR19B1, MIR19B2, MIR2000, MIR206, MIR20A, MIR22, MIR221, MIR2355, MIR23A, MIR26A1, MIR26A2, MIR27A, MIR27B, MIR296, MIR29C, MIR31, MIR320A, MIR329-1, MIR329-2, MIR361, MIR410, MIR424, MIR483, MIR487B, MIR494, MIR495, MIR497, MIR503, MIRLET7F1, MIRLET7F2, MMRN2, NOTCH1, NR2E1, NR4A1, NRP1, PDCD10, PIK3C2A, PIK3R3, PLK2, PTGS2, RHOA, RHOJ, ROBO1, SLIT2, SPRED1, SRF, SRPX2, STARD13, TBXA2R, TDGF1, THBS1, VEGFA; or
(h) ACVRL1, AGGF1, AMOT, ANG, ANGPTL3, ANGPTL4, ATP5IF1, BTG1, C1GALT1, CANX, CDH13, CHRNA7, COL4A2, COL4A3, CXCL8, EGF, EMCN, EPGN, ERAP1, FOXO4, HTATIP2, IL17F, IL18, MYH9, NCL, NF1, NOTCH4, NPPB, NPR1, PF4, PLG, PML, PROK2, RHOB, RNH1, ROBO4, RUNX1, SCG2, SERPINF1, SHH, SPHK1, SPINK5, STAB1, TGFB2, THY1, TNFSF12, TNNI3, VEGFA; or
(i) ACTB, ACTG1, ACTN1, ACTN2, ACTN3, ACTN4, AFDN, ARHGAP35, ARHGAP5, BCAR1, CD99, CDC42, CDH5, CLDN1, CLDN10, CLDN11, CLDN14, CLDN15, CLDN16, CLDN17, CLDN18, CLDN19, CLDN2, CLDN20, CLDN22, CLDN23, CLDN3, CLDN4, CLDN5, CLDN6, CLDN7, CLDN8, CLDN9, CTNNA1, CTNNA2, CTNNA3, CTNNB1, CTNND1, CXCL12, CXCR4, CYBA, CYBB, ESAM, EZR, F11R, GNAI1, GNAI2, GNAI3, ICAM1, ITGA4, ITGAL, ITGAM, ITGB1, ITGB2, ITK, JAM2, JAM3, MAPK11, MAPK12, MAPK13, MAPK14, MMP2, MMP9, MSN, MYL10, MYL12A, MYL12B, MYL2, MYL5, MYL7, MYL9, MYLPF, NCF1, NCF2, NCF4, NOX1, NOX3, OCLN, PECAM1, PIK3CA, PIK3CB, PIK3CD, PIK3CG, PIK3R1, PIK3R2, PIK3R3, PIK3R5, PLCG1, PLCG2, PRKCA, PRKCB, PRKCG, PTK2, PTK2B, PTPN11, PXN, RAC1, RAC2, RAP1A, RAP1B, RAPGEF3, RAPGEF4, RASSF5, RHOA, RHOH, ROCK1, ROCK2, SIPA1, THY1, TXK, VASP, VAV1, VAV2, VAV3, VCAM1, VCL; or
(j) ADAM12, ADAP2, CHN1, COL5A3, EGFL6, ZD10, OPX, HSPA6, LCP2, MMP9, MXRA5, PAMR1, PLXDC1, PMAIP1, POSTN, RPS11, STC1, TNFAIP6, TNFRSF21, TWIST1, CAN, WDR77; or
(k) FLT1, FLT3, FLT4, KDR, NRP1, NRP2, PDGFRA; or
(l) ADAM17, AKT3, APLN, CCL2, COL4A3, FGF2, FGFR1, FLT1, GHRL, GHSR, HMGB1, IGF2, ITGA4, MDK, MEF2C, MIR126, MIR129-1, MIR129-2, MIR130A, MIR132, MIR133B, MIR135B, MIR152, MIR15B, MIR193A, MIR20B, MIR21, MIR24-1, MIR24-2, MIR27A, MIR29A, MIR29C, MIR30B, MIR30E, MIR329-1, MIR329-2, MIR34A, MIR424, MIR487B, MIR492, MIR495, MIR499A, MIR503, MIR98, PDPK1, PLCG1, PPARG, SIRT6, SP1, STAT3;
(m) ARNT, CADM4, CNMD, DAB2IP, EMILIN1, EPN2, FGF10, FGF18, FGF9, FZD4, GRB10, HGS, HHEX, HIF1A, IL1B, ITGA5, ITGB3, MIR10A, MIR10B, MIR1224, MIR2000, MIR296, MMRN2, MT3, MYOF, NEDD4, NIBAN2, PDCD6, PRKCB, PRKD2, PTPN1, TMEM204, TNMD, VEGFC, VTN; or
(n) ALOX5, CCL21, CCL25, CCL28, CCR2, CXCL12, ELANE, ETS1, FUT4, FUT7, FUT9, ICAM1, IL6, IRAK1, ITGA4, ITGB2, KLF4, MDK, MIR125A, MIR141, MIR146A, MIR21, MIR221, MIR222, MIR31, MIR92A1, MIR92A2, MIRLET7E, MIRLET7G, NFAT5, PTAFR, RELA, RHOA, SELE, SELP, TNF, TRAF6; or
(o) ALOX5, KLF4, MIR92A1, MIR92A2, TNF; or
(p) ADAMTS12, ALOX12, CXCL10, FGF1, FOXP1, ITGAX, MIR21; or
(q) CXCL13, FGF1, FGF16, FGF18, FGF2, FGF4, FGFR1, HRG, HSPB1, KDR, LGMN, MET, MIR149, MIR16-1, MIR16-2, MIR424, NOTCH1, P2RX4, PRKD1, PRKD2, SEMA5A, SMOC2, THBS1, TMSB4X, VEGFA; or
(r) ADAMTS12, ADAMTS3, ADGRA2, ATP2B4, CADM4, CCBE1, CD63, DAB2IP, DCN, DLL1, HRG, IL12A, IL12B, JCAD, MIR16-1, MIR16-2, MIR199A1, MIR199A2, MIR199B, MIR21, MIR329-1, MIR329-2, MIR342, MIR424, MYO1C, PTP4A3, ROBO1, SEMA6A, SMOC2, SPRY2, TSPAN12, XDH; or
(s) ADAMTS3, CCBE1, JCAD, MIR21, MYO1C, ROBO1, SMOC2;
(t) ARNT, FGF10, FGF18, FGF9, GRB10, HIF1A, IL1B, ITGA5, ITGB3, MIR10A, MIR10B, MIR1224, MIR296, MT3, PRKCB, PRKD2, VTN;
(u) ADORA2B, ARNT, ATF4, BRCA1, C3, C3AR1, C5, C5AR1, CCBE1, CXCL17, CYP1B1, EIF2AK3, FLT4, GATA4, HIF1A, HPSE, IL1A, IL1B, IL6, IL6ST, ISL1, NODAL, NOX1, PTGS2, RORA, SULF1, SULF2, TGFB1; or
(v) ADAM17, AKT3, APLN, FGF2, FGFR1, GHRL, HSR, HMGB1, IGF2, ITGA4, MDK, MIR126, MIR130A, MIR132, MIR135B, MIR21, MIR27A, MIR29A, MIR495, MIR499A, PDPK1, PLCG1, SIRT6, SP1, STAT3; or
(w) ALOX5, CCR2, ELANE, ETS1, FUT4, FUT7, ICAM1, IL6, IRAK1, ITGA4, ITGB2, MDK, MIR21, MIR92A1, MIR92A2, NFAT5, PTAFR, RELA, RHOA, SELE, SELP, TNF, TRAF6; or
(x) ACVRL1, ADAM17, AGGF1, AGTR1, AKT1, AKT3, ANG, APELA, APLN, APLNR, ARG1, ARNT, BMP2, BMP4, BMP6, BMPR2, CAV2, CCL11, CCL24, CCL26, CCR3, CDH13, CXCL12, CYBA, DYSF, ECM1, EGFL7, EGR3, EMC10, F3, GF2, FGFBP1, GFR1, LT4, GATA2, GDF2, GHRL, GHSR, HIF1A, HMGB1, HMGB2, HMOX1, HTR2B, IGF2, IL10, ITGA4, ITGB3, JCAD, JUN, KDR, LRG1, MDK, MIR101-1, MIR101-2, MIR10A, MIR10B, MIR126, MIR130A, MIR132, MIR135B, MIR146A, MIR21, MIR23A, MIR27A, MIR27B, MIR29A, MIR487B, MIR495, MIR499A, MIR503, MIRLET7B, MTOR, MYDGF, NF1, NR4A1, NRARP, NRAS, NRP1, NRP2, PDCD6, PDCL3, PDGFB, PDPK1, PGF, PLCG1, PLXNB3, PPP1R16B, PRKCA, PRKD1, PRKD2, PROX1, RICTOR, RPTOR, SCG2, SEMA5A, SIRT1, SIRT6, SP1, STAT3, STAT5A, TEK, TGFBR1, THBS4, TNFSF12, VASH2, VEGFA, VEGFB, VEGFC, VEGFD, VIP, WNT2, WNT5A, ZNF580; or
(y) AAMP, ABL1, ADAM17, ADGRA2, AGT, AKT1, AKT3, ALOX12, AMOTL1, ANGPT1, ANGPT4, ANXA1, ANXA3, ATOH8, ATP5F1A, ATP5F1B, BCAR1, BCAS3, BMP4, BMPR2, CALR, CCBE1, CD40, CIB1, EDN1, EMC10, ETS1, FGF1, FGF16, FGF18, FGF2, FGFBP1, FGFR1, FLT4, FOXC2, FOXP1, FUT1, GATA2, GATA3, GPI, GPLD1, GRN, HDAC7, HDAC9, HIF1A, HMGB1, HMOX1, HSPB1, ITGB1BP1, ITGB3, JCAD, KDR, LGMN, MAP2K3, MAP3K3, MET, MIR101-1, MIR101-2, MIR10A, MIR10B, MIR126, MIR132, MIR135B, MIR143, MIR146A, MIR150, MIR1908, MIR199A1, MIR199A2, MIR199B, MIR200A, MIR21, MIR210, MIR221, MIR23A, MIR27A, MIR27B, MIR296, MIR29A, MIR30A, MIR31, MIR342, MIR487B, MIR499A, MIR939, MIRLET7F1, MIRLET7F2, NFE2L2, NOS3, NRP1, NRP2, NUS1, P2RX4, PDCD6, PDGFB, PDPK1, PIK3C2A, PLCG1, PLK2, PLPP3, PRKCA, PRKD1, PRKD2, PROX1, PTGS2, PTK2B, RHOB, RHOJ, RIN2, ROCK2, RRAS, SASH1, SCARB1, SEMA5A, SIRT1, SMOC2, SP1, SPARC, SRPX2, STAT5A, TDGF1, TEK, TGFB1, THBS1, TMSB4X, TSTA3, VEGFA, VEGFC, WNT5A, WNT7A, ZC3H12A, ZNF580; or
(z) HSPB1, KDR, PRKD1, PRKD2, VEGFA; or
(aa) FGF16, FGF18, FGF2, FGFR1, HSPB1, KDR, LGMN, MET, P2RX4, PRKD1, PRKD2, SEMA5A, SMOC2, TMSB4X, VEGFA; or
(bb) GAB1, HSPB1, KDR, MYO1C, PRKD1, PRKD2, VEGFA; or
(cc) AGTR1, APELA, APLNR, FGFBP1, GATA2, HMOX1, JCAD, MIR101-1, MIR101-2, MIR10A, MIR10B, MIR126, MIR132, MIR146A, MIR21, MIR23A, MIR27A, MIR27B, MIR487B, MIR503, MIRLET7B, PPP1R16B, VEGFA; or
(dd) ADAM17, AKT1, AKT3, ALOX12, AMOTL1, ANGPT1, ANGPT4, ANXA1, ATP5F1A, ATP5F1B, CD40, CIB1, ETS1, FGF18, FGF2, FGFBP1, FGFR1, FOXC2, GATA2, HDAC7, HDAC9, HIF1A, HMGB1, HMOX1, HSPB1, JCAD, KDR, MAP2K3, MAP3K3, MIR101-1, MIR101-2, MIR10A, MIR10B, MIR126, MIR132, MIR135B, MIR143, MIR146A, MIR150, MIR200A, MIR210, MIR221, MIR23A, MIR27A, MIR27B, MIR296, MIR30A, MIR31, MIR342, MIR487B, MIR499A, MIR939, MIRLET7F1, MIRLET7F2, NFE2L2, NOS3, NRP1, NUS1, P2RX4, PDGFB, PDPK1, PIK3C2A, PLCG1, PLK2, PRKCA, PRKD1, PRKD2, PTGS2, RHOJ, SIRT1, SP1, SRPX2, STAT5A, TGFB1, THBS1, TMSB4X, VEGFA, VEGFC; or
(ee) ADD2, ALOX5, CCL21, CCL25, CCL28, CCR2, CX3CR1, CXCL12, ELANE, ETS1, FUT4, FUT7, FUT9, GCNT1, GOLPH3, ICAM1, IL6, IRAK1, ITGA4, ITGB1, ITGB2, ITGB7, JAM2, KLF4, LEP, MADCAM1, MDK, MIR125A, MIR141, MIR146A, MIR21, MIR221, MIR222, MIR31, MIR92A1, MIR92A2, MIRLET7E, MIRLET7G, NFAT5, PODXL2, PTAFR, RELA, RHOA, ROCK1, SELE, SELL, SELP, SELPLG, SLC39A8, SPN, TNF, TRAF6, VCAM1; or
(ff) ALOX5, KLF4, MIR92A1, MIR92A2, SLC39A8, TNF; or
(gg) AAMP, ABL1, ACVRL1, ADAM17, ADAMTS9, ADGRA2, ADGRB1, ADTRP, AGT, AGTR2, AKT1, AKT3, LOX12, AMOT, AMOTL1, ANGPT1, ANGPT2, ANGPT4, ANXA1, ANXA3, APOA1, APOE, APOH, ATOH8, ATP2B4, ATP5F1A, ATP5F1B, BCAR1, BCAS3, BMP10, BMP4, BMPER, BMPR2, CALR, CARD10, CCBE1, CCN3, CD40, CDH13, CDH5, CEACAM1, CIB1, CLEC14A, CORO1B, CSNK2B, CXCL13, CYP1B1, DAB2IP, DCN, DLL4, DNAJA4, DPP4, EDN1, EFNA1, EFNB2, EGR3, EMC10, EMP2, EPHA2, EPHB4, ETS1, FAP, FBXW7, FGF1, FGF16, FGF18, FGF2, FGF4, FGFBP1, FGFR1, FLT4, FOXC2, FOXP1, FUT1, GADD45A, GATA2, GATA3, GDF2, GIPC1, GLUL, GPI, GPLD1, GPX1, GREM1, GRN, HDAC5, HDAC7, HDAC9, HIF1A, HMGB1, HMOX1, HRG, HSPB1, ID1, ITGB1, ITGB1BP1, ITGB2, ITGB3, JCAD, JUP, KDR, KLF4, KRIT1, LGALS12, LGALS8, LGMN, LOXL2, LPXN, MAP2K3, MAP2K5, MAP3K3, MECP2, MEF2C, MEOX2, MET, MIA3, MIR101-1, MIR101-2, MIR10A, MIR10B, MIR126, MIR129-1, MIR129-2, MIR132, MIR133B, MIR135B, MIR137, MIR143, MIR146A, MIR149, MIR150, MIR152, MIR15A, MIR15B, MIR16-1, MIR16-2, MIR188, MIR1908, MIR193A, MIR196A1, MIR196A2, MIR199A1, MIR199A2, MIR199B, MIR19B1, MIR19B2, MIR200A, MIR200B, MIR2000, MIR204, MIR206, MIR20A, MIR21, MIR210, MIR212, MIR22, MIR221, MIR2355, MIR23A, MIR24-1, MIR24-2, MIR26A1, MIR26A2, MIR27A, MIR27B, MIR296, MIR29A, MIR29C, MIR30A, MIR31, MIR320A, MIR329-1, MIR329-2, MIR342, MIR361, MIR410, MIR424, MIR483, MIR487B, MIR492, MIR494, MIR495, MIR497, MIR499A, MIR503, MIR505, MIR640, MIR92A1, MIR92A2, MIR939, MIRLET7F1, MIRLET7F2, MMRN2, MYH9, NF1, NFE2L2, NOS3, NOTCH1, NR2E1, NR2F2, NR4A1, NRP1, NRP2, NUS1, P2RX4, PAXIP1, PDCD10, PDCD6, PDGFB, PDPK1, PECAM1, PIK3C2A, PIK3CA, PIK3R3, PLCG1, PLEKHG5, PLK2, PLPP3, PLXND1, PPARG, PRCP, PRKCA, PRKD1, PRKD2, PRKX, PROX1, PRSS3, PRSS3P2, PTEN, PTGS2, PTK2, PTK2B, PTN, PTP4A3, PTPRM, PXN, RAB13, RGCC, RHOA, RHOB, RHOJ, RIN2, ROBO1, ROCK2, RRAS, S100A2, S100P, SASH1, SCARB1, SCG2, SEMA4A, SEMA5A, SERPINF1, SH3BP1, SIRT1, SLIT2, SMOC2, SOX18, SP1, SP100, SPARC, SPRED1, SRF, SRPX2, STARD13, STAT5A, STC1, SVBP, SYNJ2BP, TBXA2R, TDGF1, TEK, TGFB1, TGFBR1, THBS1, TMSB4X, TNF, TNFSF12, TSTA3, VASH1, VEGFA, VEGFC, WNT5A, WNT7A, ZC3H12A, ZNF580; or
(hh) CCN3, CORO1B, CXCL13, EGR3, FGF1, FGF16, FGF18, FGF2, FGF4, GFR1, HRG, HSPB1, KDR, LGMN, MET, MIR149, MIR16-1, MIR16-2, MIR424, NOTCH1, NR4A1, NRP1, P2RX4, PLEKHG5, PRKD1, PRKD2, RAB13, SEMA5A, SMOC2, THBS1, TMSB4X, VEGFA; or
(ii) APOLD1, BMPER, CXCL10, FOXP1, HOXA9, MIR92A1, MIR92A2, P2RX4, PRMT5, SMAD4, TCIM, TGFBR1;
(jj) ABL1, ACVRL1, ADAM17, ADTRP, AGTR2, AKT1, AKT3, ALOX12, AMOT, AMOTL1, ANGPT1, ANGPT2, ANGPT4, ANXA1, APOA1, APOE, ATP2B4, ATP5F1A, ATP5F1B, CARD10, CD40, CDH5, CIB1, CLEC14A, CSNK2B, DLL4, EFNA1, EFNB2, EGR3, EMP2, EPHA2, EPHB4, ETS1, FBXW7, FGF18, FGF2, FGFBP1, FGFR1, FOXC2, GADD45A, GATA2, GDF2, GPLD1, GPX1, GREM1, HDAC5, HDAC7, HDAC9, HIF1A, HMGB1, HMOX1, HRG, HSPB1, ID1, ITGB1, ITGB1BP1, JCAD, JUP, KDR, KLF4, MAP2K3, MAP2K5, MAP3K3, MECP2, MEF2C, MEOX2, MIA3, MIR101-1, MIR101-2, MIR10A, MIR10B, MIR126, MIR129-1, MIR129-2, MIR132, MIR133B, MIR135B, MIR137, MIR143, MIR146A, MIR149, MIR150, MIR152, MIR15A, MIR15B, MIR16-1, MIR16-2, MIR188, MIR193A, MIR196A1, MIR196A2, MIR199A1, MIR199A2, MIR199B, MIR19B1, MIR19B2, MIR200A, MIR200B, MIR2000, MIR204, MIR206, MIR20A, MIR210, MIR212, MIR22, MIR221, MIR2355, MIR23A, MIR24-1, MIR24-2, MIR26A1, MIR26A2, MIR27A, MIR27B, MIR296, MIR29C, MIR30A, MIR31, MIR320A, MIR329-1, MIR329-2, MIR342, MIR361, MIR410, MIR424, MIR483, MIR487B, MIR492, MIR494, MIR495, MIR497, MIR499A, MIR503, MIR505, MIR640, MIR92A1, MIR92A2, MIR939, MIRLET7F1, MIRLET7F2, MMRN2, MYH9, NF1, NFE2L2, NOS3, NOTCH1, NR2E1, NR4A1, NRP1, NUS1, P2RX4, PDCD10, PDGFB, PDPK1, PIK3C2A, PIK3R3, PLCG1, PLK2, PPARG, PRCP, PRKCA, PRKD1, PRKD2, PTGS2, PTK2B, RGCC, RHOA, RHOJ, ROBO1, SCARB1, SH3BP1, SIRT1, SLIT2, SOX18, SP1, SPRED1, SRF, SRPX2, STARD13, STAT5A, TBXA2R, TDGF1, TGFB1, THBS1, TMSB4X, TNF, VASH1, VEGFA, VEGFC;
or any combination thereof; and
(ii) genes that impact T-cell function selected from:
(a) ADAM10, ADAM17, CCL21, CCL26, CCL27, CCL3, CCL5, CCR2, CXCL10, CXCL11, CXCL13, CXCL16, CXCR3, DEFA1, DEFA1B, GPR183, OXSR1, PIK3CD, PIK3CG, S100A7, SLC12A2, STK39, TMEM102, TNFSF14, WNK1, WNT5A, XCL1, XCL2; or
(b) AGER, AIRE, ARG1, AZGP1, B2M, BTN3A2, BTN3A3, CCR2, CD1A, CD1B, CD1C, CD1D, CD1E, CD46, CD55, CD70, CD81, CD8A, CEACAM1, CLC, CLEC4G, CTSC, CTSH, CYRIB, DENND1B, DLG1, DUSP22, EMP2, FADD, FBXO38, FCGR2B, FOXP3, FUT7, FZD5, GATA3, GNL1, GZMM, HFE, HLA-A, HLA-B, HLA-E, HLA-F, HLA-G, HLA-H, HMGB1, HPRT1, HSPD1, ICAM1, IFNA2, IFNB1, IL12A, IL12B, IL12RB1, IL18, IL18R1, IL18RAP, IL1B, IL1R1, IL20RB, IL23A, IL23R, IL31RA, IL4, IL6, IL7R, JAG1, KDELR1, LILRB1, MALT1, MAP3K7, MICA, MR1, MYO1G, NECTIN2, NLRP3, P2RX7, PPP3CB, PRF1, PRKCZ, PTPRC, PVR, RAB27A, RFTN1, RIPK3, RSAD2, SASH3, SCART1, SLC11A1, SLC22A13, SMAD7, STARD7, STX7, TBX21, TNFRSF1B, TNFSF4, TRAF2, TRAF6, TREX1, TRPM4, TSTA3, WAS, XCL1, ZBTB1, ZP3; or
(c) FBXO38, HLA-A, HMGB1, HSPD1, MR1, SLC22A13; or
(d) AGER, AZGP1, B2M, CD1A, CD1B, CD1C, CD1D, C1DE, CEACAM1, CTSC, CTSH, CYRIB, EMP2, FADD, FCGR2B, GZMM, HLA-A, HLA-B, HLA-E, HLA-F, HLA-G, HLA-H, HPRT1, IL12A, IL12B, IL12RB1, IL23A, IL23R, IL7R, LILRB1, MICA, MR1, NECTIN2, P2RX7, PPP3CB, PRF1, PTPRC, PVR, RAB27A, RIPK3, SLC22A13, STX7, TSTA3, XCL1; or
(e) CCL2, CCR2, CD99, CD99L2, CRK, CRKL, F11R, FADD, ICAM1, IL27RA, ITGAL, RIPK3, XG; or
(f) ANXA1, ATP7A, BATF, BCL3, BCL6, CCL19, CD46, CD80, CD86, CLEC4D, CLEC4E, EOMES, FCER1G, FGL2, FOXP1, FOXP3, GATA3, GPR183, HLX, HMGB1, IFNG, IFNL1, IL12B, IL12RB1, IL18, IL18R1, IL2, IL21, IL23A, IL23R, IL27, IL4, IL4R, IL6, IRF4, JAK3, LEF1, LGALS9, LOXL3, LY9, MALT1, MIR21, MTOR, MYB, NFKBID, NFKBIZ, NLRP3, PCK1, PRKCZ, PTGER4, RARA, RC3H1, RC3H2, RELB, RIPK2, RORA, RORC, SEMA4A, SLAMF6, SMAD7, SOCS5, SPN, STAT3, STAT6, TBX21, TMEM98, TNFSF4, TSC1, ZBTB7B, ZC3H12A, ZFPM1; or
(g) ADA, ADAM8, AKT1, ARG2, BAK1, BAX, BCL10, BCL2L11, BMP4, CCL5, CD27, CD274, CLC, DFFA, DNAJA3, DOCK8, EFNA1, FADD, FAS, FASLG, GIMAP8, GLI3, GPAM, HIF1A, IDO1, IL2RA, IL7R, JAK3, KDELR1, LGALS13, LGALS14, LGALS16, LGALS3, LGALS9, LMBR1L, NFKBID, PDCD1, PIP, PRELID1, PRKCQ, PTCRA, RAG1, RIPK1, RIPK3, SLC46A2, TP53, TSC22D3, WNT5A, ZC3H8; or
(h) HFE, ICAM1, RFTN1, TREX1, WAS; or
(i) ANXA1, APBB1IP, ATP7A, BATF, BCL3, BCL6, CCL19, CD1C, CD46, CD74, CD80, CD81, CD86, CEACAM1, CLEC4D, CLEC4E, EIF2AK4, EOMES, F2RL1, FCER1G, FCGR2B, FGL2, FOXP1, FOXP3, GATA3, GPR183, HAVCR2, HLA-DMB, HLX, HMGB1, ICAM1, IFNA1, IFNA10, IFNA13, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNW1, IL12B, IL12RB1, IL18, IL18R1, IL2, IL21, IL23A, IL23R, IL27, IL4, IL4R, IL6, IRF4, ITGAL, JAK3, LCP1, LEF1, LGALS3, LGALS9, LILRB1, LOXL3, LY9, MALT1, MDK, MIR21, MTOR, MYB, NFKBID, NFKBIZ, NLRP3, PCK1, PRKCZ, PSEN1, PTGER4, RAB27A, RARA, RC3H1, RC3H2, RELB, RIPK2, RORA, RORC, SEMA4A, SLAMF6, SLC11A1, SMAD7, SOCS5, SPN, STAT3, STAT6, TBX21, TMEM98, TNFSF18, TNFSF4, TP53, TSC1, ZBTB7B, ZC3H12A, ZFPM1; or
(j) ADA, BCL10, BTN2A2, CACNA1F, CARD11, CCR7, CD160, CD226, CD300A, CD81, CEACAM1, CYLD, DGKZ, DUSP22, DUSP3, ELF1, EZR, GBP1, KCNN4, LCK, LGALS3, LILRB4, MALT1, NECTIN2, PAWR, PHPT1, PRKD2, PRNP, PTPN2, PTPN22, PTPN6, PTPRJ, PVRIG, RAB29, RC3H1, RELA, RPS3, SH2D1A, SLA2, TESPA1, THY1, TRAT1, UBASH3A; or
(k) ADAM10, ADAM17, ADAM8, AIF1, AIRE, APOD, APP, C10orf99, CCL20, CCL21, CCL27, CCL5, CCR2, CCR6, CD200, CD200R1, CD99L2, CRK, CRKL, CXCL10, CXCL12, CXCL13, DOCK8, ECM1, FADD, IL27RA, ITGA4, LRCH1, OXSR1, PYCARD, RHOA, RIPK3, RIPOR2, S100A7, SELENOK, SPN, STK39, TMEM102, TNFRSF14, TNFSF14, WNK1, WNT5A, XCL1, XCL2; or
(l) ARG1, AZGP1, B2M, CCR2, CD1A, CD1B, CD1C, CD1D, CD1E, CD81, CEACAM1, CLC, CLEC4G, CYRIB, DUSP22, FADD, FBXO38, FCGR2B, FOXP3, FUT7, FZD5, GATA3, HFE, HLA-A, HLA-B, HLA-E, HLA-F, HLA-G, HLA-H, HMGB1, HSPD1, IFNA2, IFNB1, IL12A, IL12B, IL12RB1, IL18, IL18R1, IL1B, IL1R1, IL20RB, IL23A, IL23R, IL6, IL7R, LILRB1, MALT1, MAP3K7, MR1, NECTIN2, NLRP3, P2RX7, PPP3CB, PRKCZ, PTPRC, PVR, RIPK3, RSAD2, SASH3, SLC22A13, SMAD7, STX7, TBX21, TNFRSF1B, TNFSF4, TRAF2, TRAF6, TRPM4, WAS, XCL1, ZBTB1, ZP3;
(m) FBXO38, HMGB1, HSPD1, MR1, SLC22A13; or
(n) AGER, AZGP1, B2M, CD1A, CD1B, CD1C, CD1D, CD1E, CEACAM1, CYRIB, FADD, FCGR2B, HLA-A, HLA-B, HLA-E, HLA-F, HLA-G, HLA-H, IL12A, IL12B, IL12RB1, IL23A, IL23R, IL7R, LILRB1, MR1, NECTIN2, P2RX7, PPP3CB, PTPRC, PVR, RIPK3, SLC22A13, STX7, XCL1; or
(o) CCR2, CD99L2, FADD, IL27RA, RIPK3; or
(p) ABL1, ADA, ADAM8, ANXA1, AP3B1, AP3D1, BAD, BCL6, BMP4, BTN2A2, CAMK4, CARD11, CBFB, CCL19, CCR2, CD2, CD27, CD28, CD46, CD74, CD80, CD83, CD86, CLPTM1, CR1, CRTAM, CTLA4, CYLD, CYP26B1, DROSHA, DTX1, DUSP10, EGR3, ERBB2, FANCA, FANCD2, FGL2, FOXJ1, FOXN1, FOXP3, GATA3, GLI2, GLI3, HLA-DOA, HLA-G, HLX, HMGB1, IFNA2, IFNB1, IFNG, IFNL1, IHH, IL12A, IL12B, IL12RB1, IL15, IL18, ILA, IL1B, IL1RL2, IL2, IL23A, IL23R, IL27, IL2RA, IL36B, IL4, IL4R, IL7, IL7R, IRF1, IRF4, ITPKB, JAK3, KAT2A, LAG3, LEF1, LGALS9, LILRB2, LILRB4, LOXL3, MALT1, MDK, METTL3, MIR21, MIR30B, MYB, NCKAP1L, NFATC2, NFKBID, NFKBIZ, NKAP, NLRP3, NRARP, PCK1, PIK3R6, PNP, PRDM1, PRDX2, PRELID1, PRKCZ, PTPN2, PTPRC, RAG1, RARA, RASGRP1, RC3H1, RC3H2, RHOA, RHOH, RIPK2, RUNX1, RUNX3, SART1, SASH3, SH3RF1, SHH, SLC46A2, SMAD7, SOCS1, SOCS5, SOD1, SOS1, SOX12, SOX13, SOX4, SPINK5, STAT5B, SYK, TBX21, TCF7, TESPA1, TGFBR2, TMEM131L, TNFRSF18, TNFSF4, TNFSF9, TOX, VNN1, VSIR, XBP1, ZAP70, ZBTB1, ZBTB16, ZBTB7B, ZC3H12A, ZC3H8, ZEB1, ZMIZ1, ZNF683; or
(q) B2M, CCR2, CD81, CLC, FOXP3, FZD5, GATA3, HFE, HLA-A, HLA-F, IFNA2, IFNB1, IL18, IL18R1, IL1B, IL1R1, IL6, MALT1, MAP3K7, NLRP3, PRKCZ, RSAD2, SASH3, SMAD7, TBX21, TNFRSF1B, TNFSF4, TRAF2, TRAF6, TRPM4, XCL1; or
(r) ADAM10, ADAM17, CCL21, CCL27, CCL5, CCR2, CXCL10, CXCL13, OXSR1, S100A7, STK39, TMEM102, TNFSF14, WNK1, WNT5A, XCL1, XCL2; or
(s) CD81, HAVCR2, HLA-DMB, LGALS3, LGALS9, LILRB1; or
(t) ABL1, ADA, ADAM8, ADORA2A, AGER, AIF1, AKT1, ANXA1, AP3B1, AP3D1, ARG1, ARG2, BAD, BCL10, BCL6, BMP4, BTLA, BTN2A2, CAMK4, CARD11, CASP3, CAV1, CBFB, CCDC88B, CCL19, CCL2, CCL21, CCL5, CCR2, CCR7, CD160, CD1D, CD2, CD209, CD24, CD27, CD274, CD276, CD28, CD300A, CD3E, CD4, CD40LG, CD46, CD47, CD5, CD55, CD6, CD70, CD74, CD80, CD81, CD83, CD86, CDC42, CEACAM1, CEBPB, CGAS, CLC, CLEC4G, CLECL1, CLPTM1, CORO1A, CR1, CRTAM, CSK, CTLA4, CTNNB1, CYLD, CYP26B1, CYRIB, DLG1, DLG5, DNAJA3, DOCK8, DPP4, DROSHA, DTX1, DUSP10, DUSP22, DUSP3, EBI3, EFNB1, EFNB2, EFNB3, EGR3, EPO, ERBB2, FADD, FANCA, FANCD2, FCGR2B, FGL1, FGL2, FLOT2, FOXJ1, FOXN1, FOXP3, FYN, GATA3, L12, GLI3, GLMN, GNRH1, GPAM, GPNMB, GRAP2, GRB2, GSN, HAVCR2, HES1, HFE, HHLA2, HLA-A, HLA-DMB, HLA-DOA, HLA-DPA1, HLA-DPB1, HLA-DRB1, HLA-E, HLA-G, HLX, HMGB1, HSPD1, HSPH1, ICOS, ICOSLG, IDO1, IFNA2, IFNB1, IFNG, IFNL1, IGF1, IGF2, IGFBP2, IHH, IL10, IL12A, IL12B, IL12RB1, IL15, IL18, ILA, IL1B, IL1RL2, IL2, IL20RB, IL21, IL23A, IL23R, IL27, IL27RA, IL2RA, IL36B, IL4, IL4R, IL6, IL6ST, IL7, IL7R, IRF1, IRF4, ITPKB, JAK3, KAT2A, KLRC4-KLRK1, KLRK1, LAG3, LAT, LAX1, LCK, LEF1, LEP, LGALS1, LGALS3, LGALS7B, LGALS9, LGALS9B, LGALS9C, LILRB1, LILRB2, LILRB4, LMO1, LOXL3, LRRC32, LYN, MAD1L1, MALT1, MAP3K8, MAPK81P1, MARCHF7, MDK, METTL3, MIR181C, MIR21, MIR27A, MIR30B, MYB, NCK1, NCK2, NCKAP1L, NDFIP1, NFATC2, NFKBID, NFKBIZ, NKAP, NLRP3, NOD2, NRARP, PAG1, PAK1, PAK2, PAK3, PAWR, PCK1, PDCD1, PDCD1LG2, PDE5A, PDPK1, PELI1, PIK3CA, PIK3R1, PIK3R6, PLA2G2D, PLA2G2F, PNP, PRDM1, PRDX2, PRELID1, PRKAR1A, PRKCQ, PRKCZ, PRNP, PTPN11, PTPN2, PTPN22, PTPN6, PTPRC, PYCARD, RAC1, RAC2, RAG1, RARA, RASAL3, RASGRP1, RC3H1, RC3H2, RHOA, RHOH, RIPK2, RIPK3, RIPOR2, RPS3, RUNX1, RUNX3, SART1, SASH3, SCGB1A1, SCRIB, SDC4, SELENOK, SFTPD, SH3RF1, SHH, SIRPA, SIRPB1, SIRPG, SIT1, SLC46A2, SLC7A1, SMAD7, SOCS1, SOCS5, SOCS6, SOD1, SOS1, SOX12, SOX13, SOX4, SPINK5, SPN, SPTA1, SRC, STAT5B, SYK, TARM1, TBX21, TCF7, TESPA1, TFRC, TGFBR2, THY1, TIGIT, TMEM131L, TMIGD2, TNFAIP8L2, TNFRSF13C, TNFRSF14, TNFRSF18, TNFRSF1B, TNFRSF21, TNFSF11, TNFSF13B, TNFSF14, TNFSF18, TNFSF4, TNFSF8, TNFSF9, TOX, TRAF6, TREX1, TWSG1, VAV1, VCAM1, VNN1, VSIG4, VSIR, VTCN1, XBP1, XCL1, YES1, ZAP70, ZBTB1, ZBTB16, ZBTB7B, ZC3H12A, ZC3H8, ZEB1, ZMIZ1, ZNF683, ZP3, ZP4; or
(u) EGR3, LEF1, LILRB1, NCKAP1L, NOD2, PTPRC, SOX13, SOX4, STAT5B, SYK, TCF7; or
(v) CBFB, NCKAP1L, RUNX1, RUNX3, SOCS1, ZBTB7B: or
(w) ANXA1, BCL6, CBFB, CCL19, CD80, CD83, CD86, FOXP3, GATA3, HLX, HMGB1, IFNG, IL12B, IL12RB1, IL18, IL2, IL23A, IL23R, IL27, IL4, IL4R, IRF4, JAK3, LGALS9, LOXL3, MALT1, MIR21, MYB, NCKAP1L, NFKBID, NFKBIZ, NLRP3, PRKCZ, RARA, RC3H1, RC3H2, RIPK2, RUNX1, RUNX3, SASH3, SH3RF1, SMAD7, SOCS1, SOCS5, TBX21, TNFSF4, ZBTB7B, ZC3H12A; or
(x) AGER, ANXA1, ARG2, BCL6, CBFB, CCL19, CD160, CD274, CD55, CD80, CD81, CD83, CD86, FOXP3, GATA3, HLX, HMGB1, IFNG, IL12B, IL12RB1, IL18, IL2, IL23A, IL23R, IL27, IL4, IL4R, IRF4, JAK3, LGALS7B, LGALS9, LGALS9B, LGALS9C, LOXL3, MALT1, MIR21, MYB, NCKAP1L, NFKBID, NFKBIZ, NLRP3, PRKCQ, PRKCZ, RARA, RC3H1, RC3H2, RIPK2, RUNX1, RUNX3, SASH3, SH3RF1, SMAD7, SOCS1, SOCS5, TARM1, TBX21, TGFBR2, TNFSF4, TWSG1, VSIR, XCL1, ZBTB7B, ZC3H12A; or
(y) CBFB, CD274, CRTAM, HFE, HLA-A, HLA-E, IRF1, LILRB1, MAPK81P1, NCKAP1L, PTPN22, RUNX1, RUNX3, SH3RF1, SOCS1, VSIR, XCL1, ZBTB7B: or
(z) ADA, ANXA1, AP3B1, AP3D1, BCL6, CBFB, CCL19, CD80, CD83, CD86, FOXP3, GATA3, GLI3, HLX, HMGB1, IFNG, IHH, IL12B, IL12RB1, IL18, IL2, IL23A, IL23R, IL27, IL4, IL4R, IRF4, ITPKB, JAK3, LGALS9, LOXL3, MALT1, MIR21, MYB, NCKAP1L, NFKBID, NFKBIZ, NKAP, NLRP3, PNP, PRDM1, PRKCZ, RARA, RC3H1, RC3H2, RHOA, RIPK2, RUNX1, RUNX3, SASH3, SH3RF1, SHH, SMAD7, SOCS1, SOCS5, SYK, TBX21, TGFBR2, TNFSF4, ZAP70, ZBTB16, ZBTB7B, ZC3H12A, ZNF683; or
(aa) ADA, ADORA2A, AGER, ANXA1, AP3B1, AP3D1, ARG2, BCL6, CBFB, CCL19, CCR2, CD160, CD274, CD28, CD300A, CD3E, CD55, CD80, CD81, CD83, CD86, CRTAM, EB13, FOXP3, GATA3, GL13, HFE, HLA-A, HLA-E, HLX, HMGB1, HSPH1, IFNG, IHH, IL12A, IL12B, IL12RB1, IL18, IL2, IL23A, IL23R, IL27, IL4, IL4R, IRF1, IRF4, ITPKB, JAK3, LGALS7B, LGALS9, LGALS9B, LGALS9C, LILRB1, LOXL3, MALT1, MAPK81P1, MIR21, MYB, NCKAP1L, NFKBID, NFKBIZ, NKAP, NLRP3, PNP, PRDM1, PRKCQ, PRKCZ, PTPN22, PTPRC, RARA, RASAL3, RC3H1, RC3H2, RHOA, RIPK2, RUNX1, RUNX3, SASH3, SH3RF1, SHH, SMAD7, SOCS1, SOCS5, SYK, TARM1, TBX21, TGFBR2, TNFRSF14, TNFSF4, TWSG1, VSIR, XCL1, ZAP70, ZBTB16, ZBTB7B, ZC3H12A, ZNF683; or
(bb) BATF, BCL11B, BCL2, CD3D, CD3E, CD3G, CD74, CTSL, CYLD, DOCK2, FOXN1, FOXP3, IL12B, IL12RB1, IL23A, IL23R, IL6, IRF4, ITPKB, LOXL3, LY9, MTOR, PTPN2, PTPRC, SHH, SLAMF6, SPN, SRF, STAT3, STAT6, STK11, TBX21, THEMIS, TOX, ZAP70, ZFPM1; or
(cc) CD3E, FOXP3, HLA-G, IDO1, LILRB2, TGFBR2; or
(dd) ADA, CARD11, CCR7, CD226, CD81, CYLD, KCNN4, LCK, NECTIN2, PRKD2, RAB29, RELA, RPS3, TESPA1, TRAT1; or
(ee) AGER, AIF1, ANXA1, CARD11, CCDC88B, CCL19, CCL5, CCR2, CD1D, CD209, CD24, CD274, CD276, CD28, CD3E, CD4, CD40LG, CD46, CD55, CD6, CD70, CD80, CD81, CD86, CLECL1, CORO1A, DNAJA3, EB13, EFNB1, EPO, FADD, FOXP3, GPAM, HAVCR2, HES1, HHLA2, HLA-A, HLA-DMB, HLA-DPA1, HLA-DPB1, HLA-E, HMGB1, ICOSLG, IGF1, IGF2, IGFBP2, IL12A, IL12B, IL12RB1, IL15, IL18, ILA, IL1B, IL2, IL21, IL23A, IL23R, IL27RA, IL2RA, IL4, IL6, IL6ST, JAK3, LEP, LGALS9, LILRB2, MIR21, MIR30B, NCK1, NCK2, NCKAP1L, PDCD1LG2, PNP, PRKCQ, PTPN22, PTPRC, PYCARD, RASAL3, RIPK2, RPS3, SASH3, SELENOK, SHH, SLC7A1, SPTA1, STAT5B, SYK, TFRC, TGFBR2, TMIGD2, TNFRSF13C, TNFSF13B, TNFSF4, TNFSF9, TRAF6, VCAM1, VTCN1, XCL1, ZAP70, ZP3, ZP4; or
(ff) ADAM10, ADAM17, ADAM8, AIF1, APP, CCL20, CCL21, CCL27, CCL5, CCR2, CD99L2, CXCL10, CXCL12, CXCL13, DOCK8, FADD, ITGA4, OXSR1, PYCARD, RHOA, S100A7, SELENOK, SPN, STK39, TMEM102, TNFRSF14, TNFSF14, WNK1, WNT5A, XCL1, XCL2; or
(gg) AZGP1, B2M, CD1A, CD1B, CD1C, CD1D, CD1E, CD81, CYRIB, FADD, FBXO38, FOXP3, FZD5, GATA3, HLA-A, HLA-B, HLA-E, HLA-F, HLA-G, HLA-H, HSPD1, IL12A, IL12B, IL12RB1, IL18, IL18R1, IL1B, IL1R1, IL23A, IL23R, IL6, MALT1, MAP3K7, MR1, NECTIN2, NLRP3, P2RX7, PRKCZ, PTPRC, PVR, RSAD2, SASH3, SLC22A13, STX7, TNFSF4, TRAF2, TRAF6, XCL1, ZBTB1, ZP3; or
(hh) AZGP1, B2M, CD1A, CD1B, CD1C, CD1D, C1E, CYRIB, FADD, HLA-A, HLA-B, HLA-E, HLA-F, HLA-G, HLA-H, IL12A, IL12B, IL12RB1, IL23A, IL23R, MR1, NECTIN2, P2RX7, PTPRC, PVR, SLC22A13, STX7, XCL1; or
(ii) B2M, CD81, FZD5, GATA3, HLA-A, IL18, IL18R1, IL1B, IL1R1, IL6, MALT1, MAP3K7, NLRP3, PRKCZ, RSAD2, SASH3, TNFSF4, TRAF2, TRAF6, XCL1; or
(jj) BCL6, BTN2A2, CD46, DUSP10, FOXP3, HLA-G, IFNG, IL2, LGALS9, LILRB2, LILRB4, SOCS1, SOX12, VSIR; or
(kk) FOXP3, IL1A, IL1B, RIPK2, SHH; or
(ll) LEF1, NCKAP1L, PTPRC, SOX13, SOX4, STAT5B, SYK; or
(mm) LEF1, LILRB1, NCKAP1L, NOD2, PTPRC, SOX13, SOX4, STAT5B, SYK; or
(nn) ADA, ANXA1, AP3B1, AP3D1, CBFB, CCL19, CD80, CD83, CD86, FOXP3, GLI3, HLX, IFNG, IHH, IL12B, IL12RB1, IL18, IL23A, IL23R, IL4R, ITPKB, LGALS9, MALT1, MIR21, MYB, NCKAP1L, NFKBID, NFKBIZ, NKAP, NLRP3, PNP, PRKCZ, RARA, RHOA, RIPK2, RUNX1, RUNX3, SASH3, SHH, SOCS1, SOCS5, SYK, TGFBR2, TNFSF4, ZAP70, ZBTB16, ZBTB7B; or
(oo) ADA, ANXA1, AP3B1, AP3D1, CBFB, CCL19, CCR2, CD160, CD28, CD3E, CD55, CD80, CD81, CD83, CD86, EBI3, FOXP3, GLI3, HLA-A, HLA-E, HLX, HSPH1, IFNG, IHH, IL12A, IL12B, IL12RB1, IL18, IL23A, IL23R, IL4R, ITPKB, LGALS9, MALT1, MIR21, MYB, NCKAP1L, NFKBID, NFKBIZ, NKAP, NLRP3, PNP, PRKCQ, PRKCZ, PTPN22, PTPRC, RARA, RASAL3, RHOA, RIPK2, RUNX1, RUNX3, SASH3, SHH, SOCS1, SOCS5, SYK, TGFBR2, TNFSF4, XCL1, ZAP70, ZBTB16, ZBTB7B; or
(pp) AGER, CD24, EPO, FADD, GPAM, HHLA2, HMGB1, ICOSLG, IGF1, IGF2, IGFBP2, IL12B, IL12RB1, IL18, IL2, IL23A, IL23R, IL27RA, IL2RA, MIR21, MIR30B, PYCARD, RPS3, STAT5B, TMIGD2, TNFSF9; or
(qq) ABL1, ADA, ADAM17, ADAM8, ADORA2A, AGER, AIF1, AIRE, AKT1, ANXA1, AP3B1, AP3D1, APBB1IP, ARG1, ARG2, ATG5, ATP7A, AZI2, B2M, BAD, BATF, BAX, BCL10, BCL11B, BCL2, BCL3, BCL6, BMP4, BTLA, BTN2A2, BTN3A1, CAMK4, CARD11, CASP3, CASP8, CAV1, CBFB, CCDC88B, CCL19, CCL2, CCL21, CCL5, CCND3, CCR2, CCR6, CCR7, CCR9, CD151, CD160, CD1C, CD1D, CD2, CD209, CD24, CD27, CD274, CD276, CD28, CD300A, CD3D, CD3E, CD3G, CD4, CD40LG, CD44, CD46, CD47, CD5, CD55, CD6, CD7, CD70, CD74, CD80, CD81, CD83, CD86, CD8A, CD8B, CDC42, CDH26, CDK6, CEACAM1, CEBPB, CGAS, CHD7, CLC, CLEC4A, CLEC4D, CLEC4E, CLEC4G, CLEC7A, CLECL1, CLPTM1, CORO1A, CR1, CRTAM, CSK, CTLA4, CTNNB1, CTPS1, CTSL, CXADR, CYLD, CYP26B1, CYRIB, DDOST, DLG1, DLG5, DLL4, DNAJA3, DOCK2, DOCK8, DPP4, DROSHA, DTX1, DUSP10, DUSP22, DUSP3, EBI3, EFNB1, EFNB2, EFNB3, EGR1, EGR3, EIF2AK4, ELF4, EOMES, EPO, ERBB2, F2RL1, FADD, FANCA, FANCD2, FCER1G, FCGR2B, FGL1, FGL2, FKBP1A, FKBP1B, FLOT2, FOXJ1, FOXN1, FOXP1, FOXP3, FUT7, FYN, FZD5, FZD7, FZD8, GATA3, GLI2, GLI3, GLMN, GNRH1, GPAM, GPNMB, GPR18, GPR183, GPR89A, GPR89B, GRAP2, GRB2, GSN, HAVCR2, HES1, HFE, HHLA2, HLA-A, HLA-DMB, HLA-DOA, HLA-DPA1, HLA-DPB1, HLA-DRB1, HLA-E, HLA-G, HLX, HMGB1, HSH2D, HSPD1, HSPH1, ICAM1, ICOS, ICOSLG, IDO1, IFNA1, IFNA10, IFNA13, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNW1, IGF1, IGF2, IGFBP2, IHH, IL10, IL12A, IL12B, IL12RB1, IL15, IL18, IL18R1, IL1A, IL1B, IL1RL2, IL2, IL20RB, IL21, IL23A, IL23R, IL27, IL27RA, IL2RA, IL36B, IL4, IL4R, IL6, IL6ST, IL7, IL7R, INS, IRF1, IRF4, ITGAL, ITK, ITPKB, JAG2, JAK3, JAML, JMJD6, KAT2A, KDELR1, KIF13B, KIT, KLRC4-KLRK1, KLRK1, LAG3, LAT, LAX1, LCK, LCP1, LEF1, LEP, LEPR, LFNG, LGALS1, LGALS3, LGALS7B, LGALS9, LGALS9B, LGALS9C, LIG4, LILRB1, LILRB2, LILRB4, LMBR1L, LMO1, LOXL3, LRRC32, LY9, LYN, MAD1L1, MAFB, MALT1, MAP3K8, MAPK8IP1, MARCHF7, MDK, METTL3, MICA, MICB, MIR181C, MIR21, MIR27A, MIR30B, MR1, MSN, MTOR, MYB, MYH9, NCAPH2, NCK1, NCK2, NCKAP1L, NCSTN, NDFIP1, NEDD4, NFATC2, NFKBID, NFKBIZ, NHEJ1, NKAP, NKX2-3, NLRC3, NLRP3, NOD2, NRARP, P2RX7, PAG1, PAK1, PAK2, PAK3, PATZ1, PAWR, PAX1, PCK1, PDCD1, PDCD1LG2, PDE5A, PDPK1, PELI1, PIK3CA, PIK3CD, PIK3CG, PIK3R1, PIK3R6, PKNOX1, PLA2G2D, PLA2G2F, PNP, PPP3CA, PPP3CB, PRDM1, PRDX2, PRELID1, PREX1, PRKAR1A, PRKCQ, PRKCZ, PRKDC, PRNP, PRR7, PSAP, PSEN1, PSMB10, PSMB11, PTGER4, PTPN11, PTPN2, PTPN22, PTPN6, PTPRC, PYCARD, RAB27A, RAB29, RABL3, RAC1, RAC2, RAG1, RAG2, RARA, RASAL3, RASGRP1, RC3H1, RC3H2, RELB, RHOA, RHOH, RIPK2, RIPK3, RIPOR2, RORA, RORC, RPS3, RSAD2, RUNX1, RUNX2, RUNX3, SART1, SASH3, SATB1, SCGB1A1, SCRIB, SDC4, SELENOK, SEMA4A, SFTPD, SH3RF1, SHH, SIRPA, SIRPB1, SIRPG, SIT1, SLA2, SLAMF6, SLC11A1, SLC46A2, SLC7A1, SMAD3, SMAD7, SOCS1, SOCS5, SOCS6, SOD1, SOS1, SOX12, SOX13, SOX4, SP3, SPINK5, SPN, SPTA1, SRC, SRF, STAT3, STAT5B, STAT6, STK11, STOML2, SYK, TARM1, TBX21, TCF7, TCIRG1, TESPA1, TFRC, TGFBR2, THEMIS, THY1, TIGIT, TMEM131L, TMEM98, TMIGD2, TNFAIP8L2, TNFRSF13C, TNFRSF14, TNFRSF18, TNFRSF1B, TNFRSF21, TNFRSF4, TNFSF11, TNFSF13B, TNFSF14, TNFSF18, TNFSF4, TNFSF8, TNFSF9, TOX, TP53, TRAF6, TREML2, TREX1, TSC1, TWSG1, VAV1, VCAM1, VNN1, VSIG4, VSIR, VTCN1, WAS, WNT1, WNT4, XBP1, XCL1, YES1, ZAP70, ZBTB1, ZBTB16, ZBTB7B, ZC3H12A, ZC3H8, ZEB1, ZFP36L1, ZFP36L2, ZFPM1, ZMIZ1, ZNF683, ZP3, ZP4; or
(rr) BMP4, ERBB2, FOXP3, GNRH1, IHH, IL1A, IL1B, RIPK2, SHH, TMEM131L, WNT4; or
(ss) HLA-A, HLA-E, IRF1, MAPK81P1, PTPN22, SH3RF1, VSIR, XCL1; or
(tt) BCL2, CBFB, EOMES, GPR18, IRF1, NCKAP1L, PAX1, PSMB11, RUNX1, RUNX3, SATB1, SOCS1, TNFSF8, TOX, ZBTB7B; or
(uu) BCL2, CBFB, CD274, CLEC4A, CRTAM, EOMES, GPR18, HFE, HLA-A, HLA-E, IRF1, LILRB1, MAPK8IP1, NCKAP1L, PAX1, PSMB11, PTPN22, RUNX1, RUNX3, SATB1, SH3RF1, SOCS1, TNFSF8, TOX, VSIR, XCL1, ZBTB7B; or
(vv) BATF, BCL2, CTSL, CYLD, FOXP3, IL12B, IL12RB1, IL23A, IL23R, IL6, IRF4, LOXL3, LY9, MTOR, SHH, SLAMF6, SPN, STAT3, STAT6, TBX21, TOX, ZFPM1; or
(ww) FOXP3, FUT7, IFNG, LGALS9, PLA2G2D, TOX; or
(xx) ARG2, CD274, CD55, CD81, LGALS7B, LGALS9, LGALS9B, LGALS9C, TGFBR2, TWSG1, VSIR, XCL1; or
(yy) ARG1, CD55, CD81, DENND1B, GATA3, IFNA2, IFNB1, IL12A, IL12B, IL18, IL18R1, IL18RAP, IL1B, IL1R1, IL31RA, IL4, IL6, NLRP3, PRKCZ, RSAD2, STARD7, TBX21, XCL1; or
(zz) AGER, ANXA1, ARG2, ATP7A, BATF, BCL3, BCL6, CBFB, CCL19, CD160, CD274, CD55, CD80, CD81, CD83, CD86, CDH26, CTSL, FOXP1, FOXP3, FUT7, GATA3, GPR183, HLX, HMGB1, IFNG, IL12A, IL12B, IL12RB1, IL18, IL18R1, IL2, L21, IL23A, IL23R, IL27, IL4, IL4R, IL6, IRF4, JAK3, LEF1, LGALS7B, LGALS9, LGALS9B, LGALS9C, LOXL3, LY9, MALT1, MIR21, MTOR, MYB, NCKAP1L, NFKBID, NFKBIZ, NKX2-3, NLRP3, PAX1, PLA2G2D, PRKCQ, PRKCZ, PTGER4, RARA, RC3H1, RC3H2, RELB, RIPK2, RORA, RORC, RSAD2, RUNX1, RUNX3, SASH3, SATB1, SEMA4A, SH3RF1, SLAMF6, SMAD7, SOCS1, SOCS5, SPN, STAT3, STAT6, STOML2, TARM1, TBX21, TCIRG1, TGFBR2, TMEM98, TNFSF4, TOX, TWSG, VSIR, XCL1, ZBTB7B, ZC3H12A, ZFPM1; or
(aaa) ARG2, CCR2, CD274, CD28, CD3E, CD55, CD81, DOCK2, EBI3, ELF4, HLA-A, HLA-E, IL12B, IL15, IL18, IL23A, IRF1, LGALS7B, LGALS9, LGALS9B, LGALS9C, MAPK81P1, PTPN22, PTPRC, RASAL3, RIPK2, SH3RF1, SYK, TGFBR2, TNFRSF14, TNFSF4, TWSG1, VSIR, XCL1, ZAP70, ZBTB7B; or
(bbb) ANXA1, ATP7A, BATF, BCL3, BCL6, CCL19, CD80, CD86, EOMES, FOXP1, FOXP3, GATA3, GPR183, HLX, HMGB1, IFNG, IL12B, IL12RB1, IL18, IL18R1, IL2, IL21, IL23A, IL23R, IL27, IL4, IL4R, IL6, IRF4, JAK3, LEF1, LGALS9, LOXL3, LY9, MALT1, MIR21, MTOR, MYB, NFKBID, NFKBIZ, NLRP3, PRKCZ, PTGER4, RARA, RC3H1, RC3H2, RELB, RIPK2, RORA, RORC, SEMA4A, SLAMF6, SMAD7, SOCS5, SPN, STAT3, STAT6, TBX21, TMEM98, TNFSF4, ZBTB7B, ZC3H12A, ZFPM1; or
(ccc) ABL1, AGER, ARG1, BTN2A2, BTN3A1, CASP3, CD24, CD274, CLC, CRTAM, EPO, FADD, FOXP3, FYN, GPAM, HHLA2, HMGB1, ICOSLG, IGF1, IGF2, IGFBP2, IL12B, IL12RB1, I1L18, I1L2, I1L23A, IL23R, IL27RA, IL2RA, LGALS9, LRRC32, MIR181C, MIR21, MIR30B, PDCD1LG2, PRKAR1A, PRNP, PYCARD, RC3H1, RIPK3, RPS3, SATB1, SCRIB, STAT5B, TMIGD2, TNFSF9;
or any combination thereof.

23. (canceled)

24. (canceled)

25. (canceled)

26. (canceled)

27. The method of claim 21, wherein the cancer is a non-hematological solid tumor selected from Adrenocortical carcinoma (ACC), Bladder Urothelial Carcinoma (BLCA), Brain Lower Grade Glioma (LGG), Breast invasive carcinoma (BRCA), Colon adenocarcinoma (COAD), Cervical squamous cell carcinoma and endocervical adenocarcinoma (CESC), Cholangiocarcinoma (CHOL), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Uterine Carcinosarcoma (UCS), Uterine Corpus Endometrial Carcinoma (UCEC), and Uveal Melanoma (UVM).

28. The method of claim 21, wherein the biological sample is: (a) a cancer or tumor biopsy; or (b) a tumor microenvironment (TME) aspirate.

29. (canceled)

30. The method of claim 21, wherein the ICB therapy is a PD-1 inhibitor therapy or a CTLA4 inhibitor therapy.

31. The method of claim 2, wherein an angio-immune score above the angio-immune baseline score is indicative of a high angiogenic signature and a low T cell function signature in the subject; and wherein the subject identified as having the high angiogenic signature and the low T cell function signature is administered a treatment comprising a combination of ICB therapy and a tyrosine kinase inhibitor.

32. The method of claim 21, further comprising administering the subject an ICB therapy.

33. (canceled)
